SEQUENCE LISTING (146 sequences)

SEQ ID NO: 1 - protein, 316 aa, Saccharomyces cerevisiae
  misc_feature: Corresponds to SEQ ID NO: 74
MGEVKVKVQPPDADPVEIENRIIELCHQFPHGITDQVIQNEMPHIEAQQRAVAINRLLSMGQLDLLRSNTGLLYRIKDSQNAGKMKGSDNQEKLVYQIIEDAGNKGIWSRDIRYKSNLPLTEINKILKNLESKKLIKAVKSVAASKKKVYMLYNLQPDRSVTGGAWYSDQDFESEFVEVLNQQCFKFLQSKAETARESKQNPMIQRNSSFASSHEVWKYICELGISKVELSMEDIETILNTLIYDGKVEMTIIAAKEGTVGSVDGHMKLYRAVNPIIPPTGLVRAPCGLCPVFDDCHEGGEISPSNCIYMTEWLEF

SEQ ID NO: 2 - protein, 330 aa, Candida albicans
  misc_feature: Corresponds to SEQ ID NO: 75
MSEMLVSDKARHLYTKMREYPTSKLFDQDELQTLFDIKKGSELMEYLQELVNGKYVKISKMGDQLKFQTVAEEEAKKVSSMSDDEAMIYSYIEASGREGIWTKTIKAKTNLHQHIVQKCLKNLENNRYIKSIKSVKHPTRKIYMLYNLQPSIDVTGGPWFTDSELDTEFIETLLEVCWRFIVGKTMYIKDEEADNEDINPLQTTYHNHHPGVNLDQLVEFINNSNITSVELGINDIRSLCDVLIYDDRIEEVGGNQENSGIFKATWQSIIDKGNTILQNNYQDLKNVVSEDCFNYLQQNQSDFSVFQYKSTIQDLQDESDLVYLDSWMNE

SEQ ID NO: 3 - protein, 317 aa, Homo sapiens
  ref <308>: DATABASE ACCESSION NUMBER: Human Genbank/U93869
  ref <309>: DATABASE ENTRY DATE: 1997-06-23
  ref <313>: RELEVANT RESIDUES: (1)..(317)
MSGMIENGLQLSDNAKTLHSQMMSKGIGALFTQQELQKQMGIGSLTDLMSIVQELLDKNLIKLVKQNDELKFQGVLESEAQKKATMSAEEALVYSYIEASGREGIWSKTIKARTNLHQHVVLKCLKSLESQRYVKSVKSVKFPTRKIYMLYSLQPSVDITGGPWFTDGELDIEFINSLLTIVWRFISENTFPNGFKNFENGPKKNVFYAPNVKNYSTTQEILEFITAAQVANVELTPSNIRSLCEVLVYDDKLEKVTHDCYRVTLESILQMNQGEGEPEAGNKALEDEEEFSIFNYFKMFPASKHDKEVVYFDEWTI

SEQ ID NO: 4 - protein, 195 aa, Saccharomyces cerevisiae
  misc_feature: Corresponds to SEQ ID NO: 77
MSGSLKSLDKKIAKRRQVYKPVLDNPFTNEAHMWPRVHDQPLIWQLLQSSIINKLIHIQSKENYPWELYTDFNEIVQYLSGAHGNSDPVCLFVCNKDPDVPLVLLQQIPLLCYMAPMTVKLVQLPKSAMDTFKSVSKYGMLLLRCDDRVDKKFVSQIQKNVDLLQFPWLNAIKYRPTSVKLLKTTVPIVSKKRQK

SEQ ID NO: 5 - protein, 220 aa, Candida albicans
  misc_feature: Corresponds to SEQ ID NO: 78
MNKSNKVKKPSVAKVSTKAASSSLKSQEAKRQVFRPILDNSFTQSNQWPFIEPTIANDIVDLLEVLLKMQDSTFKYRGFNPTVSALEKQAAANRGIHKNACVQIKYVFVCKYDISPATLTNVFPTLCFTASKSAEDRVKLIQLPRGSLERLSKALGVDRVGIFGLTKDTEGAQPLFDLINENVKDIEAPWLDCIFREEMVFNQPNTKHVASTVGRKKNKK

SEQ ID NO: 6 - protein, 328 aa, Saccharomyces cerevisiae
  misc_feature: Corresponds to SEQ ID NO: 79
MSKNRDPLLANLNAFKSKVKSAPVIAPAKVGQKKTNDTVITIDGNTRKRTASERAQENTLNSAKNPVLVDIKKEAGSNSSNAISLDDDDDDEDFGSSPSKKVRPGSIAAAALQANQTDISKSHDSSKLLWATEYIQKKGKPVLVNELLDYLSMKKDDKVIELLKKLDRIEFDPKKGTFKYLSTYDVHSPSELLKLLRSQVTFKGISCKDLKDGWPQCDETINQLEEDSKILVLRTKKDKTPRYVWYNSGGNLKCIDEEFVKMWENVQLPQFAELPRKLQDLGLKPASVDPATIKRQTKRVEVKKKRQRKGKITNTHMTGILKDYSHRV

SEQ ID NO: 7 - protein, 284 aa, Candida albicans
  misc_feature: Corresponds to SEQ ID NO: 80
MSDLSAQLSAFKNKIKSGPSVIVPRKATFTQSPSSPLSSSTTTTTSKNDANVKKRSTTDSVTRVLKKQKANMGEMTGSHLSTQLHLAVEYIKEHDQPISVEKLQNYLSFDISHTLLPLLNEIDRVKYDESKGTLEYVSLHNIRSSDDVLEFLRRQTTFKGTSVKELKDGWAGCVAAIDELESQGKILVLRNKKENAPRLVWANNGGELGYIDTEFKDMWDQVKLPEPDVLYQKLLDQGLKPTGADPNLIKKQPQQKEKKQKKARRGKITNTHMKGILKDYSQLV

SEQ ID NO: 8 - protein, 291 aa, Homo sapiens
  misc_feature: Corresponds to SEQ ID NO: 81
  ref <308>: DATABASE ACCESSION NUMBER: Human Genbank/NP_002086
  ref <309>: DATABASE ENTRY DATE: 2000-10-31
  ref <313>: RELEVANT RESIDUES: (1)..(291)
MDPSLLRERELFKKRALSTPVVEKRSASSESSSSSSKKKKTKVEHGGSSGSKQNSDHSNGSFNLKALSGSSGYKFGVLAKIVNYMKTRHQRGDTHPLTLDEILDETQHLDIGLKQKQWLMTEALVNNPKIEVIDGKYAFKPKYNVRDKKALLRLLDQHDQRGLGGILLEDIEEALPNSQKAVKALGDQILFVNRPDKKKILFFNDKSCQFSVDEEFQKLWRSVTVDSMDEEKIEEYLKRQGISSMQESGPKKVAPIQRRKKPASQKKRRFKTHNEHLAGVLKDYSDITSSK

SEQ ID NO: 9 - protein, 480 aa, Saccharomyces cerevisiae
  misc_feature: Corresponds to SEQ ID NO: 82
MSQEQYTENLKVIVAEKLAGIPNFNEDIKYVAEYIVLLIVNGGTVESVVDELASLFDSVSRDTLANVVQTAFFALEALQQGESAENIVSKIRMMNAQSLGQSDIAQQQQQQQQQQQPDIAQQQPQQQPQLQPLQPQLGTQNAMQTDAPATPSPISAFSGVVNAAAPPQFAPVDNSQRFTQRGGGAVGKNRRGGRGGNRGGRNNNSTRFNPLAKALGMAGESNMNFTPTKKEGRCRLFPHCPLGRSCPHAHPTKVCNEYPNCPKPPGTCEFLHPNEDEELMKEMERTREEFQKRKADLLAAKRKPVQTGIVLCKFGALCSNPSCPFGHPTPANEDAKVIDLMWCDKNLTCDNPECRKAHSSLSKIKEVKPISQKKAAPPPVEKSLEQCKFGTHCTNKRCKYRHARSHIMCREGANCTRIDCLFGHPINEDCRFGVNCKNIYCLFRHPPGRVLPEKKGAAPNSNVPTNERPFALPENAIIEN

SEQ ID NO: 10 - protein, 418 aa, Candida albicans
  misc_feature: Corresponds to SEQ ID NO: 83
MQFAPDNQIGKELQQNLIQEIQRRFNKPADDAVDIADYIIYLIVAKKSEQEIVAEVKDIADISIDVGFIGDVYLEIRKLEVKYNQPPAAVEEASQPQQEQQQQSQASVVAPQIPIGPKKQLTEEEKIALRSQRFGTTTRLSGRGGRGGITKTRTDFRNGHNNKNFLDPKKLDQIISGANNGAIKFVPLPPKGRCPDFPYCKNQNCEKAHPTKNCFNYPDCPNPPGTCNFLHPDQDQELIAKLETSKKEFEEKKKNQLMVKQGSCKYGLKCAKENCPFAHPTPANPESGKIETLEWCPQGKNCQDRNCTKSHPPPPTANSEKLLSAADLALEQCKFGSQCTNLKCPRRHATSAVPCRAGAECRRVDCTFSHPLKEPCRFGTKCTNKVCMYQHPEGRTIASHTWTRDGSGNNNSTSNRSF

SEQ ID NO: 11 - protein, 156 aa, Homo sapiens
  misc_feature: Corresponds to SEQ ID NO: 84
  ref <308>: DATABASE ACCESSION NUMBER: Human Genbank/AAD42873
  ref <309>: DATABASE ENTRY DATE: 2000-01-05
  ref <313>: RELEVANT RESIDUES: (1)..(156)
PQQLHLLSRQLEDPNGSFSNAEMSELSVAQKPEKLLERCKYWPACKNGDECAYHHPISPCKAFPNCKFAEKCLFVHPNCKYDAKCTKPDCPFTHVSRRIQLCRYFPACKKMECPFYHPKHCRFNTQCTRPDCTFYHPTINVPPRHALKWIRPQTSE

SEQ ID NO: 12 - protein, 360 aa, Saccharomyces cerevisiae
  misc_feature: Corresponds to SEQ ID NO: 85
MANSPKKPSDGTGVSASDTPKYQHTVPETKPAFNLSPGKASELSHSLPSPSQIKSTAHVSSTHNDAAGNTDDSVLPKNVSPTTNLRVESNGDTNNMFSSPAGLALPKKDDKKKNKGTSKADSKDGKASNSSGQNAQQQSDPNKMQDVLFSAGIDVREEEALLNSSINASKSQVQTNNVKIPNHLPFLHPEQVSNYMRKVGKEQNFNLTPTKNPEILDMMSSACENYMRDILTNAIVISRHRRKAVKINSGRRSEVSAALRAIALIQKKEEERRVKKRIALGLEKEDYENKIDSEETLHRASNVTAGLRAGSKKQYGWLTSSVNKPTSLGAKSSGKVASDITARGESGLKFREAREEPGIV

SEQ ID NO: 13 - protein, 358 aa, Candida albicans
  misc_feature: Corresponds to SEQ ID NO: 86
MSHKSMTSTPQESSNLKRQLENSEDSSSPNKRSKTETTTENQSSWESDFNSLPVELLQTETNGTSPAPAPATPIDTTNASSTKERDQDTSKLNDAIAAAGVDIQQEEEILLQQQLNRKSAEGMASNLKSVIRSSKLPPFLHNYHLAAFIDKVAKQNGIQQNFLMDGEMLELISAACETWLSNLATKTIILSRHRRRGIPVINKKSGSSSVPRSEISKELRSLALKQKEMEEKRVNKRVMLGLEKSTKDASKNDENGESKAGAEETLHRAANATAAMMTMNPGRKKYSWMTSSATAGGGSDFGKSSGGSSKDSGKHQSPIISVRGDNGLRFREIRSGNSIIMKDLLGAIEDEKMGTRNA

SEQ ID NO: 14 - protein, 1023 aa, Homo sapiens
  misc_feature: Corresponds to SEQ ID NO: 87
  ref <308>: DATABASE ACCESSION NUMBER: Human Genbank/CAA72189
  ref <309>: DATABASE ENTRY DATE: 1997-06-25
  ref <313>: RELEVANT RESIDUES: (1)..(1023)
MAAGSDLLDEVFFNSEVDEKVVSDLVGSLESQLAASAAHHHHLAPRTPEVRAAAAGALGNHVVSGSPAGAAGAGPAAPAEGAPGAAPEPPPAGRARPGGGGPQRPGPPSPRRPLVPAGPAPPAAKLRPPPEGSAGACAPVPAAAAVAAGPEPAPAGPAKPAGPAALAARAGPGPGPGPGPGPGPGKPAGPGAAQTLNGSAALLNSHHAAAPAVSLVNNGPAALLPLPKPAAPGTVIQTPPFVGAAAPPAPAAPSPPAAPAPAAPAAAPPPPPPAPATLARPPGHPAGPPTAAPAVPPPAAAQNGGSAGAAPAPAPAAGGPAGVSGQPGPGAAAAAPAPGVKAESPKRVVQAAPPAAQTLAASGPASTAASMVIGPTMQGALPSPAAVPPPAPGTPTGLPKGAAGAVTQSLSRTPTATTSGIRATLTPTVLAPRLPQPPQNPTNIQNFQLPPGMVLVRSENGQLLMIPQQALAQMQAQAHAQPQTTMAPRPATPTSAPPVQISTVQAPGTPIIARQVTPTTIIKQVSQAQTTVQPSATLQRSPGVQPQLVLGGAAQTASLGTATAVQTGTPQRTVPGATTTSSAATETMENVKKCKNFLSTLIKLASSGKQSTETAANVKELVQNLLDGKIEAEDFTSRLYRELNSSPQPYLVPFLKRSLPALRQLTPDSAAFIQQSQQQPPPPTSQATTALTAVVLSSSVQRTAGKTAATVTSALQPPVLSLTQPTQVGVGKQGQPTPLVIQQPPKPGALIRPPQVTLTQTPMVALRQPHNRIMLTTPQQVNLSEESARILATNSELVGTLTRSCKDETFLLQAPLQRRILEIGKKHGITELHPDVVSYVSHATQQRLQNLVEKISETAQQKNFSYKDDDRYEQASDVRAQLKFFEQLDQIEKQRKDEQEREILMRAAKSRSRQEDPEQLRLKQKAKEMQQQELAQMRQRDANLTALAAIGPRKKRKVDCPGPGSGAEGSGPGSVVPGSSGVGTPRQFTRQRITRVNLRDLIFCLENERETSHSLLLYKAFLK

SEQ ID NO: 15 - protein, 184 aa, Saccharomyces cerevisiae
  misc_feature: Corresponds to SEQ ID NO: 88
MNTNSNTMVMNDANQAQITATFTKKILAHLDDPDSNKLAQFVQLFNPNNCRIIFNATPFAQATVFLQMWQNQVVQTQHALTGVDYHAIPGSGTLICNVNCKVRFDESGRDKMGQDATVPIQPNNTGNRNRPNDMNKPRPLWGPYFGISLQLIIDDRIFRNDFNGVISGFNYNMVYKPEDSLLKI

SEQ ID NO: 16 - protein, 181 aa, Candida albicans
  misc_feature: Corresponds to SEQ ID NO: 89
MNQDPTQQLEPFLKRFLASLDLLYTQPTSQPFPNVESYATQLGSNLKRSSAIIVNGQPIIPSPQEDCKLQFQKKWLQTPLSSHQLTSYDGHLIPGTGTFVVHFSAKVRFDQSGRNRLGESADLFQENNSIVSKTNQRPIWGSWFGVDVNLVVDENVMQDGEIINSMDYRFTYVPNDSIIKV

SEQ ID NO: 17 - protein, 244 aa, Saccharomyces cerevisiae
  misc_feature: Corresponds to SEQ ID NO: 90
MNALYNHAVKQKNQLQQELARFEKNSVTAPISLQGSISATLVSLEKTVKQYAEHLNRYKEDTNAEEIDPKFANRLATLTQDLHDFTAKFKDLKQSYNENNSRTQLFGSGASHVMDSDNPFSTSETIMNKRNVGGASANGKEGSSNGGGLPLYQGLQKEQSVFERGNAQLDYILEMGQQSFENIVEQNKILSKVQDRMSNGLRTLGVSEQTITSINKRVFKDKLVFWIALILLIIGIYYVLKWLR

SEQ ID NO: 18 - protein, 238 aa, Candida albicans
  misc_feature: Corresponds to SEQ ID NO: 91
MNSIYNHGLKQTQTITKDLTQFEKNLSTSPLSLQGAITTSLTAFRKTIKEYSDLLEKNVNDTSYTKHENRLNKFNQDLNEFTLKFDTLKKQRDIQVQEANKQELLGRRHISTTATAALGSTSSDNPYESSSNPSQQQQQQLQDEQNTMSYREGLYHEKNSLERGSEQLDRILEMGQQAFEDIVEQNEILRKVQTKFEESLITLGVSQGTIRSVERRAKQDKWLFWFCVVVMLVVFYYI

SEQ ID NO: 19 - protein, 261 aa, Homo sapiens
  misc_feature: Corresponds to SEQ ID NO: 92
  ref <308>: DATABASE ACCESSION NUMBER: Human Genbank/NP_003560
  ref <309>: DATABASE ENTRY DATE: 2000-11-01
  ref <313>: RELEVANT RESIDUES: (1)..(261)
MSYTPGVGGDPTQLAQRISSNIQKITQCSVEIQRTLNQLGTPQDSPELRQQLQQKQQYTNQLAKETDKYIKEFGSLPTTPSEQRQRKIQKDRLVAEFTTSLTNFQKVQRQAAEREKEFVARVRASSRVSGSFPEDSSKERNLVSWESQTQPQVQVQDEEITEDDLRLIHERESSIRQLEADIMDINEIFKDLGMMIHEQGDVIDSIEANVENAEVHVQQANQQLSRAADYQRKSRKTLCIIILILVIGVAIISLIIWGLNH

SEQ ID NO: 20 - protein, 258 aa, Saccharomyces cerevisiae
  misc_feature: Corresponds to SEQ ID NO: 93
  ref <301>: AUTHORS: Bauer and Burgers
  ref <302>: TITLE: Molecular cloning, structure and expression of the yeast proliferating cell nuclear antigen gene.
  ref <303>: JOURNAL: Nucleic Acids Research
  ref <304>: VOLUME: 18
  ref <305>: ISSUE: 2
  ref <306>: PAGES: 261-265
  ref <307>: DATE: 1990
  ref <308>: DATABASE ACCESSION NUMBER: X16676
  ref <309>: DATABASE ENTRY DATE: 1993-09-30
MLEAKFEEASLFKRIIDGFKDCVQLVNFQCKEDGIIAQAVDDSRVLLVSLEIGVEAFQEYRCDHPVTLGMDLTSLSKILRCGNNTDTLTLIADNTPDSIILLFEDTKKDRIAEYSLKLMDIDADFLKIEELQYDSTLSLPSSEFSKIVRDLSQLSDSINIMITKETIKFVADGDIGSGSVIIKPFVDMEHPETSIKLEMDQPVDLTFGAKYLLDIIKGSSLSDRVGIRLSSEAPALFQFDLKSGFLQFFLAPKFNDEE

SEQ ID NO: 21 - protein, 259 aa, Canidia albicans
  misc_feature: Corresponds to SEQ ID NO: 94
MLEGKFEEAALLKKVVEAIKDCVKKCNFNCSEHGITVQAVDDSRVLLVSLLIGQTSFSERCDRDVTLGIDLESFSKIIKSANNEDFLTLLAEDSPDQIMAILEEKQKEKISEYSLKLMDIDSEFLQIDDMEYDAVVNMPSSDFAKLVRDLKNLSESLRVVVTKDSVKFTSEGDSGSGSVILKPYTNLKNERESVTISLDDPVDLTFGLKYLNDIVKAATLSDVITIKLADKTPALFEFKMQSGGYLRFYLAPKFDDDEY

SEQ ID NO: 22 - protein, 261 aa, Homo sapiens
  misc_feature: Corresponds to SEQ ID NO: 95
  ref <301>: AUTHORS: Almendral, Huebsch, Blundell, MacDonald-Bravo, and Bravo
  ref <302>: TITLE: Cloning and sequence of the human nuclear protein cyclin: Homology with DNA-binding proteins
  ref <303>: JOURNAL: Proc. Natl. Acad. Sci. U.S.A.
  ref <304>: VOLUME: 84
  ref <305>: ISSUE: 6
  ref <306>: PAGES: 1575-1579
  ref <307>: DATE: 1987
  ref <308>: DATABASE ACCESSION NUMBER: m15796
  ref <309>: DATABASE ENTRY DATE: 1993-04-27
  ref <308>: DATABASE ACCESSION NUMBER: Human Genbank/P12004
  ref <309>: DATABASE ENTRY DATE: 2001-10-16
  ref <313>: RELEVANT RESIDUES: (1)..(261)
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 23 - protein, 511 aa, Saccharomyces cerevisiae
  misc_feature: Corresponds to SEQ ID NO: 96
MSKRSIEVNEEQDRVVSAKTESHSVPAIPASEEQDAPKNDLEEQLSDEFDSDGEIIEIDGDDEINDEDDLRKKQEEAETLVQKDQSEGNKEKIQELYLPHMSRPLGPDEVLEADPTVYEMLHNVNMPWPCLTLDVIPDTLGSERRNYPQSILLTTATQSSRKKENELMVLALSNLAKTLLKDDNEGEDDEEDDEDDVDPVIENENIPLRDTTNRLKVSPFAISNQEVLTATMSENGDVYIYNLAPQSKAFSTPGYQIPKSAKRPIHTVKNHGNVEGYGLDWSPLIKTGALLSGDCSGQIYFTQRHTSRWVTDKQPFTVSNNKSIEDIQWSRTESTVFATAGCDGYIRIWDTRSKKHKPAISVKASNTDVNVISWSDKIGYLLASGDDNGTWGVWDLRQFTPSNADAVQPVAQYDFHKGAITSIAFNPLDESIVAVGSEDNTVTLWDLSVEADDEEIKQQAAETKELQEIPPQLLFVHWQKEVKDVKWHKQIPGCLVSTGTDGLNVWKTISV

SEQ ID NO: 24 - protein, 420 aa, Candida albicans
  misc_feature: Corresponds to SEQ ID NO: 97
MSKRSAEDDLSGNGSTSHTAVKTNKDSLPTTTNGKEEEPDNMDIGEFEDPYGDEFESDEIIELDDNNDEEDDEMIDENSTQAKIEELEAKEQEQEQQSSIYLPHKSKPLGPDEVLEADPTVYEMLHNINLPWPCLTVDILPDSLGNERRSYPATVYLATATQAAKAKDNELLAMKASSLAKTLVKDENEEDEEDEDDDDDVDSDPILDSESIPLRHTTNRIRVSPHAQQTGEYLTASMSENGEVYIFDLLAQYKAFDTPGYMIPKSSKRPIHTIRAHGNVEGYGLDWSPLVNTGALLSGDMSGRIYLTNRTTSSWTTDKTPFFASQSSIEDIQWSTGETTVFATGGCDGYICIWDTRSKKHKPALSVIASKSDVNVISWSSKINHLLASGHDDGSWGVWDLRNFTNNTTSNPSPVANYDF

SEQ ID NO: 25 - protein, 425 aa, Homo sapiens
  misc_feature: Corresponds to SEQ ID NO: 98
  ref <308>: DATABASE ACCESSION NUMBER: Human Genbank/NP_005601
  ref <309>: DATABASE ENTRY DATE: 2001-12-20
  ref <313>: RELEVANT RESIDUES: (1)..(425)
MADKEAAFDDAVEERVINEEYKIWKKNTPFLYDLVMTHALEWPSLTAQWLPDVTRPEGKDFSIHRLVLGTHTSDEQNHLVIASVQLPNDDAQFDASHYDSEKGEFGGFGSVSGKIEIEIKINHEGEVNRARYMPQNPCIIATKTPSSDVLVFDYTKHPSKPDPSGECNPDLRLRGHQKEGYGLSWNPNLSGHLLSASDDHTICLWDISAVPKEGKVVDAKTIFTGHTAVVEDVSWHLLHESLFGSVADDQKLMIWDTRSNNTSKPSHSVDAHTAEVNCLSFNPYSEFILATGSADKTVALWDLRNLKLKLHSFESHKDEIFQVQWSPHNETILASSGTDRRLNVWDLSKIGEEQSPEDAEDGPPELLFIHGGHTAKISDFSWNPNEPWVICSVSEDNIMQVWQMAENIYNDEDPEGSVDPEGQGS

SEQ ID NO: 26 - protein, 431 aa, Saccharomyces cerevisiae
  misc_feature: Corresponds to SEQ ID NO: 99
MEPQEEFITTEEVEQEIVPTVEVEQDVPVDIEGENDDDDEMMNDDEEALEVDMSNNSLTYFDKHTDSVFAIGHHPNLPLVCTGGGDNLAHLWTSHSQPPKFAGTLTGYGESVISCSFTSEGGFLVTADMSGKVLVHMGQKGGAQWKLASQMQEVEEIVWLKTHPTIARTFAFGATDGSVWCYQINEQDGSLEQLMSGFVHQQDCSMGEFINTDKGENTLELVTCSLDSTIVAWNCFTGQQLFKITQAEIKGLEAPWISLSLAPETLTKGNSGVVACGSNNGLLAVINCNNGGAILHLSTVIELKPEQDELDASIESISWSSKFSLMAIGLVCGEILLYDTSAWRVRHKFVLEDSVTKLMFDNDDLFASCINGKVYQFNARTGQEKFVCVGHNMGVLDFILLHPVANTGTEQKRKVITAGDEGVSLVFEVPN

SEQ ID NO: 27 - protein, 417 aa, Candida albicans
  MISC_FEATURE (326)..(326): X can be any amino acid
  MISC_FEATURE (367)..(367): X can be any amino acid
  MISC_FEATURE (378)..(378): X can be any amino acid
  misc_feature: Corresponds to SEQ ID NO: 100
MSHQQEDVVDDTQEEYINVNEVAEEVADDDQAPPDEEDEEMELDDEHETLEIDMSNNSWTYFDKHTDSIFTIFSHPKLPMVLTEGGDNTAYLWTTHTQPPRFVGEITGHKESVISGGFTADGKFVVTADMNGLIQVFKATKGGEQWVKFGELDEVEEVLFVTVHPTLPFFAFGATDGSIWVYQIDESSKLLVQIMSGFSHTLKCNGAVFIQGKDENDLTLVSISEDGTVVNWNCFTGQVNYKLQPHDDFKGVESPWVTVKVHGNLVAIGGRDGQLSIVNNDTGKIVHTLKTLDNVDDIAELSIEALSWCESKNINLLAVGLVSGDXLLFDTQQWRLRKNLKVDDAITKLQFVGETPILVGNSMDGKXYKWEPRTGEKXFAGVGTNMGSYGLCYFKIEVKNWLLLVDERCFHWSLFMK

SEQ ID NO: 28 - protein, 611 aa, Homo sapiens
  misc_feature: Corresponds to SEQ ID NO: 101
  ref <308>: DATABASE ACCESSION NUMBER: Human Genbank/NP_001078
  ref <309>: DATABASE ENTRY DATE: 2001-12-18
  ref <313>: RELEVANT RESIDUES: (1)..(611)
MDSGRRLGPEKWIRRLRRMESESESGAAADTPPLETLSFHGDEEIIEVVELDPGPPDPDDLAQEMEDVDFEEEEEEEGNEEGWVLEPQEGVVGSMEGPDDSEVTFALHSASVFCVSLDPKTNTLAVTGGEDDKAFVWRLSDGELLFECAGHKDSVTCAGFSHDSTLVATGDMSGLLKVWQVDTKEEVWSFEAGDLEWMEWHPRAPVLLAGTADGNTWMWKVPNGDCKTFQGPNCPATCGRVLPDGKRAVVGYEDGTIRIWDLKQGSPIHVLKGTEGHQGPLTCVAANQDGSLILTGSVDCQAKLVSATTGKVVGVFRPETVASQPSLGEGEESESNSVESLGFCSVMPLAAVGYLDGTLAIYDLATQTLRHQCQHQSGIVQLLWEAGTAVVYTCSLDGIVRLWDARTGRLLTDYRGHTAEILDFALSKDASLVVTTSGDHKAKVFCVQRPDRDFSPDGALLATASYDTRVYIWDPHNGDILMEFGHLFPPPTPIFAGGANDRWVRSVSFSHDGLHVASLADDKMVRFWRIDEDYPVQVAPLSNGLCCAFSTDGSVLAAGTHDGSVYFWATPRQVPSLQHLCRMSIRRVMPTQEVQELPIPSKLLEFLSYRI

SEQ ID NO: 29 - protein, 240 aa, Saccharomyces cerevisiae
  misc_feature: Corresponds to SEQ ID NO: 102
MSAPTMRSTSILTEHLGYPPISLVDDIINAVNEIMYKCTAAMEKYLLSKSKIGEEDYGEEIKSGVAKLESLLENSVDKNFDKLELYVLRNVLRIPEEYLDANVFRLENQKDLVIVDENELKKSEEKLREKVNDVELAFKKNEMLLKRVTKVKRLLFTIRGFKQKLNELLKCKDDVQLQKILESLKPIDDTMTLLTDSLRKLYVDSESTSSTEEVEALLQRLKTNGKQNNKDFRTRYIDIR

SEQ ID NO: 30 - protein, 314 aa, Candida albicans
  misc_feature: Corresponds to SEQ ID NO: 103
MSDKTLDERTTAILTEHLEFAPLTLIDDVINAVNEIMYKGTTAIETYLKEQKQLMKNGITKVTEDEIEIGMGKLESLLESTIDKNFDKFELYCLRNIFNIPKDLIPYIQLSHQQGIEFKSDNVEQKREFDQQIKNLQLKIMQELQLRKILKLQLVKVQKLIKVLIAIDNDFKKIDFASGGGGNEESIRILKNLQPIDETLYFLISQIKNLINQIEQLSNKVNTNLKTQKFIPNLRDKFIDGRTFRVLQQTGIWKDLEKNDIKILVQGNDNNNNNNNNNNNTLTDLQNQDDIDMIIPEQDDIDVDAIKNINAQIF

SEQ ID NO: 31 - protein, 600 aa, Saccharomyces cerevisiae
  misc_feature: Corresponds to SEQ ID NO: 104
MSHSGAAIFEKVSGIIAINEDVSPAELTWRSTDGDKVHTVVLSTIDKLQATPASSEKMMLRLIGKVDESKKRKDNEGNEVVPKPQRHMFSFNNRTVMDNIKMTLQQIISRYKDADIYEEKRRREESAQHTETPMSSSSVTAGTPTPHLDTPQLNNGAPLINTAKLDDSLSKEKLLTNLKLQQSLLKGNKVLMKVFQETVINAGLPPSEFWSTRIPLLRAFALSTSQKVGPYNVLSTIKPVASSENKVNVNLSREKILNIFENYPIVKKAYTDNVPKNFKEPEFWARFFSSKLFRKLRGEKIMQNDRGDVIIDRYLTLDQEFDRKDDDMLLHPVKKIIDLDGNIQDDPVVRGNRPDFTMQPGVDINGNSDGTVDILKGMNRLSEKMIMALKNEYSRTNLQNKSNITNDEEDEDNDERNELKIDDLNESYKTNYAIIHLKRNAHEKTTDNDAKSSADSIKNADLKVSNQQMLQQLSLVMDNLINKLDLNQVVPNNEVSNKINKRVITAIKINAKQAKHNNVNSALGSFVDNTSQANELEVKSTLPIDLLESCRMLHTTCCEFLKHFYIHFQSGEQKQASTVKKLYNHLKDCIEKLNELFQDV

SEQ ID NO: 32 - protein, 670 aa, Candida albicans
  misc_feature: Corresponds to SEQ ID NO: 105
MDIIRGACSVDKIGGMVYIREDLAPLMLEWKPIDEQEEDRAISIPLNSLTTLQSTKETSPKMILKIVYKLTSGPPNTNADGTDNGGGGGGEQKSFKLTFTNRPTMNTIKDSLQTIVARSRTKGGLKVPVLQLQLQHQLQHLGSAPQADSTRDSTSSSTPIPPTTSGTSTSSSLLSLAASQSLSDANLLKNFELQQKLLLEDRQLRDVFTKSVMQFKLSPQVFWSSRLNQLRTFALTISQHKGPYNVLSTIKPVATSDNQVNVNVTRDTINEIFTIYPIIKKAFDDLVPNKFNEGEFWSRFFNSKLFRRLRGDKISISNSRGDVVLDKYLYIDQNYQEKLQKSSTLENNGSGGGGGGAGGGSGNSEQGIQTLESPHVKKFLDLMGNQQDNSQKLGNRPDFTMRYDEDNVDDDNKKPTLGNENEMIILMKNMNRLSSKMMSMSSTNGPEKPSETTIDGLSAAELNEYEEELDLHDLNDSENLQYIKLNINTDIAKGTKLDSYEGSNTNNKISQDELHKYLQSQTFQGQIELTETYTCKSEEIEKTSMEIAMLIKQNFRTFKLINKENDIAGTNIVPNSLIQEIITYNITIVEFLSHFWKIFLHGNNPGQLKKIFTSLKNCQSGLIELENKAIDQFKSMDILQKNQKLQDKVLKDFASCLQPMKIALDKACNE

SEQ ID NO: 33 - protein, 498 aa, Homo sapiens
  misc_feature: Corresponds to SEQ ID NO: 106
  ref <308>: DATABASE ACCESSION NUMBER: Human Genbank/W19128
  ref <309>: DATABASE ENTRY DATE: 1996-05-03
  ref <313>: RELEVANT RESIDUES: (1)..(498)
MATSSEEVLLIVKKVRQKKQDGALYLMAERIAWAPEGKDRFTISHMYADIKCQKISPEGKAKIQLQLVLHAGDTTNFHFSNESTAVKERDAVKDLLQQLLPFKRANKELEKNRCCKILFCFSFIKLRTGEEQMLEDPVLFQLYKDVSQVISAEEFWNRLNVNATDSSTSNHKQDVGISAAFLADVRPQTDGCNGLRYNLTSDIIESIFRTYPAVKMKYAENVPHNMTEKEFWTRFFQSHYFHRDRLNTGSKDLFAECAKIDEKGLKTMVSLGVKNPLLDLTALEDKPLDEGYGISSVPSSNSKSIKENSNAAIIKRFNHHSAMVLAAGLRKQEAQNEQTSEPSNMDGNSGDADCFQPAVKRAKLQESIEYEDLGKNNSVKTIALNLKKSDRYYHGPTPIQSLQYATSQDIINSFQSIRQEMEAYTPKLTQVLSSSAASSTITALSPGGALMQGGTQQAINQMVPNDIQTNLVSHIEEMLQTAYNKLHTWQSRRLMKKT

SEQ ID NO: 34 - protein, 846 aa, Saccharomyces cerevisiae
  misc_feature: Corresponds to SEQ ID NO: 107
MELEPTLFGIIEALAPQLLSQSHLQTFVSDVVNLLRSSTKSATQLGPLIDFYKLQSLDSPETTIMWHKIEKFLDALFGIQNTDDMVKYLSVFQSLLPSNYRAKIVQKSSGLNMENLANHEHLLSPVRAPSIYTEASFENMDRFSERRSMVSSPNRYVPSSTYSSVTLRQLSNPYYVNTIPEEDILKYVSYTLLATTSALFPFDHEQIQIPSKIPNFESGLLHLIFEAGLLYQSLGYKVEKFRMLNISPMKKALIIEISEELQNYTAFVNNLVSSGTVVSLKSLYREIYENIIRLRIYCRFTEHLEELSGDTFLIELNIFKSHGDLTIRKIATNLFNSMISLYYEYLMNWLTKGLLRATYGEFFIAENTDTNGTDDDFIYHIPIEFNQERVPAFIPKELAYKIFMIGKSYIFLEKYCKEVQWTNEFSKKYHVLYQSNSYRGISTNFFEIINDQYSEIVNHTNQILNQKFHYRDVVFALKNILLMGKSDFMDALIEKANDILATPSDSLPNYKLTRVLQEAVQLSSLRHLMNSPRNSSVINGLDARVLDLGHGSVGWDVFTLDYILYPPLSLVLNVNRPFGRKEYLRIFNFLWRFKKNNYFYQKEMLKSNDIIRSFKKIRGYNPLIRDIINKLSRISILRTQFQQFNSKMESYYLNCIIEENFKEMTRKLQRTENKSQNQFDLIRLNNGTIELNGILTPKAEVLTKSSSSKPQKHAIEKTLNIDELESVHNTFLTNILSHKLFATNTSEISVGDYSGQPYPTSLVLLLNSVYEFVKVYCNLNDIGYEIFIKMNLNDHEASNGLLGKFNTNLKEIVSQYKNFKDRLYIFRADLKNDGDEELFLLSKSLR

SEQ ID NO: 35 - protein, 712 aa, Candida albicans
  misc_feature: Corresponds to SEQ ID NO: 108
MALNKVQLIKLYSNRLVKSLVPVEFGEAFIQSIINDLQTTLLNTSSEEQNLSIIINKLKMQFLSNNLKNEWVEFQNIVNSLSKFKSLDQICNYLAFLDALRDEKPEDILSTSTASLSPGKQNVMINTVNTALTLSQLIEPYYDTLSEQTILTYLPYTMLGLDSKIFTFSNNYTRLEIPKDINNSFSSLLREVFEFAILYKQLAIVVDRYKGTLVLAIKTAYIAILEAQLNKYVNDINNIFNNKPNSILVVYNSIFPWISILRFLYRVSNRLNRLDGYEFLTFIYSFTNHGDPKIRGIAVTAFTEVVKPYYNIVEHWIVKGELIDNNNEFFIIFDQEQNEFNSIIKLLPKKIPAFIKSSDKIFQIGTTLIFLNKYCRELKWVNQYNVKYSAILFNNHQGLASMTTNEMIKLIDLQYNEILTFLTQIIQGNNKLLTHVYNIKRYYFMETNDFIDAIMVKGKDVFNESSVNISSTYLRKVLQDAIQISSVKNFEYVDRLDSRVLNPQHGNLGWESFTIEYKIDDLPMSYLFEGHQHLQYLKMFHFLWKLRQLNNLLNWHFEMFNELNHNVVTKLSSRNRRPLAKSLSIITSIRFHFTQFLNELIAYLSYDVIEENFQQHIVRKLFYNKNDQDLLLNKLFMNLLEIDPNNDLPKFNVNLLTIDELVELHGTYIDSIINSSLLNEKLKGNETNISYIDQIFDILQTIFNFIIQVRNS

SEQ ID NO: 36 - protein, 880 aa, Homo sapiens
  misc_feature: Corresponds to SEQ ID NO: 109
  ref <308>: DATABASE ACCESSION NUMBER: Human Genbank/AAC39727
  ref <309>: DATABASE ENTRY DATE: 1998-05-06
  ref <313>: RELEVANT RESIDUES: (1)..(880)
MATPDQKSPNVLLQNLCCRILGRSEADVAQQFQYAVRVIGSNFAPTVERDEFLVAEKIKKELIRQRREADAALFSELHRKLHSQGVLKNKWSILYLLLSLSEDPRRQPSKVSSYATLFAQALPRDAHSTPYYYARPQTLPLSYQDRSAQSAQSSGSVGSSGISSIGLCALSGPAPAPQSLLPGQSNQAPGVGDCLRQQLGSRLAWTLTANQPSSQATTSKGVPSAVSRNMTRSRREGDTGGTMEITEAALVRDILYVFQGIDGKNIKMNNTENCYKVEGKANLSRSLRDTAVRLSELGWLHNKIRRYTDQRSLDRSFGLVGQSFCAALHQELREYYRLLSVLHSQLQLEDDQGVNLGLESSLTLRRLLVWTYDPKIRLKTLAALVDHCQGRKGGELASAVHAYTKTGDPYMRSLVQHILSLVSHPVLSFLYRWIYDGELEDTYHEFFVASDPTVKTDRLWHDKYTLRKSMIPSFMTMDQSRKVLLIGKSINFLHQVCHDQTPTTKMIAVTKSAESPQDAADLFTDLENAFQGKIDAAYFETSKYLLDVLNKKYSLLDHMQAMRRYLLLGQGDFIRHLMDLLKPELVRPATTLYQHNLTGILETAVRATNAQFDSPEILRRLDVRLLEVSPGDTGWDVFSLDYHVDGPIATVFTRECMSHYLRVFNFLWRAKRMEYILTDIRKGHMCNAKLLRNMPEFSGVLHQCHILASEMVHFIHQMQYYITFEVLECSWDELWNKVQQAQDLDHIIAAHEVFLDTIISRCLLDSDSRALLNQLRAVFDQIIELQNAQDAIYRAALEELQRRLQFEEKKKQREIEGQWGVTAAEEEEENKRIGEFKESIPKMCSQLRILTHFYQGIVQQFLVLLTTSSDESLRFLSFRL

SEQ ID NO: 37 - protein, 534 aa, Saccharomyces cerevisiae
  misc_feature: Corresponds to SEQ ID NO: 110
MEKSLADQISDIAIKPVNKDFDIEDEENASLFQHNEKNGESDLSDYGNSNTEETKKAHYLEVEKSKLRAEKGLELNDPKYTGVKGSRQALYEEVSENEDEEEEEEEEEEKEEDALSFRTDSEDEEVEIDEEESDADGGETEEAQQKRHALSKLIQQETKQAINKLSQSVQRDASKGYSILQQTKLFDNIIDLRIKLQKAVIAANKLPLTTESWEEAKMDDSEETKRLLKENEKLFNNLFNRLINFRIKFQLGDHITQNEEVAKHKLSKKRSLKELYQETNSLDSELKEYRTAVLNKWSTKVSSASGNAALSSNKFKAINLPADVQVENQLSDMSRLMKRTKLNRRNITPLYFQKDCANGRLPELISPVVKDSVDDNENSDDGLDIPKNYDPRRKDNNAIDITENPYVFDDEDFYRVLLNDLIDKKISNAHNSESAAITITSTNARSNNKLKKNIDTKASKGRKLNYSVQDPIANYEAPITSGYKWSDDQIDEFFAGLLGQRVNFNENEDEEQHARIENDEELEAVKNDDIQIFG

SEQ ID NO: 38 - protein, 480 aa, Candida albicans
  misc_feature: Corresponds to SEQ ID NO: 111
MSFFGLHFQLNSLTLNISNMAKKSLSEQISSLYTPKTDYDIEDHDLDVSKDNGIFQHHDGGSENESEDEDTGLRNEHYVESSKSKLRQQNEGVNLGEKYVGNVTSRSKLYDDEDDKQPTEASSGEELDAESAEEEEDEESEDVADDDEDDQESDRSSSSDAENDEDENISHKRELLKQLMSKERSHIVNRLSQSATNDALKGYSIQQQNKTFEKIIDVRLKFQKSVTSSNMLPINTSTYSETKSEDSDELVTKAKKQLYSLLDHLFTLRNELDESTSVKTPKKRSFAKYSEVTSAADAQLNSRRNQILTKWSAKVANSSGRNAMNANKFKTINQSFEQQVNNNLSDMDRLIKRTKLNRRNVTPIGYTTKEEDDHENGNKNKSIDEDDDDIPEDTSVRKKTQGLENDYIFDDEDFYRVLLNDLVDKKVQTSDPTSGITISLRAAQKSNKLKNNVDTKASKGRKLRYHVQEPIANFETSRGS

SEQ ID NO: 39 - protein, 558 aa, Homo sapiens [sequence omitted]

SEQ ID NO: 40 - protein, 300 aa, Saccharomyces cerevisiae
  misc_feature: Corresponds to SEQ ID NO: 113
MATLHFVPQHEEEQVYSISGKALKLTTSDDIKPYLEELAALKTCTKLDLSGNTIGTEASEALAKCIAENTQVRESLVEVNFADLYTSRLVDEVVDSLKFLLPVLLKCPHLEIVNLSDNAFGLRTIELLEDYIAHAVNIKHLILSNNGMGPFAGERIGKALFHLAQNKKAASKPFLETFICNTFTKHASLILAKALPTWKDSLFELNLNDCLLKTAGSDEVFKVFTEVKFPNLHVLKFEYNEMAQETIEVSFLPAMEKGNLPELEKLEINGNRLDEDSDALDLLQSKFDDLEVDDFEEVDS

SEQ ID NO: 41 - protein, 415 aa, Candida albicans
  misc_feature: Corresponds to SEQ ID NO: 114
MASVEVELGVTPETTYSISGKQLKFDSESDIAPYIKELTEKENVKKVDFSGNTIGIEASKALSEALLKHKDTIVEINFSDLYTGRLNTEIPQSLEYLLPALSKLPNLKLINLSDNAFGLQTIDPIEAYLAKAVSIEHLILSNNGMGPFAGSRIGGSLFKLAKAKKAEGKESLKTFICGRNRLENGSVNYLSVGLRNHKDLEVVRLYQNGIRPAGISKLVEQGLSNNKKLKVLDLQDNTITTRGAIHIAESLSNWPLLVELNLNDSLLKNKGSLKLVEAFHAGDEKPQLITLKLQYNELETDSLRVLADAIASKLPQLKFLELNGNRFEEDSEHIDKINGIFEERGYGEIDELDELEELDSEEEEDDEDDEGEDDTLEEDLDLTQLEEELAGVSLEDKDGNVDEIAEELSKTHIKZ

SEQ ID NO: 42 - protein, 587 aa, Homo sapiens
  misc_feature: Corresponds to SEQ ID NO: 115
  ref <308>: DATABASE ACCESSION NUMBER: Human Genbank/CAA57714
  ref <309>: DATABASE ENTRY DATE: 1995-05-11
  ref <313>: RELEVANT RESIDUES: (1)..(587)
MASEDIAKLAETLAKTQVAGGQLSFKGKSLKLNTAEDAKDVIKEIEDFDSLEALRLEGNTVGVEAARVIAKALEKKSELKRCHWSDMFTGRLRTEIPPALISLGEGLITAGAQLVELDLSDNAFGPDGVQGFEALLKSSACFTLQELKLNNCGMGIGGGKILAAALTECHRKSSAQGKPLALKVFVAGRNRLENDGATALAEAFRVIGTLEEVHMPQNGINHPGITALAQAFAVNPLLRVINLNDNTFTEKGAVAMAETLKTLRQVEVINFGDCLVRSKGAVAIADAIRGGLPKLKELNLSFCEIKRDAALAVAEAMADKAELEKLDLNGNTLGEEGCEQLQEVLEGFNMAKVLASLSDDEDEEEEEEGEEEEEEAEEEEEEDEEEEEEEEEEEEEEPQQRGQGEKSATPSRKILDPNTGEPAPVLSSPPPADVSTFLAFPSPEKLLRLGPKSSVLIAQQTDTSDPEKVVSAFLKVSSVFKDEATVRMAVQDAVDALMQKAFNSSSFNSNTFLTRLLVHMGLLKSEDKVKAIANLYGPLMALNHMVQQDYFPKALAPLLLAFVTKPNSALESCSFARHSLLQTLYKV

SEQ ID NO: 43 - protein, 381 aa, Saccharomyces cerevisiae [sequence omitted]

SEQ ID NO: 44 - protein, 365 aa, Candida albicans
  misc_feature: Corresponds to SEQ ID NO: 117
MSKLLTPEILALIDPVVSSLKRHQLVDDKEIALTIAQLLMKVISAARWSNTYDLIELIRQVGVIFTEAYPRKVIPGNIVRRVLALIRDETETETETETEQTDNIPMMSSMFSLLATHNKNETIKEQTQLQLKKQTSDMRAIIIQGIRDLVDEISNVNDGIETMAVDLIHDDEILLTPTPNSETVQHFLIKARLKRKFTVVVTENYPNDIKAAHKFVKTLAEHNIETILIPDTTIYAVMSRVGKVIIGTNAVFANGGCLSNSGVANVVECAKEHRTPVFAVAGLFKLSPLYPFTRNDLIEVGNSGKVLNYDDFELVQNVDVVTNPLEDYIPPQHIDIFMTNIGGFSPSFIYRIVLDNYKAEDNKLE

SEQ ID NO: 45 - protein, 349 aa, Homo sapiens
  misc_feature: Corresponds to SEQ ID NO: 118
  ref <308>: DATABASE ACCESSION NUMBER: Human Genbank/AAC42002
  ref <309>: DATABASE ENTRY DATE: 1995-07-14
  ref <313>: RELEVANT RESIDUES: (1)..(349)
MPGSAAKGSELSERIESFVETLKRGGGPRSSEEMARETLGLLRQIITDHRWSNAGELMELIRREGRRMTAAQPSETTVGNMVRRVLKIIREEYGRLHGRSDEDQQESLHKLLTSGGLNEDFSFHYAQLQSNIIEAINELLVELEGTMENIAAQALEHIHSNEVIMTIGFSRTVEAFLKEAARKRKFHVIVAECAPFCQGHEMAVNLSKAGIETTVMTAAIFAVMSRVNKVIIGTKTILANGALRAVTGTHTLALAAKHHSTPLIVCAPMFKLSPQFPNEEDSFHKFVAPEEVLPFTEGDILEKVSVHCPVFDYVPPELITLFISNIGGNAPSYIYRLMSELYHPDDHVL

SEQ ID NO: 46 - protein, 246 aa, Saccharomyces cerevisiae
  misc_feature: Corresponds to SEQ ID NO: 119
MSRLEIYSPEGLRLDGRRWNELRRFESSINTHPHAADGSSYMEQGNNKIITLVKGPKEPRLKSQMDTSKALLNVSVNITKFSKFERSKSSHKNERRVLEIQTSLVRMFEKNVMLNIYPRTVIDIEIHVLEQDGGIMGSLINGITLALIDAGISMFDYISGISVGLYDTTPLLDTNSLEENAMSTVTLGVVGKSEKLSLLLVEDKIPLDRLENVLAIGIAGAHRVRDLMDEELRKHAQKRVSNASAR

SEQ ID NO: 47 - protein, 180 aa, Candida albicans
  misc_feature: Corresponds to SEQ ID NO: 120
MELYSPEGLRIDGRRWNELRRFECRINTHPNSSDGSSYVEQGNTKVMCTVQGPIEPALRSQQHSERANIEVNLNIASFSTFERKKRSRNERRLVELKTTLEKTFEESVMINLYPRTNIVINVQVLCQDGGMLAAVINSITLALIDAGISMYDYVSGVSCGLYDQTPLLDVNNLEEHDMSC

SEQ ID NO: 48 - protein, 245 aa, Homo sapiens
  misc_feature: Corresponds to SEQ ID NO: 121
  ref <308>: DATABASE ACCESSION NUMBER: Human Genbank/BAA91279
  ref <309>: DATABASE ENTRY DATE: 2000-02-22
  ref <313>: RELEVANT RESIDUES: (1)..(245)
MAGLELLSDQGYRVDGRRAGELRKIQARMGVFAQADGSAYIEQGNTKALAVVYGPHEIRSRARALPDRALVNCQYSSATFSTGERKRRPHGDRKSCEMGLQLRQTFEAAILTQLHPRSQIDIYVQVLQADGGTYAACVNAATLAVLDAGIPMRDFVCACSAGFVDGTALADLSHVEEAAGGPQLALALLPASGQIALLEMDARLHEDHLERVLEAAAQAARDVHTLLDRVVRQHVREASILLGDG

SEQ ID NO: 49 - protein, 720 aa, Saccharomyces cerevisiae
  misc_feature: Corresponds to SEQ ID NO: 122
MSRFFSSNYEYDVASSSSEEDLLSSSEEDLLSSSSSESELDQESDDSFFNESESESEADVDSDDSDAKPYGPDWFKKSEFRKQGGGSNKFLKSSNYDSSDEESDEEDGKKVVKSAKEKLLDEMQDVYNKISQAENSDDWLTISNEFDLISRLLVRAQQQNWGTPNIFIKVVAQVEDAVNNTQQADLKNKAVARAYNTTKQRVKKVSRENEDSMAKFRNDPESFDKEPTADLDISANGFTISSSQGNDQAVQEDFFTRLQTIIDSRGKKTVNQQSLISTLEELLTVAEKPYEFIMAYLTLIPSRFDASANLSYQPIDQWKSSFNDISKLLSILDQTIDTYQVNEFADPIDFIEDEPKEDSDGVKRILGSIFSFVERLDDEFMKSLLNIDPHSSDYLIRLRDEQSIYNLILRTQLYFEATLKDEHDLERALTRPFVKRLDHIYYKSENLIKIMETAAWNIIPAQFKSKFTSKDQLDSADYVDNLIDGLSTILSKQNNIAVQKRAILYNIYYTALNKDFQTAKDMLLTSQVQTNINQFDSSLQILFNRVVVQLGLSAFKLCLIEECHQILNDLLSSSHLREILGQQSLHRISLNSSNNASADERARQCLPYHQHINLDLIDVVFLTCSLLIEIPRMTAFYSGIKVKRIPYSPKSIRRSLEHYDSLKTYFFSFKRFYSSFSVAKLAELFDLPENKVVEVLQSVIAELEIPAKLNDEKTIFVVEK

SEQ ID NO: 50 - protein, 874 aa, Candida albicans
  misc_feature: Corresponds to SEQ ID NO: 123
MSRFFVSGYTSDSSSEEEDLLSTSEEELLSSSDEGEDNESDSSFFGEDDDESEESSSDDEDGRPSGPAYFLKKSFLKGAGGDDSDSDSDDEGRKVVKSAKDKLLDDMKSSIEIINSNKYNNNWSIVLGEFDKFGRFLIRCNQTNLGTPKFYIKLLTSLDNSITETSNNERDDKTLKADEARAFNTLRQRIKKQIREFQVYYDLYKENPEEFDENEDEPLESVQAGLNDNVKNEADNSNVGALASNRVLSPIFHTLKTISESRGKKNIDKLEQIATLEKLLEANVSKSSPFELISIYQMLLSVRFDASSNQAFMPLEQWQKNEHDLGKLLDLLEANVDTYQVSELGSTTDDIDIEPVANAQGVKVIFGSITSSIDRLDDELTKSLQHTDPHSIEYVERLKDESTIYNLIVRGQAYVESITPEDVKYNSEQLARIVLRRLEHIYYKPKQLIKANEEEAWRNIEYNSSIVSKGSSVDEVIDQLTEFLQKQQKNKTYGKHAILFSIYYYAVNSQYEKAKELFLRSQFYSNINSAESSLQVQYNRALVQLGLSAFRAGSIEESHKILNEIVNSQRSKELLGQGFNSKFPNQATVLERQKLLPFHQHINLELLECVFMTCSLLIEIPTLAAIANNHKDSKRKNASLKSFKSKLDFHDRQFFTGPPESIKDHIVHASIALQKGDWLKSYNLLSSIKIWKLFPDNDKLLAMMKNQLQIEGLRTYIFTYKSVFKKLSIEKLQQIFQLSKDEVVSILEKMITTGNVSGGEIIDNKFISFTSTTEPQRSKLQELAIVLNEKIQLLTEKNEKTQSNGYGKKQQNKDQQNQQQQNQNQNQQQQQNQQQQQQQQSSQQQSNNILSEESANKFRYANVNSNNDEFQATA

SEQ ID NO: 51 - protein, 853 aa, Homo sapiens
  misc_feature: Corresponds to SEQ ID NO: 124
  ref <308>: DATABASE ACCESSION NUMBER: Human Genbank/AAD03462
  ref <309>: DATABASE ENTRY DATE: 1999-01-08
  ref <313>: RELEVANT RESIDUES: (1)..(853)
MSRFFTTGSDSESESSLSGEELVTKPVGGNYGKQPLLLSEDEEDTKRVVRSAKDKRFEELTNLIRTIRNAMKIRDVTKCLEEFELLGKAYGKAKSIVDKEGVPRFYIRILADLEDYLNELWEDKEGKKKMNKNNAKALSTLRQKIRKYNRDFESHITSYKQNPEQSADEDAEKNEEDSEGSSDEDEDEDGVSAATFLKKKSEAPSGESRKFLKKMDDEDEDSEDSEDDEDWDTGSTSSDSDSEEEEGKQTALASRFLKKAPTTDEDKKAAEKKREDKAKKKHDRKSKRLDEEEEDNEGGEAAENNLGEGVIVKIKFNIIASLYDYNPNLATYMKPEMWGKCLDCINELMDILFANPNIFVGENILEESENLHNADQPLRVRGCILTLVERMDEEFTKIMQNTDPHSQEYVEHLKDEAQVCAIIERVQRYLEEKGTTEEVCRIYLLRILHTYYKFDYKAHQRQLTPPEGSSKSEQDQAENEGEDSAVLMERLCKYIYAKDRTDRIRTCAILCHIYHHALHSRWYQARDLMLMSHLQDNIQHADPPVQILYNRTMVQLGICAFRQGLTKDAHNALLDIQSSGRAKELLGQGLLLRSLQERNQEQEKVERRRQVPFHLHINLELLECVYLVSAMLLEIPYMAAHESDARRRMISKQFHHQLRVGERQPLLGPPESMREHVVAASKAMKMGDWKTCHSFIINEKMNGKVWDLFPEADKVRTMLVRKIQEESLRTYLFTYSSVYDSISMETLSDMFELDLPTVHSIISKMIINEELMASLDQPTQTVVMHRTEPTAQQNLALQLAEKLGSLVENNERVFDHKQGTYGGYFRDQKDGYRKNEGYMRRGGYRQQQSQTAY

SEQ ID NO: 52 - protein, 297 aa, Saccharomyces cerevisiae
  misc_feature: Corresponds to SEQ ID NO: 125
MSELNALLKDINGSLTATSESLERLSGIYSNSATDEIPESNQLHEHLFYDAKKPAEKVSLLSLKNGSMLGYINSLLMLIGNRLDDECKDPSAMDARERSIQHRVVLERGVKPLEKKLAYQLDKLTRAYVKMEKEYKDAEKRALEKSTLVNHSGNDDSEDDESSEDEIAYRPNTSGIINTNKKSSAYRVEETAKQENGEENDDNETGVYKPPKITAVLPPQQTHFEDRFDAREHKDRSNKSNKAEKRKQKQRERNARMNVIGGEDFGIFSSKRKLEDSTSRRGAKKTRSAWDRAQRRL

SEQ ID NO: 53 - protein, 300 aa, Candida albicans
  misc_feature: Corresponds to SEQ ID NO: 126
MSKVDTVLKEIISSTKSTEASVKELIAFVKDSSSQHPELVRNLLAKSNSSLEGVSLLGLKNESLVSYINNIVLVVLSHLERLESDSETGSSAVERSIIQRVTLEKGVKPLEKKLSYQLDKMIRAYGRMEQDEIKAEQKLNDRGSGENDENDENDSEEDSEEDSEDDSEDDELAYRPDASSFAKLTSAKTKSKPTSSAVSTSNEKYRPPKISAMAPPTAVKSHDLDANTTSSKNRKLQSMEEYLQEQSDMPMVEASVGSTIVEHGRGGVKTQHDRKKEREIQTYEEDNFVRLPTSQTKKSF

SEQ ID NO: 54 - protein, 311 aa, Homo sapiens
  misc_feature: Corresponds to SEQ ID NO: 127
  ref <308>: DATABASE ACCESSION NUMBER: Human Genbank/AL050003
  ref <309>: DATABASE ENTRY DATE: 2000-02-18
  ref <313>: RELEVANT RESIDUES: (1)..(311)
MAALGVLESDLPSAVTLLKNLQEQVMAVTAQVKSLTQKVQAGAYPTEKGLSFLEVKDQLLLMYLMDLTHLILDKASGGSLQGHDAVLRLVEIRTVLEKLRPLDQKLKYQIDKLIKTAVTGSLSENDPLRFKPHPSNMMSKLSSEDEEEDEAEDDQSEASGKKSVKGVSKKYVPPRLVPVHYDETEAEREKKRLERAKRRALSSSVIRELKEQYSDAPEEIRDARHPHVTRQSQEDQHRINYEESMMVRLSVSKREKGRRKRANVMSSQLHSLTHFSDISALTGGTVHLDEDQNPIKKRKKIPQKGRKKKGQ

SEQ ID NO: 55 - protein, 221 aa, Saccharomyces cerevisiae
  misc_feature: Corresponds to SEQ ID NO: 128
MSATESSSIFTLSHNSNLQDILAANAKWASQMNNIQPTLFPDHNAKGQSPHTLFIGCSDSRYNENCLGVLPGEVFTWKNVANICHSEDLTLKATLEFAIICLKVNKVIICGHTDCGGIKTCLTNQREALPKVNCSHLYKYLDDIDTMYHEESQNLIHLKTQREKSHYLSHCNVKRQFNRIIENPTVQTAVQNGELQVYGLLYNVEDGLLQTVSTYTKVTPK

SEQ ID NO: 56 - protein, 281 aa, Candida albicans
  misc_feature: Corresponds to SEQ ID NO: 129
MGRENILKYQLEHDHESDLVTEKDQSLLLDNNNNLNGMNNTIKTHPVRVSSGNHNNFPFTLSSESTLQDFLNNNKFFVDSIKHNHGNQIFDLNGQGQSPHTLWIGCSDSRAGDQCLATLPGEIFVHRNIANIVNANDISSQGVIQFAIDVLKVKKIIVCGHTDCGGIWASLSKKKIGGVLDLWLNPVRHIRAANLKLLEEYNQDPKLKAKKLAELNVISSVTALKRHPSASVALKKNEIEVWGMLYDVATGYLSQVEIPQDEFEDLFHVHDEHDEEEYNPH

SEQ ID NO: 57 - protein, 281 aa, Saccharomyces cerevisiae
  misc_feature: Corresponds to SEQ ID NO: 130
MKARKSQRKAGSKPNLIQSKLQVNNGSKSNKIVKCDKCEMSYSSTSIEDRAIHEKYHTLQLHGRKWSPNWGSIVYTERNHSRTVHLSRSTGTITPLNSSPLKKSSPSITHQEEKIVYVRPDKSNGEVRAMTEIMTLVNNELNAPHDENVIWNSTTEEKGKAFVYIRNDRAVGIIIIENLYGGNGKTSSRGRWMVYDSRRLVQNVYPDFKIGISRIWVCRTARKLGIATKLIDVARENIVYGEVIPRYQVAWSQPTDSGGKLASKYNGIMHKSGKLLLPVYI

SEQ ID NO: 58 - protein, 260 aa, Candida albicans
  misc_feature: Corresponds to SEQ ID NO: 131
MGSINSQKAQKIQSILALPSNFKKITCSTCDMTYNPHISQDKLLHNKYHTNFINGIPWNYKTDNDVLIIENFTLVETPKLNSTGKSLKLTKTRQTFKGSIICINKSNKRHIQKVELLLNMVNQELNASQDSGQWKKPEFDRSKAFVIIIDSKAIGLCTTDTIQPDQGRWMIHKTQSIVPNQINKNVVIGISRIWISRKWRQYGLGKKLLNVVLKNSIYSVQLLKNQVAFSQPSFSGGMLAKSFNGVKHKSGEMLLPVYIE

SEQ ID NO: 59 - protein, 620 aa, Saccharomyces cerevisiae
  misc_feature: Corresponds to SEQ ID NO: 132
MLNGEDFVEHNDILSSPAKSRNVTPKRVDPHGERQLRRIHSSKKNLLERISLVGNERKNTSPDPALKPKTPSKAPRKRGRPRKIQEELTDRIKKDEKDTISSKKKRKLDKDTSGNVNEESKTSNNKQVMEKTGIKEKREREKIQVATTTYEDNVTPQTDDNFVSNSPEPPEPATPSKKSLTTNHDFTSPLKQIIMNNLKEYKDSTSPGKLTLSRNFTPTPVPKNKKLYQTSETKSASSFLDTFEGYFDQRKIVRTNAKSRHTMSMAPDVTREEFSLVSNFFNENFQKRPRQKLFEIQKKMFPQYWFELTQGFSLLFYGVGSKRNFLEEFAIDYLSPKIAYSQLAYENELQQNKPVNSIPCLILNGYNPSCNYRDVFKEITDLLVPAELTRSETKYWGNHVILQIQKMIDFYKNQPLDIKLILVVHNLDGPSIRKNTFQTMLSFLSVIRQIAIVASTDHIYAPLLWDNMKAQNYNFVFHDISNFEPSTVESTFQDVMKMGKSDTSSGAEGAKYVLQSLTVNSKKMYKLLIETQMQNMGNLSANTGPKRGTQRTGVELKLFNHLCAADFIASNEIALRSMLREFIEHKMANITKNNSGMEIIWVPYTYAELEKLLKTVLNTL

SEQ ID NO: 60 - protein, 600 aa, Candida albicans
  misc_feature: Corresponds to SEQ ID NO: 133
MSHSNALPNSPFRSPKKQRMEVIGPLNASRFSFSPVKTPPHGRAGLSSPEKRLVKDLDKARKRANNSLYNRLMDEYLDTDDYLDEQDRILADRIIKQSRGEPDEVNYGSDVELEIDLTQQRRTRRREKKVVYSSDSSNEYEDTGMPEESSSEEEEADDDDGNVEFVYGPPKERKTSLSSSPPTVKPTVRRTKRGRPSKSELVLGQIKSIFHQDDVLFSTDRKTFTPTKPTAAKKPVSNYLTSIFDQNFDRSKVPSLSGIPKSTNTHEEKKTFVPLPIPTLDADGNITDKEYISKYFDGVDPAKFKEGRFVDEKVFYLEGPEGYFEQQTTRVKQSGNSLTALAPQIEYKDFARLVKLGDNLSFQRKRHLFELHKYIYHQWCFEMSQGFNLNFYGVGSKIDLLRDFATNYFGIWWENVVHADLPKVLVVNGFNPSINIKKLILEIASILLPNELYPKHIAGTVPFVVDYLNNHRLPCGSIGFHKPKILLIIHNLDGEVFRVDKTQTLLSQLMTLPEVWAMSSTDHINASLLWDLSKVKNLNFIWHNLTTYATYQRETSFRDVISLGKSKKFVGGLGAKYVLRSLTDNHRNLYRELLIAQLDK

SEQ ID NO: 61 - protein, 577 aa, Homo sapiens
  misc_feature: Corresponds to SEQ ID NO: 134
  ref <308>: DATABASE ACCESSION NUMBER: Human Genbank/Q13416
  ref <309>: DATABASE ENTRY DATE: 2000-05-30
  ref <313>: RELEVANT RESIDUES: (1)..(577)
MSKPELKEDKMLEVHFVGDDDVLNHILDREGGAKLKKERAQLLVNPKKIIKKPEYDLEEDDQEVLKDQNYVEIMGRDVQESLKNGSATGGGNKVYSFQNRKHSEKMAKLASELAKTPQKSVSFSLKNDPEITINVPQSSKGHSASDKVQPKNNDKSEFLSTAPRSLRKRLIVPRSHSDSESEYSASNSEDDEGVAQEHEEDTNAVIFSQKIQAQNRVVSAPVGKETPSKRMKRDKTSDLVEEYFEAHSSSKVLTSDRTLQKLKRAKLDQQTLRNLLSKVSPSFSAELKQLNQQYEKLFHKWMLQLHLGFNIVLYGLGSKRDLLERFRTTMLQDSIHVVINGFFPGISVKSVLNSITEEVLDHMGTFRSILDQLDWIVNKFKEDSSLELFLLIHNLDSQMLRGEKSQQIIGQLSSLHNIYLIASIDHLNAPLMWDHAKQSLFNWLWYETTTYSPYTEETSYENSLLVKQSGSLPLSSLTHVLRSLTPNARGIFRLLIKYQLDNQDNPSYIGLSFQDFYQQCREAFLVNSDLTLRAQLTEFRDHKLIRTKKGTDGVEYLLIPVDNGTLTDFLEKEEEEA

SEQ ID NO: 62 - protein, 385 aa, Saccharomyces cerevisiae
  misc_feature: Corresponds to SEQ ID NO: 135
MSSVNANGGYTKPQKYVPGPGDPELPPQLSEFKDKTSDEILKEMNRMPFFMTKLDETDGAGGENVELEALKALAYEGEPHEIAENFKKQGNELYKAKRFKDARELYSKGLAVECEDKSINESLYANRAACELELKNYRRCIEDCSKALTINPKNVKCYYRTSKAFFQLNKLEEAKSAATFANQRIDPENKSILNMLSVIDRKEQELKAKEEKQQREAQERENKKIMLESAMTLRNITNIKTHSPVELLNEGKIRLEDPMDFESQLIYPALIMYPTQDEFDFVGEVSELTTVQELVDLVLEGPQERFKKEGKENFTPKKVLVFMETKAGGLIKAGKKLTFHDILKKESPDVPLFDNALKIYIVPKVESEGWISKWDKQKALERRSV

SEQ ID NO: 63 - protein, 300 aa, Candida albicans
  misc_feature: Corresponds to SEQ ID NO: 136
MSKIEPVTEKEEEYVSEWDRRRYVPKAGEPELPPQLSEFSNKTTDEVIEELNRLPFFMTLDETDGDGGENVNLEALKSLAYEGDPDEIASNFKNQGNNCYKFKKYKDAIIFYTKGLEVNCDVDAINSALYLNRAACNLELKNYRRCIEDCKKVLMLDEKNIKACFRSGKAFFAIEKYDEAIKVLEYGLNIEPENKDLQKLLQQVQKRQETLAQIKAKKAQEEEQERLKNIVLENSIKLRHIEIVKSSSPPEVLKTAKIRLEDPKDYQSQLIFPAMILYPTTDEFDFIAEISELTTPLELL

SEQ ID NO: 64 - protein, 356 aa, Homo sapiens
  misc_feature: Corresponds to SEQ ID NO: 137
  ref <308>: DATABASE ACCESSION NUMBER: Human Genbank/NP_004614
  ref <309>: DATABASE ENTRY DATE: 2000-11-01
  ref <313>: RELEVANT RESIDUES: (1)..(356)
MEQPGQDPTSDDVMDSFLEKFQSQPYRGGFHEDQWEKEFEKVPLFMSRAPSEIDPRENPDLACLQSIIFDEERSPEEQAKTYKDEGNDYFKEKDYKKAVISYTEGLKKKCADPDLNAVLYTNRAAAQYYLGNFRSALNDVTAARKLKPCHLKAIIRGALCHLELIHFAEAVNWCDEGLQIDAKEKKLLEMRAKADKLKRIEQRDVRKANLKEKKERNQNEALLQAIKARNIRLSEAACEDEDSASEGLGELFLDGLSTENPHGARLSLDGQGRLSWPVLFLYPEYAQSDFISAFHEDSRFIDHLMVMFGETPSWDLEQKYCLIIWRSTLRMRTGQNYTGCLPRAPCYRFYSTRGTL

SEQ ID NO: 65 - protein, 167 aa, Saccharomyces cerevisiae
  misc_feature: Corresponds to SEQ ID NO: 138
MSTIPSEIINWTILNEIISMDDDDSDFSKGLIIQFIDQAQTTFAQMQRQLDGEKNLTELDNLGHFLKGSSAALGLQRIAWVCERIQNLGRKMEHFFPNKTELVNTLSDKSIINGINIDEDDEEIKIQVDDKDENSIYLILIAKALNQSRLEFKLARIELSKYYNTNL

SEQ ID NO: 66 - protein, 184 aa, Candida albicans
  misc_feature: Corresponds to SEQ ID NO: 139
MSEDKLQKLQDSGLVDWAVFSEIVTMDEDEEGFSKSLVEVFVSQVEETFEEIDKYLKEKNLEKLSSSGHFLKGSAAALGLTKISNQCERIQNYGHKINFDNFQLEDIKTKGDSAVSAENVAVNDGETNPENGSNGNETSNNKTNTSNIPDESSDDFWIALIEDALAKARDGFDQSRRALDEYYE

SEQ ID NO: 67 - protein, 240 aa, Homo sapiens
  misc_feature: Corresponds to SEQ ID NO: 140
  ref <308>: DATABASE ACCESSION NUMBER: Human Genbank/CAA78727
  ref <309>: DATABASE ENTRY DATE: 1993-01-10
  ref <313>: RELEVANT RESIDUES: (1)..(240)
TDKLSNMQKDLENSNAKLQEKIQELKANEHQLITLKKDVNETQKKVSEMEQLKKQIKDQSLTLSKLEIENLNLAQELHENLEEMKSVMKERDNLRRVEETLKLERDQLKESLQETKARDLEIQQELKTARMLSKEHKETVDKLREKISEKTIQISDIQKDLDKSKDELQKKIQELQKKELQLLRVKEDVNMSHKKINEMEQLKKQFEPNYLCKCEMDNFQLTKKLHESLEEIRIVAKERD

SEQ ID NO: 68 - protein, 93 aa, Saccharomyces cerevisiae
  misc_feature: Corresponds to SEQ ID NO: 141
MSFLGFGGGQPQLSSQQKIQAAEAELDLVTDMFNKLVNNCYKKCINTSYSEGELNKNESSCLDRCVAKYFETNVQVGENMQKMGQSFNAAGKF

SEQ ID NO: 69 - protein, 91 aa, Candida albicans
  misc_feature: Corresponds to SEQ ID NO: 142
MFGLGGTTPQISSQQKLQAAEAELDMVTGMFNALVSQCHTKCINKSYNEADISKQESLCLDRCVAKYFETNVQVGENMQKLGQSGQFMGRR

SEQ ID NO: 70 - protein, 90 aa, Homo sapiens
  misc_feature: Corresponds to SEQ ID NO: 143
  ref <308>: DATABASE ACCESSION NUMBER: Human Genbank/NP_036588
  ref <309>: DATABASE ENTRY DATE: 2001-12-19
  ref <313>: RELEVANT RESIDUES: (1)..(90)
MDPLRAQQLAAELEVEMMADMYNRMTSACHRKCVPPHYKEAELSKGESVCLDRCVSKYLDIHERMGKKLTELSMQDEELMKRVQQSSGPA

SEQ ID NO: 71 - protein, 600 aa, Saccharomyces cerevisiae
  misc_feature: Corresponds to SEQ ID NO: 144
MTTEDPDSNHLSSETGIKLALDPNLITLALSSNPNSSLHSPTSDEPVPESAGKADTSIRLEGDELENKTKKDNDKNLKFLKNKDSLVSNPHEIYGSMPLEQLIPIILRQRGPGFKFVDLNEKELQNEIKQLGSDSSDGHNSEKKDTDGADENVQIGEDFMEVDYEDKDNPVDSRNETDHKTNENGETDDNIETVMTQEQFVKRRRDMLEHINLAMNESSLALEFVSLLLSSVKESTGMSSMSPFLRKVVKPSSLNSDKIPYVAPTKKEYIELDILNKGWKLQSLNESKDLLRASFNKLSSILQNEHDYWNKIMQSISNKDVIFKIRDRTSGQKLLAIKYGYEDSGSTYKHDRGIANIRNNIESQNLDLIPHSSSVFKGTDFVHSVKKFLRVRIFTKIESEDDYILSGESVMDRDSESEEAETKDIRKQIQLLKKIIFEKELMYQIKKECALLISYGVSIENENKVIIELPNEKFEIELLSLDDDSIVNHEQDLPKINDKRANLMLVMLRLLLVVIFKKTLRSRISSPHGLINLNVDDDILIIRPILGKVRFANYKLLLKKIIKDYVLDIVPGSSITETEVEREQPQENKNIDDENITKLN

SEQ ID NO: 72 - protein, 587 aa, Candida albicans
  misc_feature: Corresponds to SEQ ID NO: 145
MVEKQFNIDLELNDTGHIDPFLQDEYVCFLTLLVFLVLFFSLLTLPRDKLKLEELIPRIFERKSFLNVTEDSLRKEIDNSLKISEEDALDTEESREDTVEADQQEVFNKHKFELSKNINNALNETQLSLDFVSLLISSVKPSLAKSTISPHLSKFVKPTSLNSDRLGQDSNDNQESKATDSFGQGWKLESLGKITDLFREASTNLNDQVIKERRYWNMINLVLANDEVLFRMRDPQNNARAIGVKYGYGDSGSNFHDQGLALLRKDNQTGEISFHPISSINNAKIVEKVSRFIRVKILSQIDGDYMLTGQSIFNFDFEKSKQSIINDIEKARFFLFEEDLFHQLIREAKLLVNYNVSIISNKIIIEINNIIIEIESIVYDELNEEELENYYQNVNEYSTLHNKKCQLILNYLKLMLCCYYKYNLKLKQKVPTALTKWKQSNSHPLILRPLVGNMRHELNLLNMKSVLDRLMHAHESELSYSKLDVEKFINLATRSKKQNPFQKSIEKPISKFHLVLCNKTSNMLDVNIQLDNYELFVNLIINMTIIRFETEHDFKNNVNGINVLQLGFSDFNEIEECLDWSIQNFVL

SEQ ID NO: 73 - protein, 888 aa, Homo sapiens
  misc_feature: Corresponds to SEQ ID NO: 146
  ref <308>: DATABASE ACCESSION NUMBER: Human Genbank/BAA88763
  ref <309>: DATABASE ENTRY DATE: 2000-01-06
  ref <313>: RELEVANT RESIDUES: (1)..(888)
MYGSARSVGKVEPSSQSPGRSPRLPRSPRLGHRRTNSTGGSSGSSVGGGSGKTLSMENIQSLNAAYATSGPMYLSDHENVGSETPKSTMTLGRSGGRLPYGVRMTAMGSSPNIASSGVASDTIAFGEHHLPPVSMASTVPHSLRQARDNTIMDLQTQLKEVLRENDLLRKDVEVKESKLSSSMNSIKTFWSPELKKERALRKDEASKITIWKEQYRVVQEENQHMQMTIQALQDELRIQRDLNQLFQQDSSSRTGEPCVAELTEENFQRLHAEHERQAKELFLLRKTLEEMELRIETQKQTLNARDESIKKLLEMLQSKGLSAKATEEDHERTRRLAEAEMHVHHLESLLEQKEKENSMLREEMHRRFENAPDSAKTKALQTVIEMKDSKISSMERGLRDLEEEIQMLKSNGALSTEEREEEMKQMEVYRSHSKFMKNKIGQVKQELSRKDTELLALQTKLETLTNQFSDSKQHIEVLKESLTAKEQRAAILQTEVDALRLRLEEKETMLNKKTKQIQDMAEEKGTQAGEIHDLKDMLDVKERKVNVLQKKIENLQEQLRDKEKQMSSLKERVKSLQADTTNTDTALTTLEEALAEKERTIERLKEQRDRDEREKQEEIDNYKKDLKDLKEKVSLLQGDLSEKEASLLDLKEHASSLASSDESSKAQAEVDRLLEILKEVENEKNDKDKKIAELESLTSRQVKDQNKKVANLKHKEQVEKKKSAQMLEEARRREDNLNDSSQQLQVEELLMAMEKVKQELESMKAKLSSTQQSLAEKETHLTNLRAERRKHLEEVLEMKQEALLAAISEKDANIALLELSSSKKKTQEEVAALKREKDRLVQQLKQQTQNRMKLMADNYEDDHFKSSHSNQTNHKPSPDQDEEEGIWA

SEQ ID NO: 74 - DNA, 900 bp, Saccharomyces cerevisiae
  ref <308>: DATABASE ACCESSION NUMBER: Genbank/CAA96279.1
  ref <309>: DATABASE ENTRY DATE: 1997-08-11
  ref <313>: RELEVANT RESIDUES: (1)..(900)
atgagtggaatgatagaaaatgggttacagctatcggacaatgctaaaaccttacatagccagatgatgtcgaaaggaataggcgcattatttacacagcaagaactccaaaaacaaatgggaatcgggtcgttaacagacttgatgtccattgtacaggaattgctagacaagaacttgatcaaattagtaaaacaaaacgacgaattaaaatttcaaggtgtcttagaatctgaggcgcaaaagaaagccaccatgtcggctgaagaggcactggtatattcttatatcgaggctagcggtagagaagggatatggtccaagactatcaaggcaagaaccaatctccatcagcatgtagttcttaaatgcttgaagagtttagaatcccaaagatacgtgaagagtgttaagagtgtaaagtttcccacaaggaaaatctacatgttgtacagcttacaaccctctgtggacatcacaggaggtccatggttcacagatggagagctggatatagaatttatcaatagtttattgactattgtttggaggttcatatcagagaacaccttccctaatggcttcaagaatttcgaaaatggacccaaaaaaaacgtcttttatgctccaaacgtaaaaaattactctaccacacaagaaattttggaatttattacagcggcacaagtggccaatgtcgagttaaccccttcaaatatcagatctttgtgtgaagtcttagtgtacgacgacaagctggaaaaagtcacgcatgactgctatagagtgaccttagagagcattctacaaatgaaccaaggtgagggcgagccggaggcaggtaataaggctttggaggatgaagaagaattttccatctttaactacttcaagatgttt

SEQ ID NO: 75 - DNA, 993 bp, Candida albicans
atgagtgagatgttagtatcagataaagcacgtcatctttatacaaagatgagggagtatccaacttccaaactttttgatcaagatgaattacaaacactatttgatattaaaaagggatcagaattaatggaatatttacaagaattagtcaatggtaaatatgttaaaattagtaaaatgggagatcaattaaaatttcaaactgttgctgaagaagaagccaaaaaagtatcgtcaatgtctgatgatgaagcaatgatttattcttatattgaagcttcaggtcgtgaagggatttggactaaaaccattaaagctaaaactaatttacatcaacatattgttcaaaaatgtttaaaaaatttagaaaataatcgatacattaaaagtattaaatcagtgaaacatccaacaagaaaaatttatatgttgtataatttacaacctagtattgatgttactggtggtccttggtttactgattcagaattagatactgaatttatagaaactttattggaagtgtgttggagatttattgttgggaaaaccatgtatataaaggatgaagaagctgataatgaagatataaatccacttcaaacaacatatcacaatcatcatccaggggtgaatttggatcaacttgttgaatttataaacaatagtaatatcaccagtgttgagttgggtattaatgatattagatcattatgtgatgtgctaatctatgacgatagaatagaagaagttggtgggaatcaagaaaatagtgggatttttaaagctacttggcaaagtataatagataaaggtaacactattttgcaaaataattatcaggatttgaaaaatgttgtttctgaagattgttttaattatttacaacaaaatcaatcagattttagtgtttttcaatataaatctactattcaagatcttcaagatgaatcggatctagtgtatttagatagctggataaatgaataa

SEQ ID NO: 76 - DNA, 2203 bp, Homo sapiens
  misc_feature (1657)..(1658): n is unknown
  misc_feature (1661)..(1661): n is unknown
  ref <308>: DATABASE ACCESSION NUMBER: Human Genbank/U93869
  ref <309>: DATABASE ENTRY DATE: 1997-06-23
  ref <313>: RELEVANT RESIDUES: (1)..(2203)
cgacccgggttccgccgcttgctaccgggctgctccgtgcatctttccccccaggcgtcaggaactgcgcctcatgggcgaggtgaaggtgaaggtgcagccgcctgacgccgatccggtcgaaatagaaaacaggattatagaattatgtcaccagttccctcatggaatcacagaccaagtaattcagaatgaaatgcctcaatatagaagcccagcagcgggcagtagcatcaataggttgttgtctatgggtcagttggatctcttaaggagcaatacgggccttttatatagaataaaggactctcagaatgctggtaaaatgaagggatccgataaccaagaaaaactagtatatcaaatcatagaggatgcaggaaataaaggaatatggagcagagatatccgctataaaagtaatttgccattaacagaaatcaacaaaattctgaagaatctggaaagtaaaaagcttatcaaagctgttaagtctgtagcagcctcaaaaaagaaggtgtatatgctctataacctgcagccagaccggtctgtgactggtggagcctggtacagtgaccaggattttgaatctgaatttgtagaggtgcttaaccaacagtgttttaaattcctacagtccaaggcagaaacagcacgagaaagcaaacagaacccaatgatacaaagaaatagttcatttgcctcatcacatgaagtgtggaaatatatctgcgaattgggaatcagtaaggtagagttatccatggaagacattgaaaccatcctgaatacactcatttatgatggaaaagtggagatgacgattattgcctgcaaaagaaggcacagttggcagtgtagatggacacatgaaactgtacagggcagtcaatccaatcatccctcccacaggtttggtccgggccaccctgtggactctgccccggtttttgatgactgccacgaaggtggtgagatttcaccatctaactgtatttacatgacagagtggctcgaattttaatagagagctatgaactttattgacattttgcaaatgaagttacttagggagcagataatttaattcatgatggaacacgaaatctccttgaaagcaaacttcacaataatggacgtagacttgctgctatgaaaacatattttttttatttatgaagactaaatttatattggtaaaatagccagtagaatatgaaagaaataaggttagtagtgaaattcattcttcaataaataaaacactttgaaactccggaggaccacatctttcaagacttctgatgggcgaagcccccggcttcaaaacacgacaaggaagtggtctatttcgatgaatggacaatttgaaaagatgccaacatacccgtatttaccaagtactatgataatggctagagtataaaaatgttctttttaaagttatttattaagttcttcattggacgcttttttttatatctggttcactaccaccattttctgtttcctactttctcagtggtttcattgaaaagaaattagaaggggttaaaggcaggaatagcaaagagtgcaaacttggggtatgactgggggagagtggaacatgccttttccgcacaatattaattcctttttgtatcagaaaggnnctnttaggagttatgctaccatacttacttcaaacccaatgactactgtcaaggtcatattttcagtacataaatactatcattttcattctaaagaatattttcactgttccttctttcttaaagtcttatgtttcactctttaactcaaatgtattctttgttagaatttaccctagattcttatttaatgtctgcagtagactgaatgtttgtgtgcccccagaattctaatgttgaaatctcatttccaatgtgatggtatttggaggtggggcttttggtaagtgataggtcaggagagtaacagcgctcatgaatgggattagtgcccttatataaagagacccagagagctccatcaccccttctgccatgtgaaagggagaagacaaacatccacgaaccaggaagtgggtcctcaccagaaaacaaatctgtaagcaccttgatcttggacttcccagcctccagaattgtgagaaataaatttctgttgttgatttttttttttttttttttttttttttttttttttttttttt

SEQ ID NO: 77 - DNA, 588 bp, Saccharomyces cerevisiae
  ref <308>: DATABASE ACCESSION NUMBER: Genbank/CAA96194.1
  ref <309>: DATABASE ENTRY DATE: 1997-08-11
  ref <313>: RELEVANT RESIDUES: (1)..(588)
atgtccgggtcgttaaaatctctagacaagaaaatagctaaaagaaggcaggtgtataagcccgtgctagacaatccgttcacaaacgaagcacatatgtggccgcgcgtgcatgatcagccattgatttggcagctgctgcaatcctctatcataaataagttgattcacattcaatcgaaggagaactacccttgggagctgtatacagatttcaatgaaattgtgcagtatttgagcggcgctcacggaaacagcgacccagtatgtctatttgtgtgcaataaggaccctgatgtaccgcttgtgctcttgcagcaaatcccgctattatgctatatggcgcccatgacggttaaactggtgcagttgcccaagagtgccatggataccttcaagtcggtttctaaatatggaatgctgctgctgcggtgcgacgatagggtcgacaagaaattcgtatcgcagatccagaagaacgttgatctgcttcagtttccctggttaaatgctatcaagtatcggcccacatctgtcaagctgttgaaaactacagtgccaattgtctcgaagaagaggcaaaagtag

SEQ ID NO: 78 - DNA, 663 bp, Candida albicans
atgaataaatcaaataaagtcaagaaaccttcggtggccaaagtctcaactaaagctgcttcatcatcactcaagtctcaggaagcaaagagaaaacaagttttccgtccaattctcgataactcatttacacaatcaaaccaatggccatttatagaaccaactattgcaaacgatattgttgatctactagaagtattgctaaaaatgcaagactctacatttaaataccgtgggtttaatccaactgtgtctgctcttgaaaaacaagcagctgctaatcgtggtatacataaaaatgcttgtgtacaaataaagtatgtatttgtgtgcaagtacgatatatccccagcaacgctcacaaatgtgtttcctacgttgtgtttcacggcgtcaaaaagtgctgaagatcgggttaagctaatccagttaccaagaggaagtctagaacggttatcgaaagcacttggggtagatagagttggtatatttggtctaactaaagatactgaaggggcacaaccgttatttgatcttataaatgaaaatgtcaaagatattgaagctccttggctagactgtattttccgtgaggagatggtatttaatcaacctaacacaaagcatgtggcaagtactgtaggtagaaagaaaaacaagtag

SEQ ID NO: 79 - DNA, 960 bp, Saccharomyces cerevisiae
  ref <308>: DATABASE ACCESSION NUMBER: Genbank/CAA82141.1
  ref <309>: DATABASE ENTRY DATE: 1997-08-11
  ref <313>: RELEVANT RESIDUES: (1)..(960)
atgagtaaaaacagggaccctctactggctaatttgaacgctttcaaaagcaaagtgaagtctgccccggtgatcgcacccgctaaagttggacagaagaagaccaatgacacagtgattactatagatggaaacactaggaagaggacggcctccgaacgtgcgcaagaaaacactttgaactctgcgaaaaatcctgtgttagtggatatcaagaaagaagctgggagcaatagctctaatgctatttcattagatgacgacgatgacgacgaagattttggtagctctccttcaaaaaaagtaaggcctggctctattgctgcagccgctttacaagcaaatcaaacagatatttccaagagtcacgattcttcaaagttgctttgggcgactgaatacattcaaaagaaaggtaagcccgttttggtgaatgagttattggactacttgtcaatgaaaaaagatgacaaggttattgagcttttaaaaaaattagatagaatagagtttgaccccaagaaggggactttcaaatacctttccacctacgatgtccattccccttcggaactgctgaagttgttacgttcacaagtaacattcaaaggtatttcctgcaaagacttgaaagacggttggccacaatgcgatgaaacgattaaccaactggaggaagacagcaaaattttggtgttaagaactaaaaaggataaaactccaagatacgtttggtataacagcggtggtaacttgaaatgtattgacgaggagtttgttaaaatgtgggaaaatgtgcaattaccgcaatttgcagaattgccaagaaagctgcaagatttaggtctaaagcctgctagtgtcgatcctgctactatcaaaagacaaacaaagagagttgaagttaaaaagaagagacaaagaaagggtaagattactaacactcatatgaccggtatc

SEQ ID NO: 80 - DNA, 855 bp, Candida albicans
atgtccgacttatcagctcaactttcagcttttaagaataagatcaaaagtggaccatcggtgattgttcctagaaaggcaacttttactcaatctccatcatcaccattatcatcatcaaccacaacaacaacactgaagaatgacgccaatgtgaagaagagatcaacgacggattcagtaacccgagtattgaagaaacaaaaggcaaatatgggagaaatgacgggatcacatttatcgacacaattacaccttgctgttgaatatatcaaggaacatgaccaaccaatatcggtggagaagttgcagaattatttatcatttgatatatcacatactttattgccattattgaatgaaattgatcgagtgaaatacgacgaatctaagggtacattggaatatgtttcattgcataatattcgtagtagtgatgatttattggaatttttgagacgtcaaaccacattcaagggcacttccgtaaaagaattaaaagatggttgggctggttgtgttgccgctatagacgaattagaatcacaaggcaaaattttggtgttgcgtaacaagaaggaaaatgctccaagattagtatgggctaataatggtggtgagttgggttatattgacacagaattcaaggatatgtgggatcaagtgaaattgccggaaccagatgtattgtatcagaaattattggatcaaggattgaaacctacgggagctgatcctaatttgatcaaaaagcaaccacaacaaaaggaaaagaaacaaaagaaagcaagaagaggaaagattacaaatacacatatgaaaggtattttgaaggattattctcaattagtttga

SEQ ID NO: 81 - DNA, 1500 bp, Homo sapiens
  ref <308>: DATABASE ACCESSION NUMBER: Human Genbank/NM_002095.1
  ref <309>: DATABASE ENTRY DATE: 2000-10-30
  ref <313>: RELEVANT RESIDUES: (1)..(1500)
cttaaattacccactacgttgtccagtcgccgcctcagctaccgccgctgccgccgccgccgccgccaccgccagtggtgagaccccgacctggcgggtcagcgctgggcgtgcgtgcgggcaggcgggggcgctgacgagaagcaggaagagggtgcagtgccggcgtgggcggccggccgaggcggaggcgcaggaagggggcggcgagtcgtgcgaggctgcccttctcactcagcattatggatccaagcctgttgagagaaagggagctgttcaaaaaacgagctctttctactcctgtagtagaaaaacgttcagcatcttctgagtcatcatcatcatcgtcaaagaagaagaaaacaaaggtagaacatggaggatcgtcaggctctaaacaaaattctgatcatagcaatggatcatttaacttgaaagctttgtcaggaagctctggatataagtttggtgttcttgctaagattgtgaattacatgaagacacggcatcagcgaggagatacgcatcctctaaccttagatgaaattttggatgaaacacaacatttagatattggactcaagcagaaacaatggctaatgactgaggctttagtcaacaatcccaaaattgaagtaatagatgggaagtatgctttcaagcccaagtacaacgtgagagataagaaggccctacttaggctcttagatcagcatgaccagcgaggattaggaggaattcttttagaagacatagaagaagcactgcccaattcccagaaagctgtcaaggctttgggggaccagatactatttgtaaatcgtcccgataagaagaaaatacttttcttcaatgataagagctgtcagttttctgtggatgaagaatttcagaaactgtggaggagtgtcactgtagattccatggacgaggagaaaattgaagaatatctgaagcgacagggtatttcttccatgcaggaatctggaccaaagaaagtggcccctattcagagaaggaaaaagcctgcttcacagaaaaagcgacgctttaagactcataacgaacacttggctggagtgctgaaggattactctgacattacttccagcaaatagggaacagttttgccctggaacagagttacagatacacaatcaagagtgttcttgctgatgctcggggtctgaagactgtcttcctatctgcttcttgcggctgaggagaggagcagttcagtttacaaaacaagtgcaaattaccaaactcaaagcttatttgagtagaatgggctcatgggcaatgtgatgttccctgttaaccttctgttactccctgggagaaaggcgctgagcgtggcatgcaggtgtctttgctgtgtttttctccacttctaaatggttcctggttcctttcttcctcgtttgttactttagagcaagtttgcccatagtcttgaatgcaatatttgtttattccaaaagaacatatttataataa

SEQ ID NO: 82 - DNA, 1560 bp, Saccharomyces cerevisiae
  ref <308>: DATABASE ACCESSION NUMBER: Genbank/CAA96830.1
  ref <309>: DATABASE ENTRY DATE: 1997-08-11
  ref <313>: RELEVANT RESIDUES: (1)..(1560)
atgtctcaagaacagtacacagaaaacttgaaggttatcgttgccgaaaaactggctggtataccaaactttaacgaagatatcaagtatgttgcggagtatattgtcttattgatcgttaacggtggtactgttgaatctgtcgtagacgagctggctagtttgtttgatagtgtttcgagagatacgcttgcaaatgttgttcaaacagcctttttcgcattagaagctctgcaacagggagaaagtgctgaaaatattgtttccaaaattagaatgatgaatgcgcaaagcttgggacaatcggatatcgcacaacagcaacaacagcaacaacaacaacagcaaccagacatcgcgcaacagcaacctcaacagcaacctcaacagcaacctcaacagcaacctcaacagcaacctcaacagcaacctcaacagcaacctcaacagcaacctcaacagcaacctcaacttcaaccacttcagccacaactagggacccagaatgcaatgcagacagatgctcctgcaactccatcccccatatcagccttttccggcgttgttaacgctgcagctccccctcagtttgcgcctgtagataacagccaaaggttcactcaacgtggcggaggcgccgttggaaagaatcgtagaggtggtcgcggtgggaaccgtggaggacgcaacaataattccacacgttttaatccgttagcaaaagcacttggaatggcgggtgagagtaatatgaacttcactccaaccaagaaagaggggcgttgcagattgtttcctcactgtcctcttggtagatcatgcccacatgcacacccaactaaggtatgtaatgaatatccaaattgtccaaagcctcccggaacttgtgagtttttacatccaaatgaagatgaagagttgatgaaggaaatggaaagaactcgtgaagaatttcaaaaaagaaaagctgatttattggcggcaaaaaggaaaccggtacaaactggtatcgttctgtgtaaatttggggctctgtgttccaatccatcatgcccatttggtcatccaacaccagcaaatgaagatgcgaaagtcattgatctaatgtggtgtgacaagaatttgacatgtgataatcctgagtgtagaaaggcccactcttcattgtcgaagatcaaggaagtaaaaccaataagccagaagaaagcagctccacctccggttgaaaagtccttagaacaatgtaagttcggtacgcactgcaccaataaacgttgcaaatatagacatgctcgttctcatattatgtgccgtgaaggagcaaactgtactagaattgattgtttatttggccatccaattaatgaagattgtagatttggtgtcaattgtaagaatatttactgtctattcagacatcctccaggcagagtacttccggaaaagaaaggcgctgcacccaattcaaacgttcctaccaatgaaaggccatttgcattgccagaaaacgcaataattgaaaatgctcctccgcaaaccagttttacgcaccaagaacaa

SEQ ID NO: 83 - DNA, 1296 bp, Candida albicans
atgcaatttgctccagataaccaaataggcaaagagttacagcaaaacttgattcaagaaatacaaaggcgtttcaataaaccggctgatgatgccgtagatattgctgactatatcatctacttgattgtggcaaaaaagagcgaacaagaaatagtcgcagaagtcaaagatattgctgacatatctattgatgttgggtttattggggatgtttatctggaaatcagaaagttggaagtaaaatataatcaacctcctgctgcagtggaggaagcttctcaacctcaacaagaacagcaacagcaatctcaagcttctgtagtggctccacaaattcctattggtcctaagaaacaattaactgaggaagagaagattgcccttcgaagtcaaagatttggaactactactagattgagtgggcgaggtggacgtggtggtataactaaaactagaaccgatttcagaaatgggcacaataataagaacttcctagaccctaaaaaattagaccaaataatttctggtgccaataatggggctattaagtttgtaccactcccaccaaaaggtagatgtccagatttcccatattgtaagaatcagaattgtgaaaaagctcatccaacaaaaaactgtttcaactacccggattgccctaacccaccgggaacatgtaattttttgcatccggatcaagaccaagagttgattgctaaattagaaacatctaaaaaagaatttgaagaaaagaaaaagaatcaacttatggtcaaacaaggctcatgtaaatatggtttgaaatgtgctaaagaaaattgtccatttgctcacccaacaccagctaatcctgaatctggtaagattgaaactttggaatggtgtccacaaggtaagaattgtcaagatagaaattgtactaaatcacatccacctccacctacggcaaactcagaaaaattattatcagctgctgacttggcattggaacaatgtaaatttggttcacaatgtactaatctcaaatgtccaagaagacatgcaacttcggctgtgccatgtcgtgctggtgctgaatgtagaagagtcgattgtacattttcccatccattgaaagaaccatgccgttttggaacaaaatgtacaaataaagtgtgtatgtaccaacatcctgaaggaagaactattgcctctcacacttggaccaaggatggtagtggcaataataacagtacctcaaatcgatcatttgctgtttctgaagatcagattatggaacaagttgctcaatag

SEQ ID NO: 84 - DNA, 680 bp, Homo sapiens
  ref <308>: DATABASE ACCESSION NUMBER: Human Genbank/AF155107.1
  ref <309>: DATABASE ENTRY DATE: 2000-01-05
  ref <313>: RELEVANT RESIDUES: (1)..(680)
acccacagcagttgcacttgctgagcaggcagcttgaggacccaaatggtagcttttctaacgctgagatgagtgaactgagtgtggcacagaaaccagaaaaacttttggagcgctgcaagtactggcctgcttgtaaaaatggggatgagtgtgcctaccatcaccccatctcaccctgcaaagccttccccaattgtaaatttgctgaaaaatgtttgtttgttcacccaaattgtagatacggaaatgaactgaaatatgatgcaaagtgtactaaaccagattgtcccttcactcatgtgagtagaagaattccagtactgtctccaaaaccagttgcaccaccagcaccaccttccagtagtcagctctgccgttacttccctgcttgtaagaagatggaatgtcccttctatcatccaaaacattgtaggtttaacactcaatgtacaagaccggactgcacattctaccatcccaccattaatgtcccaccacgacatgccttgaaatggattcgacctcaaaccagcgaatagcacccagtcctgcctggcagaagatcatgcagtttggaagttttcatgtctgatgaaagatctctacagaacttgtcaaatctttgaaacttggaatatattgctttcataatatgaaggtttattggctatctaaaa

SEQ ID NO: 85 - DNA, 1140 bp, Saccharomyces cerevisiae
  ref <308>: DATABASE ACCESSION NUMBER: Genbank/CAA88520.1
  ref <309>: DATABASE ENTRY DATE: 1999-02-10
  ref <313>: RELEVANT RESIDUES: (1)..(1140)
atggcaaattcgccgaaaaagccatctgatggcactggagtatcagcgtcagacacgcctaaatatcaacataccgtcccagaaacgaaaccagcatttaatttgtcaccaggtaaagctagtgagctatcacatagccttccgtcgcctagccagataaaatcaaccgcacatgtatcttcaactcacaatgatgcggcaggtaatacggatgattctgttcttcctaaaaatgtatcacccacaactaatttgagagttgaaagtaatggagatacaaacaatatgttctctagccctgctggactagctctaccaaaaaaggatgataaaaaaaaaaacaagggtacgagtaaagcagattctaaagatggcaaagcatccaactcctcaggacagaatgcacaacaacaatcagacccaaataaaatgcaagatgtccttttttccgcaggtatcgatgttagggaggaggaggctcttctaaattcatctattaatgcctcaaaatcccaagttcaaacaaataacgttaagatccccaaccatttaccattccttcacccggaacaagtttccaattatatgaggaaagtcggaaaagagcaaaacttcaacctgacccctacaaagaatcctgaaattttggacatgatgtcaagtgcctgcgaaaactatatgagagatatcctaacaaatgccattgtcatctcccgacatagaagaaaagcagtcaagataaattctggtagaagaagtgaagtttctgcggctttaagagccattgcactaattcaaaaaaaagaagaagaaaggcgtgtgaaaaaaagaattgcgttgggactcgagaaggaagattatgaaaataagattgattccgaagagacgttacacagagcatcgaacgttacggctggccttagagcaggtagtaaaaaacagtatggttggctaacttcatcagtaaataagccgacgtccttgggagcaaaatcttcaggcaaagtcgcctccgacatcacggctagaggagaaagtgggctaaagtttagagaagctagagaggagcctggtatagtaatgagggatttactctttgctctcgaaaataggcgcaacagcgttcagactattatttca

SEQ ID NO: 86 - DNA, 1119 bp, Candida albicans
aatcaccgaattatatcacataaatccatgacaagtacacctcaagaatcctctaatttaaagagacaattagaaaacagtgaggactccagctcaccaaataaggaatctaaaacagagactaccacggaaaaccagagctcatgggagtctgactttaatagtttaccagtggaattactacaaactgaaacaaatggtacatcaccagcaccagcaccagcaacaccgatcgataccaccaatgcatcaagcacaaaggaacgtgatcaggatacttctaaattaaatgacgcgattgctgctgcaggagttgatattcaacaagaagaagagatattattacaacaacaattaaatagaaaatctgcagagggtatggcaagcaatctaaaaagtgtgatcaggtccagcaaactgcctccatttctacacaattaccatttagctgcctttattgataaagtggctaaacaaaatggaattcaacagaatttcttaatggatggtgagatgttggaattaatttcagctgcttgtgagacttggttaagtaatctagcaacaaaaacgataatcttgtcacgccacaggagaaggggaatacctgttattaataagaagtcaggaagtagttcagttccaagatcagaaatttcaaaagaattgagaagcttggccttaaaacaaaaggaaatggaagagaaacgagtgaataaaagagtgatgttggggttggaaaaaagcaccaaagacgcatccaaaaatgacgaaaatggtgaatcaaaagctggtgctgaagaaacattacatcgtgcagcaaatgctacagctgcaatgatgactatgaatcccgggagaaagaaatatagttggatgacttcaagtgctacagcaggcggtgggtcagactttggtaaatcaagtggtggctcatcaaaggactcgggaaaacaccaaagtcctattatttcagtacgtggtgataatggccttaggtttagagaaataaggtcaggtaattccattattatgaaagatttgttaggcgcaattgaagatgaaaaaatgggtacgagaaatgctgtaataaaaggatatgcaaaattgaaagattaa

SEQ ID NO: 87 - DNA, 2307 bp, Homo sapiens
  ref <308>: DATABASE ACCESSION NUMBER: Human Genbank/Y11354.1
  ref <309>: DATABASE ENTRY DATE: 1997-06-25
  ref <313>: RELEVANT RESIDUES: (1)..(2307)
atggcggcgggctcggatctgctggacgaggtcttcttcaacagcgaggtggacgagaaagtggtgagcgacctggtgggctcgctggagtcgcagctggcggccagcgcggcccaccaccaccacctcgcgccgcgcacgcccgaggtgcgggccgcggccgccggcgcgctcgggaaccatgttgtgagcggcagcccggccggagccgcgggcgcagggccggccgcccccgccgagggcgcgcccggagcggcgccggagccgccccccgcaggtagagcgcggccggggggcggggggccgcagcgcccgggccccccctcaccgcgccgcccccttgtccccgcagggcccgcgccgcccgccgcgaagctgaggccgccgcccgagggcagcgcgggggcctgcgccccggtgcccgccgccgccgccgtcgccgcggggcccgagcccgcccccgccggccccgccaagcccgccggccccgccgcgctggccgcccgcgccggccccggccccgggcccggccccggccccggccccggccctggcaagcccgccggccccggcgccgcgcaaactttgaatgggagcgccgcgctgctgaactcgcaccacgccgccgcacctgctgtcagcctggtcaacaacgggcccgccgcgctgctgccgctgcccaagcccgccgcccccggcactgtcatccagacgccccccttcgtgggcgccgccgcgccccccgcgcccgccgcgccctcgccccccgccgcccccgcgcccgccgcccccgccgccgccccgcccccgccaccccccgcgcccgccaccctggcccggccgcccggccaccccgccggacccccgaccgccgcgcccgccgtgccgccccccgccgccaagggttatgccaagatcagagattaagcccagaacgggggcagcgccggggcagcccccgcccccgccccggccgccgggggccccgctggggtcagcggccagcccgggcccggcgcggcggctgcggcgccggcgccgggggtcaaggccgagtcgcccaagagggtggtgcaggcggcgcccccggcggcgcagaccctggcggccagcggcccggccagcacggcggccagcatggtcatcgggccaactatgcaaggggcgctgcccagcccggccgccgtcccgccgcccgcccccgggacccccaccgggctgcccaaaggcgcggccggcgcagtgacccagagcctgtcccggacgcccacggccaccaccagcgggattcgggccaccctgacgcccaccgtgctggccccccgcttgccgcagccgcctcagaacccgaccaacatccagaacttccagctgcccccaggaatggtcctcgtccgaagtgagaatgggcagttgttaatgattcctcagcaggccttggcccagatgcaggcgcaggcccatgcccagcctcagaccaccatggcgcctcgccctgccacccccacaagtgcccctcccgtccagatctccaccgtacaggcacctggaacacctatcattgcacggcaggtgaccccaactaccataattaagcaagtgtctcaggcccagacaacggtgcagcccagtgcaaccctgcagcgctcgcccggcgtccagcctcagctcgttctgggtggcgctgcccagacggcttcacttgggacggcgacggctgttcagacggggactcctcagcgcacggtaccaggggcgaccaccacttcctcagctgccacggaaactatggaaaacgtgaagaaatgtaaaaatttcctatctacgttaataaaactggcttcatctggcaagcagtctacagagacagcagctaatgtgaaagagctcgtgcagaatttactggatggaaaaatagaagcagaagatttcacaagcaggttataccgagaacttaattcttcacctcaaccttaccttgtgcctttcctgaagaggagcttacccgccttgagacagctgacccccgactccgcggccttcatccagcagagccagcagcagccgccaccgcccacctcgcaggccaccactgcgctcacggccgtggtgctgagtagctcggtccagcgcacggccgggaagacggcggccaccgtgaccagtgccctccagccccctgtgctcagcctcacgcagcccacgcaggtcggcgtcggcaagcaggggcaacccacaccgctggtcatccagcagcctccgaagccaggagccctgatccggcccccgcaggtgacgttgacgcag

SEQ ID NO: 88 - DNA, 555 bp, Saccharomyces cerevisiae
  ref <308>: DATABASE ACCESSION NUMBER: Genbank/CAA82029.1
  ref <309>: DATABASE ENTRY DATE: 1997-08-11
  ref <313>: RELEVANT RESIDUES: (1)..(555)
atgaacaccaatagtaatactatggtaatgaatgacgcaaatcaagcacaaataacggccacatttacgaagaagatattagcgcatttggatgatccggactccaacaaattggcccaattcgtacagctttttaatccaaacaactgcagaataatatttaatgctacccccttcgcgcaagcaacagtttttctgcaaatgtggcaaaaccaggtcgtacaaacacaacatgccctaacaggagtagactatcacgctattccgggatccggcacgttgatatgcaacgtcaattgcaaagtcagattcgacgaaagcggcagagacaagatggggcaagacgcgactgttcccattcaaccaaataacactgggaacagaaatcgacccaacgatatgaacaagccaagacctctatggggtccatattttggcatttccctgcagctgatcatcgacgaccgcatatttagaaatgattttaatggtgtaatatcggggtttaactataacatggtttacaaacccgaggattctctgctaaaaatttag

SEQ ID NO: 89 - DNA, 540 bp, Candida albicans
catcctatagcacaacaactagagccgtttctcaaacgatttcttgcatcgttagatttactgtacacacagccaacatcacaaccattccccaacgttgaatcgtatgccactcagttaggatcaaacttaaagcggtcaagtgcaattatagtgaacggccagcctattataccgagcccacaagaagactgtaaattacaattccaaaagaaatggttacaaactccgttatcgtcacaccaattgacaagttacgatgggcatttaattccaggcacggggacctttgtcgttcatttttcagcaaaagtaagatttgatcaaagtggaaggaaccggttaggtgaatctgccgacttgtttcaggaaaataattcaattgtttccaaaaccaatcaaagacctatttggggttcgtggtttggagtcgacgtcaatttggttgttgacgaaaacgttatgcaagatggagagattataaatagtatggattatagatttacctatgtacctaacgatagcattataaaagtataa

SEQ ID NO: 90 - DNA, 720 bp, Saccharomyces cerevisiae
  ref <308>: DATABASE ACCESSION NUMBER: Genbank/CAA97636.1
  ref <309>: DATABASE ENTRY DATE: 1997-06-17
  ref <313>: RELEVANT RESIDUES: (1)..(720)
atgaacgctctttacaaccatgctgtgaagcaaaaaaatcaactacaacaagagttggccaggtttgaaaagaattctgtgaccgcccctatttctttacaagggtccatctctgcaactctggtctcactggagaaaacagttaagcaatatgcagaacatttaaacagatataaagaagatactaatgcagaggaaattgatcctaagttcgctaatcgactagcaactttaacacaggatctgcacgactttactgccaagtttaaggatttaaaacaatcctacaacgaaaataattccagaactcagttgtttggctcaggagcatcgcatgttatggactccgataacccctttagtacatcagagaccatcatgaataaaaggaacgttggtggtgcgagtgcaaatggtaaagagggctctagcaacggtgggggactaccgttgtaccaagggctacaaaaggaacagtctgttttcgaaaggggtaacgctcaattagattacattctagaaatgggccaacaatcattcgaaaatatagtggaacaaaacaaaattttatccaaggtacaagatagaatgtcaaatggcctaagaacattgggtgtttcggaacaaactatcacctctatcaataaacgggtgttcaaagataaactagtcttttggatcgcgttaattctcttgatcataggtatttattatgtgttg

SEQ ID NO: 91 - DNA, 483 bp, Candida albicans
atgaattcaatatataatcatggtttaaaacaaacccaaactataactaaagatttaactcaattcgagaaaaacttatccacatcaccattatcattacaagtgcaatcacaacatcattaactgcattcaggaaaactatcgaagaatatgatgatttattggaagtaaatgtctatgatacatctgataccatagatgagggtagattagatatattcaatccagatttaaatgaatacactctgaaatatgatactttaaataagctacgtgagtttcttctccatcaagctaataaacaagaattattaggagaaggacacttatcaccaacagcaacagcagcattggatcgacatcatcagataatccgtatgaatctagctcaaatccatctcaacaacaacaacagcaattacaagatgaacaaaacaccatgtcttatagagaaggattatatcatgaaaagaattctctaga

SEQ ID NO: 92 - DNA, 1560 bp, Homo sapiens
  ref <308>: DATABASE ACCESSION NUMBER: Human Genbank/NM_003569.1
  ref <309>: DATABASE ENTRY DATE: 2000-11-01
  ref <313>: RELEVANT RESIDUES: (1)..(1560)
gagggagccgtggaggtccaggtgactgcttagaaaactgcacagcatctgatgaaattagcgaataagaacatcaaccatgtcttacactccaggagttggtggtgaccccacccagttggcccagaggatctcttctaacatccagaagatcacacagtgttctgtggaaatacaaagaactctgaatcaacttggaacacctcaagattcacctgaattgaggcaacagttgcaacagaagcagcagtatactaaccagcttgccaaagaaacagataagtacattaaagagtttggatctctgcccaccacccccagtgaacagcgtcaaaggaaaatacagaaggatcgcttagtggcagagttcacaacatcactgacaaacttccagaaggtccagaggcaggctgctgagcgagagaaagagtttgttgctcgagtaagagccagttccagagtgtctggcagttttcctgaggacagctcaaaagaaaggaatcttgtatcctgggaaagccaaactcaacctcaagtgcaggtgcaggatgaagaaattacagaggatgacctccgtcttattcatgagagagaatcttctatcaggcaacttgaagctgatattatggatattaatgaaatatttaaagatttgggaatgatgattcatgaacaaggagatgtaatagatagcatagaagccaatgtggaaaatgcagaggtgcacgttcagcaagcaaatcagcagctgtcaagggcagcagattatcagcgcaaatccagaaaaaccctgtgcatcatcattcttatccttgtcattggagttgcgattatcagtctcatcatatggggattgaaccactgaagttataaaggagcacactgtcgcactacattgtctaaattatgtaggaagattcctgtaatcatgtttttttaattattattttaaagctattgtataaaggatggttcccatactttgttatttttattgggggggttgggcgggttcctttggattaaatctgatattttctaatactgaaagattttctaaatgtcactgctgacataactcccttggtcttcaatttaatagttgttaagttttgggccacattgcatatgcctttcatttataatttatttaccctgcttgacttagtttggggaattcggaaatttaaggtgtgtgtattctgttgggatctccctgccacgtgaacacaccaagatgtgtgttacttcaagttaaaactccccaaaatttaatttttgatttgcttccaccaggggaaaatattctccaataatgtaaaataattaaggtccaatacatgggttgtatttttctggttcacaacagcacaaagtgtctttcatttttttgttggatttcctttaagatcttttttaccctgaagtcggtgaacacttttctagttaatttgatactctttctgtgtatataataagcttttgctgtagattgcctagtaaaattactaaggataggttgtttttacatatggtctatttaagtctgatgtttacggg

SEQ ID NO: 93 - DNA, 720 bp, Saccharomyces cerevisiae
  ref <308>: DATABASE ACCESSION NUMBER: Genbank/CAA85038.1
  ref <309>: DATABASE ENTRY DATE: 1999-06-24
  ref <313>: RELEVANT RESIDUES: (1)..(720)
atgttagaagcaaaatttgaagaagcatcccttttcaagagaataattgatggtttcaaagattgtgtccagttggtcaatttccaatgtaaagaagatggtatcattgcacaagctgtcgatgactcaagagttctattggtctccttggaaataggtgtcgaagccttccaagaatatagatgtgaccatcctgttacgttaggtatggatctaacctcactaagtaaaatcctacgttgtggtaacaacaccgatacattaacactaattgctgacaacacaccggattccatcatcttattatttgaggataccaagaaagaccgtatagccgaatactctctgaaattgatggatatcgatgctgatttcttaaagattgaagaattacagtacgactccaccctgtcattgccatcttccgaattctctaaaattgttcgtgacttgtcccaattgagtgattctattaatatcatgatcaccaaagaaacaataaagtttgtagctgacggtgatatcggatcaggttcagtcataataaaaccattcgtggatatggaacatcctgaaacaagcatcaaacttgaaatggatcaacctgtcgacttgacgttcggagctaaatatttattggacatcattaagggctcctccctttctgatagagttggtatcaggctctccagcgaagctcctgctttattccaatttgat

SEQ ID NO: 94 - DNA, 780 bp, Candida albicans
atgttagaaggtaaatttgaagaagctgctttattaaaaaaagttgttgaagccattaaagattgtgttaaaaaatgtaacttcaattgttcagagcatgggattactgtacaagcagtggatgattctcgtgtattattagtttcattattaattggtcaaacttctttcagtgaatatagatgtgacagagacgttacattaggtattgacttggaaagtttcagtaagattatcaaatctgctaacaatgaagatttcttgacccttttagctgaagattcaccagatcaaataatggctattcttgaagaaaaacaaaaagagaaaatcagtgaatattctttaaaattaatggatattgattctgaatttttacaaattgatgatatggaatacgatgctgttgtgaatatgccaagtagtgattttgctaaacttgtgagggatttgaaaaatttaagtgaatctttacgtgttgttgttactaaagattccgtcaagtttacatctgaaggtgattctggttccggaagtgttatcttgaaaccttacaccaacttgaaaaatgaaagagaaagtgtcactattagtttagatgacccagttgatttgacttttggtttgaaatacttgaatgatattgtgaaggcagctacattatccgatgtcatcaccatcaaattggccgataaaactcctgcattgtttgaatttaaaatgcaatctggaggttatttgagattctacttggcaccaaaattcgatgatgatgagtag

SEQ ID NO: 95 - DNA, 1200 bp, Homo sapiens
  ref <308>: DATABASE ACCESSION NUMBER: Human Genbank/GI:181271
  ref <309>: DATABASE ENTRY DATE: 1993-04-27
  ref <313>: RELEVANT RESIDUES: (1)..(1200)
aggtctcagccggtcgtcgcgacgttcgcccgctcgctctgaggctcctgaagccgaaactagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtacagctgtgtagtaaagatgccttctggtgaatttgcacgtatatgccgagatctcagccatattggagatgctgttgtaatttcctgtgcaaaagacggagtgaaattttctgcaagtggagaacttggaaatggaaacattaaattgtcacagacaagtaatgtcgataaagaggaggaagctgttaccatagagatgaatgaaccagttcaactaacttttgcactgaggtacctgaacttctttacaaaagccactccactctcttcaacggtgacactcagtatgtctgcagatgtaccccttgttgtagagtataaaattgcggatatgggacacttaaaatactacttggctcccaagatcgaggatgaagaaggatcttaggcattcttaaaattcaagaaaataaaactaagctctttgagaactgcttctaagatgccagcatatactgaagtcttttctgtcaccaaatttgtacctctaagtacatatgtagatattgttttctgtaaataacctattttttttctctattctctccaatttgtttaaagaataaagtccaaagtctgatctggtctagttaacctagaagtatttttgtctcttagaaatacttgtgatttttataatacaaaagggtcttgactctaaatgcagttttaagaagtgttttt

SEQ ID NO: 96 - DNA, 1500 bp, Saccharomyces cerevisiae
  ref <308>: DATABASE ACCESSION NUMBER: Genbank/CAA88556.1
  ref <309>: DATABASE ENTRY DATE: 1997-08-11
  ref <313>: RELEVANT RESIDUES: (1)..(1500)
atgtcgaaaaggtctatcgaggtcaacgaggaacaagatagagtggtctctgctaaaacagaatctcactctgttcctgctattcccgcctctgaagagcaagatgctcccaagaatgacctagaagaacaattgagtgatgaatttgatagtgatggtgaaattattgaaattgatggcgatgatgagattaatgacgaagatgaccttaggaaaaagcaagaagaagctgaaactttagtacaaaaggaccaatccgaaggcaacaaagaaaagatccaggagctttacttaccccatatgtctcgtccattagggccagatgaagtccttgaggctgatcccactgtttatgaaatgctacataatgtcaatatgccatggccatgcttgacattagatgtcattccagatacactaggttctgaacgtagaaactatccacagtctattttgttgaccacggctactcaatcttccaggaaaaaggagaatgaactaatggttctagcactttctaatttagcgaaaacacttttgaaagacgataatgaaggtgaagatgatgaagaggatgatgaagatgatgtggatccagtcattgagaatgaaaatataccattgagagatacaaccaatagattaaaggtttctccttttgccatttctaatcaagaggtgttaaccgctacaatgagcgaaaatggtgatgtttatatatacaatctagctccacaaagcaaagctttttccacaccaggttatcagattccgaagtctgctaagcgtcctattcacactgtaaaaaatcatgggaatgttgaaggctacgggttggattggtcaccattgatcaagactggtgcgttactatcaggtgattgctcaggacaaatatattttacccaaaggcacacatcgagatgggtgactgataaacaaccatttactgtttcaaacaataaatccatagaagatatccagtggtctcgcactgaatccaccgtttttgcaaccgcaggatgtgatggatatataaggatttgggacacaagatcaaaaaaacataaacctgctatctctgttaaagcttctaatactgacgtaaatgtcataagttggagtgataaaattggttacttgctagcaagcggtgacgataacggtacatggggagtatgggatttaagacagtttacgccaagtaatgctgacgccgtccaaccggttgctcaatatgacttccataagggagccattacttccattgcattcaacccattagatgagtctatcgttgcggtaggctcagaagataatactgtgactttgtgggatttgtctgtagaagctgacgatgaggaaattaaacaacaggccgccgaaacaaaagagctacaagaaattccaccacaattattgtttgttcactggcaaaaggaagttaaagatgtcaaatggcataagcaaatcccaggttgtttagtaagtaccggtact

SEQ ID NO: 97 - DNA, 1554 bp, Candida albicans
atgtcaaaaagatcagctgaagatgatttaagtggcaatagatccaccagtcatactgccattaaaactaataaagattctcttccaactactacaaatggaaaggaagaagaaccagacaatatggatattggggaatttgaagatccatacggtgatgaatttgaaagtgatgaagaaattatagaattagacgataacaatgatgaagaagatgatgaaatgattgatgaaaattcaacacaagccaaaattgaagaattagaagccaaagaacaagaacaagaacaacaatcatcaatatatttacctcataaatcaaaaccattaggaccagatgaagtcttagaagccgatccaacagtctatgaaatgttgcataatatcaatttaccatggccatgtttgactgttgatattttaccagattctttaggtaatgaaagaagatcatatccagcaacagtttatttagctactgcgactcaagctgctaaagccaaagataatgaattgttagctatgaaagcatcttcattggccaaaacattagttaaagatgaaaatgaagaagatgaggaagatgaagacgatgacgatgatgttgatagtgatccaatattagattcagaatctattccattaagacatactacaaatagaataagagtaagtcctcatgctcaacaaactggggaatacttaactgcttcaatgtcagaaaatggtgaagtttatatatttgatttactggcacaatataaggcatttgacacaccaggttatatgattcctaaatcatcgaaaagaccaattcatactattcgtgcccatgggaatgttgaaggttatggattagattggtctccattagtaaatacaggggctttattatctggagatatgtcagggagaatttatttaactaatagaacgacatcaagttggaccactgataaaactccattttttgcatcacaatcttcaattgaagatattcaatggtcaactggtgaaactacagtgtttgccacgggtggatgtgatggatatatttgtatttgggatacaagatcgaaaaaacataaacctgcattatcagtaattgcttctaaatctgatgttaatgtgatatcttggagttctaaaatcaatcatttattggcatcaggacatgacgatggtagttggggtgtatgggatttaagaaatttcacaaacaataccaccagtaatccttcacctgtggctaattatgatttccataaatcgccaatcacatcaatttcattcaatccattagatgaatcaatcattgctgtttcatcagaagataatactgttacattatgggatcttgctgttgaagctgatgatgaagaaatttctcaacaaagaaaagaagctcaagaattacatgatattccaccacaattattatttgtccattggcaaagagatgttaaagatgttagatggcatccacaaattcctggttgtttggtatctactggtggtgatggattaaacatttggaaaactatatctgtgtaa

SEQ ID NO: 98 - DNA, 2280 bp, Homo sapiens
  ref <308>: DATABASE ACCESSION NUMBER: Human Genbank/NM_005610.1
  ref <309>: DATABASE ENTRY DATE: 2001-12-20
  ref <313>: RELEVANT RESIDUES: (1)..(2280)
cgcgcgcacagagcgagctcttgcagcctccccgcccctcccgcaacgctcgaccccaggattcccccggctcgcctgcccgccatggccgacaaggaagcagccttcgacgacgcagtggaagaacgagtgatcaacgaggaatacaaaatatggaaaaagaacaccccttttctttatgatttggtgatgacccatgctctggagtggcccagcctaactgcccagtggcttccagatgtaaccagaccagaagggaaagatttcagcattcatcgacttgtcctggggacacacacatcggatgaacaaaaccatcttgttatagccagtgtgcagctccctaatgatgatgctcagtttgatgcgtcacactacgacagtgagaaaggagaatttggaggttttggttcagttagtggaaaaattgaaatagaaatcaagatcaaccatgaaggagaagtaaacagggcccgttatatgccccagaacccttgtatcatcgcaacaaagactccttccagtgatgttcttgtctttgactatacaaaacatccttctaaaccagatccttctggagagtgcaacccagacttgcgtctccgtggacatcagaaggaaggctatgggctttcttggaacccaaatctcagtgggcacttacttagtgcttcagatgaccataccatctgcctgtgggacatcagtgccgttccaaaggagggaaaagtggtagatgcgaagaccatctttacagggcatacggcagtagtagaagatgtttcctggcatctactccatgagtctctgtttgggtcagttgctgatgatcagaaacttatgatttgggatactcgttcaaacaatacttccaaaccaagccactcagttgatgctcacactgctgaagtgaactgcctttctttcaatccttatagtgagttcattcttgccacaggatcagctgacaagactgttgccttgtgggatctgagaaatctgaaacttaagttgcattcctttgagtcacataaggatgaaatattccaggttcagtggtcacctcacaatgagactattttagcttccagtggtactgatcgcagactgaatgtctgggatttaagtaaaattggagaggaacaatccccagaagatgcagaagacgggccaccagagttgttgtttattcatggtggtcatactgccaagatatctgatttctcctggaatcccaatgaaccttgggtgatttgttctgtatcagaagacaatatcatgcaagtgtggcaaatggcagagaacatttataatgatgaagaccctgaaggaagcgtggatccagaaggacaagggtcctagatatgtctttacttgttgtgattttagactccccttttttcttctcaaccctgagagtgatttaacactggttttgagacagactttattcagctatccctctatataataggtaccaccgataatgctattagcccaaaccgtgggtttttctaaatattaataggggggcttgattcaacaaagccacagacttaacgttgaaattttcttcaggaattttctagtaacccaggtctaaagtagctacagaaaggggaatattatgtgtgattatttttcttcttatgctatatccccaagtttttcagactcatttaagtaaaggctagagtgagtaaggaatagagccaaatgaggtaggtgtctgagccatgaagtataaatactgaaagatgtcacttttattcaggaaatagggggagttcaagtcgtatagattcctactcgaaaatcttgacacctgactttccaggatgcacattttcatacgtagaccagtttcctcttggtttcttcagttaagtcaaaacaacacgttcctctttccccatatattcatatatttttgctcgttagtgtatttcttgagctgttttcatgttgtttatttcctgtctgtgaaatggtgtttttttttttgttgttggttttttttttttttttttaacttgggaccaccaagttgtaaagatgtatgtttttacctgacagttataccacaggtagactgtcaagttgagaagagtgaatcaataacttgtatttgttttaaaaattaaattaatccttgataagagttgctttttttttttaggagttagtccttgaccactagtttgatgccatctccattttgggtgacctgtttcaccagcaggcctgttactctccatgactaactgtgtaagtgcttaa

SEQ ID NO: 99 - DNA, 1144 bp, Saccharomyces cerevisiae
  ref <301>: AUTHORS: Bauer and Burgers
  ref <302>: TITLE: Molecular cloning, structure ande expression of the yeast proliferating cell nuclear antigen gene
  ref <303>: JOURNAL: Nucleic Acids Research
  ref <304>: VOLUME: 18
  ref <305>: ISSUE: 2
  ref <306>: PAGES: 261-265
  ref <307>: DATE: 1990
  ref <308>: DATABASE ACCESSION NUMBER: x16676
  ref <309>: DATABASE ENTRY DATE: 1993-09-30
acgcgtaactttttttttttggatttcaactgatagttttcgtactttgcttcctctggtacataaaattatatataagaaacacttttgctttagccttcctttctttccacttgcacctttcactttcgccgtcctttttcactcacagcaacaagcagcaagcactaagtacgcagtcaaaagagagaaaaaatgttagaagcaaaatttgaagaagcatcccttttcaagagaataattgatggtttcaaagattgtgtccagttggtcaatttccaatgtaaagaagatggtatcattgcacaagctgtcgatgactcaagagttctattggtctccttggaaataggtgtcgaagccttccaagaatatagatgtgaccatcctgttacgttaggtatggatctaacctcactaagtaaaatcctacgttgtggtaacaacaccgatacattaacactaattgctgacaacacaccggattccatcatcttattatttgaggataccaagaaagaccgtatagccgaatactctctgaaattgatggatatcgatgctgatttcttaaagattgaagaattacagtacgactccaccctgtcattgccatcttccgaattctctaaaattgttcgtgacttgtcccaattgagtgattctattaatatcatgatcaccaaagaaacaataaagtttgtagctgacggtgatatcggatcaggttcagtcataataaaaccattcgtggatatggaacatcctgaaacaagcatcaaacttgaaatggatcaacctgtcgacttgacgttcggagctaaatatttattggacatcattaagggctcctccctttctgatagagttggtatcaggctctccagcgaagctcctgctttattccaatttgatttgaagagtgggttcctacagtttttcttggctcctaaatttaatgacgaagaataaatgtaaattatctatatagttgtatactaaaaataataaacaaaaaaaaaacagtaaagtttgttttaaatgaaaataaataacaaagaaaataaagactaagtagtcagttaatatcagcatttttgtgtgacttatacagtatttatgacatatcttacattaatctaga

SEQ ID NO: 100 - DNA, 1245 bp, Candida albicans
atgtcacaccaacaagaagacgtcgtagacgatactcaagaagaatatatcaatgttaatgaagtggctgaggaagttgcagatgatgatcaagcgccacccgatgaagaagatgaggagatggaattagatgatgagcatgagactttagaaattgacatgtccaacaattcatggacttattttgataaacataccgatagtatatttactattttttcacatcctaaattgccaatggtattgactgagggtggtgacaacacggcatacttatggaccacacacacccaaccaccaagatttgttggcgaaatcactggacataaagagtctgttatatctggagggtttactgcagacggcaagtttgttgttactgcagacatgaatggattaattcaagttttcaaagccacaaaaggaggtgaacagtgggtgaaatttggtgaattggacgaagttgaagaagtgttgtttgttactgtgcatccaacattaccattctttgcctttggtgctaccgatggatctatatgggtctaccaaatagacgaatccagtaaactgctagtgcaaattatgtctgggttctcacacacattaaaatgtaatggtgctgtatttatacaaggaaaagatgaaaatgatttgacattggtctctataagtgaagatggtactgtggtgaactggaactgttttacaggacaagtgaattataaattgcaacctcatgatgactttaaaggagttgagagtccgtgggtcacggtcaaagtacatggtaatcttgtggccattggtggcagagatggccagctatcaattgtgaacaatgacactggtaaaatcgttcatactcttaaaacattggataatgtcgacgacattgcagaactctcaattgaggcattgagttggtgtgaaagcaaaaatattaacctcttggcagtgggtttggtttctggtgacgtttattatttgatactcagcaatggagattgagaaagaacttgaaagttgacgatgccatcaccaaattacaatttgttggcgaaacccccattttggtgggaaatagtatggatggtaaaattacaaatgggaacctagaactggtgaaaaatgtttgctggtgtgggaacaaacatgggagtattggactttgctattttagatggaggtaaaaagttggttactgctggtgatgaaggtgttgcattggtctttgtacatgaatag

SEQ ID NO: 101 - DNA, 1231 bp, Homo sapiens
  ref <301>: AUTHORS: Almendral, Huebsch, Blundell, MacDonald-Bravo and Bravo
  ref <302>: TITLE: Cloning and sequence of the human nuclear protein cyclin: Homology with DNA-binding protein
  ref <303>: JOURNAL: Proc. Natl. Acad. Sci. U.S.A.
  ref <304>: VOLUME: 84
  ref <305>: ISSUE: 6
  ref <306>: PAGES: 1575-1579
  ref <307>: DATE: 1987
  ref <308>: DATABASE ACCESSION NUMBER: M15796
  ref <309>: DATABASE ENTRY DATE: 1993-04-27
aggtctcagccggtcgtcgcgacgttcgcccgctcgctctgaggctcctgaagccgaaactagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtacagctgtgtagtaaagatgccttctggtgaatttgcacgtatatgccgagatctcagccatattggagatgctgttgtaatttcctgtgcaaaagacggagtgaaattttctgcaagtggagaacttggaaatggaaacattaaattgtcacagacaagtaatgtcgataaagaggaggaagctgttaccatagagatgaatgaaccagttcaactaacttttgcactgaggtacctgaacttctttacaaaagccactccactctcttcaacggtgacactcagtatgtctgcagatgtaccccttgttgtagagtataaaattgcggatatgggacacttaaaatactacttggctcccaagatcgaggatgaagaaggatcttaggcattcttaaaattcaagaaaataaaactaagctctttgagaactgcttctaagatgccagcatatactgaagtcttttctgtcaccaaatttgtacctctaagtacatatgtagatattgttttctgtaaataacctattttttttctctattctctccaatttgtttaaagaataaagtccaaagtctgatctggtctagttaacctagaagtatttttgtctcttagaaatacttgtgatttttataatacaaaagggtcttgactctaaatgcagttttaagaagtgtttttgaatttaaataaagttacttgaatttcaaac

SEQ ID NO: 102 - DNA, 840 bp, Saccharomyces cerevisiae
  ref <308>: DATABASE ACCESSION NUMBER: Genbank/BAA77792.1
  ref <309>: DATABASE ENTRY DATE: 1999-07-29
  ref <313>: RELEVANT RESIDUES: (1)..(840)
atgtctgctcccactatgagatccacctcaatattgacagagcatttgggatatccgcccatctcgcttgttgatgatatcattaatgctgtaaatgaaattatgtacaagtgcactgctgccatggaaaaatatctgctatccaagagcaaaatcggcgaggaagattatggagaagagatcaaaagtggagttgctaagttggaatcacttttggaaaactccgtggataagaattttgacaaactagaactatatgttttgaggaacgtccttcgaatccctgaagagtatttggacgccaatgtttttagattggagaaccaaaaggatctggtcattgtagatgagaatgagttgaagaaaagtgaggagaaacttcgagagaaagtgaacgacgtggagttagcgttcaaaaagaatgaaatgctattgaaaagagttacaaaagtgaaaagactgttgtttacgataagaggattcaaacaaaagctaaacgagttactgaaatgcaaagacgatgtacaattgcagaaaattttggagtcgttaaaacctatagatgacacaatgactctactgactgattcattacgtaaactatatgttgatagtgaaagtaccagttcaacagaggaggtagaggcactactgcagagattgaagaccaacgggaagcaaaataataaggatttcagaacacgatatatcgatataaggacgaataatgtcctacgaaaattggggctactaggtgataaagaggacgaaaaacagtctgccaagccggatgcgaggacgcaagcaggggatatagttagtatagatattgaagagcct

SEQ ID NO: 103 - DNA, 945 bp, Candida albicans
atgtcagataaaactttagacgaacgtactacagcaattcttactgaacatttagaatttgctcccttgacacttattgatgacgtgatcaatgcggtgaatgaaatcatgtacaagggaacaacagctattgaaacatatttaaaagaacaaaaacaattaatgaaaaatgggatatttaccaaagttactgaagatgaaatagaaattggtatggggaaattagaatcattattagaatcgactatagataagaattttgataaatttgaattatattgtttaagaaatattttcaatatacctaaagatctaataccatatatacagttaagccatcaacaaggaattgaatttaaaagtgataatgttgaacaaaaacgtgaatttgatcaacaaattaaaaatttacaattgaaaatcatgcaagaattacaacttcgaaaaatcttaaaattacaacttgtcaaagtccaaaaattaattaaagtattaatagccattgataatgatttcaagaaaatagattttgctagtggtggtggtggtaatgaagaatcaataagaattttgaaaaatcttcaacctattgatgaaacattatattttttaattagtcaaattaaaaatctaataaatcaaattgaacaattatcaaataaagttaataccaatttgaaaactcaaaaatttatacccaatttgcgtgataaattcattgatggtagaacatttagagttttacaacaaacggggatttggaaagatttggaaaaaaatgatatcaagattctggtgcagggaaatgacaataataataataataataataataataataataccttaacagatttacaaaatcaagacgacattgatatgataataccagaacaagacgatatagatgtggatgcaataaagaatataaatgctcaaatttaa

SEQ ID NO: 104 - DNA, 5718 bp, Saccharomyces cerevisiae
  ref <308>: DATABASE ACCESSION NUMBER: Genbank/AAB64747.1
  ref <309>: DATABASE ENTRY DATE: 1997-08-01
  ref <313>: RELEVANT RESIDUES: (1)..(5718)
atgtcacattccggagctgccatttttgagaaagtttctgggataattgccataaatgaggatgtttcacccgcagaattgacatggaggtctacggacggtgacaaggttcacacagttgtcttatccactattgacaagttacaagctacccctgcttccagtgaaaaaatgatgttgaggctaatcgggaaagtggatgagtcaaaaaagagaaaagacaacgaaggaaatgaggttgtgcccaaaccgcaacgtcatatgttttcgtttaacaatagaacagttatggataatatcaagatgacccttcaacaaatcatctcacggtataaagatgcagatatctacgaagaaaagagaagaagagaggagtctgcgcaacacacagaaacaccaatgagctcttcttctgttactgcagggactcccacaccacatctcgatacaccacaattgaataatggggctccgttgattaatacagccaaactagatgattctctctctaaagaaaaattgttgaccaatttaaagctacagcaatctttactgaaaggaaacaaagttctaatgaaggtttttcaggaaacagtcattaacgccggtttgcctccatctgaattttggtcaactagaattccgttattgagggcttttgccttatctacttctcaaaaagttgggccttacaacgttttgtcaactatcaagccggtggcttcatcggaaaacaaagtcaatgttaatttgtcaagagaaaaaattttgaatatttttgagaactatccaattgtaaagaaagcttacactgataatgtgcccaaaaatttcaaagaaccagagttctgggcaaggttcttctcttcgaagttattcagaaaattaaggggtgaaaagatcatgcaaaatgatagaggtgacgtaatcattgacaggtacttgacattggatcaagagttcgacagaaaagatgatgacatgctattgcatcctgtgaaaaaaattatagatttagatggtaacatacaggacgacccagttgtacgaggcaacaggcccgacttcactatgcagccaggtgtggatattaatggtaatagcgatggtaccgtggacatcttaaagggtatgaatagattgagtgaaaaaatgattatggctttgaagaatgagtattcaaggacaaatctacagaacaaatctaatattacaaacgatgaggaagatgaagataatgatgaaagaaatgaactgaaaatcgatgacttaaacgaaagctacaagacaaactatgcaatcatacatctgaaaaggaacgcacatgaaaagacaaccgacaacgatgcgaaaagctcggcagactcgataaagaatgcagatttgaaggtttctaatcaacaaatgttacaacagttgtcattggtcatggataatttaattaataagctagacttgaaccaagtagttcctaacaacgaagtcagcaacaagatcaataaaagagtcataactgcaatcaagattaacgccaaacaggctaagcataacaatgttaattcagcactcggctcttttgtcgacaacacttctcaagcaaatgaattagaggtgaaaagtaccctaccaatagacctattagaaagttgtagaatgctacacacaacgtgctgtgaatttctaaagcacttttatattcattttcagagcggtgaacaaaagcaagccagtaccgtcaaaaaactttataatcatttgaaggactgtattgaaaagctgaatgagctatttcaagacgtccttaatggtgatggtgaatctatgtcaaacacatgtaccgcctatttgaagccagttttgaactccattactttggctactcataagtacgatgagtacttcaacgaatataacaacaattcgaactaggatgtttcacccgcagaattgacatggaggtctacggacggtgacaaggttcacacagttgtcttatccactattgacaagttacaagctacccctgcttccagtgaaaaaatgatgttgaggctaatcgggaaagtggatgagtcaaaaaagagaaaagacaacgaaggaaatgaggttgtgcccaaaccgcaacgtcatatgttttcgtttaacaatagaacagttatggataatatcaagatgacccttcaacaaatcatctcacggtataaagatgcagatatctacgaagaaaagagaagaagagaggagtctgcgcaacacacagaaacaccaatgagctcttcttctgttactgcagggactcccacaccacatctcgatacaccacaattgaataatggggctccgttgattaatacagccaaactagatgattctctctctaaagaaaaattgttgaccaatttaaagctacagcaatctttactgaaaggaaacaaagttctaatgaaggtttttcaggaaacagtcattaacgccggtttgcctccatctgaattttggtcaactagaattccgttattgagggcttttgccttatctacttctcaaaaagttgggccttacaacgttttgtcaactatcaagccggtggcttcatcggaaaacaaagtcaatgttaatttgtcaagagaaaaaattttgaatatttttgagaactatccaattgtaaagaaagcttacactgataatgtgcccaaaaatttcaaagaaccagagttctgggcaaggttcttctcttcgaagttattcagaaaattaaggggtgaaaagatcatgcaaaatgatagaggtgacgtaatcattgacaggtacttgacattggatcaagagttcgacagaaaagatgatgacatgctattgcatcctgtgaaaaaaattatagatttagatggtaacatacaggacgacccagttgtacgaggcaacaggcccgacttcactatgcagccaggtgtggatattaatggtaatagcgatggtaccgtggacatcttaaagggtatgaatagattgagtgaaaaaatgattatggctttgaagaatgagtattcaaggacaaatctacagaacaaatctaatattacaaacgatgaggaagatgaagataatgatgaaagaaatgaactgaaaatcgatgacttaaacgaaagctacaagacaaactatgcaatcatacatctgaaaaggaacgcacatgaaaagacaaccgacaacgatgcgaaaagctcggcagactcgataaagaatgcagatttgaaggtttctaatcaacaaatgttacaacagttgtcattggtcatggataatttaattaataagctagacttgaaccaagtagttcctaacaacgaagtcagcaacaagatcaataaaagagtcataactgcaatcaagattaacgccaaacaggctaagcataacaatgttaattcagcactcggctcttttgtcgacaacacttctcaagcaaatgaattagaggtgaaaagtaccctaccaatagacctattagaaagttgtagaatgctacacacaacgtgctgtgaatttctaaagcacttttatattcattttcagagcggtgaacaaaagcaagccagtaccgtcaaaaaactttataatcatttgaaggactgtattgaaaagctgaatgagctatttcaagacgtccttaatggtgatggtgaatctatgtcaaacacatgtaccgcctatttgaagccagttttgaactccattactttggctactcataagtacgatgagtacttcaacgaatataacaacaattcgaactagatggaactagagcccactctttttggtataatagaggcattggctcctcaattattgtcgcagagtcatttgcagacatttgtatctgatgtagtcaatttactgcgatcatccaccaaatcggcaactcaattaggccctttaattgatttttacaaattacaatcactagattcgcctgaaacaacaattatgtggcataaaattgagaaatttctcgatgctttatttggaatccagaacaccgatgatatggtaaagtacctctctgtctttcaatctttgcttccatcaaattacagagcaaaaattgtccaaaaatcatctgggctcaatatggagaaccttgctaaccatgaacatttacttagcccagtgcgggctccaagtatatatacagaagcttcatttgaaaacatggaccgattttctgaaagaaggtccatggtatcttcgcctaatcgttacgttccctcttcaacctacagttctgttactttgagacagttgtcaaatccttattatgtgaacactatacccgaggaagatatcctaaaatacgtatcatatacattattagctacgacatcggcactatttccgtttgatcatgagcaaatacaaattccgtctaagatacccaattttgagagtggacttttacatttaatatttgaagcgggtttattatatcaaagtttgggttataaagtggagaagtttaggatgttgaatatatctccaatgaaaaaagcattgattatagaaatttcagaagaattacaaaactacacagcatttgtgaacaatctggtctcttcagggacagtagtgtcattgaaatcgttatatcgtgaaatatatgaaaatataataaggcttcgaatatactgtaggtttacagaacaccttgaagaattgagcggagatacattcttgattgaattaaatattttcaaatcccacggagatcttactataagaaaaatagcaacgaatttgtttaattcaatgatttctctttattatgagtatttaatgaattggttgactaaaggtctactccgagctacttatggagaattcttcattgctgaaaacactgatacaaatggtacagacgatgattttatttaccacattcctatagagttcaaccaagaaagagttccggccttcataccgaaagagttggcatataaaatattcatgatcggcaaatcgtatatcttcctagaaaagtactgtaaagaggttcaatggacaaacgaattttctaaaaagtatcatgtcctgtaccagagcaattcttatcggggaatatcaacgaacttttttgaaattataaatgatcaatattctgaaattgttaatcatactaatcaaattctaaatcagaagtttcattacagagacgtggtatttgcgttaaagaatattcttctcatgggtaaatctgattttatggatgctcttatagaaaaggccaatgatattctcgcgacaccatcggattcattgccaaattataagttaacaagggttttacaggaagccgtgcagctttcttccttaagacatttaatgaatagtccccgtaatagttctgtcattaatggattggatgcgagggtactcgatcttggacatggatccgtgggttgggatgtttttactttagattacatcctctacccccctttgagtttagtattaaacgtaaatcgtccttttggcaggaaagagtatctacgaattttcaattttttatggagatttaaaaagaacaattatttctatcaaaaggaaatgttgaagagtaatgatataatcagatcattcaagaaaatcagaggttacaacccgctcatccgtgatattatcaataaactttctagaatcagtatacttagaactcaa

SEQ ID NO: 105 - DNA, 2082 bp, Candida albicans
atggatataattagaggtgcatgttcagttgataaaattggggggatggtgtatattagagaagatttagcaccgctgatgttggaatggaaaccaattgatgaacaagaagaagatagagcaatttcaatcccattgaattctttaactacattacaaagtaccaaagaaacctcaccgaaaatgatactaaaaattgtatacaaactaacatctggtccacctaatacaaatgcagatggaactgacaatggtggtggtggtggtggtgaacaaaaatcatttaaattgacatttactaatagaccaaccatgaacactattaaagattctctacaaacaattgttgctagatcaagaactaagggtttgaaggtaccagtactccaactccagctccagcaccagcttcaacatttggggtcagcaccacaagctgattctaccagagattcgacatcatcatcaacaccaataccacctacaacatctggaacttctactagttcatcattattatcattagcagcatcacaatcattatctgatgcaaatttattgaaaaatttcgaactacagcaaaaacttttattagaagatcgtcaattacgtgatgttttcactaaatcagtcatgcaatttaaattatctcctcaagtattttggtcatcaagattaaatcaattacgaacatttgctttgacaatatctcaacataaaggtccatataatgtattgagtacaattaaaccggtggccacttctgataatcaagtgaatgttaatgttacgcgtgataccattaatgaaatatttactatttaccccatcataaagaaagcatttgatgatttggttcctaacaagtttaatgaaggagaattttggtcgagatttttcaattctaaattgtttagacgcttaagaggtgataaaatcagtattagtaatagtcgaggagatgttgtattggacaaatatttgtatatagatcaaaactatcaagaaaaattacaaaaatcatctactttggaaaacaacggttctggtggtggtggtggtggcgctggtggtggtagtggtaattcagaacaaggaatacaaacattggaatctccacatgttaaaaaatttcttgatttgatgggaaatcaacaagataattcacaaaaattggggaatagaccagattttactatgagatatgatgaagacaccaatgtagatgatgataataaaaaacctactttaggaaatgaaaatgaaatgattatattgatgaaaaatatgaatcgattatcgtcgaaaatgatgagtatgagttctactaatggaccagagaaaccttcagaaactacaattgatggattatctgctgctgaattgaatgaatatgaagaagaattagatttgcatgatttaaatgattcagaaaatttacaatatataaaattaaacattaatactgatattgccaagggaacaaaacttgattcatatgaaggatcaaatactaataacaagatttctcaagatgaattacataaatatttacaatctcaaactttccaaggacaaatagaattaacagaaacttatacttgtaaaagtgaagaaattgaaaaaacctccatggaaatagccatgcttattaaacaaaatttccgaacatttaaattaattaataaagaaaatgatattgcggggacaaacattgttcctaattcattaatacaagaaatcattacttataatattacgatagttgaatttttatctcatttttggaagatttttttacatgggaataatcctggtcaattaaagaaaattttcaccagtttgaaaaattgtcaatctggtttaatagaattagaaaataaagcgattgatcaattcaaatctatggatatattacaaaaaaatcaaaaattacaagataaagttttaaaagattttgcatcatgtcttcaacccatgaaaatagcattagataaagcatgtaatgaatatgttgaagcagtaaagaaagctaaacctgaattaaatgaaaatggtaaacgtcctctaccagaggagtga

SEQ ID NO: 106 - DNA, 466 bp, Homo sapiens
  misc_feature (1)..(466): n is unknown
  ref <308>: DATABASE ACCESSION NUMBER: Human Genbank/W19128
  ref <309>: DATABASE ENTRY DATE: 1996-05-03
  ref <313>: RELEVANT RESIDUES: (1)..(466)
ngncacattctgcnnagagatcctttgaccctgnatncagccgatccctgtgaaaataatgggantggaaaaaacgtgtccagnattccttctctgtcatgtngtgggnaacattttctgcatatttcattttnactgctggataggtcctnaatatggactcaatgatancagaagttaaattatatcttagaccgttanagccatcagtttggggccggacatcagcnagaaatgcagcaganatgccaanatcctgcttatgattggatntggaagaactatctgttgcattcacatttaaaccgattggnccagaattcctcagcactgatcacttgactcacgaacaaggtctttataaagctgaaacaaaaccaggatcttcttgcagcattctgttcatnccctccagtncctgnatttgcnttccncttgaatttgggcagcanctgctgangaaggt

SEQ ID NO: 107 - DNA, 2460 bp, Saccharomyces cerevisiae
  ref <308>: DATABASE ACCESSION NUMBER: Genbank/CAA96007.1
  ref <309>: DATABASE ENTRY DATE: 1997-08-11
  ref <313>: RELEVANT RESIDUES: (1)..(2460)
atggaactagagcccactctttttggtataatagaggcattggctcctcaattattgtcgcagagtcatttgcagacatttgtatctgatgtagtcaatttactgcgatcatccaccaaatcggcaactcaattaggccctttaattgatttttacaaattacaatcactagattcgcctgaaacaacaattatgtggcataaaattgagaaatttctcgatgctttatttggaatccagaacaccgatgatatggtaaagtacctctctgtctttcaatctttgcttccatcaaattacagagcaaaaattgtccaaaaatcatctgggctcaatatggagaaccttgctaaccatgaacatttacttagcccagtgcgggctccaagtatatatacagaagcttcatttgaaaacatggaccgattttctgaaagaaggtccatggtatcttcgcctaatcgttacgttccctcttcaacctacagttctgttactttgagacagttgtcaaatccttattatgtgaacactatacccgaggaagatatcctaaaatacgtatcatatacattattagctacgacatcggcactatttccgtttgatcatgagcaaatacaaattccgtctaagatacccaattttgagagtggacttttacatttaatatttgaagcgggtttattatatcaaagtttgggttataaagtggagaagtttaggatgttgaatatatctccaatgaaaaaagcattgattatagaaatttcagaagaattacaaaactacacagcatttgtgaacaatctggtctcttcagggacagtagtgtcattgaaatcgttatatcgtgaaatatatgaaaatataataaggcttcgaatatactgtaggtttacagaacaccttgaagaattgagcggagatacattcttgattgaattaaatattttcaaatcccacggagatcttactataagaaaaatagcaacgaatttgtttaattcaatgatttctctttattatgagtatttaatgaattggttgactaaaggtctactccgagctacttatggagaattcttcattgctgaaaacactgatacaaatggtacagacgatgattttatttaccacattcctatagagttcaaccaagaaagagttccggccttcataccgaaagagttggcatataaaatattcatgatcggcaaatcgtatatcttcctagaaaagtactgtaaagaggttcaatggacaaacgaattttctaaaaagtatcatgtcctgtaccagagcaattcttatcggggaatatcaacgaacttttttgaaattataaatgatcaatattctgaaattgttaatcatactaatcaaattctaaatcagaagtttcattacagagacgtggtatttgcgttaaagaatattcttctcatgggtaaatctgattttatggatgctcttatagaaaaggccaatgatattctcgcgacaccatcggattcattgccaaattataagttaacaagggttttacaggaagccgtgcagctttcttccttaagacatttaatgaatagtccccgtaatagttctgtcattaatggattggatgcgagggtactcgatcttggacatggatccgtgggttgggatgtttttactttagattacatcctctacccccctttgagtttagtattaaacgtaaatcgtccttttggcaggaaagagtatctacgaattttcaattttttatggagatttaaaaagaacaattatttctatcaaaaggaaatgttgaagagtaatgatataatcagatcattcaagaaaatcagaggttacaacccgctcatccgtgatattatcaataaactttctagaatcagtatacttagaactcaattccagcaattcaactcgaagatggaatcttattatttgaactgcattatagaggaaaattttaaagaaatgacccggaaactgcaacgcacagagaataaaagccaaaaccaattcgacttaattagattaaataatggcaccatagaattaaatgggattttaaccccaaaagctgaagtactaacaaagtcttcaagcagtaaaccccaaaaacacgcaatcgaaaagacgctgaatattgatgaattagaaagtgtacataacacgttcttgacgaatattctttctcataagctttttgcaactaacacaagtgaaataagcgttggtgattattctgggcaaccatacccaacttcattggttttacttttaaattcggtttacgagttcgtcaaagtttattgtaatttgaacgacattggatacgaaatcttcattaaaatgaatctcaatgatcacgaagcatctaacggattattgggaaaatttaatacgaatttaaaggaaattgttagccagtataaaaattttaaa

SEQ ID NO: 108 - DNA, 1921 bp, Candida albicans
atggcgttaaacaaggtacaactaataaaattatattccaatcgattagtgaaatcattggttcctgtggaattcggtgaggcattcatccaaagtataatcaatgacttgcaaaccactttactaaatacttcttctgaagaacaaaatttgtcaataattataaacaagcttaaaatgcaatttttaagtaacaatttaaaaaatgaatgggtcgaatttcaaaacattgttaattcattaagcaaattcaagtcgttggatcagatttgtaattatctcgcatttcttgatgctttaagagatgagaaaccagaagatatattatcaacatcaacagcgagcttgtctcccggtaagcaaaatgtaatgatcaatacggtaaacacagcattgacgttatcacagttaatcgagccttactatgatactttatcggaacaaaccattttaacctacttaccctacacgatgttaggtctggattccaaaatattcaccttcagcaataattatacacgattggagataccgaaagatataaacaacagtttcagctcattgctacgcgaagtttttgagtttgcaatactatataaacaattggcaattgttgttgataggtataaaggaactttagtactggccataaagacagcttacatagcaatactagaggctcaattgaacaaatatgtgaatgatattaacaatatcttcaataataaaccgaattccatattagttgtttacaattccattttcccctggatatctatactacgatttttatatcgagtctcaaacagactaaacagattagatggttatgaatttctcacatttatttatagtttcaccaaccatggagatcccaaaatacggggcattgctgtgactgcattcaccgaggttgtcaaaccgtattataatattgtggaacattggatagtgaaaggggagttgattgataataataacgagtttttcattatctttgatcaagagcagaatgaattcaatagtataattaaattattgcccaaaaaaataccagcctttattaaatcgagtgataaaatatttcagattgggaaaacattaatttttctaaataaatattgtcgtgaactaaaatgggtaaatcagtataacgtgaaatattctgctatattgttcaataaccatcaaggcttggcatccatgacaacaaatgaaatgatcaaattgattgatctgcaatataatgagatattaacgtttctcacccaaataatccaaggaaacaataaattgtttactcatgtttataatttcaagaggttttattttatggagaccaatgattttattgatgcgattatggtgaaagggaaggacgtttttaatgagtcttctgttaatatttcatcaacctatcttaggaaagtcttacaagacgctatacaaatttcgtcggtgaaaaattttgagtatgttgacagactcgattcgagagtgttgaatccccaacacgggaatttgggctgggaatcgttcaccattgaatacaaaattgatgatcttcccatgagttatttatttgaaggtcaccaacatttacaatatttaaaaatgtttcattttctatggaaattaagacaattgaataatttattaaattggcattttgagatgtttaatgagttgaatcataatgtggtgacgaagttgtcaagcagaaatagaagacctttggcgaaatcattgagcataatcaccagtataagattccattttacccagtttcttaacgaactaatagcttatttgtcttatgatgttattgaagaaaattttcgacagactgtatatttttagggcagatttaaagaacgatggcgatgaagagcttttcttattgagcaaatcgctccgttaa

SEQ ID NO: 109 - DNA, 3829 bp, Homo sapiens
  ref <308>: DATABASE ACCESSION NUMBER: Human Genbank/AF042378.1
  ref <309>: DATABASE ENTRY DATE: 1998-05-07
  ref <313>: RELEVANT RESIDUES: (1)..(3829)
caggaagggcgcgggccgcggtccctgcgcgtgcggcggcagtggcggctctgcccggaccaccgtgcacggctccgggcgaggatggcgaccccggaccagaagtcgccgaacgttctgctgcagaacctgtgctgcaggatcctgggcaggagcgaagctgatgtagcccagcagttccagtatgctgtgcgggtgattggcagcaacttcgccccaactgttgaaagagatgaatttttagtagctgaaaaaatcaagaaagagcttattcgacaacgaagagaagcagatgctgcattattttcagaactccacagaaaacttcattcacagggagttttgaaaaataaatggtcaatactctacctcttgctgagcctcagtgaggacccacgcaggcagccaagcaaggtttctagctatgctacgttatttgctcaggccttaccaagagatgcccactcaaccccttactactatgccaggcctcagacccttcccctgagctaccaagatcggagtgcccagtcagcccagagctccggcagcgtgggcagcagtggcatcagcagcattggcctgtgtgccctcagtggccccgcgcctgcgccacaatctctcctcccaggacagtctaatcaagctccaggagtaggagattgccttcgacagcagttggggtcacgactcgcatggactttaactgcaaatcagccttcttcacaagccactacctcaaaaggtgtccccagtgctgtgtctcgcaacatgacaaggtccaggagagaaggggatacgggtggtactatggaaattacagaagcagctctggtaagggacattttgtacgtctttcagggcatagatggcaaaaacatcaaaatgaacaacactgaaaattgttacaaagtagaaggaaaggcaaatctaagtaggtctttgagagacacagcagtcaggctttctgagttgggatggttgcataataaaatcagaagatacacggaccagaggagcctggaccgctcattcggactcgtcgggcagagcttttgtgctgccttgcaccaggaactcagagaatactatcgattgctctctgttttacattctcagctacaactagaggatgaccagggtgtgaatttgggacttgagagtagtttaacacttcggcgcctcctggtttggacctatgatcccaaaatacgactgaagacccttgcggccctagtggaccactgccaaggaaggaaaggaggtgagctggcctcagctgtccacgcctacacaaaaacaggagacccgtacatgcggtctctggtgcagcacatcctcagcctcgtgtctcatcctgttttgagcttcctgtaccgctggatatatgatggggagcttgaggacacttaccacgaattttttgtagcatcagatccaacagttaaaacagatcgactgtggcacgacaagtatactttgaggaaatcgatgattccttcgtttatgacgatggatcagtctaggaaggtccttttgataggaaaatcaataaatttcttgcaccaagtttgtcatgatcagactcccactacaaagatgatagctgtgaccaagtctgcagagtcaccccaggacgctgcagacctattcacagacttggaaaatgcatttcaggggaagattgatgctgcttattttgagaccagcaaatacctgttggatgttctcaataaaaagtacagcttgctggaccacatgcaggcaatgaggcggtacctgcttcttggtcaaggagactttataaggcacttaatggacttgctaaaaccagaacttgtccgtccagctacgactttgtatcagcataacttgactggaattctagaaaccgctgtcagagccaccaacgcacagtttgacagtcctgagatcctgcgaaggctggacgtgcggctgctggaggtctctccaggtgacactggatgggatgtcttcagcctcgattatcatgttgacggaccaattgcaactgtgtttactcgagaatgtatgagccactacctaagagtatttaacttcctctggagggcgaagcggatggaatacatcctcactgacatacggaagggacacatgtgcaatgcaaagctcctgagaaacatgccagagttctccggggtgctgcaccagtgtcacattttggcctctgagatggtccatttcattcatcagatgcagtattacatcacatttgaggtgcttgaatgttcttgggatgagctttggaacaaagtccagcaggcccaggatttggatcacatcattgctgcacacgaggtgttcttagacaccatcatctcccgctgcctgctggacagtgactccagggcacttttaaatcaacttagagctgtgtttgatcaaattattgaacttcagaatgctcaagatgcaatatacagagctgctctggaagaattgcagagacgattacagtttgaagagaaaaagaaacagcgtgaaattgagggccagtggggagtgacggcagcagaggaagaggaggaaaataagaggattggagaatttaaagaatctataccaaaaatgtgctcacagttgcgaatattgacccatttctaccagggtatcgtgcagcagtttttggtgttactgacgaccagctctgacgagagtcttcggtttcttagcttcaggctggacttcaacgagcattacaaagccagggagcccaggctccgtgtgtctctgggtaccagggggcggcgcagctcccacacgtgaagctcgcggtcctcccagggagctgcgggtgatgttcgttgcactgctagacacgaaattcccattgacgtcctgcaggaactgcatgctgcaggtgtcctgcccttccgcccacgagtgcgccatgtttcagcggagcggcgtgtgggagaagccacgtcgtgtttcacatgtcggagtcgaatgcatttgtaaatccctaagtcaagtaggctggctgcactgttcacatttgtctctaaaagtcttcatcgctaaaagataccataatttgctgaggcttcttaagctttctatgttataatttatatttgtcactttaaaaaatccatttcttttagaaaaaattagggtgataggatattcattagttaagatggtaacgtcattgctatttttttaacatcctctttagaggtaatttttgttaacataaccaaaaattaaattgaaacaaaatgtcccaactaagaaaatatatagagcattttatttttttttagtgttgtaaaatattaacctctgtgagatcctttgtatcttaatgcattacctttacacatatttattcttattttctctcctttcagagtttacatttttatatttaatttactatttcagatttttaaaatagtatagaaaaaagtaggagtgatagagaacaaaaatactcttatacagtgcaacccaaataccgcgaatgcatcagctaaagcagcgtgtaaataggagtgatgagaaagttaatggagtattttattttcaaagttcctgataagcattggaaagaaatcgacatggataatgaagatttcctttttccttgcctattttttcattgtaaatatttatatactactgaccaagatgttggggtgggggggattgttttttgtaaaaatgtcattatcaggtcacataaatctgcctttatgttgcataagtgaaaatttagaaaattaaaagcaattatctttcaaaaaaaatggaataaattgcttttctacataaaaaaaaa

SEQ ID NO: 110 - DNA, 1560 bp, Saccharomyces cerevisiae
  ref <308>: DATABASE ACCESSION NUMBER: Genbank/AAB64735.1
  ref <309>: DATABASE ENTRY DATE: 1997-08-01
  ref <313>: RELEVANT RESIDUES: (1)..(1560)
atggaaaaatcactagcggatcaaatttccgatatcgccattaaaccggtcaataaagacttcgatattgaagatgaggaaaatgcatctttatttcaacacaatgaaaaaaatggagaaagtgatttaagcgactatggaaatagcaacacagaagaaaccaagaaggcgcactatttggaggtggaaaagtctaagttaagagcagaaaaaggtttagaactaaacgatccaaaatatacaggtgttaaaggttcaagacaagcattatatgaagaagtttccgagaatgaggacgaagaagaagaagaagaagaggaagaagaaaaagaggaagatgctctttcattcaggacagattctgaagatgaagaagtagagattgatgaagaagaatcagacgcggacggcggtgaaacggaggaggctcaacagaaaaggcatgcactatcgaaactaattcaacaagagactaaacaagctattaacaaactgtctcaatcagttcaaagagatgcttcgaagggttattccattttacaacagacaaaattatttgacaacatcattgatttgagaataaaactacaaaaagctgtaattgcagcaaataagctcccattaactacagagtcctgggaagaggctaaaatggatgattcagaggaaacaaagcgtttgctgaaggaaaacgaaaaactgttcaataatttattcaatcggttgataaatttcagaataaaattccaacttggcgatcatatcactcaaaatgaagaggtggcgaagcataaattgtccaaaaaaagatctctcaaagagctttaccaagaaactaatagcttagactcagaactaaaagagtacaggactgccgtattaaacaagtggtctaccaaagtttcttctgcatcaggtaacgctgctttatcatctaacaaattcaaagctatcaacttacctgcagatgtacaagtcgaaaaccaattatccgatatgtcccgtttgatgaaaagaacaaagttgaacaggagaaacataacgcctttgtatttccaaaaagactgtgctaatggcaggctaccagaattgatttctcccgttgtcaaagatagtgttgatgacaatgagaattcggatgatgggcttgatatcccgaaaaactatgacccaagaagaaaggataacaatgccattgacattaccgaaaacccatatgtttttgatgacgaagatttttaccgtgttttactaaacgatttaattgacaaaaagatttccaacgctcacaattctgaaagtgcagcaattacaatcacctcaactaatgctcgttcgaacaacaagctaaagaagaatatcgatactaaggcttccaagggtaggaaattgaactactcagttcaagatccaattgcgaattatgaagcccccatcacatccggatacaaatggtcagacgaccaaatcgatgaattctttgcgggattgttaggtcaacgagtgaactttaatgaaaatgaggatgaggaacaacatgccagaatagaaaatgacgaa

SEQ ID NO: 111 - DNA, 1596 bp, Candida albicans
atgagcttcttcggcttacactttcaacttaattcattgacattgaacatttcaaatatggcaaaaaagtctttatcagagcaaatttctagtttatatacaccaaagactgattatgatattgaggatcatgatttagatgtatctaaagacaatggcatttttcagcatcatgacggtggttctgaaaacgaatctgaagacgaggatactggcttaagaaatgagcattatgttgaatcttcaaaatcaaagttgagacaacagaatgaaggtgtgaacttgggggaaaaatacgtgggcaatgtcacaagcagaagcaaattgtatgacgatgaggatgacaaacaaccaacagaagctagctccggagaggagttagatgctgaatcagcggaagaagaagaggatgaagaatctgaagatgtagcagatgatgatgaagatgaccaagagtcagatcgcagtagctcaagtgatgcagagaatgacgaggacgagaacatttcacacaaaagggaattattaaaacaattaatgagcaaagagagaagtcacatcgttaacagattatcccaatcagcaacaaatgatgcattaaaaggttattcaatacaacagcaaaacaaaacttttgaaaaaatcattgatgtgaggttgaaatttcagaaatcggtaacttcaagtaatatgttacctataaatacaagtacatattcagaaaccaaatctgaagatagcgatgaattagtgactaaagccaagaaacaattgtatagtttgttggatcatttattcacacttagaaacgaactagacgaaagtacctcagtcaagacccccaaaaaacgatcatttgctaaatattcggaggttacatctgctgcagatgcacaattgaattcccgtcgtaaccaaatattaaccaagtggtcagctaaagttgccaattcatccggtagaaatgccatgaatgctaataaattcaaaactataaaccaatcttttgaacaacaggttaacaacaacttgtctgacatggatagattaatcaaaagaacaaaattgaaccgaagaaacgtaactcccattggttataccaccaaagaggaggatgatcatgaaaatggcaataaaaacaaatctatcgacgaggacgacgacgatattcccgaagatacttctgttcgtaagaaaacccaaggcttggaaaatgattatatatttgatgacgaagatttctatagagtattgttgaatgatttagtcgacaagaaagtgcaaacaagtgatccaacatcaggtataactatcagtttaagagctgctcaaaagtccaataaattgaaaaataatgttgatacaaaagcatctaaaggtaggaaattgagatatcacgtgcaagaaccaattgctaattttgaaacttcaagaggcagctggagatggaatgatgatcaaattgacgagtttttcgcatctttattgggccaaaaggtcaatatgaatgagatagatgatgaacaagaagaagaacaagagaatgatgataatgatattattccagaggataacggaatccagttgtttggttaa

SEQ ID NO: 112 - DNA, 2444 bp, Homo sapiens
  ref <308>: DATABASE ACCESSION NUMBER: Human Genbank/NM_000055
  ref <309>: DATABASE ENTRY DATE: 2001-02-03
  ref <313>: RELEVANT RESIDUES: (1)..(2444)
agtaacagttgattgttacattcagtaacactgaatgtcagtgcagtccaatttacaggctggagcagcagctgcatcctgcatttccccgaagtattacatgattttcactccttgcaaactttaccatctttgttgcagagaatcggaaatcaatatgcatagcaaagtcacaatcatatgcatcagatttctcttttggtttcttttgctctgcatgcttattgggaagtcacatactgaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggatcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacatgtttaattaaatttcacaatataaagttctacagttaattatgtgcatattaaaacaatggcctggttcaatttctttctttccttaataaatttaagttttttccccccaaaattatcagtgctctgcttttagtcacgtgtattttcattaccactcgtaaaaaggtatcttttttaaatgaattaaatattgaaacactgtacaccatagtttacaatattatgtttcctaattaaaataagaattgaatgtcaatatgagatattaaaataagcacagaaaatc

SEQ ID NO: 113 - DNA, 1200 bp, Saccharomyces cerevisiae
  ref <308>: DATABASE ACCESSION NUMBER: Genbank/CCA90206.1
  ref <309>: DATABASE ENTRY DATE: 2001-02-03
  ref <313>: RELEVANT RESIDUES: (1)..(1200)
atggctaccttgcacttcgttcctcagcacgaggaagaacaagtttactccatctctgggaaggcactcaagttaacaaccagtgacgatatcaaaccatacctggaagaattggcagctttgaaaacctgtaccaaattagacctttcagggaatacaatcggtactgaagcttcggaagcattagctaaatgcatcgctgaaaatacacaggtcagggaatctttggttgaagtaaattttgctgacttatacacttcgaggttggttgacgaagtcgttgattcgttgaagtttttattgcctgttctgttgaaatgtcctcacttggagattgtgaacctttctgataatgcgtttgggctaagaacaatcgagttactagaagattacattgcacatgccgtgaatatcaaacatttgatcttaagtaacaatggtatgggcccttttgctggtgaaaggattggtaaggccctatttcatctcgctcaaaataagaaagctgcttccaaaccatttttggaaacttttatctgtggtagaaatagattagagaatggatccgcagtctacttagctctgggtttgaaaagccactccgaaggtttgaaagtcgtaaagctgtaccaaaatggtattaggcctaaaggtgtcgccacgctaattcattacggtttacagtacttgaaaaacttggaaatcttggatcttcaagacaatactttcacgaaacatgcttctttgatccttgctaaggccttgcctacatggaaggatagtttatttgaattgaatttgaacgactgtcttttgaaaactgctggttcagatgaagtctttaaagtattcaccgaagttaaattccccaatttgcatgtcttgaaattcgaatataatgaaatggctcaagaaaccattgaagtatccttcttaccggctatggaaaagggaaatttacctgaattggaaaagctagaaataaatggtaacagattagatgaagattctgatgctttagatttgctccaaagcaaatttgatgatttagaggttgacgattttgaagaggtcgatagtgaagatgaggaaggcgaggacgaggaagacgaggacgaggatgaaaaactcgaagaaattgaaacggaaaggcttgaaaaggaactgctagaagtacaagtagatgatcttgctgaacgt

SEQ ID NO: 114 - DNA, 1245 bp, Candida albicans
atggcatcagtagaagttgaattaggagttactccagaaaccacttattcaatttcaggaaaacaactaaaatttgattctgaatcggatattgctccatatatcaaggaattgacggaaaaagaaaatgtcaaaaaagttgatttttcaggaaatactattggtattgaagcatcaaaagcattaagtgaagcattattaaaacataaagacactatcgttgaaatcaacttttctgatttatacactggtagattgaatactgaaattcctcaatctttagagtatttgttaccagcattgtcgaaattgccaaatttgaaattgatcaacttgagtgacaatgcttttggattgcaaactattgatccaattgaagcttacttggccaaagctgtttccatcgagcatttgattttgtcaaacaatggtatgggtccatttgctgggtcaagaattggaggatctttgtttaagttagctaaggctaagaaagcagaaggaaaggagtctttgaaaacatttatttgtggtagaaacagattggaaaatggttctgttaactatttatctgttgggttaagaaatcacaaggatttggaagtggttagattgtatcaaaatggtattagacctgctggtatttctaaattggttgagcaaggtttatctaacaacaaaaaattaaaagtgcttgatttgcaagacaataccatcactaccagaggagctattcacattgcagaatcattatctaactggccacttttggttgagttgaatcttaacgattccttattgaagaacaaaggttctttgaaattagtcgaagccttccatgctggagatgaaaaaccgcaattaattaccttgaaattacaatataatgagttagaaacagatagtttaagagttttggctgatgcaattgccagtaaattaccacaattgaagttcttggaattgaacggtaatagatttgaagaggattccgaacatatcgataaaatcaatggaatcttcgaagaaagaggctatggcgaaatagatgaattggatgaattagaagagcttgatagtgaagaagaagaagatgacgaggatgacgaaggagaagacgacacattagaggaagaccttgatttgacacaattagaagaagaattggctggagtttctttggaagacaaagatggtaacgtggatgaaattgccgaagaattatccaaaactcatattaaatag

SEQ ID NO: 115 - DNA, 1788 bp, Homo sapiens
  ref <308>: DATABASE ACCESSION NUMBER: Human Genbank/X82260.1
  ref <309>: DATABASE ENTRY DATE: 1995-05-11
  ref <313>: RELEVANT RESIDUES: (1)..(1788)
atggcctcggaagacattgccaagctggcagagacacttgccaagactcaggtggccgggggacagctgagtttcaaaggcaagagcctcaaactcaacactgcagaagatgctaaagatgtgattaaagagattgaagactttgacagcttggaggctctgcgtctggaaggcaacacagtgggcgtggaagcagccagggtcatcgccaaggccttagagaagaagtcggagttgaagcgctgccactggagtgacatgttcacgggaaggctgcggaccgagatcccaccagccctgatctcactaggggaaggactcatcacagctggggctcagctggtggagctggacttaagcgacaacgcattcgggcccgacggtgtgcaaggcttcgaggccctgctcaagagctcagcctgcttcaccctgcaggaactcaagctcaacaactgtggcatgggcattggcggcggcaagatcctggctgcagctctgaccgaatgtcaccggaaatccagtgcccaaggcaagcctctggccctgaaggtctttgtggctggcagaaaccgtctggagaatgatggcgccactgccttggcagaagcttttagggtcatcgggaccctggaggaggtccacatgccacagaatgggatcaaccaccctggcatcactgccctggcccaggctttcgctgtcaaccccctgctgcgggtcatcaacctgaatgacaacaccttcactgagaagggcgccgtggccatggccgagaccttgaagaccttgcggcaggtggaggtgattaattttggggactgcctggtgcgctccaagggtgcagttgccattgcagatgccatccgcggcggcctgcccaagctaaaggagctgaacttgtcattctgtgaaatcaagagggatgctgccctggctgttgctgaggccatggcagacaaagctgagctggagaagctggacctgaatggcaacaccctgggagaagaaggctgtgaacagcttcaggaggtgctggagggcttcaacatggccaaggtgctggcgtccctcagtgatgacgaggacgaggaggaggaggaggaaggagaagaggaagaagaggaagcagaagaagaggaggaggaagatgaggaagaggaggaagaagaggaggaggaggaggaagaagagcctcagcagcgagggcagggagagaagtcagccacgccctcacggaagattctggaccctaacactggggagccagctcccgtgctgtcctccccacctcctgcagacgtctccaccttcctggcttttccctctccagagaagctcctgcgcctagggcccaagagctccgtgctgatagcccagcagactgacacgtctgaccccgagaaggtggtctctgccttcctaaaggtgtcatctgtgttcttagctgaaactgaaatcaaatagaaggacgaagctactgtgaggatggcagtgcaggatgcagtagatgccctgatgcagaaggctttcaactcctcgtccttcaactccaacaccttcctcaccaggctcctcgtgcacatgggtctgctcaagagtgaagacaaggtcaaggccattgccaacctgtacggccccctgatggcgctgaaccacatggtgcagcaggactatttccccaaggcccttgcacccctgctgctggcgttcgtgaccaagcccaacagcgccctggaatcctgctccttcgcccgccacagtctgctgcagacgctgtacaaggtctag

SEQ ID NO: 116 - DNA, 1140 bp, Saccharomyces cerevisiae
  ref <308>: DATABASE ACCESSION NUMBER: Genbank/AAB67337.1
  ref <309>: DATABASE ENTRY DATE: 1997-08-22
  ref <313>: RELEVANT RESIDUES: (1)..(1140)
atgtcctctcaagcattcacttcagtacatccgaatgcggcaacatctgatgtgaatgttaccattgacactttcgttgctaagttaaaaagaagacaagtgcaaggttcatacgccatcgccttggaaactttacaactgttaatgcgatttatctctgcagctcgttggaaccatgttaatgaccttattgaacaaatcagagatttaggtaatagtctagaaaaagctcatcctactgctttcagttgcggtaacgtaattagaagaatactggctgttttgagggatgaagtagaagaagacactatgagcacaactgtcacatccacatccgttgctgaacctttgatttcctctatgtttaatttattacagaaaccggagcaacctcatcagaatagaaaaaatagttcagggagctctagtatgaaaaccaagactgattaccgtcaagtagccattcagggtatcaaggatcttatagatgagataaaaaacattgatgaaggtattcagcaaattgctattgatttgattcacgatcatgagattttattaactcccacacctgattcaaaaaccgtattaaaatttctgattactgctcgcgaacgtagtaatagaacatttacggttttagttacagaggggttccctaataacaccaagaatgcacatgagtttgccaaaaaattagcacagcacaacatagaaaccctagtagtcccagactcagctgtttttgctttaatgtcccgtgtgggtaaggttattatcggcactaaagccgtttttgtcaatggggggactatctcgtcaaattcaggtgtatcatccgtttgtgaatgcgcccgagaatttagaacccctgtatttgctgttgcaggtttgtataagctttctcctctatatccgttcgacgtagagaagtttgtcgaatttggtgggtcccaacgtatattacctagaatggatccaagaaaaagattagatacagttaatcaaattaccgattatgttccgcctgaaaatattgatatctacattacaaacgtcggtgggttcaatccaagttttatatatcgtattgcgtgggataattacaagcaaattgatgtgcatttggataaaaataag

SEQ ID NO: 117 - DNA, 1098 bp, Candida albicans
atgtcgaaattgcttactcctgaaattctagcgctcatagacccagtggtgtctagtttgaaacgtcatcagcttgtggatgataaggagatagcattaacaattgcccagttgttgatgaaagtcatatcagcagcaagatggtctaatacatatgatttaattgaattgataagacaagttggtgttatatttaccgaagcatatcctagaaaagtcattccaggaaatattgtgagaagagtgttagctttaatacgtgatgaaaccgaaactgaaactgagacagagactgaacaaacagataacatcccaatgatgagctctatgtttagtttattggcaacacataacaaaaatgaaactataaaggaacaaacacaattacaactgaagaaacaaacaagcgatatgagagccataattatacaagggattagagatttagttgatgaaatttccaatgttaatgatgggattgaaactatggcggttgatttgattcatgacgatgaaatattattaactccaacccctaattcggaaacagtgcaacattttttaatcaaagcaagattgaaaagaaaattcacagtagttgttactgaaaactatccaaacgacatcaaggcagcccacaagtttgtaaagacactagctgaacacaacatcgaaacaattttaattccagacacaacaatttatgcagtgatgtcaagagttgggaaagttataataggtactaatgctgtatttgccaatggtggctgtttgtcagattcaggtgttgccaatgtagttgaatgtgccaaagaacacagaacacctgtgtttgctgtggcagggttattcaaattatctccattgtatccatttacaagaaacgatttgattgaagtaggaaactccgggaaggttttgaactacgacgattttgaattggtacaaaatgttgatgttgtgactaatcctttggaagattatatacctcctcaacatatcgacatttttatgaccaatattggagggttttctccttcatttatttatagaattgttttggataattataaagctgaagacaacaaacttgaataa

SEQ ID NO: 118 - DNA, 1450 bp, Homo sapiens
  misc_feature (1367)..(1367): n is unknown
  ref <308>: DATABASE ACCESSION NUMBER: Human Genbank/L40395.1
  ref <309>: DATABASE ENTRY DATE: 1997-08-22
  ref <313>: RELEVANT RESIDUES: (1)..(1450)
aaaaagggttcggagttgtcagagaggatcgagagcttcgtggagaccctgacgcggggtggtgggccgcgcaggtccgaggaaatggtcgggagaccctagggttgctgcgccagatcatcacggaccaccgatggagaaacgcgggggagctgatggagctgatccgcagagagggcaggaggatgacggccgctcagccctccgagaccaccgtgggcaacatggtgcggagagtgctcaagattatccgggaggagtatggagactccatggacgcagcgacggagagtgatcagcaggagtccctggacaaactgttgacayccggaggcctaaacgaggatttcagcttccattatgsccaactccagtccaacatcattgaggcgattaatgagctgctagtggagctggaagggacaatggagaacattgcagcccaggctctggagcacattcactccaatgaggtgatcatgaccattggcttctcccgaacagtagaggccttcctcaaagaggctgcccgaaagaggaaattccatgtcattgtagcagagtgtgctcctttctgccagggtcatgaaatggctgtgaatttgtccaaagcaggtattgagacaactgtcatgactgatgctgccatttttgccgttatgtcaagagtcaacaaggtgatcattggcaccgaagaccatcctggccaatggggccctgagagctgtgacaggaactcacactctggcactggcagcaaaacaccattccaccccactcatcgtcttgtgcacctatgttcaaactttctccacagttccccaatgaagrrgmcycatwwmataagtttggtggctcctgaagaagtcctgccattcacagaaggggamatyctggagaaggtcagcgtgcattgycctgtgtttgactacgytcccccagagctcawtaccctctttagcgtgatctccaacattggtgggaatgcaccttcttacatctaccgcctgatgagtgaactctaccatcctgatgawcatgttttatgaccgaccacacgtgtcctaagcagattgcttaggcagatacagawtgaagaggagacttgagtgttgctgctgaagcacatccttgcaatgtgggagtgcacaggagtccaccwaaaaaaaaaatccttgatactgttgcctgcctttttagtcaccccgtaacaagggcacacatmcagcaytgtgtcttgcctttcagatcttaacagagcagcagggcttaacttgttgatttkggagsctcttagtgacctggttgcgtctgtgtcaggaacttaaactttctggttcagtagtgtgktaaacataacrctgwanaccttactgggatacagatttttgctcagaaatggctatgacactttttctaggctctaccaataaaarccacttgaaggttc

SEQ ID NO: 119 - DNA, 720 bp, Saccharomyces cerevisiae
  ref <308>: DATABASE ACCESSION NUMBER: Genbank/CAA97221.1
  ref <309>: DATABASE ENTRY DATE: 1997-08-11
  ref <313>: RELEVANT RESIDUES: (1)..(720)
atgtcaagactagaaatatactcgccagaagggctacgtctcgatggacgtcgatggaatgaactccgccgttttgaaagttccatcaacacacatccgcacgctgcagacggttcatcctacatggaacaaggtaacaacaaaattatcactcttgttaaaggtccaaaagagccaagattgaaatctcaaatggatacctcaaaggctttattgaacgtatcggtaaacattacaaaattctccaaattcgaaagaagtaaatcaagccacaagaatgaaaggcgtgttcttgagatacaaacctccctggtgaggatgtttgagaagaatgtcatgctgaatatctaccccagaacagttatcgatatcgagatccatgtccttgagcaagatggcggtattatgggatctttaatcaacggtattaccctcgctttaatagatgccggtatatcaatgttcgattacataagtggtatatccgtcgggctgtacgatactaccccattattagataccaattcattagaagaaaatgctatgagtacagtgacactaggtgtggtagggaagtcagaaaaactttctcttttattggtggaagacaaaattccgttagataggttagagaacgttcttgccatcggcatcgcaggtgctcatagggtaagagatttgatggatgaagaactgaggaaacatgctcagaaaagagtc

SEQ ID NO: 120 - DNA, 723 bp, Candida albicans
atggaattatattcacctgagggacttagaatagacggaagaagatggaacgaattgcgtagatttgaatgccgtatcaacactcatccaaactcatcggatggctcctcatatgtcgaacaaggtaataccaaagtgatgtgcacagtacaaggaccaatagaaccagcattaagatctcaacaacattcagaacgagcaaatatagaagtgaatttgaatattgctagtttttcaacttttgaaaggaaaaaacgaagtagaaatgaaagaagattagttgaacttaaaactactttagaaaaaacatttgaagaaagtgttatgataaatttatatccaagaacaaatattgttataaatgttcaagtattatgccaggatggtgggatgttagctgcagttatcaactctattacattagcactcattgacgctggtatatcaatgtatgattatgtgagtggtgtatcttgtggattatatgatcaaacaccattattagatgtaaataacttagaagaacacgatatgagttgtttaacagttggtgttattggtaaaagtgagaaattggcattaatgttgttagaagataaaatgccattggatagattggaatcagtattgtcaattggtattgctggaagtcataaaataagagaattaatggatcaagaagtgaggaagcatggaattattagggcttctaaaatgcaataa

SEQ ID NO: 121 - DNA, 840 bp, Homo sapiens
  ref <308>: DATABASE ACCESSION NUMBER: Human Genbank/AK000598.1
  ref <309>: DATABASE ENTRY DATE: 2000-02-22
  ref <313>: RELEVANT RESIDUES: (1)..(840)
agagagcggacctggcggccgggcagcatggcggggctggagctcttgtcggaccagggctaccgggtggacgggcggcgcgccggggagctgcgcaagatccaggcgcggatgggcgtgttcgcgcaggctgacggctcggcctacattgagcagggcaacaccaaggcactggctgtggtctacggcccgcacgagatccggggctcccgggctcgagccctgccggacagggccctagtgaactgtcaatatagttcagcgaccttcagcacaggtgagcgcaagcgacggccacatggggaccgtaagtcctgtgagatgggcctgcagctccgccagactttcgaagcagccatcctcacacagctgcacccacgctcccagattgatatctatgtgcaggtgctacaggcagatggtgggacctatgcagcttgtgtgaatgcagccacgctggcagtgctggatgccgggatacccatgagagactttgtgtgtgcgtgctcagctggcttcgtggacggcacagccctggcggacctcagccatgtggaggaagcagctggtggcccccagctggccctggccctgctgccagcctcaggacagattgcgctgcttgagatggatgcccggctgcacgaggaccacctggagcgggtgttggaggctgctgcccaggctgcccgagatgtgcacaccctcttagatcgagtggtccggcagcatgtgcgtgaggcctctatcttgctgggggactgaccacccagccacccatgtccagaataaaaccctcctctgcccacacaaaaaaaaaaaaaaaaaaaaaaaaaaaaa

SEQ ID NO: 122 - DNA, 2340 bp, Saccharomyces cerevisiae
  ref <308>: DATABASE ACCESSION NUMBER: Genbank/A46417
  ref <309>: DATABASE ENTRY DATE: 1998-02-06
  ref <313>: RELEVANT RESIDUES: (1)..(2340)
atgtcccgtttcttttcgtctaattacgaatacgatgtagccagttcttcatccgaagaagatcttttatcttcgtctgaagaagatttgttaagctcttcctcctctgagtctgaattggaccaagaatctgacgactcctttttcaatgaaagtgaaagtgaaagtgaagctgatgtagactctgatgattctgatgcaaagccttatggtcctgactggttcaagaaatctgagttcagaaaacaaggtggaggttcaaataaatttttgaaaagctctaactatgattccagtgatgaagaatccgatgaagaagatggcaagaaggtagtcaagtctgccaaagaaaaactattggatgaaatgcaagacgtttataataagatctctcaagctgagaactctgatgactggttgactatttctaatgagtttgatttgatctcgcgtctcttagttagggctcaacaacaaaactgggggactccaaatattttcatcaaggttgttgcccaagtggaggacgctgtgaataatacacaacaagctgatttgaagaataaagctgttgcaagagcttataacactacaaagcaaagagtcaagaaagtttctagagaaaatgaagactcaatggctaaattcagaaatgatcctgaatcatttgataaggaaccaaccgcagatttggatatttctgctaatggattcacaatttcttcgtctcaaggcaatgaccaagcggtacaagaagatttcttcactagattacaaacaataattgactcaagaggtaagaagactgtcaatcaacaatccttgatttctactttggaggagttattaactgtagctgaaaaaccatatgaattcataatggcttatttgactttgattccatcaagattcgatgcctcagctaacctatcttaccaaccaattgatcaatggaaatcttcattcaacgatattagtaaattattgtctattttagaccagacaattgacacctaccaagttaatgaatttgctgatccaatcgatttcattgaagatgaacctaaagaagattctgatggtgtcaagaggattctgggttccattttctcatttgttgaaagattagatgacgaattcatgaaatccctgttaaacatcgatcctcattccagtgattatttgatccgtttaagggatgaacaatcaatctataatttgatcctaagaactcaattgtactttgaagcgactttgaaagatgaacacgacctagaaagagcattgacacgtccattcgtcaagagattggatcatatctactataaatccgaaaatttgataaaaattatggaaactgctgcttggaatatcatacctgctcaattcaaatctaaatttacttcaaaagaccagctcgattctgctgattatgtcgacaatttaatagacggattatcgacaatcttatccaagcaaaacaacattgctgttcaaaaacgtgctattttatacaacatttactacactgcattaaacaaagatttccaaactgctaaagatatgttactaacttcccaagttcaaacaaatatcaaccaattcgattcatccctacaaattttattcaacagggttgttgttcaattgggtctatccgcctttaaattatgtttgattgaagaatgtcatcaaattttgaatgatcttctgtcaagttctcacttaagagaaattttgggccaacaatccctacacagaatatctctcaattctagtaacaatgcttcagctgatgagcgtgctagacaatgtttgccatatcaccaacacatcaatctcgatttaatcgatgtcgtcttcttaacatgttccttattgatcgaaattccaagaatgactgccttctattccggtattaaggtcaagagaattccttactctccaaaatccattcgtcgttccttagaacattacgacaagttaagtttccaaggtccaccagaaactttaagagattatgtcttgtttgctgccaaatcaatgcaaaaaggtaactggagagactctgttaaatacttaagagaaataaaatcttgggctttattaccaaacatggaaacggtgttgaatagtttaacggaaagagtacaagttgaatctttgaagacttatttcttttctttcaagaggttctattcaagtttttctgttgctaaactagccgaattatttgatcttccagaaaataaggtggttgaagttttgcaatctgttatcgcagaattggaaatcccagccaaattaaacgacgagaagaccatctttgttgtcgaaaag

SEQ ID NO: 123 - DNA, 2099 bp, Candida albicans
atgtctcgtttttttgtttcaggatacacttctgactcttcttctgaagaggaggatttattgagtacttctgaagaagagttattatcttcttctgatgaaggagaagacaacgaatcagatagttcattttttggtgaagatgatgatgaatcagaagaatctagttctgatgatgaagatggtcgaccatctggtccagcatattttttaaagaaatcatttttaaaaggagctggaggagatgattctgacagtgatagtgatgatgaaggtcgtaaagttgttaaatcagctaaagataaattattagatgatatgaaatcttcaattgaaattataaattccaacaaatataataacaattggagtatagttttaggtgaatttgataagtttggtagatttttgattagatgtaatcaaaccaatttgggtacaccaaaattttatattaaattgttgactagtttagataactccataactgaaactagtaataatgaaagagatgataaaacattaaaagctgatgaagccagagctttcaatactttgagacaaagaattaaaaaacaaataagagaattccaagtttattatgatttgtataaggaaaatccagaagaatttgatgaaaatgaagatgaaccattagaatctgttcaagctggtcttaacgataatgttaaaaatgaagctgataattctaatgttggtgctcttgcgtcaaacagagtattgagtcctattttccatactttgaaaactatttccgaaagtcgtggtaaaaagaatattgataaattggaacaaattgctactttggaaaaattattagaagcaaatgtttctaaaagttcaccatttgaattgatttctatttatcagatgttattatcagttagatttgatgcttcatctaatcaagcttttatgcctttggaacaatggcaaaagaatgaacacgatttaggtaaattattggatttgttggaagctaatgttgatacttatcaagtttctgaattgggttcaactactgatgatattgatattgaaccagttgctaatgcccaaggtgttaaagttattttcggatcaatcacttcttctattgatagattggacgatgaattgaccaaatctttacaacatactgacccacattctattgaatatgttgaaagattgaaggatgaaagtactatttacaatttgattgttagaggtcaagcatatgttgaatccataactccagaagatgtcaagtataattctgaacaattggcaagaattgttttgagaagattggaacacatttattataaaccaaaacaattgattaaagctaatgaagaagaagcttggcgtaatattgaatacaattcatctattgtcagtaaaggttcttcagttgatgaagttattgatcaattgacggaatttttacaaaagcaacaaaaaaacaaaacttatgggaaacatgctatactattctccatttattattatgctgtcaatagtcaatatgaaaaggctaaagaattatttttgagatctcaattttatagtaacatcaattctgctgaatcttctttacaagttcaatataatcgtgctttagttcaattaggtttaagtgcttttagagcaggtagtattgaagaatctcataaaattttgaatgaaattgtcaattctcaaagatctaaagaattattgggtcaaggtttcaattctaaattccccaatcaagctactgttttggaaagacaaaaattattaccattccatcaacatattaatttggaattattggaatgtgtatttatgacttgttctttattaattgaaattccaactttggctgctattgctaataatcataaggattcaaaacgtaaaaatgcttcattgaaatctttcaaaagtaaattggatttccatgatagacaatttttcactggtccaccagaaagtattaaagatcatattgtggtgatgaaattactaaattggaagaagcaatggtaaaattgaacaaagaatataaaatcgctaaagaacgtcttaacccaccatcaaatcgtcgttga

SEQ ID NO: 124 - DNA, 2898 bp, Homo sapiens
  ref <308>: DATABASE ACCESSION NUMBER: Human Genbank/U46025.1
  ref <309>: DATABASE ENTRY DATE: 1999-01-08
  ref <313>: RELEVANT RESIDUES: (1)..(2898)
tgactcgcgggctcagctggtccggccgtagcacctccgcgccgtcgccatgtcgcggtttttcaccaccggttcggacagcgagtccgagtcgtccttgtccggggaggagctcgtcaccaaacctgtcggaggcaactatggcaaacagccattgttgctgagcgaggatgaagaagataccaagagagttgtccgcagtgccaaggacaagaggtttgaggagctgaccaaccttatccggaccatccgtaatgccatgaagattcgtgatgtcaccaagtgcctggaagagtttgagctcctgggaaaagcatatgggaaggccaaaagcattgtggacaaagaaggtgtcccccggttctatatccgcatcctggctgacctagaggactatcttaatgagctttgggaagataaggaagggaagaagaagatgaacaagaacaatgccaaggctctgagcaccttgcgtcagaagatccgaaaatacaaccgtgatttcgagtcccatatcacaagctacaagcagaaccccgagcagtctgcggatgaagatgctgagaaaaatgaggaggattcagaaggctcttcagatgaggatgaggatgaggacggagtcagtgctgcaactttcttgaagaagaaatcagaagctccttctggggagagtcgcaagttcctcaaaaagatggatgatgaagatgaggactcagaagattccgaagatgatgaagactgggacacaggttccacatcttccgattccgactcagaggaggaagaagggaaacaaaccgcgctggcctcaagatttcttaaaaaggcacccaccacagatgaggacaagaaggcagccgagaagaaacgggaggacaaagctaagaagaagcacgacaggaaatccaagcgcctggatgaggaggaggaggacaatgaaggcggggagtgggaaagggtccggggcggagtgccgttggttaaggagaagccaaaaatgtttgccaagggaactgagatcacccatgctgttgttatcaagaaactgaatgagatcctacaggcacgaggcaagaagggaactgatcgtgctgcccagattgagctgctgcaactgctggttcagattgcagcggaaaacaacctgggagagggcgtcattgtcaagatcaagttcaatatcatcgcctctctctatgactacaaccccaacctggcaacctacatgaagccagagatgtgggggaagtgcctggactgcatcaatgagctgatggatatcctgtttgcaaatcccaacatttttgttggagagaatattctggaagagagtgagaacctgcacaacgctgaccagccactgcgtgtccgtggctgcatcctaactctggtggaacgaatggatgaagaatttaccaaaataatgcaaaatactgaccctcactcccaagagtacgtggagcacttgaaggatgaggcccaggtgtgtgccatcatcgagcgtgtgcagcgctacctggaggagaagggcactaccgaggaggtctgccgcatctacctgctgcgcatcctgcacacctactacaagtttgattacaaggcccatcagcgacagctgaccccgcctgagggctcctcaaagtctgagcaagaccaggcagaaaatgagggcgaggactcggctgtgttgatggagagactgtgcaagtacatctacgccaaggaccgcacagaccggatccgcacatgtgccatcctctgccacatctaccaccatgctctgcactcgcgctggtaccaggcccgcgacctcatgctcatgagccacttgcaggacaacattcagcatgcagacccgccagtgcagatcctttacaaccgcaccatggtgcagctgggcatctgtgccttccgccaaggcctgaccaaggacgcacacaacgccctgctggacatccagtcgagtggccgagccaaggagcttctgggccagggcctgctgctgcgcagcctgcaggagcgcaaccaggagcaggagaaggtggagcggcgccgtcaggtccccttccacctgcacatcaacctggagctgctggagtgtgtctacctggtgtctgccatgctcctggagatcccctacatggccgcccatgagagcgatgcccgccgacgcatgatcagcaagcagttccaccaccagctgcgcgtgggcgagcgacagcccctgctgggtccccctgagtccatgcgggaacatgtggtcgctgcctccaaggccatgaagatgggtgactggaagacctgtcacagttttatcatcaatgagaagatgaatgggaaagtgtgggaccttttccccgaggctgacaaagtccgcaccatgctggttaggaagatccaggaagagtcactgaggacctacctcttcacctacagcagtgtctatgactccatcagcatggagacgctgtcagacatgtttgagctggatctgcccactgtgcactccatcatcagcaaaatgatcattaatgaggagctgatggcctccctggaccagccaacacagacagtggtgatgcaccgcactgagcccactgcccagcagaacctggctctgcagctggccgagaagctgggcagcctggtggagaacaacgaacgggtgtttgaccacaagcagggcacctacgggggctacttccgagaccagaaggacggctaccgcaaaaacgagggctacatgcgccgcggtggctaccgccagcagcagtctcagacggcctactgagctctccactctgtttcccgcctgggccatccaaccttgaagtcctaaaccacacctcagtcactaaaggtctgtttaaagttgttctggttgattgcttgttgcca

SEQ ID NO: 125 - DNA, 1020 bp, Saccharomyces cerevisiae
  ref <308>: DATABASE ACCESSION NUMBER: Genbank/AAC03225.1
  ref <309>: DATABASE ENTRY DATE: 2000-10-10
  ref <313>: RELEVANT RESIDUES: (1)..(1020)
atgtctgaacttaatgcattattaaaagatatcaacggctcgctcactgcgacatcagaatccttggagaggttgtctgggatttatagtaattctgcgaccgatgagattcctgaaagtaaccaactacatgagcatctattttacgacgctaagaagcctgctgagaaagtatcgctgctatccttaaaaaatgggagcatgctagggtacataaattctctattgatgcttataggcaataggctagacgacgagtgcaaagatccttctgctatggatgcacgtgaacgctctattcaacaccgtgtggtattagagcgtggtgttaaaccactagaaaaaaagttggcttaccaattggacaagctgactagagcatatgtgaaaatggaaaaggaatataaagacgctgagaagcgtgcactggaaaaatctaccttagtgaatcatagcggcaacgacgatagcgaagatgatgagtctagtgaggatgaaatagcatacaggccaaatacctctggaattatcaacacaaataaaaaatcatcagcatacagggtggaggaaacggctaagcaagaaaacggggaagaaaacgatgacaatgagactggcgtgtataaaccaccaaagattacggctgttctaccaccgcaacaaacgcattttgaagatagattcgatgccagagaacacaaagatcgtagtaacaaatcgcgtatgcaagccatggaagaatatattagagagtcatcggaccaaccggactggagtgcatctattggtgctgacattgtgaaccatggaagaggcggtatcaaatctttgagagacacagagaaggaacgtagagtcacttcattcgaagaagataattttaccagattgaatattacaaataaagctgaaaaaaggaagcaaaagcaacgagaaagaaatgcaaggatgaacgttatcggtggtgaagattttggtatattcagctcaaagaggaagctggaagatagcacttcgaga

SEQ ID NO: 126 - DNA, 1086 bp, Candida albicans
atgtcaaaggtagacactgtattaaaggaaatcatctcgtctaccaagtcaactgaagcttcagtgaaagagttgatagcttttgtcaaggactcgtcttcccaacatccagaattggtgcggaacttgttagcaaaatcaaacctgctgttagaaggggtatcgttgttggggttgaaaaacgaatcgttggtgtcctatatcaacaatatagtgcttgttgttttgtctcatctagagcgtctagaaagcgatctggagacgggatccagcgctgtcgaacgatcgataattcaaagggtgacattggaaaagggcgttaaacctctagaaaagaaactcagttatcagttggataaaatgatcagggcatatggacggatggaacaagacgaaatcaaagctgaacagaagttaaacgatagaggaagtggggagaacgatgagaacgatgagaacgattctgaggaagattctgaagaagattctgaagacgactctgaggacgacgaattggcttatagaccagatgcatcatcgtttgctaaattgacatcggccaaaaccaaactgaaaccaacatcatcagcagtctctacatcgaatgaaaagtatagaccaccaaagatatcagcaatggcacctccaactgcagtaaagagccacgaccttgatgccaacaccacgtcgtcaaagaaccgtaaattacagagcatggaagagtacttgcaagagcaaagtgatatgccaatggtggaggcatcggtggggtctacaattgtggagcatggaagaggtggtgttaaaacacagcacgatcgtaagaaagaacgagagatacaaacgtatgaagaggataattttgtcagactaccaaccagtcaaacaaagaaaagtttcaaggaaaaacaacgtgatatccgtaatcaatttgctggtgaagactggtcgatgtttaataataacaaggatgtgacccgtcaaggcacatcgcgaaagagaaaggcaaccaccgtttgggacaaagtcaagaaaaagaagaatacttagatggtaagtagacgctgacattttgctgcagtatag

SEQ ID NO: 127 - DNA, 1134 bp, Homo sapiens
  ref <308>: DATABASE ACCESSION NUMBER: Human Genbank/AL050003
  ref <309>: DATABASE ENTRY DATE: 2000-02-18
  ref <313>: RELEVANT RESIDUES: (1)..(1134)
gggggctttgcgaagatggcggcgctgggggtgctggagtccgacctgccaagtgccgtgacacttctgaaaaatctccaggagcaagtgatggctgtaactgcacaagtgaaatcactgacacaaaaagttcaagctggtgcctatcctacagaaaagggtctcagcttcttggaagtgaaagaccagctgctgctcatgtaccttatggatttgacccacctcattctggacaaagcctcaggaggatctcttcagggacatgatgcagttttgagactggtggagattcgcacggttttggaaaagcttcgtcccttggaccaaaagctgaagtatcaaattgacaagctgatcaagactgcagtgacaggcagccttagtgagaatgacccacttcgttttaagcctcatcccagcaatatgatgagcaagttgagctctgaggatgaggaggaagatgaagcagaagatgaccagtctgaggcttcagggaagaaatctgtgaagggagtgtctaagaaatatgttcctccacgcttggttccagtacattatgatgaaacagaagctgagcgggagaagaagcgtctagaacgagccaagagacgggcattgagcagctctgtcattcgtgaacttaaggagcagtactcagatgctccagaggaaatccgtgatgctcggcatccccatgttacccgccagagtcaggaggaccaacacaggattaactatgaggagagcatgatggtgcgtttgagcgtcagtaagcgagagaaaggacggcgaaaacgagcaaatgtcatgagctcacaacttcattcccttacacacttcagtgacatcagtgctttgacagggggaactgttcatcttgatgaggatcagaatcctattaagaagcggaagaagatacctcagaaaggtcggaagaaaaaaggccagtgaactgctgggacttaggtgatcaggtgcaaggtggggagtacaaattgagtctctttggatttgccattctgggtctcaccaagccctgtagtatctcttccatactgggcaataatctccttaggtgggcgtggggccaagaagactcgttctgcctgggatagagctcaaaggagactgtag

SEQ ID NO: 128 - DNA, 666 bp, Saccharomyces cerevisiae
  ref <308>: DATABASE ACCESSION NUMBER: Genbank/CAA95901.1
  ref <309>: DATABASE ENTRY DATE: 1997-08-11
  ref <313>: RELEVANT RESIDUES: (1)..(666)
atgagcgctaccgaatcttcatctatattcacattgagtcacaactcaaacctacaagatatcttggccgccaatgccaaatgggcctcccagatgaacaacatacagccaactttgttcccagatcacaatgcgaagggccagtcccctcacactcttttcatcggctgctccgattcgcgttacaacgaaaactgtttaggtgtcttgcccggcgaagtgttcacttggaaaaatgttgctaacatatgtcactcagaggatttaactttgaaggccactttagagtttgccattatttgtctaaaagttaacaaagttattatttgtggccacactgattgtggtggtataaagacatgtttaactaaccaaagggaagccttaccaaaagttaactgttctcatctgtacaagtacttagacgatattgacaccatgtaccatgaagagtcacaaaatttgatccatttgaaaacgcaacgtgaaaaatctcattacctgtcgcactgtaacgtcaaaaggcagtttaataggattattgaaaaccctactgtgcaaactgctgtacaaaatggagaattacaggtatacggtctgctttacaacgtagaggacggtctactgcaaacagttagcacttacacaaaagttaccccaaaatag

SEQ ID NO: 129 - DNA, 846 bp, Candida albicans
atgggtagagaaaatattttgaaatatcaattggaacatgatcatgaatctgatcttgttactgaaaaagatcaatcattattacttgataataataacaacctaaacgggatgaataataccattaaaactcatccggtacgtgttagttcaggaaatcataataattttcctttcactttatcttcagaatctacattacaagattttttaaataataataaattttttgttgattccataaaacataatcatggtaatcaaatatttgatttgaatggtcaaggtcaatctcctcatacattatggatagggtgtagtgattcaagagcaggtgatcaatgtttagctacattaccaggagaaatatttgttcatagaaacattgctaatatagtcaatgccaatgatataagtagtcaaggggttatacaatttgctattgatgtattaaaagtgaaaaaaatcattgtttgtggtcatactgattgtggtggtatttgggcatcattatcaaagaaaaaaattggtggtgttttagatttatggttaaatccagttagacatattcgtgctgctaatttaaaattattagaagaatataatcaagatcctaaattaaaggccaaaaaattggctgaattaaatgtcatttcttctgtaacagcattgaaaagacatcctagtgctagtgttgcattaaagaagaatgaaattgaagtttgggggatgttatatgatgtggcaactggttatttatctcaagtagagattcctcaagatgaatttgaggatttattccatgttcatgatgaacatgatgaagaagaatataaccctcattga

SEQ ID NO: 130 - DNA, 840 bp, Saccharomyces cerevisiae
  ref <308>: DATABASE ACCESSION NUMBER: Genbank/BAA09266.1
  ref <309>: DATABASE ENTRY DATE: 2000-04-14
  ref <313>: RELEVANT RESIDUES: (1)..(840)
atgaaagctaggaaatcgcagagaaaagcgggcagtaaaccaaatcttatccagtctaaattgcaagttaataatggttcgaaatcgaataaaatagtcaagtgtgataaatgtgagatgtcatattcctcgacatcaatagaagatcgcgccatccacgagaaataccacactttacagctgcatggacgtaaatggtcgccgaattggggttctatagtatacacagagcgaaaccattcaaggacggtgcatctatcaagatcgacagggacaataacgccattgaactcctcacctttgaaaaaaagtagtccgtctattacccatcaggaggagaagattgtatatgtgagaccagataagtcgaatggtgaagtccgagccatgacggagataatgacactagtgaataacgagctgaatgcgccacacgatgagaatgtcatttggaacagtaccacagaagaaaaaggcaaagcgtttgtatacataagaaatgacagggcggtcggaataataattatagagaacctttatgggggcaatggtaaaacatctagtcgtggacgttggatggtttatgattctagaagattggtacagaatgtgtaccccgattttaagattggcatatcgagaatttgggtgtgcaggacagcaaggaagttgggtatcgcaaccaaattgattgacgttgcaagagaaaatattgtttacggtgaagttattcctaggtaccaggtagcatggtcgcaacccacagacagcggtggaaaactggctagcaaatacaacggcattatgcataaatcaggcaagttactattgccggtatac

SEQ ID NO: 131 - DNA, 843 bp, Candida albicans
atgggctccattaattctcaaaaacctcaaaaaatccaatcaattcttgcattaccatctaatttcaaaaaaattacttgttcaacatgtgatatgacatataatccccatatatctcaagataaattactacataacaaataccacacaaatttcatcaatggaataccctggaattataaaactgataatgatgttttaataattgagaattttacattagttgaaaccccgaaattgaattccacggggaaatcattaaagctgacaaaaacgcgtcagacatttaaaggttctataatttgtataaataaatccaacaaacgacatatacaaaaagtggaactactattaaacatggtgaatcaagagttgaatgctagtcaagattcaggacaatggaagaaacctgaatttgatagaagtaaagcatttgtgataataatagacagtaaggccattggattatgcacaacagatacaattcaacctgatcaaggaaggtggatgatacataaaacacaatctatagtacctaatcagattaataaaaatgttgtcattggaatttcaagaatatggataagtcggaaatggagacaatatggattaggtaaaaaacttttaaatgttgttttgaaaaattctatttacagtgtgcaattattgaagaatcaagttgcctttagtcaaccaagttttagtggtggaatgttggcaaaatcattcaatggggtgaaacataaaagtggtgaaatgttgttacccgtatatattgaatgatcctttcaggttttcggaggcggcggtgattatgggtgtacatatttgtatattttttgt

SEQ ID NO: 132 - DNA, 1800 bp, Saccharomyces cerevisiae
  ref <308>: DATABASE ACCESSION NUMBER: Genbank/CAA85033.1
  ref <309>: DATABASE ENTRY DATE: 1998-03-16
  ref <313>: RELEVANT RESIDUES: (1)..(1800)
atgctaaatggggaagactttgtagagcataatgatatcctatcgtctccggcaaaaagcaggaatgtaaccccaaaaagggttgacccacatggagaaagacaactgagaagaattcattcatcaaagaagaatttgttggaaagaatctcgcttgtaggcaacgaaaggaaaaatacatctccagatccggcactcaaacctaaaacgccaagtaaagctccccgtaaacgtggaagaccaagaaagatacaggaagaattaactgataggatcaagaaggatgagaaagatacaatttcctctaagaaaaagaggaaattggacaaagatacatcaggtaatgtcaatgaggaaagcaagacttctaacaacaagcaggtgatggaaaagacggggataaaagagaaaagagaacgcgaaaaaatacaggtagcgaccacaacatatgaagataatgtgactccacaaactgatgataattttgtatcaaattcacccgagccaccagaacctgcaacaccatctaagaagtctttaaccactaatcatgattttacttcgcccctaaagcaaattataatgaataatttaaaagaatataaagactcaacctccccaggtaaattaaccttgagtagaaattttactccaacccctgtaccgaaaaataaaaagctctaccaaacttcggaaaccaagtcagcaagctcgtttttggatacttttgaaggatatttcgaccaaagaaaaattgtcagaactaatgcgaagtcaaggcacaccatgtcaatggcacctgacgttaccagagaagagttttccctagtatcaaactttttcaacgaaaattttcaaaaacgtcccaggcaaaagttatttgaaattcagaaaaaaatgtttccccagtattggtttgaattgactcaaggattctccttattattttatggtgtaggttcgaaacgtaattttttggaagagtttgccattgactacttgtctccgaaaatcgcgtactcgcaactggcttatgagaatgaattacaacaaaacaaacctgtaaattccatcccatgccttattttaaatggttacaaccctagctgtaactatcgtgacgtcttcaaagagattaccgatcttttggtccccgctgagttgacaagaagcgaaactaagtactggggcaatcatgtgattttgcagatccaaaagatgattgatttctacaaaaatcaacctttagatatcaaattaatacttgtagtgcataatctggatggtcctagcataaggaaaaacacttttcagacgatgctaagcttcctctccgtcatcagacaaatcgccatagtcgcctctacagaccacatttacgctccgctcctctgggacaacatgaaggcccaaaactacaactttgtctttcatgatatttcgaattttgaaccgtcgacagtcgagtctacgttccaagatgtgatgaagatgggtaaaagcgataccagcagtggtgctgaaggtgcgaaatacgtcttacaatcacttactgtgaactccaagaagatgtataagttgcttattgaaacacaaatgcagaatatggggaatctatccgctaacacaggtcctaagcgtggtactcaaagaactggagtagaacttaaacttttcaaccatctctgtgccgctgattttattgcttctaatgagatagctctaaggtcgatgcttagagaattcatagaacataaaatggccaacataactaagaacaattctggaatggaaattatt

SEQ ID NO: 133 - DNA, 2130 bp, Candida albicans
atgtcacactcaaatgctctaccaaatagtccattccggtcacctaaaaaacaacgtatggaggtcataggaccactcaatgcgtctcgtttttccttttcgccggtaaagacacctcctcatgggagagctggtctatcatctccagagaaaagattagtcaaagaccttgacaagtcggcgagaaaaagagccaacaatagcttatataaccgattaatggatgagtatctggacacagatgattatttggatgaacaagataggatattggccgacagaattatcaaacagtcgaggggagaacccgacgaagtcaattatggcagcgacgtggaattggaaattgatctaactcagcagagacgaacccgaagaagagaaaagaaagttgtttactcgagcgatagtagcaacgaatatgaggatacaggaatgccagaagaatcttcaagcgaggaagaagaggcagatgatgatgatggcaatgtggagtttgtttatggaccacccaaagaaagaaaaacgtcgttatcaagctcaccacccacagtcaagcctactgtgcgccgaaccaagcgaggtagaccaagcaagagtgagcttgttctgggtcaaatcaaaagtatattccatcaagatgacgtgttgttcagtacagatagaaaaacgttcacaccgactaaaccaaccgcagcgaaaaaaccagtcagcaattatttgacatctatttttgatcaaaatttcgatagaagcaaggtgccaagtctaagtggaattcccaaatcaaccaacacgcatgaagagaagaaaacgtttgtgccgcttcctattcccaccctcgatgctgacggaaatatcactgacaaggagtacatctccaaatactttgatggagttgaccctgcaaagttcaaagaaggcaggtttgtggacgaaaaagtattttacttagaagggccagaaggatactttgaacagcaaactaccagagttaaacaaagtggcaactctttaacagcattggcaccccagattgagtacaaagattttgccaggttagtaaagttgggcgacaacctcagtttccaacgcaaacgccaccttttcgaattgcacaagtatatctatcaccagtggtgttttgaaatgtcacaagggttcaatttgaatttctacggagtcggatccaaaatcgatctactccgagattttgccacaaactattttggcatctggtgggaaaatgtggtacacgccgatttgccaaaggttttggtggttaacggttttaaccctagcatcaatatcaaaaaactaattctcgaaatcgcttccatccttttgccaaacgaactgtacccaaaacatatagctggaacggttccctttgtggttgattatctaaacaaccatagactgccctgtggaagtatcggtttccataaacccaaaatcttgttgattattcacaatcttgatggggaagtttttagagtagacaagacacagacgcttttgtcgcaattaatgacactaccagaagtatgggccatgtcatctaccgaccacatcaatgcatcattgttatgggacctgtccaaagttaaaaacttgaatttcatctggcataatctcacaacatatgccacttaccaacgagaaacatctttccgagacgtgataagtttaggcaaatccaaaaaatttgtgggtggccttggtgccaagtatgtcttgcgctcgcttaccgacaatcaccgaaacctctaccgcgagctattgattgcacaattggataaaatggagaaagctgtcccatctgcttctggaagagtgggtttgaaaggtaatgccaaggttgctgttgacctaaaaagcctatacaatacatgtttggacgagttcattacttccaacgagatgaactttagaacattcttaaaagagtatgttgagcataaaatgtgtcagctagtaaaagatccttcaggagttgagaaggtattcattccgttcacatacgaagagatacaaaacatatataagcaagaatttgatgtatagtgggtaccctacacgtatgcggaacttgaaaaacttctgaaaaccgttttaaatactctataa

SEQ ID NO: 134 - DNA, 2640 bp, Homo sapiens
  ref <308>: DATABASE ACCESSION NUMBER: Human Genbank/GI:4433811
  ref <309>: DATABASE ENTRY DATE: 1999-03-18
  ref <313>: RELEVANT RESIDUES: (1)..(2640)
ggcgcgaattactggaaattggcttttcccgttggggccgaaggtaccttccctgcggcggcgactcagcggggtgtcgttcggccggcgtgacgcagccggatcggcgccagacggaaacctagcggtgactgtatctgaattttgcagctgcagaatgtgtagtaccttaaaaggttggcaacaatgagtaaaccagaattaaaggaagacaagatgctggaggttcactttgtgggagatgatgatgttcttaatcacattctagatagagaaggaggagctaaattgaagaaggagcgagcgcagcttttggtcaaccccaaaaaaataataaagaagccagaatatgatttggaggaagatgaccaggaggtcttaaaagatcagaactatgtggaaattatgggaagagatgttcaagaatcattgaaaaatggctctgctacaggtggtggaaataaagtttattcttttcagaatagaaaacactctgaaaagatggctaaattagcttcagaactagcaaaaacaccacaaaaaagtgtttcattcagtttgaagaatgatcctgagattacgataaacgttcctcaaagtagcaagggccattctgcttcagacaaggttcaaccgaagaacaatgacaaaagtgaatttctgtcaacagcacctcgtagtctaagaaaaagattaatagttccaaggtctcattctgacagtgaaagcgaatattctgcttccaactcagaggatgatgaaggggttgcacaggaacatgaagaggacactaatgcagtcatattcagccaaaagattcaagctcagaatagagtagtttcagctcctgttggcaaagaaacaccttctaagagaatgaaaagagataaaacaagtgacttagtagaagaatattttgaagctcacagcagttcaaaagttttaacctctgatagaacactgcagaagctaaagagagctaaactggatcagcaaactttgcgtaacttattgagcaaggtttccccttccttttctgccgaacttaaacaactaaatcaacagtatgaaaaattatttcataaatggatgctgcaattacaccttgggttcaacattgtgctttatggtttgggttctaagagagatttactagaaaggtttcgaaccactatgctgcaagattccattcacgttgtcatcaatggcttctttcctggaatcagtgtgaaatcagtcctgaattctataacagaagaagtcctcgatcatatgggtactttccgcagtatactggatcagctagactggatagtaaacaaatttaaagaagattcttctttagaactcttccttctcatccacaatttggatagccagatgttgagaggagagaagagccagcaaatcattggtcagttgtcatctttgcataacatttaccttatagcatccattgaccacctcaatgctcctctcatgtgggatcatgcaaagcagagtctttttaactggctctggtatgaaactactacatacagtccttatactgaagaaacctcctatgagaactctcttctggtaaagcagtctggatccctgccacttagctcccttactcatgtcttacgaagccttacccctaatgcaaggggaattttcaggctactaataaaataccagctggacaaccaggataacccttcttacattggcctttcttttcaagatttttaccagcagtgtcgggaggcattcctcgtcaatagtgatctgacactccgggcccagttaactgaatttagggaccacaagcttataagaacaaagaagggaactgatggagtagagtatttattaattcctgttgataatggaacattgactgatttcttggaaaaggaagaagaggaggcttgaagctttcctttattcttgaatctcccatggaagggttgtaccccagctgccactcctctagttgaaagtgttgtgtttacatctgacattaaattatttttccagcatacaagatttaaatttgggaagggggggatgtcctcaattagaactttttgatcagcctggctggtaccgtctagtactatgcagcggtcctcaagttggagaaaatgtgcctttcattcattacctctctggagacttcttgctggaatgaacagtgtgctcagggactatttggaactggatgtttttgaattattttatacttagagatattctgaattttttgagggccttttaacactccccgagctgattgtttgcaagtgtgtttgttccagagtgtggaagtataaagacatgggcatcacgtaaattggttttgtttgctattctgtgtgtcagaaccaacgagtgtaatggagagggcaggtcatctcttattgtttctaaaacaacttaaaaggtgtagattgggaagaggtgagtgatccagctttctccttttggattgaggctatgtacttggtgggggcaggggagggaatatattataatactattcagttgggataatgggaaaaacagagtatatagggtatctacccagcctagaaagcacaggaacaatacgtcatatatttggaacagttattgtctgtgccatgaccttca

SEQ ID NO: 135 - DNA, 617 bp, Saccharomyces cerevisiae
  ref <308>: DATABASE ACCESSION NUMBER: Genbank/CAA85114.1
  ref <309>: DATABASE ENTRY DATE: 1997-08-11
  ref <313>: RELEVANT RESIDUES: (1)..(617)
ccaatcaaaggattgacccagagaacaaatcaattttgaatatgttatcagtgattgatagaaaagaacaagaattgaaagcaaaagaagaaaaacagcaaagagaagctcaggaacgtgaaaacaagaaaattatgttagagagcgcaatgacgctgagaaacataactaacatcaaaactcactctccagtagagttacttaatgagggtaaaataaggctagaagacccaatggattttgaatctcaattgatctatcccgcattaattatgtaccccacgcaagatgaatttgattttgtaggtgaagtaagtgagttaactactgtgcaagaacttgttgacctagttttggaagggccgcaagaacgcttcaaaaaagaaggtaaggaaaacttcacaccaaagaaagtgttggtgttcatggaaacaaaggcaggtggtttgattaaagctggtaagaaactgacatttcacgatatcttgaagaaagagtcgccagatgtaccattgttcgataacgctttgaaaatatatattgtgccaaaggtagaaagtgaagggtggatttccaagtgggataagcaaaaagccttagaaagaagatctgtgtga

SEQ ID NO: 136 - DNA, 1173 bp, Candida albicans
atgtccaaaatagagccagtcactgaaaaagaagaagaatacgtttccgaatgggatagaagaagatatgttcccaaagcaggtgaacctgaattacctccccaattatcagaattctctaacaagaccacagacgaggttattgaggaattgaatagattgccattttttatgacaaagttagatgaaactgatggagatggcggagaaaatgtaaacttggaagcacttaaaagtttggcatatgaaggtgatcctgacgaaattgcctcaaatttcaaaaatcaggggaataattgttacaaatttaaaaaatacaaagatgcaattatattttatacgaaaggtcttgaagtaaactgtgacgtggacgcaatcaattcagcattatacttgaatcgtgctgcttgtaacttggagttgaaaaattaccgtcggtgcattgaagattgtaagaaagtattaatgcttgatgagaagaatattaaggcttgtttccgttcaggaaaggcattctttgcaattgaaaaatacgatgaagcaatcaaagtgcttgaatacggtctaaatatagaaccagaaaacaaagatttacagaaattattacagcaagttcaaaagaggcaagaaactttagctcaaataaaagctaaaaaggcacaagaagaggaacaagagcggttgaaaaatatcgtgttggagaattctataaaattaagacacattgaaatagtgaagtcctcatctcctccagaagtcttgaagactgccaagatacgattggaagaccccaaagattatcagtcacaattaatattccctgctatgatactataccccaccaccgatgaatttgactttattgcagaaataagcgaattaactactcctttggaattgctagagatggtattaaatagacctagggaatggtttgatgatccaaaacacaaggatttcaatgtcaaaaaattggaatgctttatggaaactgaatctggtgggttgattaaagtgggcaagaaaattgaagttaacaatgctttgatgaatgaaaaacctaaggcaccattgtttgataacgctttaagactttatgtcgttccaaaattagacgtcgccaaatggacatctgaatggaataaagaaaccgccttggcagctcgtaaatag

SEQ ID NO: 137 - DNA, 2005 bp, Homo sapiens
  ref <308>: DATABASE ACCESSION NUMBER: Human Genbank/NM_004623.1
  ref <309>: DATABASE ENTRY DATE: 2000-11-01
  ref <313>: RELEVANT RESIDUES: (1)..(2005)
ctgggacccgggctggaaggcagggcatcagctatggaacaacctgggcaggatcccacctcagacgacgtcatggactcgttcctggaaaagttccagagccagccttaccgtggcggctttcatgaggaccagtgggagaaggaatttgaaaaggtccccctatttatgtcgagagcgccatcagaaattgatcccagggagaatcctgacttggcttgtctccagtcaattatttttgatgaggagcgttctccagaagaacaggccaagacctataaagatgagggcaatgattactttaaagaaaaagactacaagaaagctgtaatttcatacactgaaggcttaaagaagaaatgtgcagatcctgatttgaatgctgtcctttataccaaccgggcagcagcacagtactatctgggcaattttcgttctgctctcaatgatgtgacagctgccagaaagctaaaaccctgccacctcaaagcaataataagaggtgccttatgccatctggaactgatacactttgccgaggccgtgaactggtgtgatgagggactgcaaatagatgccaaagagaagaagcttctggaaatgagggctaaagcagacaagctgaagcgaattgaacagagggatgtgaggaaagccaacttgaaagaaaagaaggagaggaatcagaatgaggctttactccaggccatcaaggctaggaatatcaggctctcagaagctgcctgtgaggatgaagattcagcctcagaaggtctaggtgagcttttcctggatggactcagcactgagaacccccatggagccaggctgagtctagatggccagggcaggctgagctggcctgtgctctttctgtacccagagtatgcccagtcggacttcatctctgcttttcatgaggactccaggtttattgatcatctaatggtgatgtttggtgaaacaccttcttgggacctagagcaaaaatattgcctgataatttggaggtctactttgaggatgaggacagggcagaactataccgggtgcctgccaagagcaccttgctacaggttctacagcaccagaggtactttgtaaaagccctgacaccagcatttttggtctgtgtaggatcctctcctttttgcaagaattttctccgggggagaaaggtgtaccagatacgatgactaagccagggcccctggatctcctcccttaccctcctctgctgggaacctagcacacctgaatcagctggacatactgctggagtccagtgctttctttccgtcaccctggggatagtccttcctggcatcgtggtgggggaggagcctctggcttccctaaactgcagctctctggctggtcttcactttcctcagttgatataaaactctggtcttggccatgatgtccttggattccatcgctaaagggaccatctgctgcagttaccacagcaactgacttgagcggcacctggtctgtggagatggactcaggatccagtgacatgattctgaacttttgtggagtttgacaccttagagaagctacccctcaaactgcacatctacacacaaacaaacaatgcataggattccaaggctttaaagctgagagaccctggcctcaagttatttcatgcgcacagagggaagccatgtggggttgctgaagatgccttgaggtgaaatgggggcaggaaagccacatcttgctctgcatttataaagaccgtacaaactcagatccttggtacccctaaaaagattgccaattttcttcatctttgccatatggaggactgtgacagactttggacagtggcctcttgagttcctctgcagttttgacatttaggattttgtgtcttttaaactggaaaatcttctagcatgttgggttgttacagagtatatttttgtctgcagctgtttgttgccccattcctaagaggagtttatccatcctgaaaaaaaaaaaaaaaaaa

SEQ ID NO: 138 - DNA, 504 bp, Saccharomyces cerevisiae
  ref <308>: DATABASE ACCESSION NUMBER: Genbank/CAA98815.1
  ref <309>: DATABASE ENTRY DATE: 1997-08-11
  ref <313>: RELEVANT RESIDUES: (1)..(504)
atgtctactattccctcagaaatcatcaattggaccatcttaaatgaaattatatctatggatgacgatgattccgatttttctaaaggtctaattattcaatttatcgaccaggcacaaacaacttttgctcaaatgcaacgacagctggacggtgaaaaaaatcttaccgaattagacaatctgggccattttttaaagggttcttctgctgcattaggcttacaaagaattgcctgggtttgtgaaagaattcaaaacttgggaagaaaaatggaacatttcttccccaacaagaccgaattggtcaacactctgagcgataaatcgattattaatggaatcaatattgatgaagatgacgaggaaataaagatacaagtggacgataaagacgaaaattccatatatctcatcttgatagcaaaagctttgaaccagtctaggttggagttcaaactggcgagaattgagttatctaaatattacaacacaaacctataa

SEQ ID NO: 139 - DNA, 555 bp, Candida albicans
atgtcagaagataaattacaaaaattacaagactcaggacttgtcgactgggcagtgtttagtgaaatagtgaccatggacgaggatgaagaagggttttccaaatcactagtagaagtctttgttagccaagtggaagaaacatttgaagaaattgataaatatttaaaggaaaagaatttggagaaattgtcatcgtcgggtcattttttgaaaggatctgctgctgctttggggttgaccaaaatttcaaatcaatgcgaacgaattcaaaactatggccataagatcaactttgacaattttcaattggaagatataaaaactaaaggcgattcggccgtaagtgcggaaaatgtggccgttaatgatggtgaaactaatccagaaaatggatccaatggcaacgaaacaagtaataacaaaacaaatactagcaatataccggatgaatcaagcgatgacttttggatagcattaattgaggatgcattagccaaggcgagagatggattcgaccaatctagaagagcattggacgaatattacgaatag

SEQ ID NO: 140 - DNA, 7992 bp, Homo sapiens
  ref <308>: DATABASE ACCESSION NUMBER: Human Genbank/Z15005.1
  ref <309>: DATABASE ENTRY DATE: 1993-01-10
  ref <313>: RELEVANT RESIDUES: (1)..(7992)
atggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattcttcagaattccttgggaggaaatccaaagacacgtattatctgcacaattactccagtatcttttgatgaaactcttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtctgttcagagtctgatgttttcagtaacactcttgatacattaagtgagatagaatggaatccagcaacaaagctactaaatcaggagaatatagaaagtgagttgaactcacttcgtgctgactatgataatctggtattagactatgaacaactacgaacagaaaaagaagaaatggaattgaaattaaaagaaaagaatgatttggatgaatttgaggctctagaaagaaaaactaaaaaagatcaagagatgcaactaattcatgaaatttcgaacttaaagaatttagttaagcatcgagaagtatataatcaagatcttgagaatgaactcagttcaaaagtagagctgcttagagaaaaggaagaccagattaagaagctacaggaatacatagactctcaaaagctagaaaatataaaaatggacttgtcatactcattggaaagcattgaagacccaaaacaaatgaagcagactctgtttgatgctgaaactgtagcccttgatgccaagagagaatcagcctttcttagaagtgaaaatctggagttgaaggagaaaatgaaagaacttgcaactacatacaagcaaatggaaaatgatattcagttatatcaaagccaattggaggcaaaaaagaaaatgcaagttgatctggagaaagaattacaatctgcttttaatgagataacaaaactcacctcccttatagatggcaaagttccaaaagatttgctctgtaatttggaattggaaggaaagattactgatcttcagaaagaactaaataaagaagttgaagaaaatgaagctttgcgggaagaagtcattttgctttcagaattgaaatctttaccttctgaagtagaaaggctgaggaaagagatacaagacaaatctgaagagctccatataataacatcagaaaaagataaattgttttctgaagtagttcataaggagagtagagttcaaggtttacttgaagaaattgggaaaacaaaagatgacctagcaactacacagtcgaattataaaagcactgatcaagaattccaaaatttcaaaacccttcatatggactttgagcaaaagtataagatggtccttgaggagaatgagagaatgaatcaggaaatagttaatctctctaaagaagcccaaaaatttgattcgagtttgggtgctttgaagaccgagctttcttacaagacccaagaacttcaggagaaaacacgtgaggttcaagaaagactaaatgagatggaacagctgaaggaacaattagaaaatagagattctccgctgcaaactgtagaaagggagaaaacactgattactgagaaactgcagcaaactttagaagaagtaaaaactttaactcaagaaaaagatgatctaaaacaactccaagaaagcttgcaaattgagagggaccaactcaaaagtgatattcacgatactgttaacatgaatatagatactcaagaacaattacgaaatgctcttgagtctctgaaacaacatcaagaaacaattaatacactaaaatcgaaaatttctgaggaagtttccaggaatttgcatatggaggaaaatacaggagaaactaaagatgaatttcagcaaaagatggttggcatagataaaaaacaggatttggaagctaaaaatacccaaacactaactgcagatgttaaggataatgagataattgagcaacaaaggaagatattttctttaatacaggagaaaaatgaactccaacaaatgttagagagtgttatagcagaaaaggaacaattgaagactgacctaaaggaaaatattgaaatgaccattgaaaaccaggaagaattaagacttcttggggatgaacttaaaaagcaacaagagatagttgcacaagaaaagaaccatgccataaagaaagaaggagagctttctaggacctgtgacagactggcagaagttgaagaaaaactaaaggaaaagagccagcaactccaagaaaaacagcaacaacttcttaatgtacaagaagagatgagtgagatgcagaaaaagattaatgaaatagagaatttaaagaatgaattaaagaacaaagaattgacattggaacatatggaaacagagaggcttgagttggctcagaaacttaatgaaaattatgaggaagtgaaatctataaccaaagaaagaaaagttctaaaggaattacagaagtcatttgaaacagagagagaccaccttagaggatatataagagaaattgaagctacaggcctacaaaccaaagaagaactaaaaattgctcatattcacctaaaagaacaccaagaaactattgatgaactaagaagaagcgtatctgagaagacagctcaaataataaatactcaggacttagaaaaatcccataccaaattacaagaagagatcccagtgcttcatgaggaacaagagttactgcctaatgtgaaaaaagtcagtgagactcaggaaacaatgaatgaactggagttattaacagaacagtccacaaccaaggactcaacaacactggcaagaatagaaatggaaaggctcaggttgaatgaaaaatttcaagaaagtcaggaagagataaaatctctaaccaaggaaagagacaaccttaaaacgataaaagaagcccttgaagttaaacatgaccagctgaaagaacatattagagaaactttggctaaaatccaggagtctcaaagcaaacaagaacagtccttaaatatgaaagaaaaagacaatgaaactaccaaaatcgtgagtgagatggagcaattcaaacccaaagattcagcactactaaggatagaaatagaaatgctcggattgtccaaaagacttcaagaaagtcatgatgaaatgaaatctgtagctaaggagaaagatgacctacagaggctgcaagaagttcttcaatctgaaagtgaccagctcaaagaaaacataaaagaaattgtagctaaacacctggaaactgaagaggaacttaaagttgctcattgttgcctgaaagaacaagaggaaactattaatgagttaagagtgaatctttcagagaaggaaactgaaatatcaaccattcaaaagcagttagaagcaatcaatgataaattacagaacaagatccaagagatttatgagaaagaggaacaacttaatataaaacaaattagtgaggttcaggaaaacgtgaatgaactgaaacaattcaaggagcatcgcaaagccaaggattcagcactacaaagtatagaaagtaagatgctcgagttgaccaacagacttcaagaaagtcaagaagaaatacaaattatgattaaggaaaaagaggaaatgaaaagagtacaggaggcccttcagatagagagagaccaactgaaagaaaacactaaagaaattgtagctaaaatgaaagaatctcaagaaaaagaatatcagtttcttaagatgacagctgtcaatgagactcaggagaaaatgtgtgaaatagaacacttgaaggagcaatttgagacccagaagttaaacctggaaaacatagaaacggagaatataaggttgactcagatactacatgaaaaccttgaagaaatgagatctgtaacaaaagaaagagatgaccttaggagtgtggaggagactctcaaagtagagagagaccagctcaaggaaaaccttagagaaactataactagagacctagaaaaacaagaggagctaaaaattgttcacatgcatctgaaggagcaccaagaaactattgataaactaagagggattgtttcagagaaaacaaatgaaatatcaaatatgcaaaaggacttagaacactcaaatgatgccttaaaagcacaggatctgaaaatacaagaggaactaagaattgctcacatgcatctgaaagagcagcaggaaactattgacaaactcagaggaattgtttctgagaagacagataaactatcaaatatgcaaaaagatttagaaaattcaaatgctaaattacaagaaaagattcaagaacttaaggcaaatgaacatcaacttattacgttaaaaaaagatgtcaatgagacacagaaaaaagtgtctgaaatggagcaactaaagaaacaaataaaagaccaaagcttaactctgagtaaattagaaatagagaatttaaatttggctcaagaacttcatgaaaaccttgaagaaatgaaatctgtaatgaaagaaagagataatctaagaagagtagaggagacactcaaactggagagagaccaactcaaggaaagcctgcaagaaaccaaagctagagatctggaaatacaacaggaactaaaaactgctcgtatgctatcaaaagaacacaaagaaactgttgataaacttagagaaaaaatttcagaaaagacaattcaaatttcagacattcaaaaggatttagataaatcaaaagatgaattacagaaaaagatccaagaacttcagaaaaaagaacttcaactgcttagagtgaaagaagatgtcaatatgagtcataaaaaaattaatgaaatggaacagttgaagaagcaatttgagccaaactatctatgcaagtgtgagatggataacttccagttgactaagaaacttcatgaaagccttgaagaaataagaattgtagctaaagaaagagatgagctaaggaggataaaagaatctctcaaaatggaaagggaccaattcatagcaaccttaagggaaatgatagctagagaccgacagaaccaccaagtaaaacctgaaaaaaggttactaagtgatggacaacagcaccttatggaaagcctgagagaaaagtgctctagaataaaagagcttttgaagagatactcagagatggatgatcattatgagtgcttgaatagattgtctcttgacttggagaaggaaattgaattccacagaatcatgaagaaactgaagtatgtgttaagctatgttacaaaaataaaagaagaacaacatgaatgcatcaataaatttgaaatggattttattgatgaagtggaaaagcaaaaggaattgctaattaaaatacagcaccttcaacaagattgtgatgtaccatccagagaattaagggatctcaaattgaaccagaatatggatctacatattgaggaaattctcaaagatttctcagaaagtgagttccctagcataaagactgaatttcaacaagtactaagtaataggaaagaaatgacacagtttttggaagagtggttaaatactcgttttgatatagaaaagcttaaaaatggcatccagaaagaaaatgataggatttgtcaagtgaataacttctttaataacagaataattgccataatgaatgaatcaacagagtttgaggaaagaagtgctaccatatccaaagagtgggaacaggacctgaaatcactgaaagagaaaaatgaaaaactatttaaaaactaccaaacattgaagacttccttggcatctggtgcccaggttaatcctaccacacaagacaataagaatcctcatgttacatcaagagctacacagttaaccacagagaaaattcgagagctggaaaattcactgcatgaagctaaagaaagtgctatgcataaggaaagcaagattataaagatgcagaaagaacttgaggtgactaatgacataatagcaaaacttcaagccaaagttcatgaatcaaataaatgccttgaaaaaacaaaagagacaattcaagtacttcaggacaaagttgctttaggagctaagccatataaagaagaaattgaagatctcaaaatgaagcttgtgaaaatagacctagagaaaatgaaaaatgccaaagaatttgaaaaggaaatcagtgctacaaaagccactgtagaatatcaaaaggaagttataaggctattgagagaaaatctcagaagaagtcaacaggcccaagatacctcagtgatatcagaacatactgatcctcagccttcaaataaacccttaacttgtggaggtggcagcggcattgtacaaaacacaaaagctcttattttgaaaagtgaacatataaggctagaaaaagaaatttctaagttaaagcagcaaaatgaacagctaataaaacaaaagaatgaattgttaagcaataatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcagtag

SEQ ID NO: 141 - DNA, 282 bp, Saccharomyces cerevisiae
  ref <308>: DATABASE ACCESSION NUMBER: Genbank/AAB68435.1
  ref <309>: DATABASE ENTRY DATE: 1997-09-03
  ref <313>: RELEVANT RESIDUES: (1)..(282)
atgtctttcttaggtttcggtggtggtcagcctcaattatcatctcaacaaaagattcaagctgcggaagctgaactagatttggtcacagacatgttcaataaattggttaataactgttataaaaaatgtatcaatacttcttattccgagggtgagctgaataagaatgaatcttcgtgcctagacagatgtgtggccaaatattttgagaccaatgttcaagtcggtgaaaacatgcagaaaatgggccaatcatttaacgcagccggtaagttttag

SEQ ID NO: 142 - DNA, 278 bp, Candida albicans
atgtttggcttaggtggtactactcctcaaatttcatctcaacaaaaacttcaagctgctgaagctgaattagatatggttactggcatgttcaatgctttagtttcccaatgtcacaccaaatgtatcaacaaatcatataatgaagctgatatttcaaagcaagaatctttatgtcttgatagatgtgttgccaaatattttgaaaccaatgttcaagttggtgaaaatatgcaaaaattaggtcaatctggtcaatttatgggtagaagataaat

SEQ ID NO: 143 - DNA, 658 bp, Homo sapiens
  ref <308>: DATABASE ACCESSION NUMBER: Human Genbank/NM_012456.1
  ref <309>: DATABASE ENTRY DATE: 2001-12-19
  ref <313>: RELEVANT RESIDUES: (1)..(658)
ggagcctcacgrgagcgkggtaacgttatagtatttgtcagaagttggggtctccgtgggcattgtgatccgtcccaggcagtggattaggaggccagaaggagatcccttccacggtgctaggctgagatggatcctctcagggcccaacagctggctgcggagctggaggtggagatgatggccgatatgtacaacagaatgaccagtgcctgccaccggaagtgtgtgcctcctcactacaaggaagcagagctctccaagggcgagtctgtgtgcctggaccgatgtgtctctaagtacctggacatccatgagcggatgggcaaaaagttgacagagttgtctatgcaggatgaagagctgatgaagagggtgcagcagagctctgggcctgcatgaggtccctgtcagtatacaccctggggtgtaccccaccccttcccactttaataaacgtgctccctgttgggtgtcatctgtgaagactgccaggcctaggctctctgtagagagtcttcaagatcccggagtggtagcgctgtctcctggtgaaggagtatttgtcacactggaatgtgactgtgtgtgtatgtatgtgtatatatatatatatatatatatataaacaagtttgttgacacctacaaaaaaaaaa

SEQ ID NO: 144 - DNA, 1980 bp, Saccharomyces cerevisiae
  ref <308>: DATABASE ACCESSION NUMBER: Genbank/AAB64555.1
  ref <309>: DATABASE ENTRY DATE: 2000-10-10
  ref <313>: RELEVANT RESIDUES: (1)..(1980)
atgacaacggaagatccagattcaaatcacttaagttccgaaactggcattaaattggcattggacccgaacttaattacattggcactaagttctaatccaaactctagccttcattcaccaacgtctgatgaacccgtacctgaatctgcaggaaaagcagatactagtattcgactagaaggtgatgagttagagaataaaactaagaaagacaatgataagaacttaaaatttttgaagaataaagattctctagtcagtaatccacacgaaatttatggctccatgccgttggagcaattgatcccaatcatcttaagacagcgtggtccaggctttaaattcgttgatttaaatgaaaaagaattgcaaaatgagattaagcagcttggtagtgatagtagtgacggtcataacagcgagaagaaggacactgatggcgctgatgagaatgtacaaattggagaagatttcatggaagtggattatgaagataaagataatccagtggattcacgaaatgaaacagaccacaaaacgaatgaaaatggcgagaccgatgataatattgaaacggtaatgacacaggaacagtttgttaaaagaaggagggatatgctagagcatataaatctggccatgaacgaatcgtctttggctttggaattcgtttctttgctactgtcgagtgttaaagagtctacaggtatgtcatcaatgtcaccatttcttaggaaagttgttaaaccttctagtttaaacagtgataaaattccatatgttgcacctacaaaaaaagaatatatcgagttggatatattgaataagggatggaagttacaaagtttaaacgaatctaaagatctcctacgcgcaagttttaataaactgagttccatattacagaacgaacatgactattggaataagataatgcagagtattagcaacaaggatgttatttttaagattagggacaggactagtggtcaaaagctgttggcaattaagtatggttacgaagactctggatctacctataagcatgacagaggtattgctaatataaggaataatatagaatcacaaaatttggatttgataccccacagtagttcagtgttcaaaggcactgatttcgtacattcagtaaagaaattcttaagggttcgtatcttcacaaaaatcgaatcagaagatgattacatattgagtggcgaaagtgtgatggatagggatagtgaaagtgaagaagctgaaacgaaagatatcagaaagcaaatccaacttttgaaaaagatcatttttgaaaaagaactgatgtaccaaataaagaaagaatgcgctttgttgatttcctatggtgtcagtattgaaaacgaaaacaaggtaataattgaactacctaacgaaaaatttgaaatcgagttgttgtcccttgacgatgactccattgtcaatcatgaacaagacttaccaaaaatcaacgacaagagagcaaatttaatgcttgttatgttgagactattattagtcgttatattcaagaaaacattacgatcgagaataagctcaccccacggactgatcaatttgaatgttgacgatgatatcttaataatacgtcccattcttggtaaagttcggtttgctaattacaaactgttactaaaaaaaatcataaaggattacgtgctcgatatagttcctggctcaagtataacagaaacggaagttgagagagaacaacctcaagaaaataaaaacattgatgatgaaaatataactaaattaaataaagagatccgtgccttcgataaactattgaatatacctagacgtgaactcaaaataaatctaccattaactgagcacaaaagccctaatctaagtttaatgctcgaaagtcctaactattgtaacgcactcattcacatcaagttttcagctggtacggaagccaacgcagtgtccttt

SEQ ID NO: 145 - DNA, 1849 bp, Candida albicans
atggtggaaaaacagtttaacatagacctagagttaaatgatactggtcatatagatccattcttacaagatgagtatgtttgctttctaactttattggtatttttggttctgttttttagtttactaaccttgaccaagagataaattgaaacttgaggaactaattccacgaattttatttgaacgtaaatcatttttgaatgtgacggaggattctttgagaaaagaaatagacaattcattgaagatttccgaagaggatgctttagacactgaagaaagtagagaggacacagttgaagcagatcaacaagaagtgttcaataaacacaagtttgaattatcgaaaaatataaacaatgcacttaatgaaacccaactttccttagattttgtatccttattaatatcttcagtgaaaccaagtttggcaaaatctaccatttcaccacacttgtcaaaatttgtcaaaccgacatctttaaattcggatagattgggtcaagatagtaatgataatcaagagagtaaggctactgattcttttggacaaggatggaaattggagtcacttggaaagataaccgatcttttcagagaagctagtactaatttaaacgatcaagttatcaaagaaagacgatattggaatatgataaatttggtgcttgccaacgacgaggttctatttcgaatgagggacccccaaaataatgctagagcaataggagtgaaatatgggtatggagattcaggatcaaattttcacgaccaagggttggcattgttacgcaaggacaaccaaacaggagaaatctcatttcaccccatatcgtcaatcaacaatgctaaaattgtagaaaaagtttcgagatttattagagtgaaaattttgagccaaatagatggggactatatgcttacaggacagtcaatttttaattttgattttgaaaaaagcaagcaaagcataattaatgacatcgaaaaggctagattctttttatttgaggaggacttgtttcatcaattgatacgcgaggccaaattgttggtaaactacaatgtgtcaatcatatcgaataaaataataattgaaatcaacaacattattattgaaatagagtctatcgtgtatgatgagttgaatgaggaggaactagaaaactattaccagaatgtaaatgaatattccaccttacacaataaaaagtgtcagcttattttaaactacttgaaacttatgctttgttgttattacaaatacaatctcaaattgaaacagaaggttccaacagcattgactaaatggaagcagagtaactcccatcctttgattttgcgtccgttagtgggtaatatgaggcatgagttaaatttgctaaatatgaagagtgttttagatcgattaatgcacgctcatgagagtgaactttcttattccaaactagatgtggagaagtttattaacttagccacaagaagcaaaaagcaaaacccattccaaaagtcaattgaaaagccaatttcaaagttccatttagttttatgcaacaaaacctctaatatgttggacgtcaacatacaattgacaactaatgagctgtttgtcaatctaatcatcaatatgacaattattagatttgaaacagaagacgattttaagaacaatgtcaatggtattaacgttctacagcttgggttcagtgatttcaatgaaatcgaagaatgcttggattggtcgatccaaaattttgtataggacacaacattttctgattttaaagaagtagaggacttcctacattttattgtcgctgagtacatccagcaaaagaaggtgtaa

SEQ ID NO: 146 - DNA, 2760 bp, Homo sapiens
  ref <308>: DATABASE ACCESSION NUMBER: Human Genbank/AB015617.1
  ref <309>: DATABASE ENTRY DATE: 2000-01-06
  ref <313>: RELEVANT RESIDUES: (1)..(2760)
atgtatggaagtgcccgctctgttgggaaggtggagccgagcagccagagccctgggcgttcacccaggcttccacgttcccctcgcttgggtcaccgtcgaaccaacagtacgggagggagttcgggaagcagtgttggaggtggcagtgggaaaaccctttcaatggaaaatatacaatctttaaatgctgcctatgccacctctggccctatgtatctaagtgaccatgaaaatgtgggttcagaaacacctaaaagcaccatgacacttggccgttctgggggacgtctgccttacggtgttcggatgactgctatgggtagtagccccaatatagctagcagtggggttgctagtgacaccatagcatttggagagcatcacctccctcctgtgagtatggcatccactgtacctcactcccttcgtcaggcgagagataacacaatcatggatctgcagacacagctgaaggaagtattaagagaaaatgatctcttgcggaaggatgtggaagtaaaggagagcaaattgagttcttcaatgaatagcatcaagaccttctggagcccagagctgaagaaggaacgagccctgagaaaagatgaagcttccaaaatcaccatttggaaggaacagtacagagttgtacaggaggaaaaccagcacatgcagatgacaatccaggctctccaggatgaattgcggatccagagggacctgaatcagctgtttcagcaggatagtagcagcaggactggcgaaccttgtgtagcagagctgacagaggagaactttcagaggcttcatgctgagcatgagcggcaggccaaagagctgtttcttcttcgaaagacattggaggaaatggagctgcgtattgagactcaaaagcagaccctaaatgctcgggatgaatccattaagaagcttctggaaatgttgcagagcaaaggactttctgccaaggctaccgaggaagaccatgagagaacaagacgactggcagaggcagagatgcacgttcatcacctagaaagccttttggagcagaaggaaaaagagaacagtatgttgagagaggagatgcatcgaaggtttgagaatgctcctgattctgccaaaacaaaagctctgcaaactgttattgagatgaaggattcaaaaatttcctctatggagcgtgggcttcgagacctggaagaggaaattcagatgctgaaatcgaatggtgctttgagtactgaggaaagggaagaagaaatgaagcaaatggaagtgtatcggagccattctaaatttatgaaaaataagattggccaggtgaaacaggagctgtccagaaaggacacagaactactcgccctgcagacaaagctagaaacactcacaaaccagttctcagatagtaaacagcacattgaagtgttgaaggagtccttgactgctaaggagcagagggctgccatcctgcagactgaggtggatgctctccgattgcgtttggaagagaaggaaaccatgttgaataaaaagacaaaacaaattcaggatatggctgaagagaaggggacacaagctggagagatacatgacctcaaggacatgttggatgtgaaggagcggaaggttaatgttcttcagaagaagattgaaaatcttcaagagcagcttagagacaaggaaaagcagatgagcagcttgaaagaacgggtcaaatccttgcaggctgacaccaccaacactgacactgccttgacaactttggaggaggcccttgcagagaaagagcggacaattgaacgcttaaaggagcagagggacagagatgagcgagagaagcaagaggaaattgataactacaaaaaagatcttaaagacttgaaggaaaaagtcagcctgttgcaaggcgacctttcagagaaagaggcttcacttttggatctgaaagagcatgcttcttctctggcatcctcaggactgaaaaaggactcacggcttaagacactagagattgctttggagcagaagaaggaggagtgtctgaaaatggaatcacaattgaaaaaggcacatgaggcagcattggaagccagagccagtccagagatgagtgaccgaatacagcacttggagagagagatcaccaggtacaaagatgaatctagcaaggcccaggcagaagttgatcgactcttagaaatcttgaaggaggtggaaaatgagaagaatgacaaagataagaagatagctgagttggaaagtctcacctcaaggcaagtgaaagaccagaataagaaggtagcaaatctgaagcacaaggaacaggtggaaaaaaagaagagtgcacaaatgttagaggaggcgcgacgacgggaggacaatctcaacgacagctctcagcagctacaggtggaggagttactgatggccatggagaaggtaaagcaggaactagaatccatgaaagcaaagctgtcctccacccagcagtctctggcagaaaaggaaactcacttgactaatcttcgggcagagagaaggaaacacttagaggaagttctggagatgaagcaagaagctcttctggctgccattagtgaaaaagacgccaatatagctctcttggagctttcgtcctctaagaagaagacccaagaggaagtggctgccctgaagcgggagaaggatcgtctggtacagcagcttaagcagcagacgcaaaatcgaatgaagctaatggccgacaactacgag

(19) United States
(12) Patent Application Publication
(10) Pub. No.: US 2003/0027243 A1
(43) Pub. Date: Feb. 6, 2003

(54) Antifungal compounds and methods of use

(75) Inventors: Craig Thompson, Arlington, MA (US); Jeffrey Moore, Brookline, MA (US); Ed T. Buurman, Arlington, MA (US); John Bradley, St. Louis, MO (US); Thamara Desilva, Brookline, MA (US); Sandra Harris, Dayton, NJ (US); Svetlana Komarnitsky, Brookline, MA (US); Marc Mendillo, Brighton, MA (US); Daniel Moore, Medford, MA (US); Melissa McCoy, Arlington, MA (US); Karen Sanderson, Charlestown, MA (US); Tariq Haq, Norwood, MA (US); Shuhao Zhu, Waltham, MA (US); Fan Long, Waltham, MA (US); Eugene Davidov, Natick, MA (US)

Correspondence Address: DARBY & DARBY P.C., POST OFFICE BOX 5257, NEW YORK, NY, 10150-5257, US

(73) Assignee: Anadys Pharmaceuticals, Inc.

(21) Appl. No.: 09/893,519

(22) Filed: Jun. 28, 2001

Related U.S. Application Data
(60) Provisional application No. 60/215,164, filed on Jun. 29, 2000.
(60) Provisional application No. 60/224,457, filed on Aug. 10, 2000.

(51) Int. Cl. C12Q 1/18

(52) U.S. Cl. 435/32

(57) ABSTRACT

The invention provides screening methods for detecting and identifying compounds that bind to fungal specific target proteins and nucleic acids, as well as compounds which, upon binding or otherwise interacting with the target protein, can inhibit fungal growth, a method of preventing or inhibiting fungal growth in culture, a method of preventing or inhibiting fungal growth in a mammal and a method of studying pathogenic mycetes using such nucleic acid and/or protein sequences. Particularly preferred is the inhibition of the fungus Candida albicans.

### PRIORITY

This application claims priority under 35 U.S.C. § 119 from Provisional Patent Application Serial No. 60/215,164, filed Jun. 29, 2000, and Provisional Patent Application Serial No. 60/224,457, filed Aug. 10, 2000, which are hereby incorporated by reference in their entireties.

### FIELD OF THE INVENTION

The invention encompasses the use of fungal cidal targets in the screening for, isolation and development of antifungal chemicals and drugs to be used in the treatment of fungal infections, such as infections with Candida albicans. The invention encompasses methods of determining fungal cidal targets. Such fungal cidal targets are encompassed by nucleic acid and protein sequences encoded by such nucleic acid sequences which are isolated from S. ceriviseae, shown to be present in other fungi such as Candida albicans, and are shown to be both essential and fungal specific in both Sacchromyces ceriviseae and Candida albicans. The essential fungal specific nucleic acid and protein sequences may also be used in studying pathogenic mycetes or fungi.

### BACKGROUND OF THE INVENTION

Fungi are a distinct class of microorganisms, of which most are free-living. They are eukaryotic organisms containing a nuclear membrane, mitochondria and endoplasmic reticulum. In addition, they are non-motile, do not contain chlorophyl and develop from spores (i.e. yeasts, molds, mushrooms and rusts). The cell structure usually includes a rigid cell wall of mannan, glucan and chitin and a cytoplasmic membrane with a large percentage of ergosterol. The size and morphology of fungi vary from monomorphic yeasts like Cryptococcus and Saccharomyces species and dimorphic fungi like Candida albicans to filamentous fungi like Aspergillus species.

In contrast to bacteria, which are generally considered mammalian pathogens, fungi tend to be plant pathogens. However, in addition to the well recognized group of dermatophytes (e.g. cause of “athlete's foot”), an increasingly large group of fungi turn out to be able to act as opportunistic human pathogens producing disease only in compromised individuals. As the result of an aging population as well as an increase in the number of immunocompromised patients, e.g., patients with acquired immunodeficiency syndrome (AIDS), patients undergoing cancer chemotherapy, or immunosuppressive therapy (e.g. treatment with corticosteroids) and patients undergoing organ transplantation, the incidence of fungal infections is increasing rapidly.

Fungi parasitize many different tissues. Most infections begin by colonization of the skin, a mucosal membrane or the respiratory epithelium. Superficial fungi and subcutaneous pathogens cause indolent lesions of the skin. Passage through the initial surface barrier is accomplished through a mechanical break in the epithelium. Although most fungi are readily killed by neutrophils, some species are resistant to phagocytic killing and can infect otherwise healthy individuals. The most virulent fungi cause systemic infections, a progressive disease leading to deep seated visceral infections in otherwise healthy individuals (see e.g. Sherris Medical Microbiology, Third Edition, Kenneth J. Ryan, ed., Appleton & Lange, Norwalk, Conn., 1994).

The major fungal pathogens in North America are Histoplasma capsulatum, Coccidioides immitis, Blastomyces dermatitidis, Cryptococcus neofonnans, Candida species, such as but not limited to Candida albicans and Aspergillus species (Medically Important Fungi, Second Edition, Davise H. Larone, Ed., American Society for Microbiology, Washington, D.C.). The yeast C. albicans (C. albicans) is one of the most pervasive fungal pathogens in humans. It is the cause of an increasing financial and logistic burden on the medical care system and its providers due to its ability to opportunistically infect a diverse spectrum of immunocompromised hosts, which are a quickly growing population of patients in today's society. Although C. albicans is a member of the normal flora of the mucous membranes in the respiratory, gastrointestinal, and female genital tracts, it may gain dominance in such locations (e.g. upon treatment with antibacterial antibiotics, in patients with diabetes or in patients using corticosteroids) and be associated with pathologic conditions. In addition, almost all HIV-positive individuals suffer from a Candida infection prior to the onset of developing full-blown AIDS.

Sometimes C. albicans produces progressive systemic disease, particularly if cell-mediated immunity is impaired. In 1994, about thirty percent of patients suffering from leukemia or undergoing organ transplants developed a systemic Candida infection of which thirty percent have been estimated to have succumbed to the infection.

Only a handful of agents are active against fungi. For life threatening disease caused by any of the pathogenic fungi, amphotericin B is the agent of choice. This drug, however, is associated with numerous severe side effects such as fever, dyspnea and tachycardia, and dosage is limited over the lifetime of the patient because of renal toxicity. An agent frequently used concurrently is flucytosine, a nucleoside analog, which cannot be used independently of other agents because of the rapid appearance of resistance. Untoward effects of treatment with flucytosine include leukopenia, thrombocytopenia, rash, nausea, vomiting, diarrhea, and severe enterocolitis.

In conditions where the patient's life is not threatened, ketoconazole can be used as a long-term therapy for blastomycosis, histoplasmosis, or coccidioidomycosis. Fluconazole also has a significant role in the treatment of superficial fungal infections. Both compounds are from the same class, the triazoles, and are cytostatic. The emergence of resistance and hepatic toxicity limits the use of triazoles such as fluconazole and ketoconazole. The newest triazole, itraconazole, has similar pharmacokinetics and spectrum of activity as fluconazole. None of the azoles can be used for life threatening or deep seated fungal infections. They are only effective in reducing colonization of fungi such as Candida species and for treating superficial mycoses.

All major antifungal agents function by attacking, either directly or indirectly, ergosterol, a component of the cell wall. Amphotericin B and other polyene macrolide compounds like nystatin interact with ergosterol in the cell membrane and form pores or channels that increase the permeability of the membrane. Resistance to amphotericin B in mutant strains is accompanied by decreased concentrations of ergosterol in their cell membranes. Imidazoles and triazoles inhibit sterol 14-″-demethylase, a microsomal cytochrome P₄₅₀-dependent enzyme system. Imidazoles and triazoles thus impair the biosynthesis of ergosterol for the cytoplasmic membrane, leading to the accumulation of 14-″-methyl sterols, which impair certain membrane-bound enzyme systems (see, The Pharmacological Basis of Therapeutics, Eighth Edition, Goodman and Gilman, Pergamon Press, 1990).

Nystatin, amphotericin B, flucytosine and the various azoles have all been used to treat oral and systemic Candida infections. However, orally administered nystatin is limited to treatment within the gut and is not applicable to systemic treatment, and resistance to flucytosine is so widespread that it is only used in combination with other drugs. Some life-threatening systemic infections are susceptible to treatment with the azoles or amphotericin B. Azoles have been the most successful drugs used for treatment of such infections in the last few years but they work relatively slowly, have to be taken for months, and are fungistatic rather than fungicidal. While such azole antifungal agents exhibit significantly lower toxicity compared to amphotericin B, their mechanism of action and inactivation of cytochrome P₄₅₀ prosthetic groups in certain enzymes preclude their use in patients that are simultaneously receiving other drugs that are metabolized by the body's cytochrome P₄₅₀ enzymes.

Widespread use of azoles has also resulted in an important change in the spectrum of Candida infections. Whereas C. albicans used to be the common cause of Candidosis, 50% of these infections are now caused by non-albicans species which tend to be less susceptible to azole treatment. In addition, a quickly rising percentage of C. albicans isolates obtained from infected patients have been found to be resistant to azoles.

There is thus an immediate need for an effective treatment of opportunistic infections caused by C. albicans and other fungi. Although the majority of life-threatening fungal infections are caused by C. albicans, infections caused by other less common fungi as discussed above, e.g., Aspergillus fumigatus have a worse prognosis. In large part this is due to the absence of diagnosis until a very late stage of infection, usually post-mortem. Therefore it is desirable that novel compounds be able to act against all pathogenic fungi, preventing the need for precise, time-consuming diagnosis.

Development of an effective method and composition for treatment of fungal infections is a critical goal of the pharmaceutical industry. The industry has made numerous efforts to identify fungal-specific drugs, with only limited success. It would be of great value to identify a new class of antifungal drugs that block a fungal target other than ergosterol. This target should be fungal-specific and should lead to development of a drug that is effective in preventing or inhibiting the growth of, and preferentially killing, the organisms that are resistant to current therapy.

Antifungal drug development often relies on the screening of a large number of compounds before one or more lead compounds are found that are effective against the target fungi. Thus, it is critical for the development of these screens to define proteins essential for survival or growth of the target fungi and to discover means of purifying or producing such proteins. Therefore, there is a need in the art to identify essential fungal structural or functional elements that can serve as targets for drug intervention, and for methods and compositions for identifying useful anti-fungal agents that interact with or inhibit essential fungal elements that can be used to treat fungal infections by preventing or inhibiting the growth of, and preferentially killing, the fungi.

### SUMMARY OF THE INVENTION

The present invention is based on the determination of Saccharomyces cerevisiae proteins which are potential targets to kill S. cerevisiae cells. The invention provides a screening method for detecting and identifying a compound that binds to a homologous target protein isolated from C. albicans, as well as compounds which can inhibit C. albicans and other fungal growth. The invention also provides a method for evaluating the toxicity of such a fungal inhibitor in mammalian cells.

The invention utilizes target proteins involved in such processes as DNA synthesis, DNA replication, DNA transcription, mRNA translation, post-translational modification of proteins, and intracellular transport of proteins, as well as target proteins whose exact cellular functions are unknown. In preferred embodiments, the invention provides for the use of S. cerevisiae target proteins listed in Table 1 together with C. albicans and human homologs, depicted therein by their respective amino acid sequences which are provided in FIG. 79. The nucleic acid sequences corresponding to these amino acid sequences are depicted in FIG. 80.

Each of the S. cerevisiae DNA sequences, and their predicted target protein sequences, which are utilized in practicing the invention are publicly available. The essentiality of each of such S. cerevisiae genes may already be known or may be determined and/or corroborated through the analysis of the ability to knock out the gene's function in S. cerevisiae. The present invention thus provides a method of determining and/or validating the essentiality of the S. ceriviseae gene and the target protein encoded by that gene. More specifically, the invention is directed to the determination of the S. ceriviseae protein as a cidal target to be used in the determination and isolation of a homologous target in C. albicans. The C. albicans target may then be used in the screening of compounds which can inhibit Candida albicans and other fungal growth.

Following the determination of the essentiality of the S. cerevisiae gene, the S. ceriviseae DNA sequence may be used to isolate a homologous fungal gene. Thus, in another aspect, the invention is based on the determination of a C. albicans nucleic acid encoding the C. albicans protein as a target which is essential for the growth of C. albicans.

In a still further aspect, the invention provides for producing a recombinant target C. albicans target protein, comprising culturing a host cell transformed with a nucleic acid encoding the C. albicans target protein under conditions sufficient to permit expression of the nucleic acid encoding the C. albicans target protein and isolating the C. albicans target protein to be used in assays described below.

Sequence alignments utilizing the S. cerevisiae nucleic acid or protein sequences and/or the C. albicans nucleic acid or protein sequences in combination with known sequences available in Genbank may be carried out in order to demonstrate any similarity or differences between different fungi, i.e., S. cerevisiae, C. albicans, and Aspergillus, and mammals. In this manner, homologous genes can be isolated. One example of such analysis would be BLAST™ analysis.

In a further embodiment, following the determination that the target protein in Saccharomyces cerevisiae is a cidal target, and that the homologous protein in Candida albicans is essential for growth, the C. albicans protein may be used as a target to isolate candidate inhibitors of fungal growth and/or infection. Detection and identification of compounds that bind to the essential protein may be performed in the presence of a plurality of candidate inhibitor compounds. In carrying out the screening methods of the invention which involve screening a plurality of candidate inhibitor compounds, the plurality of inhibitor compounds may be screened together in a single assay or individually using multiple simultaneous individual detecting steps.

In another aspect, the invention provides a method of preventing or inhibiting fungal, particularly C. albicans, growth in culture, by contacting the culture with an inhibitor compound that selectively inhibits the biological activity of a fungal target protein, particularly a C. albicans target protein.

In a further aspect, the invention provides a method of preventing or inhibiting fungal, particularly C. albicans, growth in a mammal, comprising administering to the mammal an effective amount of an inhibitor compound that selectively inhibits the biological activity of a fungal, particularly C. albicans, target protein.

In a still further aspect, the invention provides a method of preventing or inhibiting fungal, particularly C. albicans, growth in a mammal, comprising administering to the mammal an effective amount of an inhibitor compound, wherein the inhibitor selectively inhibits the biological activity of a fungal, particularly C. albicans, target protein, but inhibits the biological activity of the homologous mammalian protein to a lesser degree, or not at all.

In yet another aspect, the invention provides a method of preventing or inhibiting fungal growth, comprising administering to a fungal infection an effective amount of an inhibitor compound that selectively inhibits the biological activity of a fungal target protein.

In still another aspect, the invention provides a method of studying pathogenic mycetes using such nucleic acid and/or protein sequences.

Other features and advantages of the invention will be apparent from the description, preferred embodiments thereof, the drawings, and from the claims.

TABLE 1
Preferred target proteins

| S. cerevisiae | | | C. albicans | Human | |
|---|---|---|---|---|---|
| Gene name | ORF name¹ | Sequence | Sequence | Sequence | Genbank Acc#² |
| RPC34 | YNR003C | SEQ ID NO: 1 | SEQ ID NO: 2 | SEQ ID NO: 3 | U93869 |
| POP3 | YNL282W | SEQ ID NO: 4 | SEQ ID NO: 5 | — | n/a |
| TFA2 | YKR062W | SEQ ID NO: 6 | SEQ ID NO: 7 | SEQ ID NO: 8 | NP_002086 |
| NAB2 | YGL122C | SEQ ID NO: 9 | SEQ ID NO: 10 | SEQ ID NO: 11 | AAD42873 |
| MPT1 | YMR005W | SEQ ID NO: 12 | SEQ ID NO: 13 | SEQ ID NO: 14 | CAA72189 |
| MTR2 | YKL186C | SEQ ID NO: 15 | SEQ ID NO: 16 | — | n/a |
| BOS1 | YLR078C | SEQ ID NO: 17 | SEQ ID NO: 18 | SEQ ID NO: 19 | NP_003560 |
| POL30 | YBR088C | SEQ ID NO: 20 | SEQ ID NO: 21 | SEQ ID NO: 22 | P12004 |
| RSA2 | YMR131C | SEQ ID NO: 23 | SEQ ID NO: 24 | SEQ ID NO: 25 | NP_005601 |
| SQT1 | YIR012W | SEQ ID NO: 26 | SEQ ID NO: 27 | SEQ ID NO: 28 | NP_001078 |
| MTW1 | YAL034W-A | SEQ ID NO: 29 | SEQ ID NO: 30 | — | n/a |
| TFB1 | YDR311W | SEQ ID NO: 31 | SEQ ID NO: 32 | SEQ ID NO: 33 | W19128 |
| SPC98 | YNL126W | SEQ ID NO: 34 | SEQ ID NO: 35 | SEQ ID NO: 36 | AAC39727 |
| BFR2 | YDR299W | SEQ ID NO: 37 | SEQ ID NO: 38 | SEQ ID NO: 39 | NM_000055 |
| RNA1 | YMR235C | SEQ ID NO: 40 | SEQ ID NO: 41 | SEQ ID NO: 42 | CAA57714 |
| GCD7 | YLR291C | SEQ ID NO: 43 | SEQ ID NO: 44 | SEQ ID NO: 45 | AAC42002 |
| SK16 | YGR195W | SEQ ID NO: 46 | SEQ ID NO: 47 | SEQ ID NO: 48 | BAA91279 |
| NIP1 | YMR309C | SEQ ID NO: 49 | SEQ ID NO: 50 | SEQ ID NO: 51 | AAD03462 |
| LCP5 | YER127W | SEQ ID NO: 52 | SEQ ID NO: 53 | SEQ ID NO: 54 | AL050003 |
| NCE103 | YNL036W | SEQ ID NO: 55 | SEQ ID NO: 56 | — | n/a |
| ECO1 | YFR027W | SEQ ID NO: 57 | SEQ ID NO: 58 | — | n/a |
| ORC2 | YBR060C | SEQ ID NO: 59 | SEQ ID NO: 60 | SEQ ID NO: 61 | Q13416 |
| CNS1 | YBR155W | SEQ ID NO: 62 | SEQ ID NO: 63 | SEQ ID NO: 64 | NP_004614 |
| YPD1 | YDL235C | SEQ ID NO: 65 | SEQ ID NO: 66 | SEQ ID NO: 67 | CAA78727 |
| TIM10 | YHR005C-A | SEQ ID NO: 68 | SEQ ID NO: 69 | SEQ ID NO: 70 | NP_036588 |
| SRB4 | YER022W | SEQ ID NO: 71 | SEQ ID NO: 72 | SEQ ID NO: 73 | BAA88763 |

¹ORE = Open Reading Frame
²Acc# = Accession number

### BRIEF DESCRIPTION OF THE DRAWINGS

FIGS. 1-26 provide sequence alignments and identity determinations for the target proteins presented herein. Each figure refers to one target protein as identified in Table 2, comparing amino acid sequences from S. cerevisiae, C. albicans, and, if available, human homologs. Sequence alignment was carried out using Clustal W (Thompson et al., Nucleic Acids Res. 1994;22:4673-80), and percentage identities determined using the Genetics Computer Group (“GCG”) GAP Program (Madison, Wis.) with a gap creation penalty of 12 and a gap extension penalty of 4.

FIGS. 27-52 provide S. cerevisiae inactivation analyses of the target genes/proteins identified in Table 1. These data show the essentiality of each gene for S. cerevisiae growth. Each figure refers to one target protein. Inactivation analyses were conducted by placing the S. cerevisiae expression of a target gene under the control of a metal-sensitive element and incubating the yeast cells together with a Cu-salt, as described in the Detailed Description below and in Example 1.

FIGS. 53-78, A and B for each, provide C. albicans deletion analyses of the target genes/proteins identified in Table 1. These data indicate the essentiality of each gene for C. albicans growth. Each figure refers to one target protein. Deletion analyses were conducted as described in the Detailed Description, and C. albicans transformation as described in Example 2 below.

FIG. 79 provides amino acid sequences for each of the proteins disclosed herein and depicted in Table 1.

FIG. 80 provides nucleic acid sequences corresponding to each of the proteins disclosed in FIG. 79.

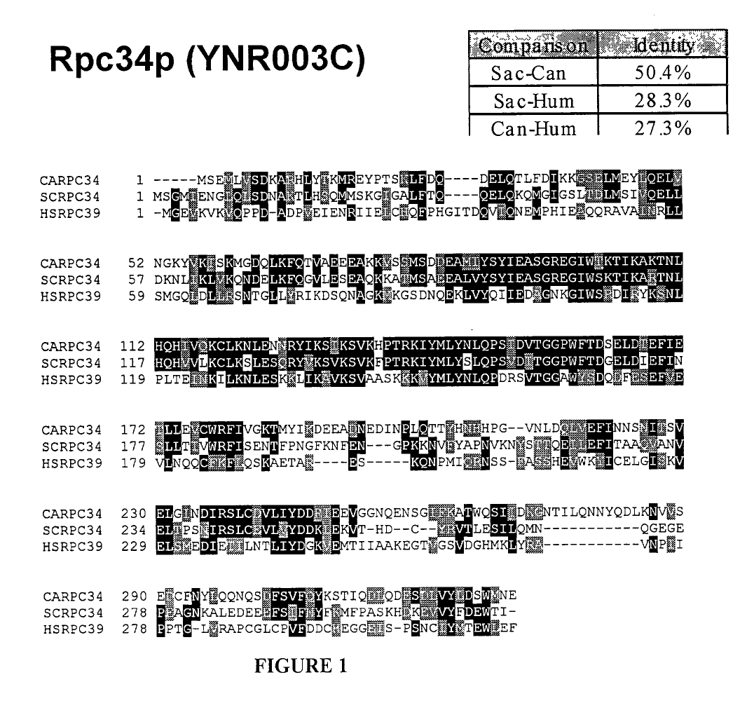

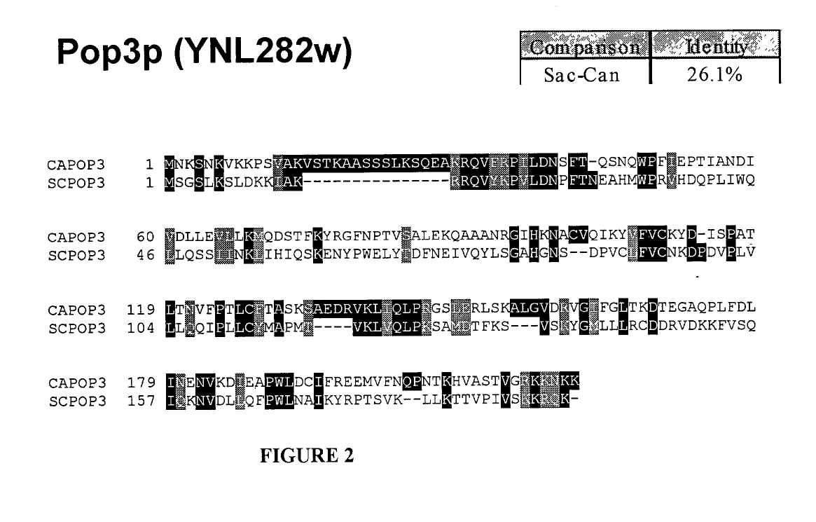

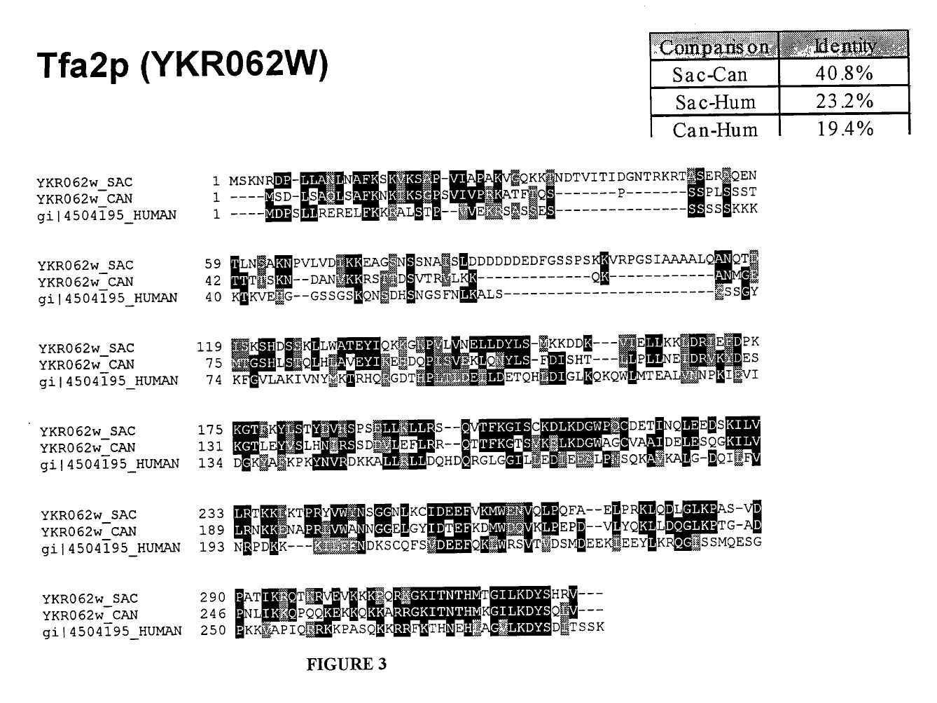

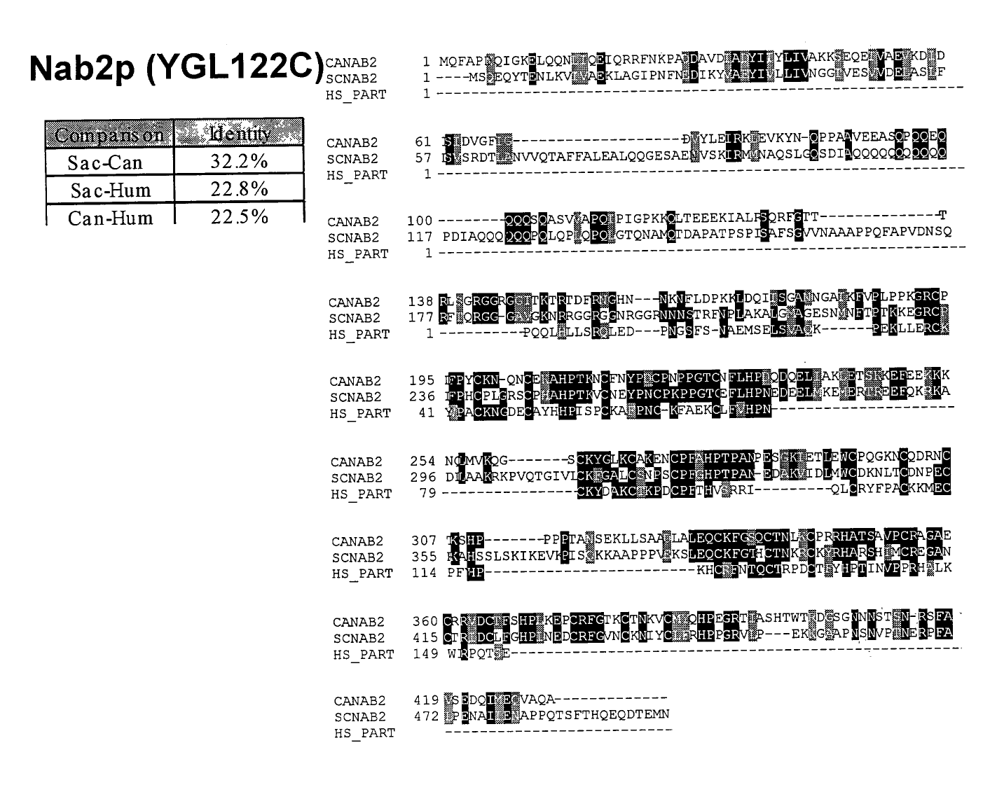

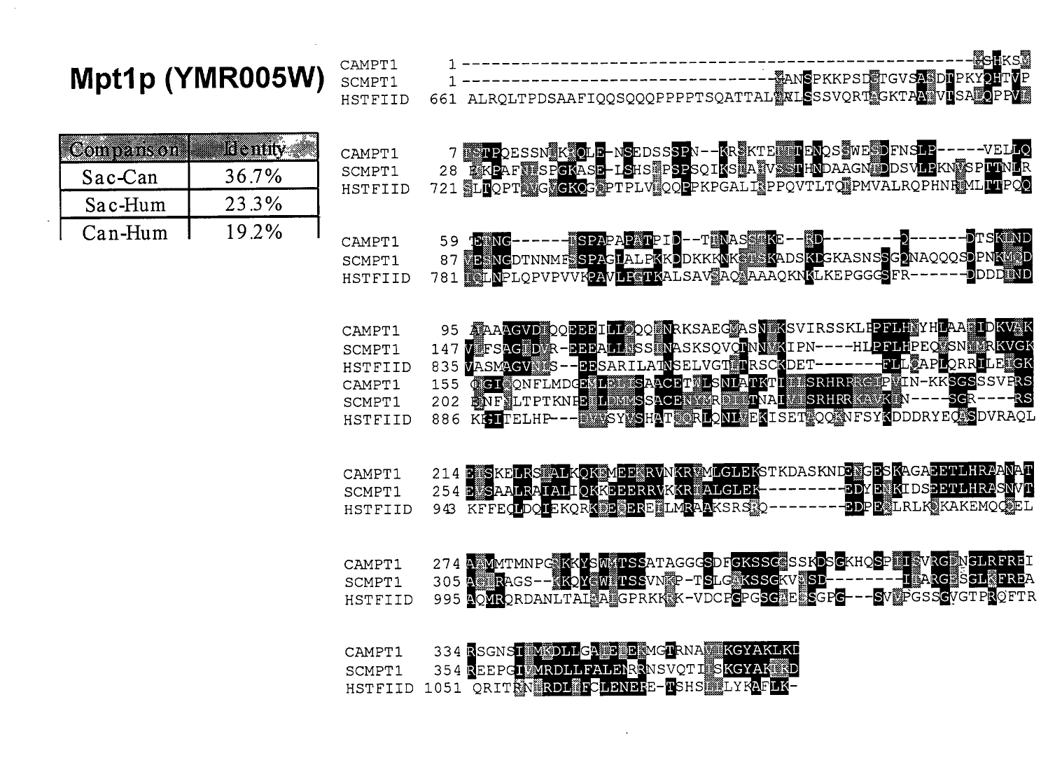

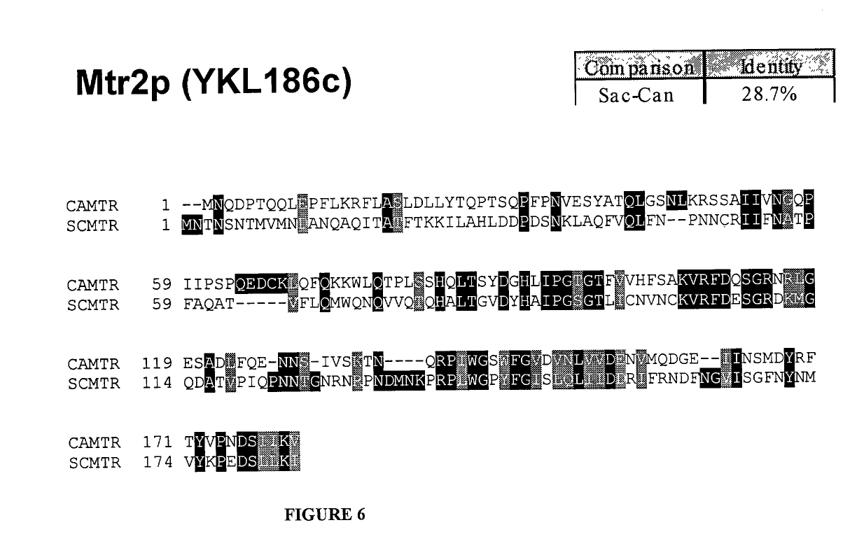

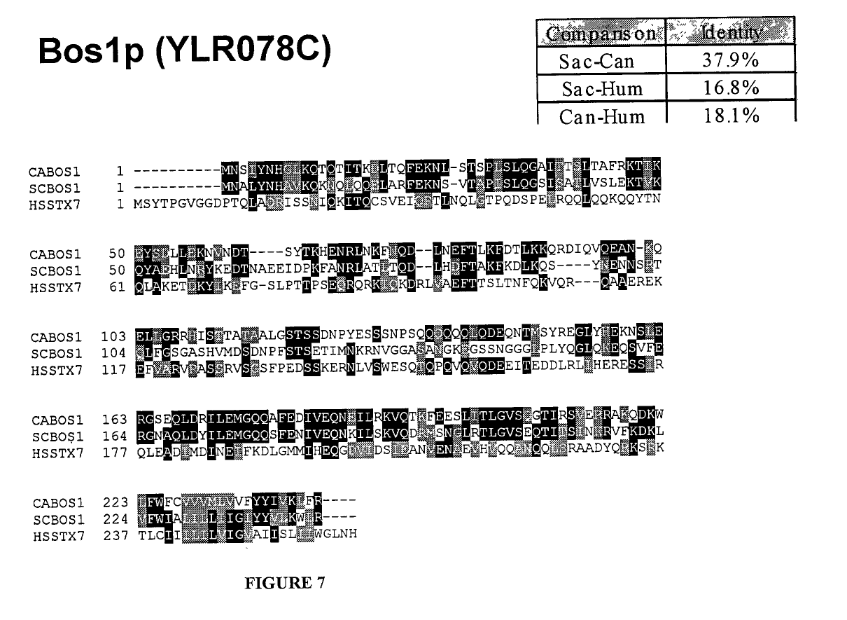

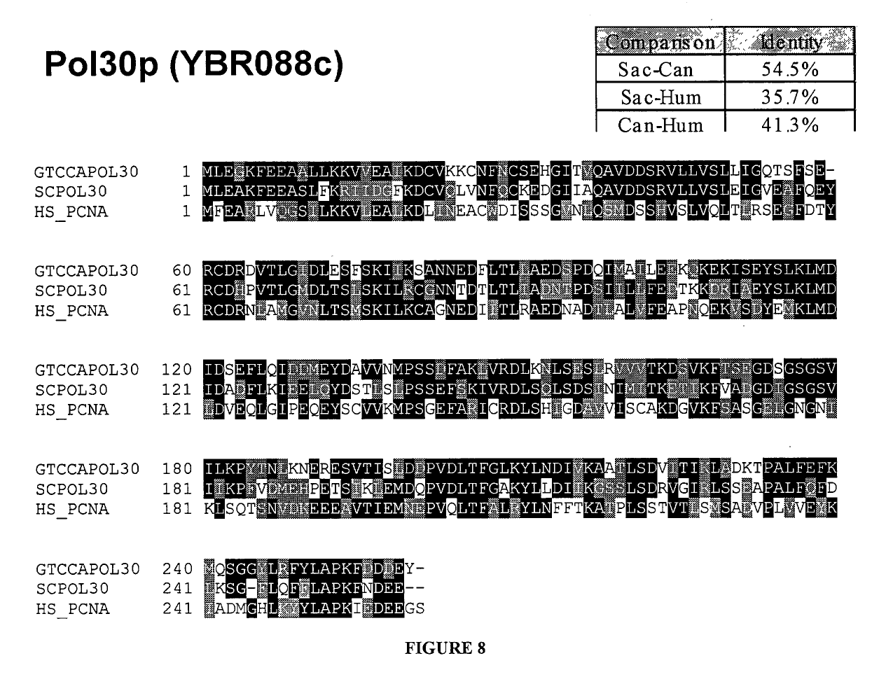

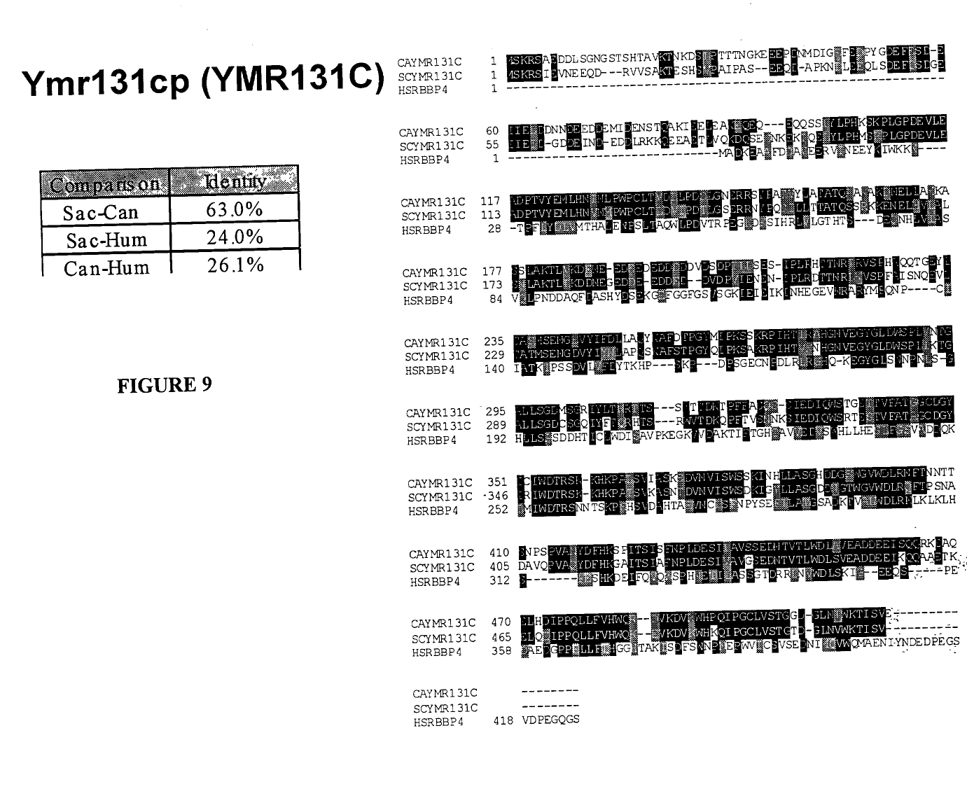

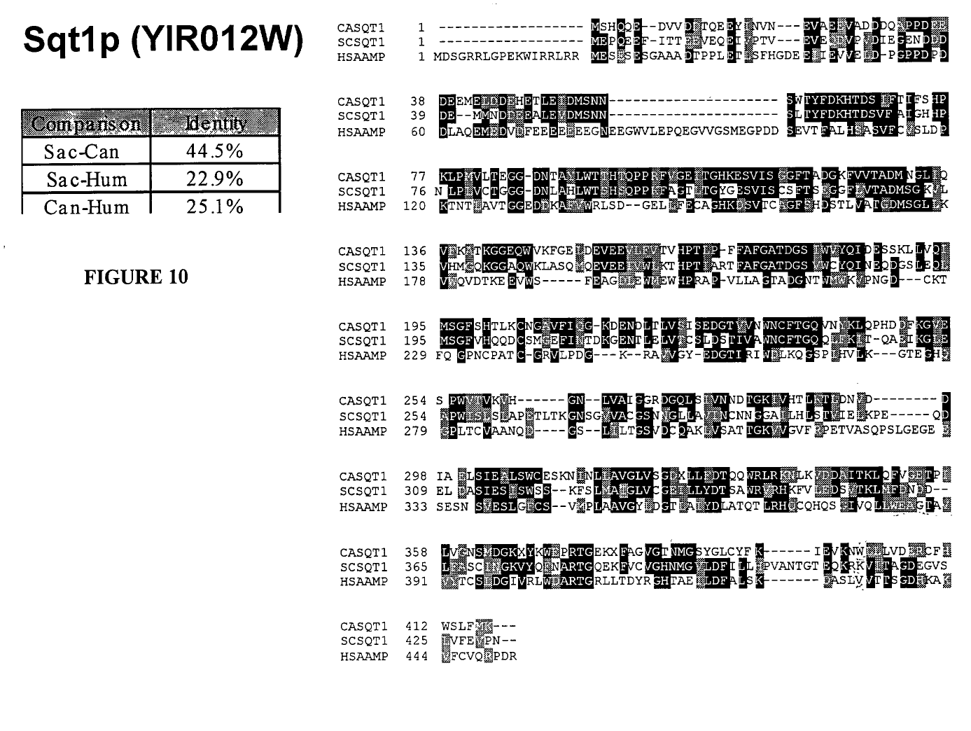

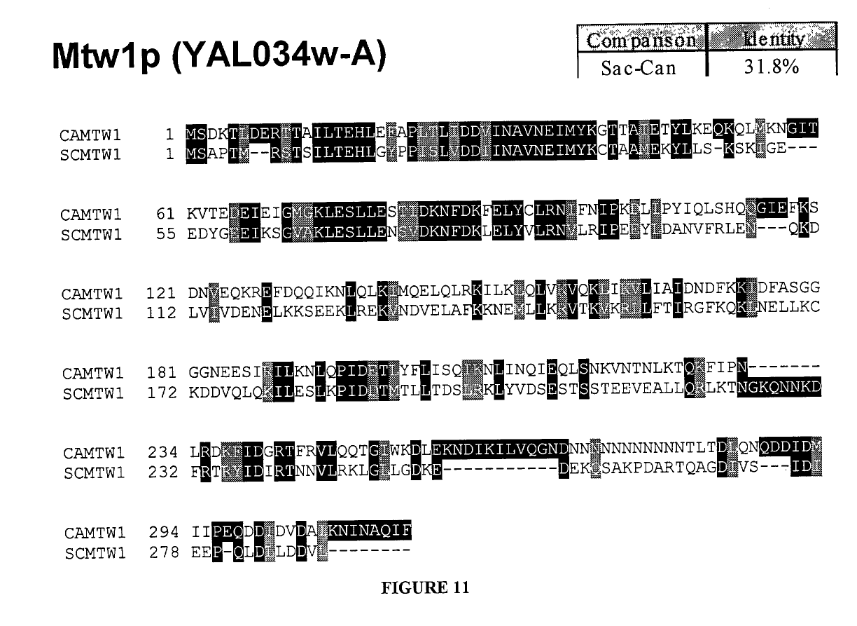

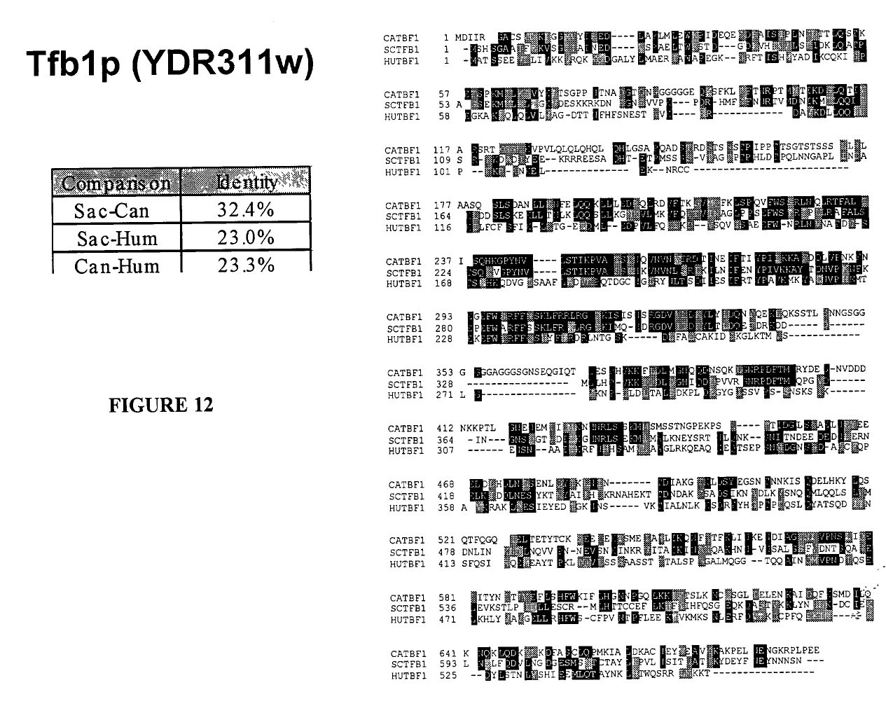

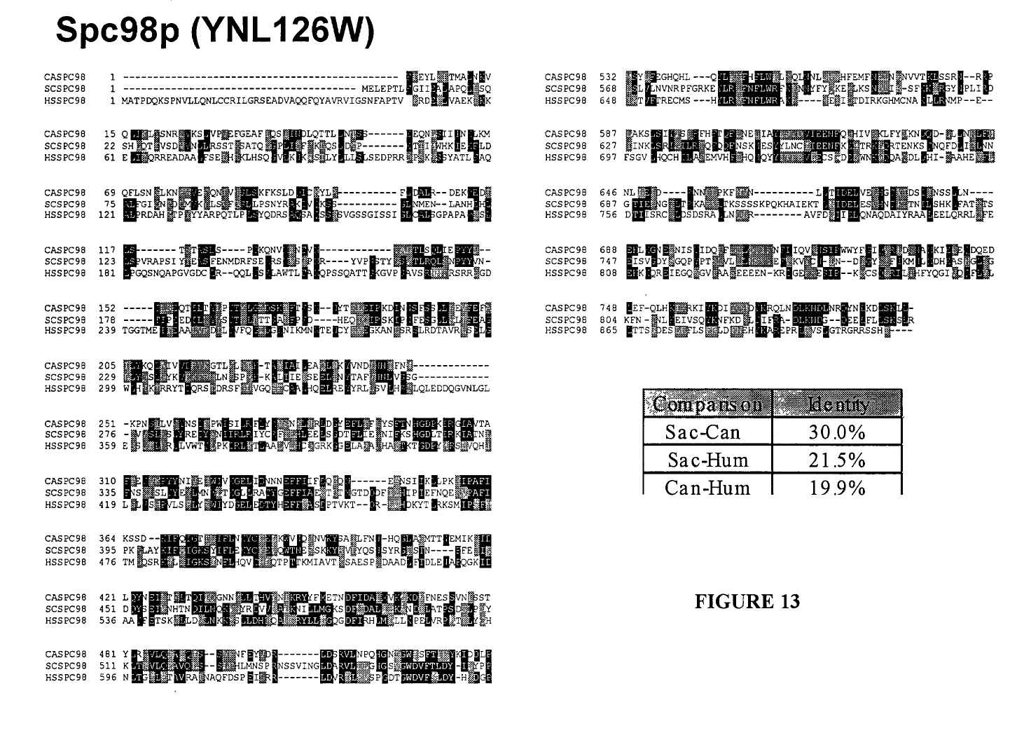

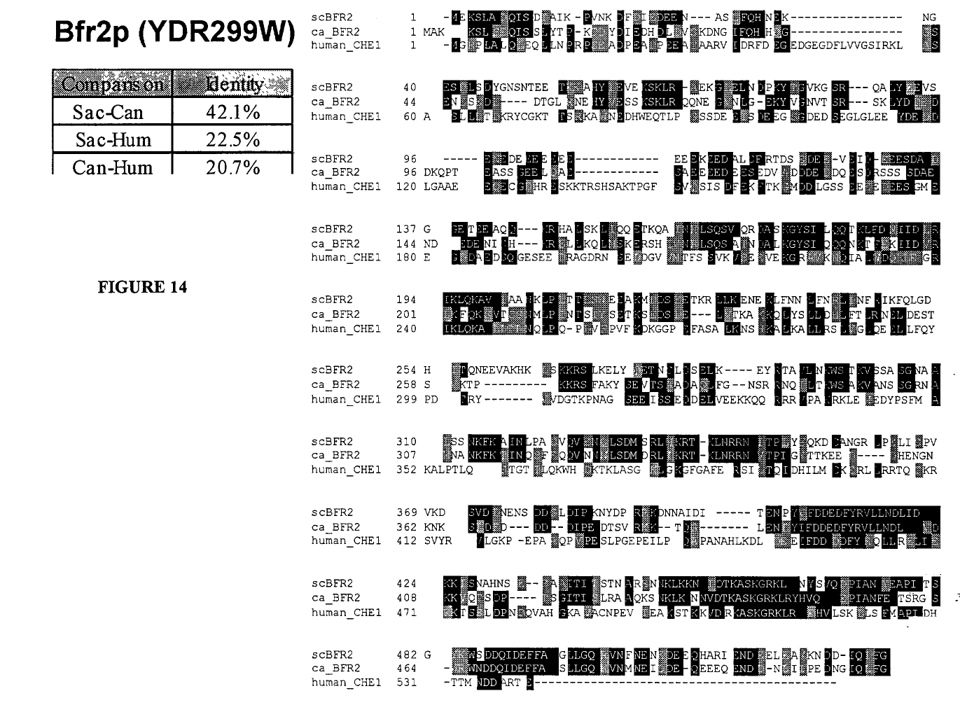

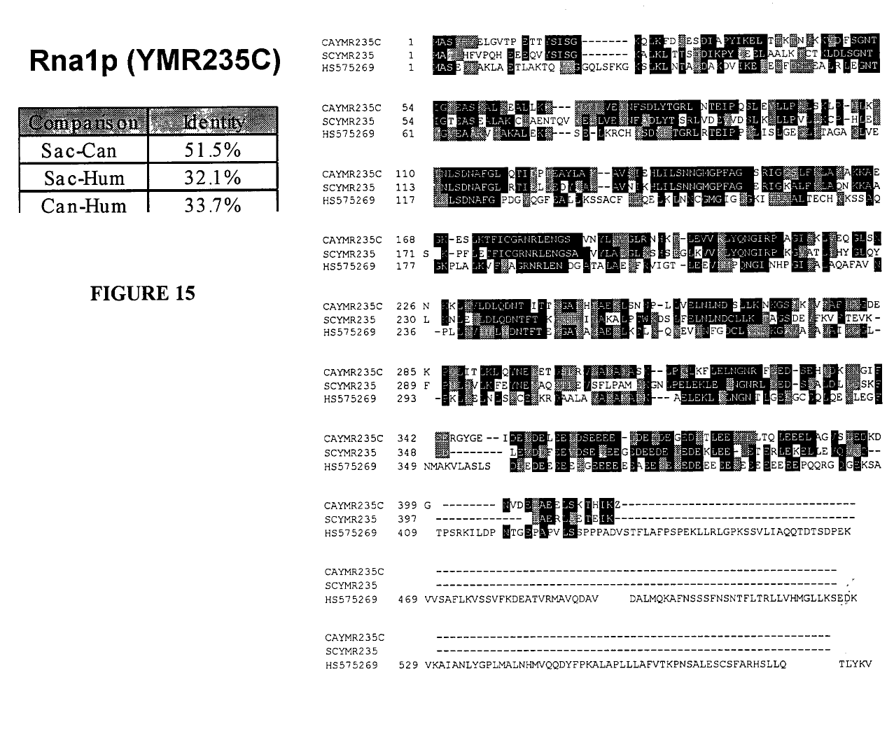

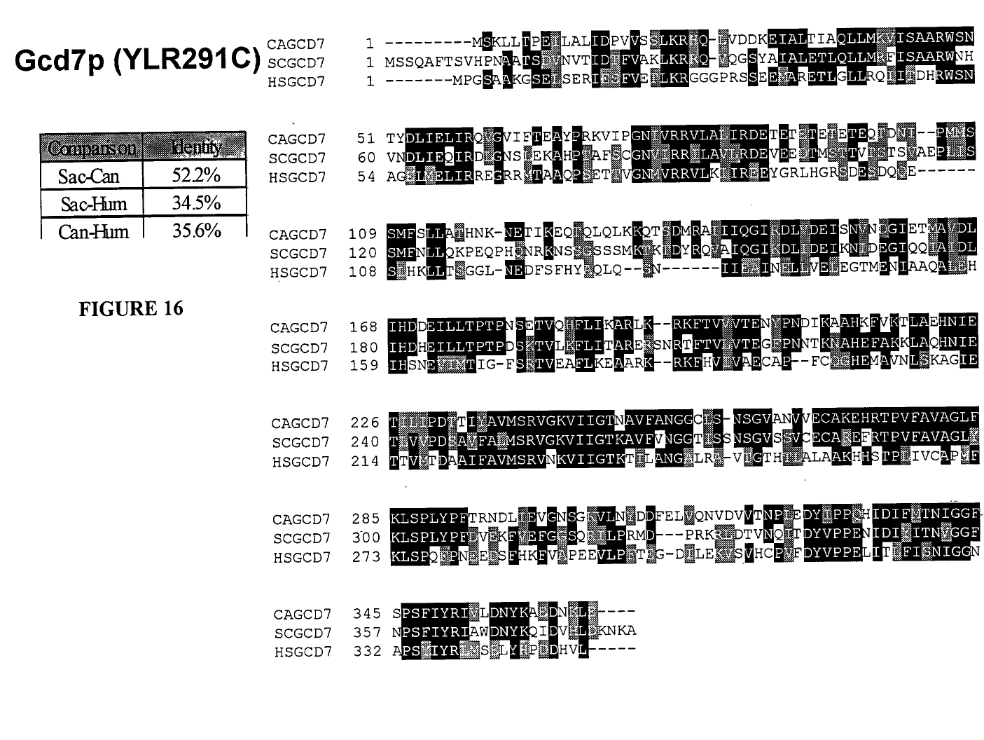

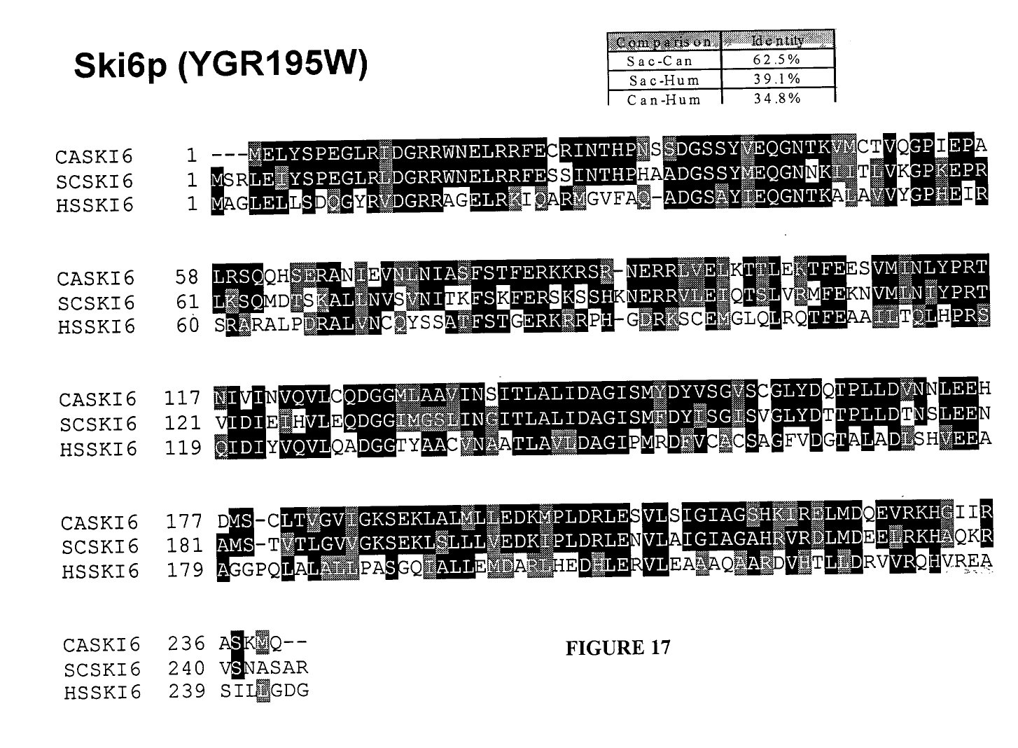

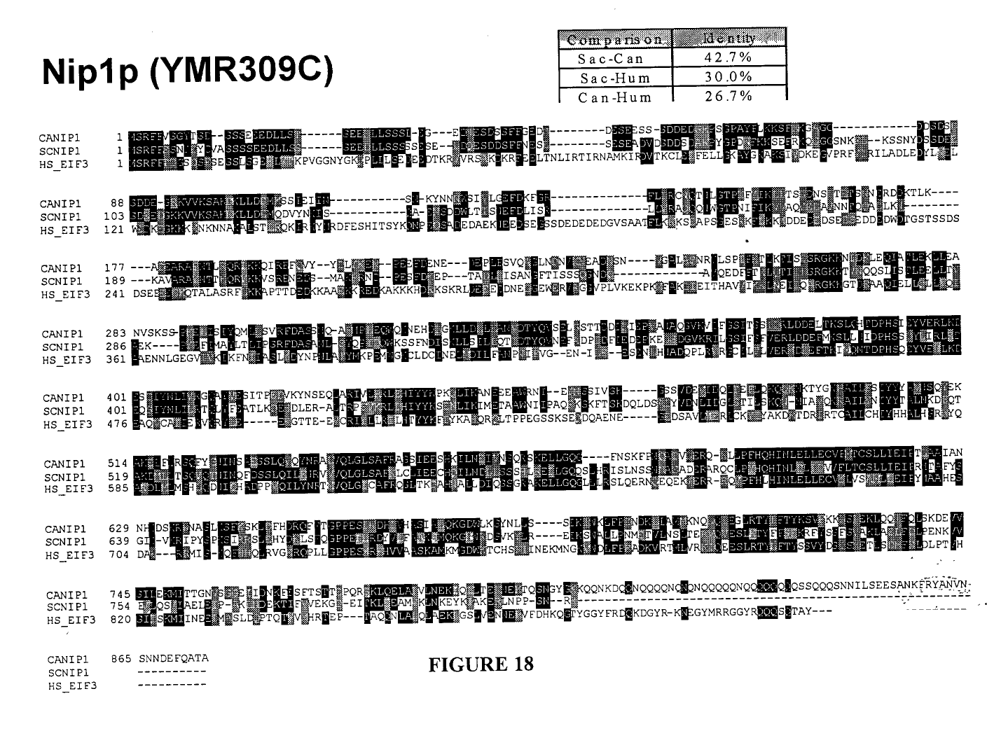

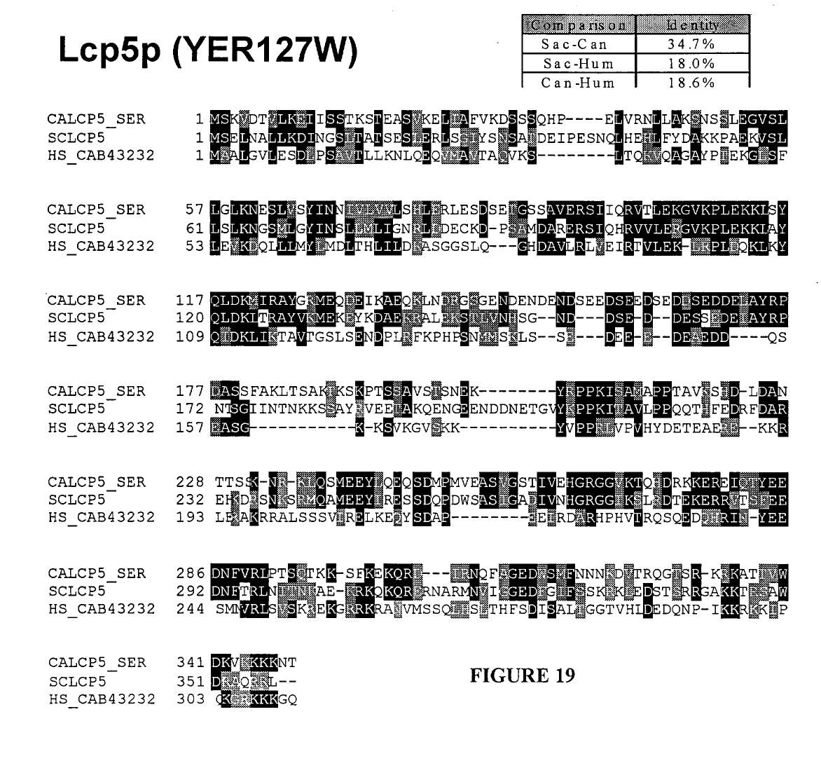

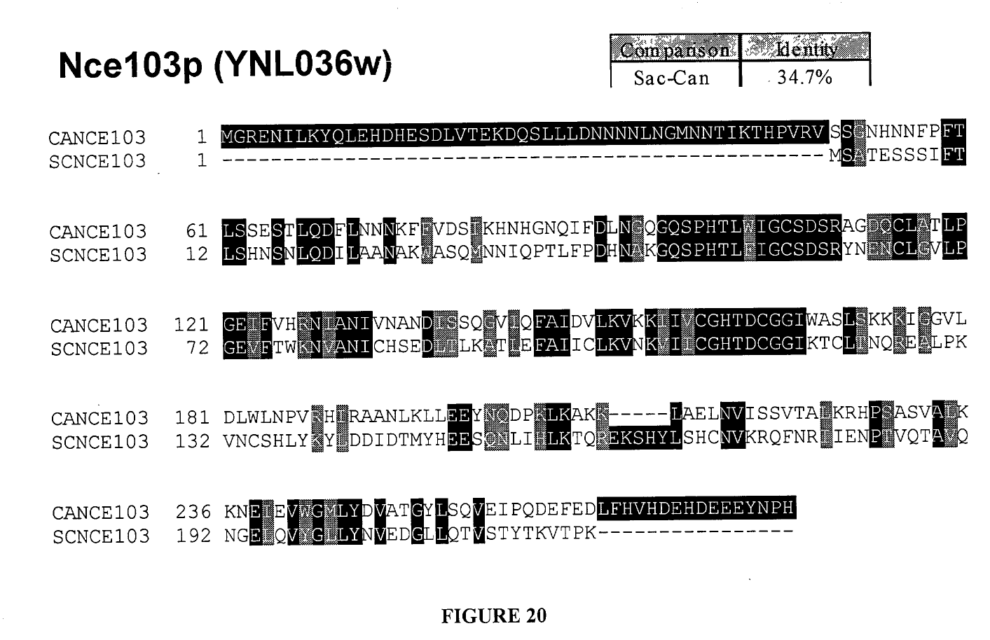

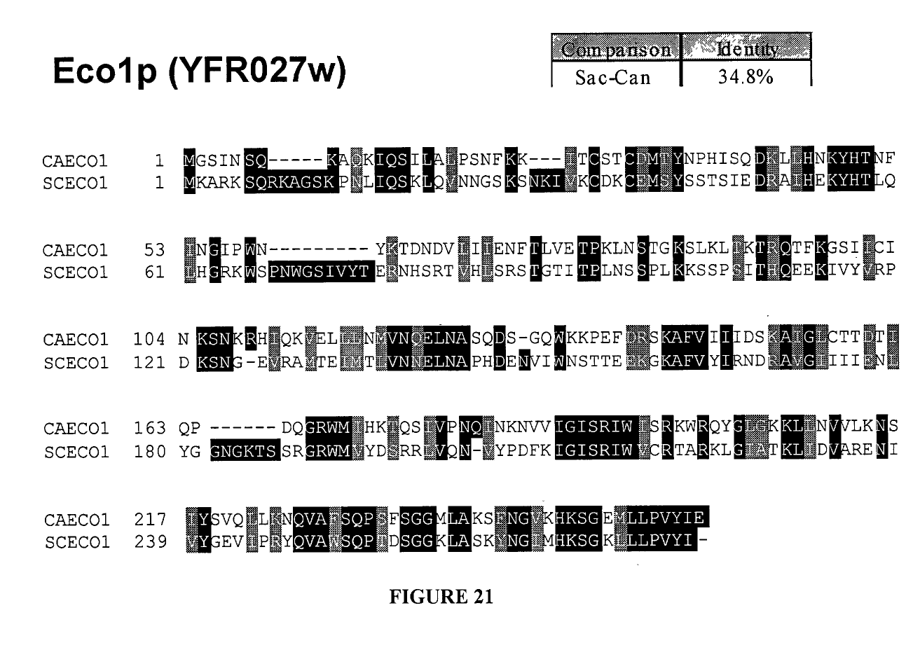

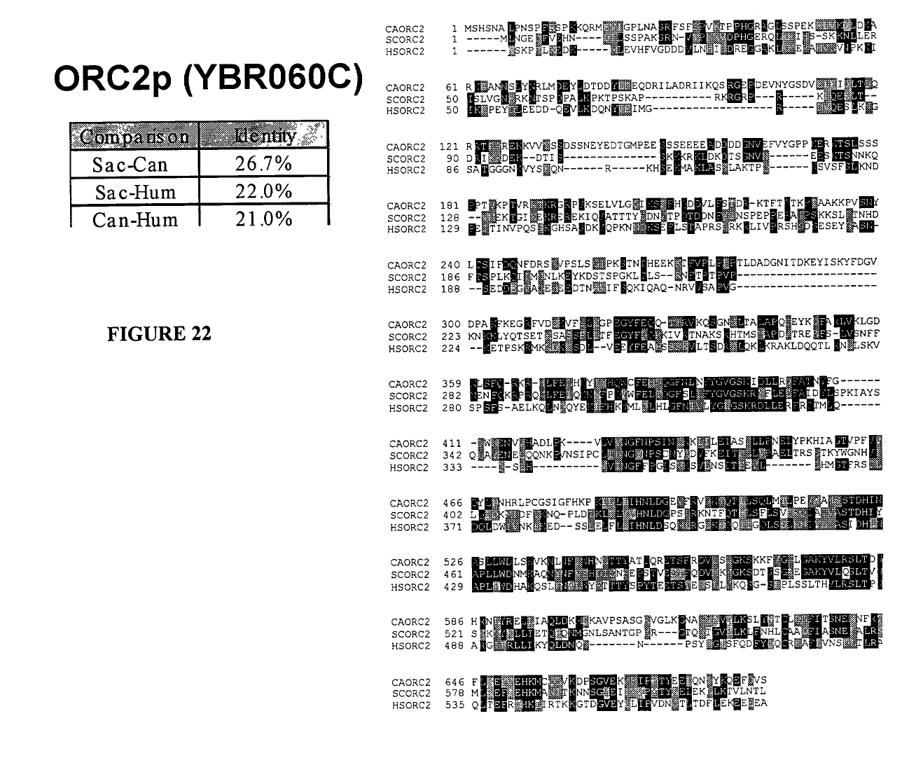

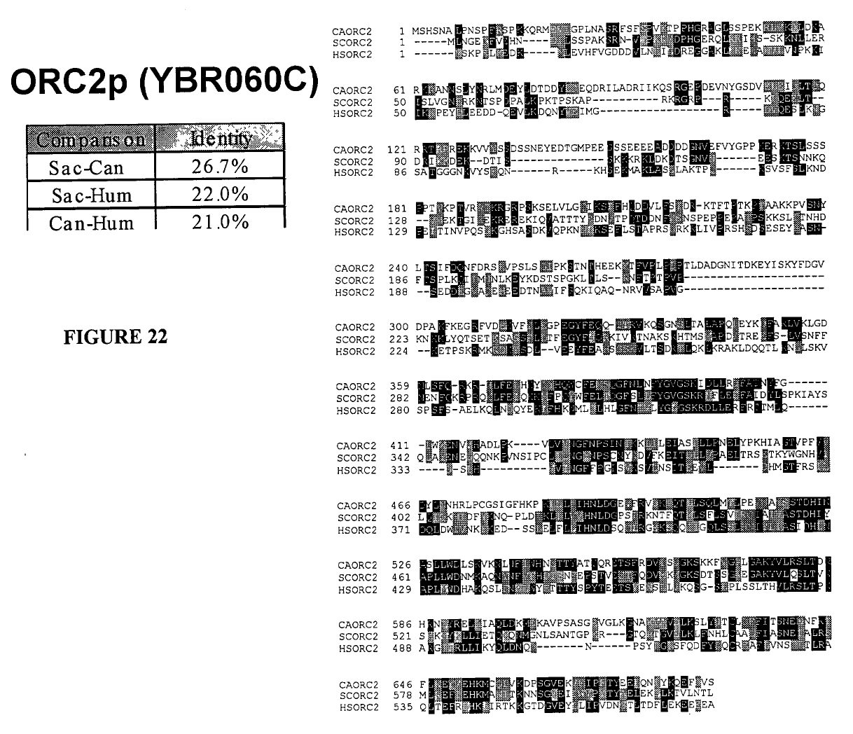

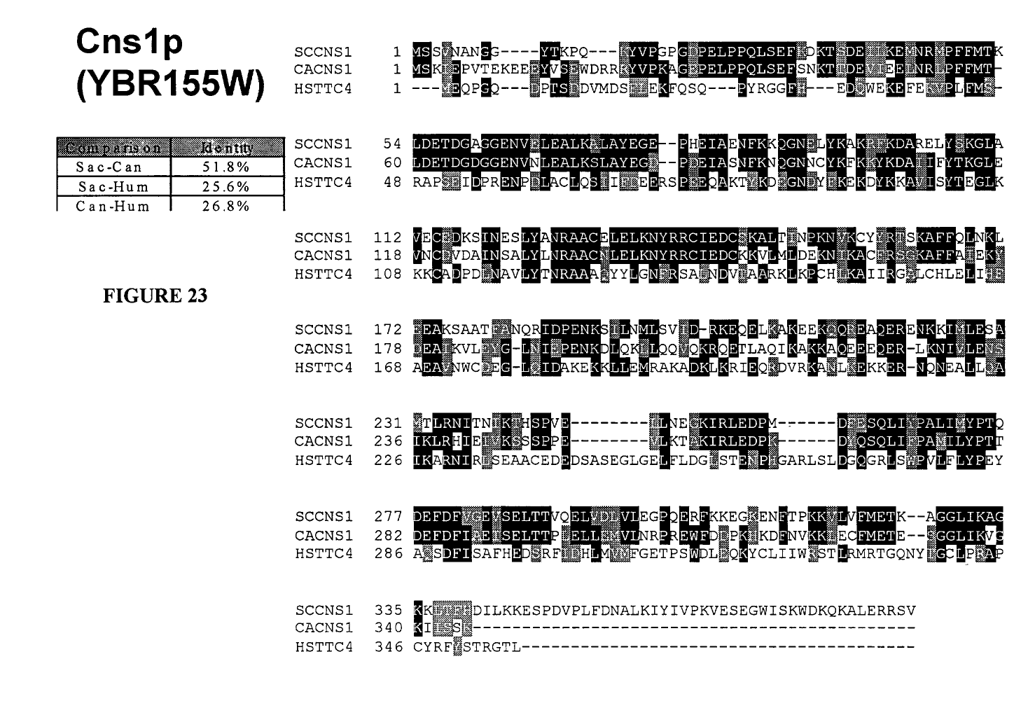

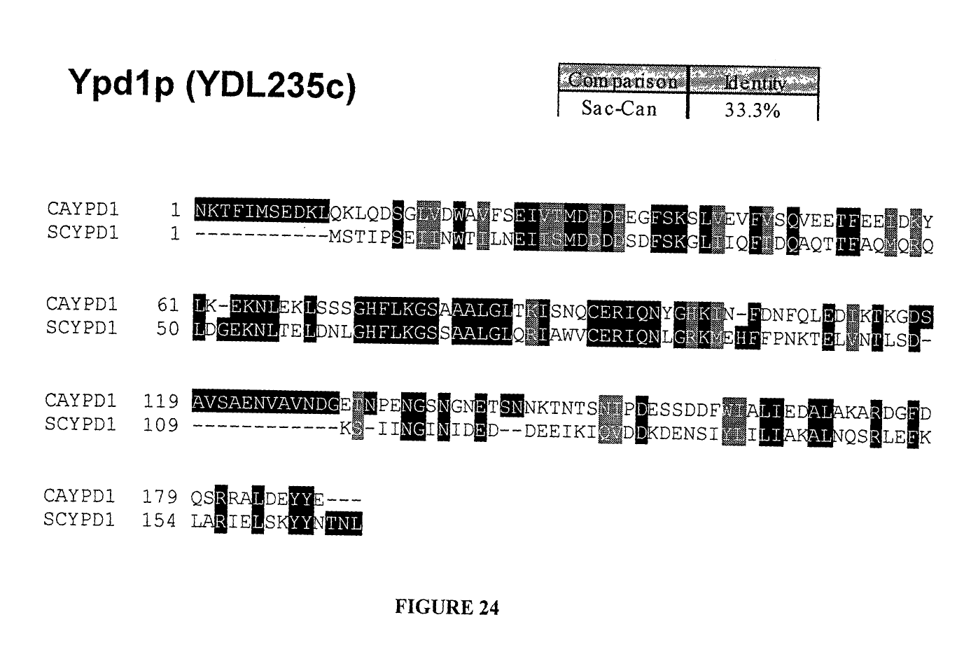

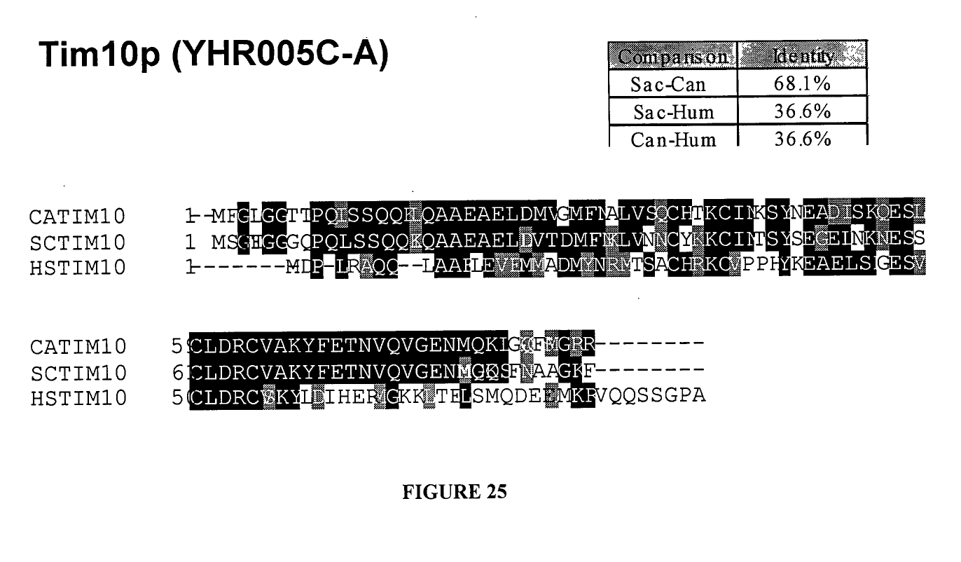

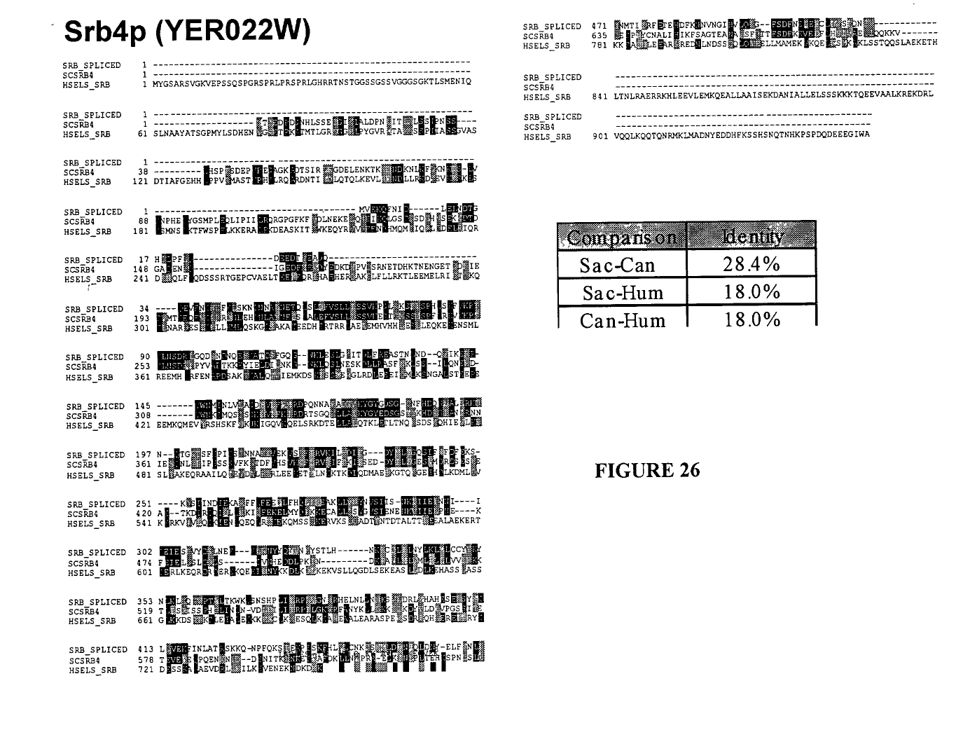

### DETAILED DESCRIPTION OF THE INVENTION

All patent applications, patents, and literature references cited in this specification are hereby incorporated by reference in their entirety.

This invention is directed to essential fungal proteins isolated from S. cerevisiae to be used in the determination and/or isolation of a homologous protein from fungi, particularly C. albicans. These fungal proteins, each of which described in more detail below, play essential roles in cell viability and/or growth, and are conserved among fungi. Because these fungal proteins are essential for viability and/or growth of fungal cells, a compound that blocks the biological activity of such a target protein would be expected to have fungicidal and/or fungistatic properties. Since amino acid sequences of any such protein from different fungal sources are likely to be more similar to one another than to the corresponding human protein, it is expected that certain compounds that bind to the fungal protein will not bind to the corresponding human protein, and so will be specific inhibitors of fungal cell growth. Therefore, the invention is also directed to assays to screen for inhibitors of these target proteins which are active against fungi.

In general, nucleic acid manipulations and other related techniques used in practicing the present invention employ methods that are well known in the art, as disclosed in, e.g., Molecular Cloning, A Laboratory Manual (2nd Ed., Sambrook, Fritsch and Maniatis, Cold Spring Harbor) and Current Protocols in Molecular Biology (Eds. Ausubel, Brent, Kingston, More, Feidman, Smith and Stuhl, Greene Publ. Assoc., Wiley-Interscience, NY, N.Y., 1997).

### Definitions

1. The terms “Prevention” and “Inhibition” as used herein may be used interchangeably. “Inhibition” as used herein refers to a reduction in the parameter being measured, whether it be fungal growth, DNA transcription, or another parameter related to a selected process relating to the biological activity of a target protein. The amount of such reduction is measured relative to a standard (control). Because of the multiple interactions of various fungal protein in cell division, growth regulation, cell cycle regulation, and other growth and/or metabolic processes, the amount of target product needed to produce a detectable inhibition will vary with respect to the particular screening assay employed. “Reduction” is defined herein as a decrease of at least 25% relative to a control, preferably of at least 50%, and most preferably of at least 75%.

2. “Growth” or “multiplication” as used herein refers to the normal growth pattern of fungi, particularly S. cerevisiae and/or C. albicans, i. e., to a cell doubling time of 60-90 minutes during the log phase of growth. In rich media, wild-type S. cerevisiae strains have a doubling time of 90 minutes, while wild type C. albicans doubling time is closer to approximately 60 minutes. Growth of the cells may be measured by following the optical density of cells in liquid media. An increasing optical density indicates growth. Growth can also be measured by colony formation from single cells on solid media plates.

3. “Viability” as used herein refers to the ability of the S. cerevisiae or C. albicans cells to resume growth following a treatment of the cells which results in cessation of growth. Examples of such treatments resulting in cessation of growth include, but are not limited to, transient inactivation of a gene product required for growth or treatment with an antifungal drug. One typical means by which viability is measured is by testing the ability of cells to form colonies on solid media plates following removal of the treatment which resulted in a cessation of growth. Cells that fail to form colonies are considered inviable.

4. “Cidal” as used herein is defined as a rapid loss in viability. Rapid is defined as a population of cells losing viability with a measured half-life of at least about 2 hours or less.

5. A “homologous” protein as used herein is defined as any protein which possesses a protein domain with at least about 30% sequence identity or similarity to a given protein, preferably at least about 40% sequence identity, and most preferably at least about 50% sequence identity. Useful sequence comparison algorithms to determine degree of sequence similarity include BLAST™, FASTA, DNA Strider, the GCG pileup program (Wisconsin Package version 10, Genetics Computer Group, Madison, Wis.), as well as alignment schemes such as Clustal W (See Thompson et al., supra), using, e.g., the default parameters provided with these algorithms. Sequences that are substantially homologous can be identified by comparing the sequences using standard software available in sequence data banks, or in a Southern hybridization experiment under, for example, stringent conditions as defined for that particular system. (See “hybridization”, below)

6. A “protein domain” as used herein is defined as a region of a protein which is at least about 50 amino acids ranging to the full length of the protein.

7. “Biological activity” as used herein refers to the ability of a protein to promote or sustain cell growth and/or metabolism through a known or unknown cellular mechanism. Biological activity need not be measured in living cells; an in vitro system consisting of the protein together with other chosen components, designed to reflect the ability of the protein to promote or sustain cell growth and/or metabolism, may also be used to evaluate biological activity.

8. “Target protein” or “cidal protein” as used herein refers to an essential protein involved in, e.g., growth and/or metabolism. Inhibition of the biological activity of a fungal target protein results in an inhibition of fungal growth. Target proteins may play essential roles in processes which include, but are not limited to, DNA synthesis, DNA repair, transcription, mRNA transport, mRNA processing, translation, protein transport, protein processing, cell cycle control, cell division, and cell signaling. The term “target protein” also includes fragments and polypeptides, as well as target proteins modified by any means known in the art, e.g., by radiolabeling, conjugation, mutations in amino acid sequence, using chemically modified amino acid residues in the target protein, and so forth.

9. “Mycete” or “fungi” as used herein refers to a eukaryotic organism which carries spores, nutrition of which takes place via absorption, which is deficient in chlorophyll and which reproduces sexually or asexually.

10. “Nucleic acid” or “polynucleotide” as used herein refers to purine- and pyrimidine-containing polymers of any length, either polyribonucleotides or polydeoxyribonucleotides or mixed polyribo-polydeoxyribo nucleotides. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases.

11. An “isolated” nucleic acid or polypeptide as used herein refers to a nucleic acid or polypeptide that is removed from its original environment (for example, its natural environment if it is naturally occurring). An isolated nucleic acid or polypeptide contains less than about 50%, preferably less than about 75%, and most preferably less than about 90%, of the cellular components with which it was originally associated.

12. A nucleic acid or polypeptide sequence that is “derived from” a designated sequence refers to a sequence that is related in nucleotide or amino acid sequence to a region of the designated sequence. For nucleic acid sequences, this encompasses sequences that are homologous or complementary to the sequence, as well as “sequence-conservative variants” and “function-conservative variants.” For polypeptide sequences, this encompasses “function-conservative variants.” Sequence-conservative variants are those in which a change of one or more nucleotides in a given codon position results in no alteration in the amino acid encoded at that position. Function-conservative variants are those in which a given amino acid residue in a polypeptide has been changed without altering the overall conformation and function of the native polypeptide, including, but not limited to, replacement of an amino acid with one having similar physical and/or chemical properties (such as, for example, acidic, basic, hydrophobic, and the like). “Function-conservative” variants of a designated polypeptide also include any polypeptides that have the ability to elicit antibodies specific to the designated polypeptide.

13. Nucleic acids are “hybridizable” to each other when at least one strand of nucleic acid can anneal to another nucleic acid strand under defined stringency conditions. Stringency of hybridization is determined, e.g., by a) the temperature at which hybridization and/or washing is performed, and b) the ionic strength and polarity (e.g., formamide concentration) of the hybridization and washing solutions, as well as other parameters. Hybridization requires that the two nucleic acids contain substantially complementary sequences; depending on the stringency of hybridization, however, mismatches may be tolerated. The appropriate stringency for hybridizing nucleic acids depends on the length of the nucleic acids and the degree of complementarity, variables well known in the art.

Hybridizable polynucleotides may be of any length. In one embodiment, such polynucleotides are at least 7, preferably at least 25 and most preferably at least 100 nucleotides long. In another embodiment, the polynucleotide that hybridizes to any of the polynucleotides of the invention is of the same length as the polynucleotide of the invention. Nucleic acids that are hybridizable to other nucleic acids are capable of hybridizing with their complements under the hybridization conditions defined herein as “high stringency” as defined below.

Prehybridization treatment of the support (nitrocellulose filter or nylon membrane), to which is bound the nucleic acid capable of being hybridized at 65EC for 6 hours with a solution having the following composition: 4×SSC, 10×Denhardt (1× Denhardt is 1% Ficoll, 1% polyvinylpyrrolidone, 1% BSA (bovine serum albumin); 1×SSC consists of 0.15M of NaCl and 0.015M of sodium citrate, pH 7);

Replacement of the pre-hybridization solution in contact with the support by a buffer solution having the following composition: 4×SSC, 1×Denhardt, 25 mM NaPO₄, pH 7, 2 mM EDTA, 0.5% SDS, 100 g/mL of sonicated salmon sperm DNA containing a nucleic acid probe, in particular as radioactive probe, and previously denatured by a treatment at 100EC for 3 minutes;

Incubation for 12 hours at 65EC;

Successive washings with the following solutions: (i) four washings with 2×SSC, 1×Denhardt, 0.5% SDS for 45 minutes at 65EC; (ii) two washings with 0.2×SSC, 0.1×SSC for 45 minutes at 65EC; and (iii) 0.1×SSC, 0.1% SDS for 45 minutes at 65EC.

14. A “promoter sequence” as used herein is defined as a DNA regulatory region capable of binding RNA polymerase in a cell and initiating transcription of a downstream (3′ direction) coding sequence.

15. A “candidate inhibitor,” as used herein, is any compound with a potential to inhibit Candida albicans or other fungal growth and/or metabolism via an activity mediated by any of the target proteins described in Table 1, and throughout the specification.

16. “ATLAS”, an abbreviation of “Any Target Ligand Assisted Screening” as used herein refers to the screening method described in the section entitled “Primary Inhibitor Screening; High-Throughput Methods for Screening Inhibitors.”

### Target proteins

The present invention is based on the isolation of DNA encoding fungal proteins involved in cellular growth and/or metabolism, particularly those derived from the S. cerevisiae and/or Candida albicans genes listed in Table 1, and the determination of the essentiality and or cidality of such fungal gene. The discovery and characterization of these S. cerevisiae target proteins and/or their C. albicans homologs, and the elucidation of differences between the fungal and mammalian target proteins, implicates these protein, particularly the C. albicans protein, as an important target for the development of new methods and compositions for the treatment of fungal infections. Agents which selectively interfere the biological activity would likely be candidates for anti-fungal, particularly anti-C. albicans and related fungi, therapeutics. The present invention also encompasses methods for identifying compounds that selectively interfere with C. albicans target protein activity and thus may comprise useful antifungal agents.

Ideally, an antifungal compound directs its action against a target that is present in fungi but absent in human cells. Such targets, however, are important for cell function and tend to be conserved in evolution and, thus, be present in both human and fungal cells. In such cases, the target protein is present in both cell types, as noted above, but the human homolog of the target protein has an amino acid sequence that distinguishes it from the fungal target protein.

If a human homolog of the target protein has been identified, such a human sequence is considered distinguishable from the fungal sequence if it has less than about 50%, preferably less than about 40%, and even more preferably less than about 30% sequence identity. The lower the sequence similarity, the higher the chance for identifying compounds that act specifically against the fungal target protein but not its human homolog. However, an important factor is also the sequence similarity between different fungal homologs of the target protein. If homologous proteins derived from two different fungal sources such as, e.g., S. cerevisiae and C. albicans, display a high sequence similarity such as, e.g., higher than 50%, more preferably 70%, and even more preferably higher than 90%, this allows for a higher chance of identifying an inhibitor specific for the fungal target proteins but not their human homolog. Thus, a higher than optimal sequence similarity between the fungal and human target protein homologs does not preclude finding a substance which only inhibits the biological activity of the fungal protein.

Each preferred target protein is described below. Non-limiting examples of some assays for some of the target proteins are also provided. Such assays are useful in identifying and/or measuring the biological activity of target proteins, e.g., in the presence of a potentially inhibitory compound. Amino acid sequences for each target protein in S. cerevisiae, C. albicans, and, where relevant, human, can be found in Table 1. Sequence identity determinations between the the S. cerevisiae, C. albicans, and, if available, human homologs, are provided in Table 2.

### RPC34

RPC34 (C34) is an essential and specific subunit of RNA polymerase III complex (Stettler, S., et al., J. Biol. Chem., 1992; 267:21390-21395). RNA polymerase III is responsible for transcription of tRNAs, 5S rRNA, and some other small RNAs. Three RNA polymerase III unique subunits, C34, C82, and C31 form a complex that interacts with 70-kDa component of transcription factor TFIIIB via C34 (Werner, M., et al., J. Biol. Chem., 1993; 268:20721-20724). C34 subunit is a major determinant of pol III recruitment by pre-initiation complex. Interaction between C34 and TFIIIB70 is essential for pre-initiation complex formation and later during promoter opening (Brun, I., et al., EMBO J., 1997; 16:5730-5741). It has been demonstrated that strains carrying temperature-sensitive or cold-sensitive mutations in RPC34 are impaired in tRNA synthesis (Stettler, S., et al., J. Biol. Chem., 1992; 267:21390-21395; Brun, I., et al., EMBO J., 1997; 16:5730-5741). RPC39 human homolog of RPC34 has been identified (Wang, Z. and Roeder, R. Gen. Dev., 1997; 11:327-7949). RPC34 and RPC39 are 27% identical and 50% similar.

RPC34 assays:

(a) ATLAS

(b) Cell-based assays in S. cerevisiae and human cells were developed utilizing the information that in the absence/inability to perform, the function of RPC34 tRNA synthesis decreases (Stettler, S., et al., J. Biol. Chem., 1992; 267:21390-21395; Brun, I., et al., EMBO J., 1997; 16:5730-5741). If the compound specifically binds to Rpc34p, a tRNA level decrease can be detected after addition of the compound to the growing media. Similar assay in human cells can be designed based on the same principle. The level of tRNA can be assayed upon addition of a compound to the cells at different time points.

(c) In vitro assays can be developed using purified RNA polymerase III transcription factors, including RPC34, to assess tRNA and 5S rRNA levels in the presence/absence of a compound (Kassavetis, G., et al., EMBO J., 1999; 18:5042-5051).

(d) A reporter-based assay can be developed utilizing a two-hybrid system, knowing that RPC34 physically interacts with C82, C31, and TFIIIB70. One of the proteins can be fused with a transcriptional activator and the other with a DNA-binding protein. The ability of the two proteins to interact with each other in the presence or absence of a compound can be measured by monitoring enzymatic activity of a reporter gene expressed from the promoter.

### POP3

Saccharomyces cerevisiae POP3 is involved in post-transcriptional processing of the large precursor RNAs into the mature functional forms of tRNA and rRNA (Dichtl, B. and D. Tollervey, EMBO Journal, 1997; 16:417-429; Chamberlain, J. R., et al., Genes and Development, 1998; 12:1678-1690). This processing of tRNA and rRNA is carried out by the RNase MRP and RNase P ribonucleoproteins, respectively, but the two complexes are known to have extensive subunit overlap (Chamberlain, J. R., et al., Genes and Development, 1998; 12:1678-1690). Mutations in POP3 result in phenotypes identical to loss of RNase MRP, including interference with the complete processing of tRNA and rRNA (Dichtl, B. and D. Tollervey, EMBO Journal, 1997; 16:417-429; Chamberlain, J. R., et al., Genes and Development, 1998; 12:1678-1690). POP3 is essential for cell growth in Saccharomyces cerevisiae (Dichtl, B. and D. Tollervey, EMBO Journal, 1997; 16:417-429).

POP3 Assays:

(a) ATLAS: CaPop3 protein could be purified and challenged with an environmental condition, such as higher temperature or reduced pH, that unfolds the protein. A compound that binds to CaPop3 protein may stabilize the native conformation of the protein.

(b) Two hybrid interruption screen using another interacting protein: CaPOP3 and a Candida albicans ortholog of another subunit of either the RNase MRP or the RNase P complex could be placed into yeast two-hybrid screening vectors, one as the bait and one as the target. Binding by the two proteins will induce expression of a reporter gene. A compound that interferes in the binding of the two proteins should disrupt the induction of the reporter gene, allowing such compounds to be identified in a screening format. Interacting proteins other than those in the RNase MRP or RNase P complex could be used in this format.

### TFA2

Saccharomyces cerevisiae TFA2 is a subunit of the general RNA polymerase II transcription initiation factor, TFIIE. The gene product of TFA2 forms a hetero-tetramer with that of TFA1, and both genes are essential for cell viability (Feaver et al., J Biol Chem, 1994, 269:27549-53). The genes for TFA1 and TFA2 were identified from the purified protein shown to have an activity required for accurately initiated transcription from promoters in vitro, and the gene sequences have significant homology to mammalian TFIIE (Feaver et al., J Biol Chem, 1994, 269:27549-53). The requirement for TFIIE to carry out transcription of a gene varies, depending on the promoter structures, (Sakur et al., J Biol Chem, 1997, 272: 15936-15942). It has been suggested that yeast GAL11 product enhances the interaction between TFIIE and the RNA polymerase II holoenzyme and thus increases transcriptional efficiency (Sakirau et al., PNAS, 1996, 93:9488-9492).

TFA2 Assays:

(a) ATLAS: CaTfa2 protein could be purified and challenged with an environmental condition, such as higher temperature or reduced pH, that unfolds the protein. A compound that binds to CaTfa2 protein may stabilize the native conformation of the protein.

(b) Two-hybrid interruption screen using another interacting protein: CaTfa2 and CaTfa1 could be placed into yeast two-hybrid screening vectors, one as the bait and one as the target. Binding by the two proteins will induce expression of a reporter gene. A compound that interferes in the binding of the two proteins should disrupt the induction of the reporter gene, allowing such compounds to be identified in a screening format. Interacting proteins other than CaTfa1p could be used in this format, notably CaGa111 protein.

### NAB2

Nascent RNA polymerase II transcripts associate with nuclear ribonucleoproteins and remain associated during the subsequent RNA processing reactions, such as pre-mRNA polyadenylation and splicing and transport to the cytoplasm. Saccharomyces cerevisiae NAB2 is one of the major proteins associated with polyadenylated RNA in vivo and is essential for cell growth (Anderson, J. T., et al., Molecular and Cellular Biology, 1993; 13:2730-2741). The NAB2 gene product is localized primarily to the nucleus (Anderson, J. T., et al., Molecular and Cellular Biology, 1993; 13:2730-2741). Two different RNA-binding motifs are identifiable in the sequence of NAB2: an RGG box observed in a variety of heterogenous nuclear RNA-binding proteins, and CCCH motif repeats related to the zinc-binding motifs of the largest subunit of RNA polymerases (Anderson, J. T., et al., Molecular and Cellular Biology, 1993; 13:2730-2741). NAB2 gene product interacts with the product of yeast KAP104, a gene encoding a karyopherin shown to function in the nuclear import of proteins, and has been shown to interact with human transportini (hTRN1), the human homolog of yeast KAP104 (Aitchison, J. D., et al., Science, 1996; 274:624-627;Truant, R., et al., Molecular and Cellular Biology, 1998; 18:1449-1458; M. C. Siomi, et al., Molecular and Cellular Biology, 1998; 18:4141-4148).

NAB2 Assays:

(a) ATLAS: CaNab2 protein could be purified and challenged with an environmental condition, such as higher temperature or reduced pH, that unfolds the protein. A compound that binds to CaNab2 protein may stabilize the native conformation of the protein.

(b) Two-hybrid interruption screen using another interacting protein: CaNAB2 and CaKAP104 could be placed into yeast two-hybrid screening vectors, one as the bait and one as the target. Binding by the two proteins will induce expression of a reporter gene. A compound that interferes in the binding of the two proteins should disrupt the induction of the reporter gene, allowing such compounds to be identified in a screening format. Interacting proteins other than CaKap104p could be used in this format.

(c) RNA-binding screen: Compounds could be screened for their ability to interfere with the binding of RNA by CaNab2 protein. The binding of RNA and CaNab2 protein could be assessed in a variety of ways: 1) through capture on a filter or capture by antibodies; 2) in homogeneous solution using fluorescently-labeled RNA and detection of a change in fluorescence polarization; or 3) detection of a gel shift when RNA is bound by the protein.

### MPT1

MPT1 is a target that has been identified in both S. cerevisiae and C. albicans. MPT 1 proteins have not been characterized in detail. ScMPT1 was isolated in a two-hybrid screen using ScPrp9 as bait (Froniont-Racine, M., et al., Nat Genet, 1997; 16:277-82). Prp9 is a subunit of a complex involved in RNA splicing. The fact that ScMPT1 would interact with Prp9 suggests that ScMPT1 would also be involved in RNA splicing. Validation data in S. cerevisiae and C. albicans indicate that MPT 1 is important for fungal cell growth and viability, which may correlate with its putative function in RNA splicing. A mammalian homolog has been proposed, but the degree of homology is too low to be confident about this. The apparent importance of MPT1 for fungal growth combined with the absence of a highly similar protein in mammalian cells make MPT1 an excellent target for antifungal drug discovery.

MPT1 assays:

(a) ATLAS. (See above).

(b) Cell-based assays: Various strains of S. cerevisiae could be constructed in which ScMPT1 would be replaced with a functional MPT1 gene (i.e., derived from cDNA when necessary) from different organisms, in particular fungi and mammals. These cells would be grown in individual wells containing defined number and mixtures of compounds, which potentially could inhibit growth. Differences in degrees of inhibition by compounds between above-mentioned strains may suggest that a compound may inhibit growth by preferentially inhibiting activity of a class of MPT1.

(c) Protein-protein interaction based assays: (i) Two-hybrid screen (Fromont-Racine, M., et al., Nat Genet, 1997; 16:277-82) using MPT1 and PRP9 (or any other protein found to interact with MPT1); (ii) Direct binding assay: The interacting protein could be fixed onto a carrier an allowed to bind easily detectable MPT1. In the absence of inhibitors, a high signal would result. However, interference with this interaction may reduce signal. The orientation of the assay could also be reversed by fixation of MPT1 and incubation with a interacting protein labeled with a reporter molecule such as, e.g., a radionucleotide or a fluorescent compound.

### MTR2

In eukaryotic cells, mRNA transport is an important cellular process for gene expression and regulation. A set of genes were identified through an attempt to isolate Saccharomyces cerevisiae temperature-sensitive mutants that accumulate poly(A) RNA in the nucleus.(Kadowaki, T., et al., J Cell Biol, 1994; 126, 649-59) One of the genes, MTR2 encodes a 21 kD nuclear protein that shows a limited homology to a E. coli protein implicated in plasmid DNA transfer.(Kadowaki, T., et al., J Cell Biol, 1994; 126, 649-59) It has been shown that Mtr2 protein can interact with a nuclei pore associated protein, Mex67p and their interaction appears to be essential for mRNA export. (Santos-Rosa, H., et al., Mol Cell Biol, 1998; 18:6826-38) Genetic and biochemical evidence also indicated that Mtr2p can interact with Nup85p, suggesting that Nup85p might be the target at nuclei pore complex (NPC) to which Mtr2p and Mex67 bind.(Santos-Rosa, H., et al., Mol Cell Biol, 1998; 18:6826-38) Given all these factors, it was proposed that Mtr2 protein is the key component of mRNA export machinery in yeast. (Santos-Rosa, H., et al., Mol Cell Biol, 1998; 18:6826-38; Schneiter, R., et al., Mol Biol Cell, 1995; 6:357-70)

Recently, a human homolog of Mex67, TAP was identified that can interact with poly(A) RNA and human nucleoporin. However, no Mtr2 human homolog was found so far. Katahira et al (The Mex67p-mediated nuclear mRNA export pathway is conserved from yeast to human. Embo Journal 18, 2593-2609 (1999)) identified a small human protein, p15 that interact with TAP. Interestingly, co-expression of TAP and p15 in yeast can functionally complement Mex67-Mtr2 complex suggesting the existence of the evolutionarily conserved pathway that is involved in mRNA transport.

MTR2 assays:

(a) ATLAS: Mtr2 protein can be purified to homogeneity. Challenging purified Mtr2 protein with different environment conditions such as higher temperature or reduced pH will result in the protein conformation change leading protein to the unfolding state. Any compound that binds to Mtr2 will potentially stabilize protein in the native state. Using ATLAS can help identify compound that binds to Mtr2.

(b) Two hybrid with Mex67. Mtr2 and Mex67 can be used as a pair of genes in yeast with one of them as the bait and the other used as target. Binding of Mtr2 and Mex67 protein in yeast will result in the induction of a reporter gene that can be detected. Any compound that interrupts the interaction of Mtr2pand Mex67p will disrupt the induction of the reporter gene and thus that compound can be identified.

(c) Two hybrid with Nup85p. Mtr2 and Nup85 can be used as a pair of genes in yeast with one of them as the bait and the other used as target. Binding of Mtr2 and Mex67 protein in yeast will result in the induction of a reporter gene that can be detected. Any compound that interrupts the interaction of Mtr2p and Nup85p will disrupt the induction of the reporter gene and thus that compound can be identified.

### BOS1

Saccharomyces cerevisiae BOS1 is an essential gene that functions in ER-to-Golgi transport. The protein is a cytoplasmically-oriented type II integral membrane protein of secretory vesicles (Newman et al., Embo J.,1992, 11:3609-3617; Lian et al., Cell, 1993, 73:735-745). Depletion of BOS1 results in a block in ER-to-Golgi protein transport and accumulation of small vesicles (Shim et al., J. Cell Biol., 1991, 13:55-64). The gene was originally isolated as a high copy suppressor of BET1 (Newman et al., Embo J., 1992, 11:3609-3617). BOS1 exhibits genetic and physical interactions with several proteins known to be involved in vesicular transport from the ER to the Golgi. In addition to suppressing BET1 defects, BOS1 overexpression can also overcome defects in SEC22 and YPT1 (Newman et al. Embo Journal 11, 3609-17(1992)). Bos1p has been shown to pair with Sec22p under the influence of Ypt1. Bos1p, Bet1p and Sec22p are V-SNARE proteins (Lian et al., Cell 73, 735-45 (1993); Pfeffer, Annu. Rev. Cell Dev. Biol. 12, 441-461 (1996)) that form a complex involved in transport vesicle docking (Ferro-Novick et al., Cell Biophys 19, 25-33 (1991)). YPT1 is a Rab protein required for SNARE complex formation (Sogaard et al., Cell 78, 937-48 (1994); Lian et al., Nature 372, 698-701 (1994); Lazar et al., Trends Biochem Sci 22, 468-472 (1997)). The V-SNAREs Bos1p and Sec22p cooperatively interact with the t-SNARE Sed5p prior to membrane fusion (Sacher et al., J Biol Chem 272, 17134-8 (1997)).

BOS1 assays:

(a) BOS1 is a good ATLAS assay target. In addition, defects in BOS1 function could be assessed in a reconstituted transport system (Lian, J. P., and Ferro-Novick, S. Bos1p, Cell, 1993; 73:735-45) or in a cell-based assay of invertase secretion (Johnson, L. M., et al.,Cell, 1987; 48:875-885) that monitors the inefficient transport of secreted protein from the ER to the Golgi (Shim, J., et al., J Cell Biol, 1991; 113:55-64).

(b) In vitro transport system (Lian et al., Cell 73, 735-45 (1993)).

(c) Cell-based assay of invertase secretion (Johnson et al., 1987) that monitors the inefficient transport of secreted protein from the ER to the Golgi (Shim et al., 1991).

(d) Protein:protein interactions. BOS1 has multiple protein partners (see above) whose interactions can be monitored by assayed by two-hybrid analysis or in vitro protein binding assays.

### POL30

References for this section are numbered at the end of the section.

Saccharomyces cerevisiae POL30 is an essential gene and encodes the yeast proliferating cell nuclear antigen (PCNA) (Bauer et al., NAR, 1990, 18: 261-5). The structure of yeast PCNA has been determined, and it appears to function as a trimer that forms a sliding clamp around the DNA double helix (Krishna et al., J Mol Biol, 1994, 241: 265-8). PCNA can load onto the ends of linear DNA molecules in vitro, but efficient loading of PCNA onto DNA requires ATP and the product of RFC1 (McAlear et al., Genetics, 1996, 142:65-78, Burgers et al., J Biol. Chem., 1993, 268: 19923-19926).

PCNA is required for both DNA synthesis and DNA repair in mammals and yeast. PCNA interacts with DNA polymerase delta or epsilon to enhance processive replication of DNA (Holmes et al., Cell, 1999, 96: 415-424). PCNA interacts with FEN-1, the product of the mammalian homolog of RAD27, a protein required for Okazaki fragment processing (Ishimi et al., J. Biol.Chem., 1988,263: 19723-19733; Li et al., J. Biol. Chem., 1995, 270:22109-22112; Turchi et al., PNAS, 1995, 91:9803-9807). PCNA is required in vitro for reconstitution of nucleotide excision repair and base excision repair reactions. (Ayyagari et al., Mol Cell Biol, 1995, 15:4420-0; Umar et al., Cell, 1996, 87:65-73; Johnson et al, J Biol Chem, 1996, 271:27987-90; Matsumoto et al., Mol Cell Bio., 1994, 14:6187-97; Nichols et al., NAR, 1992 10:2441-2446; Shivji et al., Cell, 1992, 69:367-374). Transcription silencing may also involve PCNA (Ehrenhofer-Murray et al., Genetics, 1999, 153:1171-82).

POL30 assays:

(a) ATLAS: CaPo130 protein could be purified and challenged with an environmental condition, such as higher temperature or reduced pH, that unfolds the protein. A compound that binds to CaPo130 protein may stabilize the native conformation of the protein.

(b) Two-hybrid interruption screen using CaRad27 protein or another interacting protein: CaPo130 and CaRad27 could be placed into yeast two-hybrid screening vectors, one as the bait and one as the target. Binding by the two proteins will induce expression of a reporter gene. A compound that interferes in the binding of the two proteins should disrupt the induction of the reporter gene, allowing such compounds to be identified in a screening format. Interacting proteins other than CaRad27p could be used in this format. A screen could be designed to interfere with the multimerization of CaPo130 by using the gene as both bait and prey.

(c) DNA-binding screen: Compounds could be screened for their ability to interfere with the binding of DNA to CaPo130 protein. The binding of DNA and CaPo130 protein could be assessed in a variety of ways: 1) through capture on a filter or capture by antibodies; 2) in homogeneous solution using fluorescently-labeled DNA and detection of a change in fluorescence polarization; or 3) detection of a gel shift when DNA is bound by the protein.

### YMR131C

YMR131C is an essential gene in C. albicans. Nearest human match is a 25% identity to human retinoblastoma protein RBBP4. YMR131 C protein has WD40 repeats suggesting that it may physically interact with other proteins. Recent report suggests that the protein may be involved in the nucleopore complex formation (Rout, M., et al., J. Cell Biol., 2000; 148:635-652).

YMR131C assays:

(a) ATLAS

(b) If a mammalian YMR131C Homolog is found that complements C albicans YMR131C, a cell-based assay could be set up to measure cell growth in the presence/absence of a compounds comparing strains with C. albicans YMR131C and human YMR131C Homolog.

(c) If proteins that physically interact with YMR131C are identified, two-hybrid system based assay can be developed to monitor interaction between YMR131C and another protein.

(d) If YMR131C is essential for nuclear pore transport, an assay can be set up to monitor efficiency of transport through nuclear pores.

### SQT1

Saccharomyces cerevisiae SQT1 is an essential gene, which encodes a 60S ribosomal subunit protein required for joining of 40S and 60S subunits (Eisinger et al., MCB, 17:5146-5155, 1997). SQT1 was isolated as a suppressor of dominant-negative truncation mutations of ribosomal protein QSR1 (Eisinger et al., MCB, 17:5136-5145, 1997; Eisinger et al., MCB, 17:5146-5155, 1997). The loss of SQT1 function results in the formation of half-mer polysomes whereby the 40S and 60S subunits fail to join. SQT1 may be required for the assembly of QSR1 onto the 60S ribosomal subunit (Eisinger et al., MCB, 17:5146-5155, 1997). The protein may be part of an oligomeric complex and is localized to the cytoplasm where it is loosely associated with ribosomes (Eisinger et al., MCB, 17:5146-5155, 1997).

SQT1 assays:

(a) SQT1 is a good candidate for an ATLAS assay. In addition, polysome and ribosome subunit analysis could be carried out in a low-throughput secondary assay. Interference with SQT1 function should result in half-mer polysome profiles. This type of assay would involve isolation and fractionation of ribosomal subunits, 80S ribosomes and polysomes on sucrose velocity gradients (Eisinger et al., MCB, 17:5136-5145, 1997).

(b) Polysome and ribosome subunit analysis could be carried out in a low-throughput secondary assay. Interference with SQT1 function should result in half-mer polysome profiles. This type of assay would involve isolation and fractionation of ribosomal subunits, 80S ribosomes and polysomes on sucrose velocity gradients (Eisinger et al., 1997a).

### MTW1

MTW1 is an essential protein in C. albicans with unknown function. Mtw1p (Mis twelve-like protein) is 33% identical to S. cerevisiae Mis12p. The published data suggests that S. pombe Mis12p is required for centromere structure maintenance and correct spindle morphogenesis during chromosomal segregation (Goshima et al., Gen. Dev., 13:1664-1677, 1999). It is possible that C. albicans Mtw1p has DNA-binding motifs. No true human homolog has been identified so far.

MTW5 assays:

(a) ATLAS

(b)If MTW1 binds to DNA, an assay for DNA-binding activity can be set up.

(c) If a mammalian MTW1 homolog is found which complements C albicans MTW1, a cell-based assay can be set up to measure cell growth in the presence/absence of a compound, comparing strains with C. albicans MTW1 and the human MTW1 homolog.

(d) If proteins that physically interact with MTW1 are identified, two-hybrid system based assays can be developed to monitor interaction between MTW1 and other proteins.

### TFB1

RNA polymerase II needs five additional general transcription factors for promotor dependent transcription, one of which is TFIIH (Svejstrup et al., J Biol Chem, 269:28044-8, 1994). TFIIH contains DNA-dependent ATPase activity and protein kinase activity directed against the C-terminal Repeat Domain of RNA polymerase II. TFB1 is one of the subunits of TFIIH and is needed for both transcription and nucleotide excision repair.

TFB1 genes have been found in both mammalian and fungal cells. However, the degree of conservation between fungi is higher than that between fungi and mammalian (approximately 40% vs. 20%). This difference combined with the importance for fungal cell viability makes TFB1 an excellent target for antifungal drug discovery.

TFB1 assays:

(a) ATLAS

(b) RNA polymerase II promotor-dependent transcription assay

(c) Cell-based assay: Various strains of S. cerevisiae would be constructed in which ScNIP1 would be replaced with a functional TFB1 gene (i.e. derived from cDNA when necessary) from different organisms, in particular fungi and mammals. These cells would be grown in individual wells containing defined number and mixtures of compounds, which potentially could inhibit growth. Differences in degrees of inhibition by compounds between above-mentioned strains suggest that a compound may inhibit growth by preferentially inhibiting activity of a class of TFB1.

(d) Protein-protein/DNA interaction based assay: (i) Two-hybrid screen (Fromont-Racine et al., Nat Genet, 16:277-82, 1997) using TFB1 and any protein (or DNA) found to interact with TFB1 (e.g. other TFIIH subunits); (ii) Direct binding assay: The interacting protein or DNA would be fixed onto a carrier an allowed to bind easily detectable TFB1. In the absence of inhibitors a high signal would result. However, interference with this interaction would reduce signal. Orientation of the assay could also be reversed by fixation of TFB1 and incubation with labeled interacting protein/DNA.

### SPC98

Saccharomyces cerevisiae SPC98 encodes an essential protein that has a role at the spindle pole body (SPB), the fungal equivalent of the centrosome. SPC98 was identified as a high copy suppressor of a mutation in TUB4, the yeast gene for gamma-tubulin. A conditional mutation in SPC98, when shifted to restrictive conditions, results in a cell-cycle arrest with defective mitotic spindles (Geissler, et al., Embo Journal, 15:3899-911, 1996). SPC97, a gene that has regions of sequence similarity to SPC98, was identified as a high copy suppressor of a mutation in SPC98 (Knop et al., Embo Journal, 16:1550-64, 1997). The products of both SPC97 and SPC98 have been shown to form a complex with gamma tubulin and to be responsible for microtubule nucleation (Knop, M., et al, 1997; Pereira et al., Embo Journal, 18:4180-4195, 1999; Chen et al., J Cell Biol, 141:1169-1179, 1998). The human homologs of SPC97 and SPC98 are also in a complex with ganuma-tubulin and appear to have the same functions (Tassin et al., J Cell Biol, 141:689-701, 1998; Murphy et al., J Cell Biol, 141:663-74, 1998).

SPC98 Assays:

(a) ATLAS: CaSpc98 protein could be purified and challenged with an environmental condition, such as higher temperature or reduced pH, that unfolds the protein. A compound that binds to CaSpc98 protein may stabilize the native conformation of the protein.

(b) Two hybrid interruption screen using another interacting protein: CaSpc98 and CaSpc97 could be placed into yeast two-hybrid screening vectors, one as the bait and one as the target. Binding by the two proteins will induce expression of a reporter gene. A compound that interferes in the binding of the two proteins should disrupt the induction of the reporter gene, allowing such compounds to be identified in a screening format. Interacting proteins other than CaSpc97 could be used in this format.

### BFR2

Saccharomyces cerevisiae BFR2 is an essential gene that was isolated as a high copy suppressor of the growth defects induced by Brefeldin A (BFA), a fungal metabolite that disrupts Golgi structure and function (Chabane et al., Curr. Genet, 33:21-8, 1998; Takatsuki et al., Agric. Biol. Chem., 49:899-902, 1995; Klausner et al., J. Cell Biol., 116:1071-1080, 1992). In addition, BFR2 overproduction was shown to partially suppress the growth defects of four mutants involved in the secretory pathway (Chabane et al. 1998). The mutants, sec13-1, sec16-1, sec23-1 and ypt1-1, are each involved in budding and or docking of small vesicles en route to the Golgi. Thus, it was suggested that BFR2 is involved in protein transport (Chabane et al. 1998).

BRF2 assays:

(a) BFR2 can be screened in an ATLAS assay format; and

(b) Based on the proposed function of BFR2, compound interference with BFR2 would make cells more highly sensitive to BFA. Therefore, increased cellular sensitivity to BFA is an additional assay that could be used as a secondary screen.

### RNA1

Saccharomyces cerevisiae RNA1 gene encodes the Rna1 protein, which is involved in nuclear export of all types of RNA (Sarkar et al. Mol Biol Cell, 1998, 9:3041-55). It is required for export of assembled 60S ribosomal subunits from the nucleus to the cytoplasm (Hurt et al., J Cell Biol, 1999, 144:389-401). Rna1p plays a direct role in the import of proteins into the nucleus (Corbett et al., J Cell Biol, 1995, 130:1017-26). GST-Rna1p catalytically stimulates GTP hydrolysis by purified Gsp1p (Corbett et al., J Cell Biol, 1995, 130:1017-26). It does not stimulate GTPase activity of ras or Rab7 (Becket et al., J Biol Chem, 1995, 270:11860-5). RNA1 has extensive homology to S. pombe Rna1p and to the mammalian Ran/TC4 GTPase activating protein (Corbett et al., J Cell Biol, 1995, 130:1017-26; Bischoff et al., PNCAS USA, 1995 92:1749-53; Melchior et al., Mol Biol Cell, 1993 4:569-81). The rna1-1 mutant is complemented by S. pombe rna1. It is a member of superfamily of proteins that have leucine-rich repeat motifs, which can be up to 29 amino acids in length (Melchior et al., Mol Biol Cell, 1993 4:569-81; Schneider et al., Mol Gen Genet, 1992, 233: 315-8). Cytosolic extracts made from rna1-1 mutants are completely devoid of Rna1p and the protein was found to be localized within the nucleus (Traglie et al., PNCAS USA, 1996, 93:7667-72). The mutant affects RNA processing and export from nucleus although Rna1p is cytoplasmic (Hopper et al., J Cell Biol, 1990, 111:309-21). rna1-1 mutant accumulates intron-less and intron-containing tRNA in the nucleus at the nonpermissive temperature (Sarkar et al. Mol Biol Cell, 1998, 9:3041-55). It shows altered export of RNA from nucleus to cytoplasm with RNA accumulating at the nuclear periphery (Amberg et al., GAD, 1992 6:1173-89). The temperature-sensitive mutant has accumulation of 35S pre-rRNA (Venema et al., Yeast, 1995, 11:1629-50). The rna1-1 mutant abolishes nuclear pore complex localization of Cse1p-GFP, which becomes distributed throughout the cell (Hood et al., J Biol Chem, 1998, 273:35142-35146). When the 11 amino acids from the carboxy terminal are removed, the protein retains its function (Traglia et al., Mol Cell Biol, 1989, 9:2989-99). In rna1-1 mutant, export of the small ribosomal subunit from the nucleus is directly inhibited with accompanying secondary defects in processing of pre-rRNA (Moy et al., GAD, 1999, 13:2118-2133).

RNA1 assays:

(a) ATLAS

(b) Mutants of RNA1 accumulates intron-less and intron-containing tRNA (1). This information may be useful in assaying such tRNA in presence/absence of compounds that bind and disrupt Rna1p activity.

(c) The defects in processing of ³⁵S pre-rRNA may be monitored by probing with oligonucleotides near the pre-rRNA cleavage sites by Northern Hybridization and primer extension analysis.

(d) There is accumulation of ³⁵S pre-rRNA in temperature sensitive mutants (11). This effect may be studied in a cell-based assay. Levels of ³⁵S-labeled pre-rRNA may be assayed in presence/absence of a compound.

### GCD7

Eukaryotic protein translation is initiated by acquisition of mRNA and Met-tRNAiMet by the 40S ribosomal subunit. These changes are mediated by Initiation Factors (eIF's). eIF2 forms a complex with Met-tRNAiMet and GTP, which binds to 40S ribosomes (Pavitt et al., Mol Cell Biol, 1997, 17:1298-313). After subsequent binding of mRNA to these 40S ribosomes and recognition of the AUG codon by Met-tRNAiMet, GTP hydrolysis releases eIF2-GDP. eIF2-GDP is converted to eIF2-GTP by eIF2B, a guanine nucleotide exchange factor, as a result of which protein translation can continue. Starvation for amino acids leads to phosphorylation of eIF2, reduction of recycling of eIF2-GDP by eIF2B and preferential translation of GCN4, a transcriptional activator of amino acid biosynthetic enzymes. eIF2B is composed of 5 subunits of which 4, including GCD7, are essential for growth. GCD7 seems to form part of the binding site for phosphorylated-eIF2 thereby mediating inhibition of eIF2B.

GCD7 genes have been found in both mammalian and fungal cells. However, the degree of conservation between fungi is higher than that between fungi and mammalian (approximately 50% vs. 35%). This difference combined with the importance for fungal cell viability makes GCD7 an excellent target for antifungal drug discovery.

GCD7 assays:

(a) ATLAS

(b) Protein translation assay (Colthurst, et al., J Gem Microbiol, 1991, 137:851 -857)

(c) Cell-based assays: (i) Various strains of S. cerevisiae could be constructed in which ScGCD7 would be replaced with a functional GCD7 gene (i.e., derived from cDNA when necessary) from different organisms, in particular fungi and mammals. These cells would be grown in individual wells containing defined number and mixtures of compounds, which potentially could inhibit growth. Differences in degrees of inhibition by compounds between above-mentioned strains suggest that a compound may inhibit growth by preferentially inhibiting activity of a class of GCD7; (ii) Instead of measuring growth dependent on the presence of inhibitory compounds a more specific assay aimed at expression of GCN4 could be performed. Histidine starvation would be induced with AT thereby making expression of GCN4 required for growth. Alternatively, cells could be grown to higher densities prior to addition of AT and GCN4 activation could be monitored by transcriptional (or translational) fusions of the GCN promotor (plus (part of) Gcn4p) to a suitable reporter gene/protein (Pavitt et al., Mol Cell Biol, 1997, 17:1298-313).

(d) GDP exchange assays (Cigan et al., PNAS, 1993, 90:5350-5354): eIF2 and eIF2B would be isolated from an appropriate host. eIF2 would complexed with labeled GDP. Incubation of this complex will release labeled GDP, which would be separated from the complex. Compound interference with this liberation would leave high amounts of label.

(e) Protein-protein interaction based assays: (i) A two-hybrid screen (Fromont-Racine et al., Nat Genet, 1997, 16:277-82) using GCD7 and any protein found to interact with GCD7 (e.g. other eIF2 subunits); (ii) A direct binding assay. The interacting protein would be fixed onto a carrier an allowed to bind easily detectable GCD7. In the absence of inhibitors, a high signal would result. However, interference with this interaction would reduce the signal. Orientation of the assay could also be reversed by fixation of GCD7 and incubation with labeled interacting protein.

### SKI6

Most strains of Saccharomyces cerevisiae carry one or more dsRNA viruses. Yeast harboring these viruses are called killer strains and secret toxin which is lethal to most of the ones that carry no viruses. Derepression of toxin expression results in superkiller phenotype (Ridley et al., Mol Cell Biol, 1984, 4:761-70).

SKI6 is one of the many genes that were identified by the superkiller phenotype of mutants. (Masison et al., Mol Cell Biol, 1995, 15:2763-71) It encodes an essential protein that is homologous to bacterial tRNA-processing enzyme, RNase PH. (Lussier et al., Genetics, 1997, 147:435-450;Mitchell et al., Cell, 1997, 91:457-466) Benard et. al. discovered that ski6 mutation bypassed the requirement of polyA tail for efficient mRNA translation, allowing better translation of non-polyA mRNA, including L-A virus mRNA. (Benard et al., Mol Cell Biol, 1998, 18:2688-2696) Later experiments suggested that SKI6 plays an important role in 3′-5′ mRNA decay which is consistent with the fact the ski6 mutant derepresses the virus mRNA translation. (Mitchell et al., Cell, 1997, 91:457-466;vanHoof et al., Cell, 1999, 99:347-350)

SKI6 also functions in ribosomal RNA processing. (Allmang et al., GAD, 1999, 13:2148-58) It is a part of exosome complex that functions as 3′-5′ exoribonuclease that is required for 5.8S rRNA maturation. (Mitchell et al., Cell, 1997, 91:457-466)

SKI6 Ski6p can be screened by 3 ′-5′ exoribonuclease activities. RNA substrate will be radiolabeled with P-32 and incubated with recombinant purified Ski6p. Loss of TCA precipitable radiolabeled RNA substrate is due to the activity of Ski6 protein, and inhibitors of Ski6p can thereby be screened.

(a) ATLAS: Ski6 protein can be purified to homogeneity. Challenging purified Ski6 protein with different environment conditions such as higher temperature or reduced pH will result in the protein conformation change leading to the unfolding state. Any compound that binds to Ski6 can potentially stabilize protein in the native state. Using ATLAS can help identify compound that binds to Ski6p.

(b) Luciferase assay. Luciferase messenger RNA with or without PolyA tails can be prepared and transfected into yeast through electroporation. Since Ski6p blocks translation of non-polyA mRNA, Luciferase activity will be high with mRNA that contains polyA tails and about 40 times lower with mRNA that has no polyA tails. In the presence of compound that block the activity of Ski6p, luciferase activity in the presence of mRNA that contains polyA tails should remain relatively the same while activity in the absence of polyA tail should increase about 10 times.

### NIP1

Eukaryotic protein translation is a initiated by acquisition of mRNA and Met-tRNAiMet by the 40S ribosomal subunit (Hanachi et al., J Biol Chem, 1999, 274:8546-8553). These changes are mediated by Initiation Factors (eIF's). eIF3 is composed of approximately 8-10 subunits, one of which is NIP1. No specific, enzymatic function of NIP1 within eIF3 has been described. However, validation of this gene in C. albicans and S. cerevisiae indicates that the protein is important for cell growth and viability.

NIP1 genes have been found in both mammalian and fungal cells. However, the degree of conservation between fungi is higher than that between fungi and mammalian (approx. 40% vs. 25%). This difference combined with the importance for fungal cell viability makes NIP1 an excellent target for antifungal drug discovery.

NIP1 assays:

(a) ATLAS

(b) Protein translation assay (Colthurst et al., J Gen Biol, 1991, 137:851-857)

(c) Cell-based assays: Various strains of S. cerevisiae would be constructed in which ScNIP1 would be replaced with a functional NIP1 gene (i.e. derived from cDNA when necessary) from different organisms, in particular fungi and mammals. These cells would be grown in individual wells containing defined number and mixtures of compounds, which potentially could inhibit growth. Differences in degrees of inhibition by compounds between above-mentioned strains suggest that a compound may inhibit growth by preferentially inhibiting activity of a class of NIP1.

(d) Protein-protein interaction based assays: (i) A two-hybrid screen (Fromont-Racine et al., Nat Genet, 1997, 16:277-82) using NIP1 and any protein found to interact with NIP1 (e.g. other eIF3 subunits); (ii) Direct binding assay: The interacting protein would be fixed onto a carrier an allowed to bind easily detectable NIP1. In the absence of inhibitors a high signal would result. However, interference with this interaction would reduce signal. Orientation of the assay could also be reversed by fixation of NIP1 and incubation with labeled interacting protein

### LCP5

LCP5 is an essential Saccharomyces cerevisiae gene which encodes a 40.8 Kd protein. LCP5p immunolocalizes to the nucleolus and participates in the early cleavage events at sites A0 to A2 in the pathway of pre-rRNA processing (Wiederkehr et al., RNA, 1998, 4:1357-1372). Depletion leads to reduced levels of 18S ribosomal subunits with concomitant accumulation of 60S ribosomal subunits and a sharp reduction in polysomes (Wiederkehr et al., RNA, 1998, 4:1357-1372). An lcp5-1 mutant shows increased sensitivity to the aminoglycoside antibiotics paromomycin and neomycin, and to cycloheximide, indicating a defect in translation (Wiederkehr et al., RNA, 1998, 4: 1357-1372). lcp5-1 mutant, or depletion of Lcp5p, shows sharp reduction of 18S rRNA, with accumulation of an aberrant 23S pre-rRNA species (Wiederkehr et al., RNA, 1998, 4:1357-1372).

LPC5 assays:

(a) ATLAS

(b) Lcp5 mutant shows predominant processing at site A3 and reduced cleavage at sites A0 and A2 in the 35S pre-rRNA (Wiederkehr et al., RNA, 1998, 4:1357-1372). The defects in processing of ³⁵S pre-rRNA may be monitored by probing with oligonucleotides near the pre-rRNA cleavage sites by Northern Hybridization and primer extension analysis.

(c) The rRNA metabolism may be affected by LCP5 specific compounds and this may be monitored by looking at the total RNA which will show a decrease in the steady state amounts of 18S rRNA (Wiederkehr et al., RNA, 1998, 4:1357-1372).

(d) Compounds may be assayed in presence/absence of alminoglycoside antibiotics paromomycin and neomycin, and to cycloheximide. Since mutant shows an increased sensitivity to these antibiotics (Wiederkehr et al., RNA, 1998, 4:1357-1372), a synergystic effect may be observed.

### NCE103

In a search for components of protein export machinery, Cleves et al (Cleves et al., J Cell Biol., 1996, 133(5):1017-26) discovered NCE103 gene that is involved in non-classic export pathway that functions independent of the classical pathway through ER and the Golgi compartments. (Cleves et al., J Cell Biol., 1996, 133(5):1017-26) Even though NCE103 gene appeared to be essential under normal conditions, experiments by Gotz et al suggested that it grew like wild-type under anaerobics conditions. (Gotz, et al., Yeast, 1999, 15:855-864) The predicted amino acid sequence of Nce103p shows high levels of identities to carbonic anhydrase of both prokaryotes and eukaryotes. (Gotz, et al., Yeast, 1999, 15:855-864) Expression of Medicago sativa carbonic anhydrase gene in a high-copy number plasmid complement the growth defects caused by nce103 deletion. (Gotz, et al., Yeast, 1999, 15:855-864) Given that nce103 deletion strain grow like wild-type under anaerobic conditions and null deletion can be complemented by Medicago sativa carbonic anhydrase gene, it was proposed that nce103 functions as an authentic carbonic anhyrase and is required for protection against certain products of oxidative metabolites under aerobics condition. (Gotz, et al., Yeast, 1999, 15:855-864)

NCE103 assays:

(a) ATLAS: Nce103 protein can be purified to homogeneity. Challenging purified Nce103 protein with different environment conditions such as higher temperature or reduced pH will result in the protein conformation change leading protein to the unfolding state. Any compound that binds to Nce103p can potentially stabilize protein in the native state. Using ATLAS can help identify compound that binds to Nce103p.

### ECO1

Saccharomyces cerevisiae ECO1 (also called CTF7) is an essential gene that is required to establish cohesion between sister chromatids during DNA replication. It was isolated as a mutant that can separate sister centromeres in the presence of Pds1p, an anaphase inhibitory protein (Toth et al., Genes and Dev., 13:320-333, 1999; Skibbens et al., Genes and Dev., 13:307-319, 1999). The protein is essential during S phase to establish sister chromatid cohesion but not during mitosis to maintain it (Skibbens et al., 1999). Cells harboring temperature-sensitive alleles of ECO1 arrest at restrictive temperature predominately as large budded cells with elongated spindles. There is a defect in separation of DNA such that mother cells often contain all the DNA (Skibbens et al., 1999). Some temperature-sensitive mutants display increased chormosome fragment loss at permissive temperature (Toth et al., 1999; Skibbens et al., 1999). The POL30 (DNA replication processivity factor or PCNA) gene in high copy can suppress ctf7 temperature sensitivity and chromosome loss thus lending further support of the hypothesis that CTF1/ECO1 functions in the establishment of sister chromatid cohesion (Skibbens et al., 1999).

ECO1 assays:

(a) ECO1 can be screened in an ATLAS format. Chromosome fragment loss can be assessed in a secondary assay. In this assay, faithful maintenance of a reporter chromosome fragment yields white colonies whereas loss of the reporter chromosome yields red sectored colonies (Toth et al., 1999; Skibbens, et al., 1999). In addition, the DNA content of cells can be analyzed by flow cytometry and in micrographs of cells stained with the nuclear dye, DAPI. (Toth et al., 1999).

(b) Chromosome fragment loss. Faithful maintenance of a reporter chromosome fragment yields white colonies whereas loss of the reporter chromosome yields red sectored colonies (Toth et al., 1999; Skibbens, et al., 1999).

(c) DNA content of cells can be analyzed by flow cytometry and in micrographs of cells stained with the nuclear dye, DAPI. (Toth et al., 1999).

### ORC2

Saccharomyces cerevisiae ORC2 is a component of the 6-subunit origin recognition complex (ORC) that acts at the origins of DNA replication distributed throughout the length of chromosomes (Bell et al., Nature, 1992, 357:128-134). ORC2 is required for viability, and temperature sensitive mutations in ORC2 result in cell cycle arrest consistent with defects in DNA replication (Micklem et al., Nature, 1993, 366:87-89; M. Foss et al., Science, 1993, 262:1838-1844; Bell et al., Science, 1993, 262:1844-1849). ORC has been demonstrated to bind origins of replication by DNAse footprinting, and this activity is dependent on ORC2 (Bell et al., Science, 1993, 262:1844-1849; Lee et al., Mol Cell Bio, 1993, 262:1844-1849).The gene has also been shown to be required for transcriptional silencing and telomere silencing (Micklem et al., Nature, 1993, 366:87-89; M. Foss et al., Science, 1993, 262:1838-1844; Bell et al., Science, 1993, 262:1844-1849). These appear to be separable functions for the ORC2 gene product, since the role of ORC2 in silencing can be complemented in yeast by expression of Drosophila ORC2, but its role in replication is not complemented (Ehrenhofer-Murray et al., Science, 1995, 270:1671-1674).

ORC2 assays:

(a) ATLAS: CaOrc2 protein could be purified and challenged with an environmental condition, such as higher temperature or reduced pH, that unfolds the protein. A compound that binds to CaOrc2 protein may stabilize the native conformation of the protein.

(b) Two hybrid interruption screen using another interacting protein: CaOrc2 and a Candida albicans ortholog of another member of the ORC could be placed into yeast two-hybrid screening vectors, one as the bait and one as the target. Binding by the two proteins will induce expression of a reporter gene. A compound that interferes in the binding of the two proteins should disrupt the induction of the reporter gene, allowing such compounds to be identified in a screening format. Interacting proteins other than those in the ORC could be used in this format.

(c) DNA-binding screen: Compounds could be screened for their ability to interfere with the binding of DNA to CaOrc2 protein. The binding of DNA and CaOrc2 protein could be assessed in a variety of ways: 1) through capture on a filter or capture by antibodies; 2) in homogeneous solution using fluorescently-labeled DNA and detection of a change in fluorescence polarization; or 3) detection of a gel shift when DNA is bound by the protein. These screens may be done with other proteins in the ORC present during the assay.

### CNS1

Hsp90 chaperone complexes maintain or restore activity in both heat-denatured proteins and signaling proteins prone to deactivation (Dolinski et al., Mol Cell Biol, 1998, 18:7344-7352). In present day models of Hsp90 complex interaction with signaling proteins (e.g., hormone receptors), a cycle is assumed to occur of contruction and degradation of an Hsp90-signaling protein complex into its subunits. When construction of the protein complex is complete, signaling can occur. However, if Hsp90 removal does not occur the signaling protein is degraded.

CNS1 is one of the Hsp90 chaperone complex subunits and is presumably bound via a Tetratrico Peptide Repeat (TPR) domain. CNS1 genes have been found in both mammnalian and fungal cells. However, the degree of conservation between fungi is higher than that between fungi and mammalian (approx. 55% vs. 30%). This difference combined with the importance for fungal cell viability makes CNS1 an excellent target for antifungal drug discovery

CNS1 assays:

(a) ATLAS

(b) Cell-based assays: Various strains of S. cerevisiae could be constructed in which ScCNS1 would be replaced with a functional CNS1 gene (i.e. derived from cDNA when necessary) from different organisms, in particular fungi and mammals. These cells would be grown in individual wells containing defined number and mixtures of compounds, which potentially could inhibit growth. Differences in degrees of inhibition by compounds between above-mentioned strains suggest that a compound may inhibit growth by preferentially inhibiting activity of a class of CNS1.

(c) Protein-protein interaction based assays: (i) Two-hybrid screen (Fromont-Racine et al., Nat Genet, 1997, 16:277-82) using CNS1 and any protein found to interact with CNS1 (e.g. other Hsp90 complex subunits); (ii) Direct binding assay: The interacting protein would be fixed onto a carrier an allowed to bind easily detectable CNS1. In the absence of inhibitors a high signal would result. However, interference with this interaction would reduce signal. Orientation of the assay could also be reversed by fixation of CNS1 and incubation with labeled interacting protein.

### YPD1

Saccharomyces cerevisiae YPD1 is an essential gene that functions in a two-component regulatory system in the high-osmolarity sensing MAP kinase pathway. The protein mediates a transfer of a phosphate from Sln1p to Ssk1p under normal osmolarity to inhibit the MAP kinase kinase kinases Ssk2p and Ssk22p (Posas et al., Cell, 86:865-875, 1996). Ypd1 lethality is due to constant activation of the HOG1 pathway (Posas et al., 1996). The structure of Ypd1p has been solved and consists of a four-helix bundle that makes up the central core and contains the active site residue, His64. Residues around the active site are exposed to solvent and are important for phosphotransfer activity (Xu et al., J. Mol. Biol., 292:1039-1050, 1999).

YPD1 assays:

(a) YPD1 is a good candidate for an ATLAS screen. In addition, as a secondary in vitro assay, transfer of radiolabeled phosphate from Sln1p to Ypd1 can be monitored (Li et al., 1998).

(b) Transfer of radiolabeled phosphate from Sln1p to Ypd1 can be monitored in vitro (Li et al., EMBO J., 17:6952-6962, 1998).

### TIM10

Tim10 was originally isolated as a suppressor of mrs2 mutant that is defect in mitochondria RNA splicing and respiration. (Jarosch et al., Mol Gen Genet, 1997, 255:157-65) Tim10 belongs to a group of evolutionary conserved protein called TIM family and shares extensive homology with another Tim protein, Tim9. (Bauer, et al., GEBS Lett, 1999, 464:41-47) Located in the mitochondria intermembrane space, it functions to transfer metabolic carrier proteins from cytoplasm to mitochondria. Tim10 is a soluble protein that forms a complex with Tim9 and Tim12 to bind to the precursor protein that is destined to the mitochondria and transfer them to another Tim complex, Tim 54-22-18. (Koehler et al., Science, 279:369-373, 1998; Sirrenberg et al., Nature, 391:912-915, 1998; Adam et al., Embo Journal, 18:313-319, 1999; Koehler et al., Embo J., 17:6477-6486, 1998; Endres et al., Embo J., 18:3214-3221, 1999). Tim 10 is essential for the biogenesis of mitochondria, as well as for viability of yeast cells. (Jarosch et al., Mol Gen Genet, 1997, 255:157-65) As a result of Tim10 depletion, mitochondria undergo dramatic changes in morphology and are unable to assemble cytochrome complexes. (Kubrich et al., J Biol Chem, 1998, 273:16374-16381)

TIM10 assays:

(a) ATLAS: Tim10 protein can be purified to homogeneity. Challenging purified Tim10 protein with different environment conditions such as higher temperature or reduced pH will result in the protein conformation change leading to the unfolding state. Any compound that binds to Tim10p can potentially stabilize protein in the native state. Using ATLAS can help identify compound that binds to Tim10p.

(b) Two-hybrid with Tim9. Even though, Tim10 has been shown to form a complex with Tim9 and Tim 12, only Tim10p direct interaction with Tim9p has been fully addressed. Screening compound that block Tim10 interaction with Tim9 using Two-hybrid will help identify compound that hit Tim10 protein. Tim10 and Tim9 can be used as a pair of genes in yeast with one of them as the bait and the other used as target. Binding of Tim10 and Tim9 protein in yeast will result in the induction of a reporter gene that can be detected. Any compound that interrupt binding of Tim10 protein and Tim9 protein will disrupt the induction of the reporter gene and thus that compound can be identified.

### SRB4

SRB4 is an essential component of RNA polymerase II multisubunit complex (Thompson et al., Cell, 1993, 73:1361-75). SRB is known in the art to stand for Suppressor of RNA Polymerase B. SRB4 is required for RNA polymerase II transcription at most of the promoters (Thompson et al., PNAS, 1995, 92:4587-90). It has been recently demonstrated that SRB4 is dispensable for transcriptional activation of some genes depending on activation mechanism of a particular activator (Lee et al., Gen. Dev., 1999, 13:2934-9). DNA-crosslinking immunoprecipitation assay was used to show that activator-dependent stimulation of TBP binding requires Srb4 (Li et al., Nature, 1999, 399:605-9). C. albicans Srb4 protein has an intron and it is about 30% identical to its S. cerevisiae Homolog. SRB4 has a potential human homolog which is 20% identical.

SRB4 assays:

(a) ATLAS

(b) Cell-based assays can be set up to monitor transcriptional activation of a reporter gene in wild type strain and SRB4 temperature-sensitive strain.

(c) A two-hybrid system based assay can be developed to monitor interaction between Srb4p and other SRB proteins or RNA polymerase II CTD.

(d) In vitro transcription assay (Thompson et al., Cell, 1993, 73:1361-75, Koleske et al., Nature, 1994, 368:466-469).

### Sequence Identities

The degree of sequence identity between the above S. cerevisiae (sc) genes and their C. albicans (ca) and, if available, human (hs) homologs are provided in Table 2. (See below). Multiple alignments were created using Clustal W (See Thompson et al., supra), and percentage identities caclulated using the GCG GAP program with a gap creation penalty of 12 and a gap extension penalty of 4. The sequence alignment results are also presented in the figures referred to in Table 2.

TABLE 2
Sequence Identities

| S. cerevisiae | | | | | | | | | | |
|---|---|---|---|---|---|---|---|---|---|---|
| Nominated targets | | | | | | | | | | |
| | gene | | genbank | genbank | C. albicans | Human | Sequence identities (%) | | | |
| half-life | name | orf name | DNA | protein | source | genbank # | ca v sc | sc y hs | ca y hs | FIG. |
| 0.11 | RPC34 | YNR003C | Z71618 | CAA96279.1 | stan-4-1929 | U93869 | 50.4 | 28.3 | 27.3 | 1 |
| 0.34 | POP3 | YNL282W | Z71558 | CAA96194.1 | gtc5417 | n/a | 26.1 | — | — | 2 |
| 0.35 | TFA2 | YKR062W | Z28287 | CAA82141.1 | stan-4-2738/gtc | NP_002086 | 40.8 | 23.2 | 19.4 | 3 |
| 0.36 | NAB2 | YGL122C | Z72644 | CAA96830.1 | stan-4-2144 | AAD42873 | 32.2 | 22.5 | 22.8 | 4 |
| 0.37 | MPT1 | YMR005W | Z48613 | CAA88520.1 | stan-4-2743/gtc | CAA72189 | 36.7 | 23.3 | 19.2 | 5 |
| 0.39 | MTR2 | YKL186C | Z28186 | CAA82029.1 | stan-4-3102 | n/a | 28.7 | — | — | 6 |
| 0.44 | BOS1 | YLR078C | X57792 | CAA97636.1 | stan-4-2841/gtc | NP_003560 | 37.9 | 16.8 | 18.1 | 7 |
| 0.49 | POL30 | YBR088C | Z35957 | CAA85038.1 | gtc2521 | P12004 | 54.5 | 35.7 | 41.3 | 8 |
| 0.54 | RSA2 | YMR131C | NC_001145 | CAA88556.1 | stan-4-2117 | NP_005601 | 63 | 24 | 26.1 | 9 |
| 0.68 | SQT1 | YIR012W | U75717 | AAB69630.1 | stan-4-3094 | NP_001078 | 44.5 | 22.9 | 25.1 | 10 |
| 0.81 | MTW1 | YAL034W-A | AB027473 | BAA77792.1 | stan-4-2532/gtc | n/a | 31.8 | — | — | 11 |
| 0.83 | TFB1 | YDR311W | M95750 | AAB64747.1 | stan-4-2961 | W19128 | 32.4 | 23.3 | 23 | 12 |
| 0.84 | SPC98 | YNL126W | Z71402 | CAA96007.1 | stan-4-2821 | AAC39727 | 30 | 21.5 | 19.9 | 13 |
| 0.85 | BFR2 | YDR299W | D84656 | AAB64735.1 | stan-4-3108 | NM_000055 | 42.1 | 20.7 | 22.5 | 14 |
| 1.05 | RNA1 | YMR235C | Z49939 | CAA90206.1 | stan-4-2003/gtc | CAA57714 | 51.5 | 32.1 | 33.7 | 15 |
| 1.06 | GCD7 | YLR291C | L07116 | AAB67337.1 | stan-4-2913 | AAC42002 | 52.2 | 34.5 | 35.6 | 16 |
| 1.27 | SKI6 | YGR195W | L36940 | CAA97221.1 | stan-4-3104 | BAA91279 | 62.5 | 34.8 | 39.1 | 17 |
| 1.28 | NIP1 | YMR309C | L02899 | A46417 | stan-4-2825 | AAD03462 | 42.7 | 30 | 26.7 | 18 |
| 1.32 | LCP5 | YER127W | U18916 | AAC03225.1 | stan-4-2982 | AL050003 | 34.7 | 18.6 | 18 | 19 |
| 1.63 | NCE103 | YNL036W | Z71312 | CAA95901.1 | stan-4-2981 | n/a | 34.7 | — | — | 20 |
| 1.67 | ECO1 | YFR027W | D50617 | BAA09266.1 | stan-4-2722/gtc | n/a | 34.8 | — | — | 21 |
| 1.86 | ORC2 | YBR060C | Z35929 | CAA85003,1 | stan-4-3102/gtc | Q13416 | 26.7 | 21 | 22 | 22 |
| 1.93 | CNS1 | YBR155W | Z36024 | CAA85114.1 | stan-4-3053/gtc | NP_004614 | 51.8 | 26.8 | 25.6 | 23 |
| 1.96 | YPD1 | YDL235C | Z74283 | CAA98815.1 | stan-4-2907 | n/a | 33.3 | — | — | 24 |
| 0.88* | TIM10 | YHR005C-A | Z80875 | AAB68435.1 | stan-4-3104 | NP_036588 | 68.1 | 36.6 | 36.6 | 25 |
| 1.30* | SRB4 | YER022W | L12026 | AAB64555.1 | stan-4-3098 | BAA88763 | 28.4 | 18 | 18 | 26 |

*half life determined using temperature-sensitive strain

### Production and Isolation of Target Proteins

The invention is also based on the generation of fungal target protein to be used in analysis as an antifungal target. Such generation requires the use of vectors comprising sequences encoding for S cerevisiae, C. albicans and/or human target proteins, in particular those listed in Table 1, cells comprising the vectors, and methods for producing the S cerevisiae, C albicans and/or human target protein homologs that involve culturing the cells.

A large number of vectors, including plasmid and fungal vectors, have been described for expression in a variety of eukaryotic and prokaryotic hosts. Such vectors will often include one or more replication systems for cloning or expression, one or more markers for selection in the host, e.g. antibiotic resistance, and one or more expression cassettes. The inserted target protein encoding sequences may be synthesized, isolated from natural sources, prepared as hybrids, etc. Ligation of the coding sequences to the transcriptional regulatory sequences may be achieved by known methods. Suitable host cells may be transformed/transfected/infected by any suitable method including electroporation, CaCl₂ mediated DNA uptake, fungal infection, microinjection, microprojectile, or other established methods.

A wide variety of host/expression vector combinations may be employed in expressing DNA sequences encoding the target proteins, in particular those listed in Table 1. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMa1-C2, pET, pGEX (Smith et al., Gene 67:31-40, 1988), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2 micron plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Appropriate host cells for expressing protein include bacteria, Archaebacteria, fungi, especially yeast, and plant and animal cells, especially mammalian cells. Of particular interest are E. coli , B. subtilis, S. cerevisiae , Sf9 cells, C129 cells, 293 cells, Neurospora, and CHO cells, COS cells, HeLa cells, and immortalized mammalian myeloid and lymphoid cell lines. Preferred replication systems include M13, Co1E1, SV40, baculovirus, lambda, adenovirus, and the like. A large number of transcription initiation and termination regulatory regions have been isolated and shown to be effective in the transcription and translation of heterologous proteins in the various hosts. Examples of these regions, methods of isolation, manner of manipulation, etc. are known in the art. Under the appropriate expression conditions, host cells can be used as a source of recombinantly produced target proteins. Advantageously, vectors may also include a promoter sequence operably linked to the S. cerevisiae, C. albicans, and/or human target protein encoding portion. The encoded S. cerevisiae, C. albicans, and/or human target protein may be expressed by using any suitable vectors and host cells, using methods disclosed or cited herein or otherwise known to those skilled in the relevant art. The particular choice of vector/host is not altogether critical to the invention.

For the purposes of this invention, the promoter sequence in the vector is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

Expression of S. cerevisiae, C. albicans, and/or human target protein may be controlled by any promoter/enhancer element known in the art, but these regulatory elements must be functional in the host selected for expression. Promoters which may be used to control S. cerevisiae, C. albicans, and/or human target protein gene expression include, but are not limited to, Cytomegalovirus immediate early promoter (CMV promoter; U.S. Pat. Nos. 5,385,839 and 5,168,062) the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

Nucleic acids encoding wild-type or variant S. cerevisiae, C. albicans, and/or human target proteins/polypeptides may also be introduced into cells by recombination events. For example, such a sequence can be introduced into a cell, and thereby effect homologous recombination at the site of an endogenous gene or a sequence with substantial identity to the gene. Other recombination-based methods, such as non-homologous recombinations or deletion of endogenous genes by homologous recombination, may also be used.

The invention is also based on the generation of isolated and purified S. cerevisiae, C. albicans, and/or human target proteins/polypeptides, including, e.g., a polypeptide having any of the amino acid sequences depicted in Table 1, as identified by their SEQ ID NOS, as well as function-conservative variants of these polypeptides, including fragments that retain transcriptional and/or other growth regulatory activity as described above.

S. cerevisiae, C. albicans, and/or human-derived target proteins/polypeptides according to the present invention, including function-conservative variants, may be isolated from wild-type or mutant S. cerevisiae and/or C. albicans cells, respectively, or from heterologous organisms or cells (including, but not limited to, bacteria, fungi, insect, plant, and mammalian cells) into which a S. cerevisiae, C. albicans, and/or human-derived target protein-coding sequence has been introduced and expressed. Furthermore, the polypeptides may be part of recombinant fusion proteins. Alternatively, polypeptides may be chemically synthesized by commercially available automated procedures, including, without limitation, exclusive solid phase synthesis, partial solid phase methods, fragment condensation or classical solution synthesis.

“Purification” of a S. cerevisiae, C. albicans, and/or human target protein/polypeptide refers to the isolation of the polypeptide in a form that allows its transcription and/or growth regulatory activity to be measured without interference by other components of the cell in which the polypeptide is expressed. Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against S. cerevisiae, C. albicans, and/or human target protein or against peptides derived therefrom can be used as purification reagents. Other purification methods are possible.

The polypeptides of the present invention obtained by expression of the polynucleotides of the present invention can be purified from transformed cell cultures by methods known to those of ordinary skill in the art, such as precipitation with ammonium sulphate or ethanol, extraction under acid conditions, anion or cation exchange chromatography, hydrophobic interaction chromatography, affinity chromatography, hydroxylapatite chromatography and high performance liquid chromatography (HPLC). Techniques well-known to those of ordinary skill in the art can be used to regenerate the protein if it is denatured during its isolation or purification.

The isolated polypeptides may be modified by, for example, phosphorylation, sulfation, acylation, or other protein modifications. They may also be modified with a label capable of providing a detectable signal, i.e., a reporter molecule, either directly or indirectly, including, but not limited to, radioisotopes and fluorescent compounds.

### Antibodies Directed To Target Proteins

The present invention also encompasses antibodies that bind with high affinity to the C. albicans target proteins or fragments identified as described above. As used herein, antibodies with high affinity include without limitation antibodies that bind to any C. albicans target protein identified herein in its native or denatured, i.e., folded or unfolded, conformation, particularly preferred antibodies are those which recognize either unfolded or folded target protein to be used in assays as described below. Thus, in one embodiment, the antibodies of the invention are those that are antibody preparations with high affinity for the target protein in its native conformation but not in its denatured, unfolded form, or vice versa.

Antibodies which specifically recognize a C. albicans target protein in either its native or non-native conformation, may advantageously be used in screens for potential antifungal compounds which bind or otherwise inhibit the biological activity of, the C. albicans target protein. In such a screen, antibodies specific for the C. albicans target protein in its native conformation may be used to test whether potential antifungal compounds prevent denaturation of the target protein, thus indicating a strong interaction with the target.

Following the binding of the potential antifungal compound to the C. albicans target protein, the C. albicans target protein is subjected to denaturing conditions, such as, for example, high temperature, pH, denaturing solvents, and denaturants such as, e.g., urea. Following the application of these denaturation conditions, the sample containing the C. albicans target protein and a potential antifungal compound is reacted with an antibody specific for the C. albicans target protein in either its native or non-native conformation. Binding of this antibody type indicates that the binding of the potential antifungal compounds in the screen protected the target protein from denaturation. Thus, the antibodies of the invention which are specific for either the native or the non-native target protein are useful in the screening of antifungal compounds with any C. albicans target protein.

Examples of such types of screens can be found in U.S. Pat. No. 5,585,277, issued Dec. 17, 1996, and U.S. Pat. No. 5,679,582, issued Oct. 21, 1997, each of which are incorporated herein by reference. The antibodies of the invention may be polyclonal or monoclonal, but most preferably monoclonal. The antibodies may be elicited in an animal host by immunization with a C. albicans target protein, or fragments derived therefrom which carry epitopes of the C. albicans target protein, or may be formed by in vitro immunization of immune cells. The immunogens used to elicit the antibodies may be isolated from C. albicans cells or produced in recombinant systems. The antibodies may also be produced in recombinant systems programmed with appropriate antibody-encoding DNA. Alternatively, the antibodies may be constructed by biochemical reconstitution of purified heavy and light chains. The antibodies include hybrid antibodies (i.e., containing two sets of heavy chain/light chain combinations, each of which recognizes a different antigen), chimeric antibodies (i.e., in which either the heavy chains, light chains, or both, are fusion proteins), and univalent antibodies (i.e., comprised of a heavy chain/light chain complex bound to the constant region of a second heavy chain). Also included are Fab fragments, including Fab′ and F(ab)₂ fragments of antibodies.

Methods for the production of all of the above types of antibodies and derivatives are well-known in the art and are discussed in more detail below. For example, techniques for producing and processing polyclonal antisera are disclosed in Mayer and Walker, 1987, Immunochemical Methods in Cell and Molecular Biology, Academic Press, London. Such antibodies are conveniently made using the methods and compositions disclosed in Harlow and Lane, Antibodies, A Laboratory Manual, Cold Spring Harbor Laboratory, 1988, as well as immunological and hybridoma technologies known to those of ordinary skill in the art. Where natural or synthetic peptides derived from any C. albicans target protein are used to induce an specific immune response directed against the C. albicans target protein, the peptides may be conveniently coupled to a suitable carrier such as KLH and administered in a suitable adjuvant such as Freunds. Preferably, selected peptides are coupled to a lysine core carrier substantially according to the methods of Tam (Proc. Natl. Acad. Sci. USA 1988; 85:5409).

In one embodiment, a purified recombinant C. albicans target protein is used to immunize mice, after which their spleens are removed, and splenocytes used to form cell hybrids with myeloma cells and obtain clones of antibody-secreted cells according to techniques that are standard in the art. The resulting monoclonal antibodies are screened using in vitro assays such as those described herein for binding to the C. albicans target protein or inhibiting its biological activity. The antibodies are tested for specificity of binding to the C. albicans target protein in its native conformation by screening the antibodies for target protein binding before and after subjecting the C. albicans target protein to denaturing conditions.

Antibodies specific to a target protein in an unfolded conformation are also useful in screening methods as described below.

In addition to their use in the antifungal compound screens described above, the anti-target protein antibodies of the invention, may be used to quantify a selected undenatured C. albicans target protein, using immunoassays such as, but not limited to, ELISA. The antibodies may also be used to block the native function of the chosen C. albicans target protein by inhibiting its biological activity, immunodepleting cell extracts, or interfering with other reactions related to the function of the target protein. In addition, these antibodies can be used to identify, isolate, and purify C. albicans target proteins from different sources, and to perform subcellular and histochemical localization studies as well as diagnostic analyses to determine the presence of an antigenic C. albicans target protein protein in a tissue, blood or serum sample.

### Methods for Determining the Essential Nature of a Putative Essential Gene

Various methods can be used to determine whether the product of a gene is essential to the survival of a mycete or essential to the establishment or maintenance of an infection. The identification of the essential character of a gene provides additional information regarding its function and allows selection of genes for which the product constitutes a target of interest for an antifungal substance. Examples of these methods are summarized briefly below. These methods are described in the following works, each of which are hereby incorporated by reference herein: Guthrie C. and Fink G. R. (eds.), Methods in Enzymology, Vol. 194, 1991, ‘Guide to Yeast Genetics and Molecular Biology’, Academic Press Inc.; Rose A. H., A. E. Wheals and J. S. Harrison (eds.), The Yeasts, Vol. 6, 1995, ‘Yeast Genetics’, Academic Press Inc.; Ausubel F. et al. (eds.), Short Protocols in Molecular Biology, 1995, Wiley; and Brown A. J. P. and Tuite M. F. (eds.), Methods in Microbiology, Vol. 26, 1998, ‘Yeast Gene Analysis’ Academic Press Inc.

Depending on the circumstances, one of the methods described will be used, depending on the desired result. In particular, it is possible to proceed by a method of either direct inactivation of the gene or transitory inactivation of the gene. Below, we exemplify assays useful for determining the essentiality of S. cerevisiae and C. albicans genes.

### S. cerevisiae Inactivation Analysis

In the yeast S. cerevisiae , the method used most generally comprises inactivation of the gene of interest at its site within the chromosome of the yeast. The wild type allele is inactivated by insertion of a genetic marker (for example a gene for auxotrophy or a resistance marker). This insertion is in general obtained by the method of gene conversion with the aid of linear deletion cassettes prepared by known methods, as described in Guthrie C. and Fink G. R. (eds.), Methods in Enzymology, or in Gultner et al. Nucleic Acid Research, 1996, 24: 2519-2524.

Preferred methods, yeast cells and vectors for determining if an S. cerevisiae gene and/or protein is essential for growth and viability are described in U.S. Provisional Patent Application 60/056,719, filed Aug. 22, 1997, U.S. patent application Ser. No. 09/138,024, filed Aug. 21, 1998, now allowed and awaiting issue, and U.S. patent application Ser. No. 09/573,322, filed May 18, 2000, each of which are incorporated herein by reference.

Briefly, an S. cerevisiae strain in which expression of a particular gene can be tightly regulated is generated. To do this the wild-type allele of the gene of interest is replaced with an allele that can be regulated by exogenous metal. The replacement is generally carried out utilizing a double-crossover strategy with a linear piece of DNA prepared by known methods as described in U.S. Patent and Application Nos. cited above.

The recombinant cells comprise, for example:

(i) a first gene encoding a transcriptional repressor protein, the expression of which has been placed under the control of a metal ion-responsive element, wherein expression of the repressor protein is stimulated by the addition of a metal ion to the growth medium of the cells;

(ii) a second gene encoding a selected target protein, wherein expression of the target protein is controlled by a promoter, the activity of which is inhibited by the repressor protein; and

(iii) a third gene encoding a biomineralization protein, wherein the third gene is inactivated and wherein inactivation of the third gene enhances the transcriptional response of the metal-responsive element to added metal ions.

In a preferred embodiment, the first gene is ROX1; the second gene is a gene encoding for a target protein described herein, controlled by an ANB1 promoter; and the third gene is SLF1.

In a particularly preferred embodiment, the recombinant cells comprise an additional gene such that the cells comprise:

(i) a first gene encoding a transcriptional repressor protein, the expression of which has been placed under the control of a metal ion-responsive element, wherein expression of the repressor protein is stimulated by the addition of a metal ion to the growth medium of the cells;

(ii) a second gene encoding a target protein, wherein expression of the target protein is controlled by a promoter, the activity of which is inhibited by the repressor protein;

(iii) a third gene encoding a protein that targets ubiquitin-containing polypeptides for degradation, the expression of which has been placed under the control of a metal ion-responsive element, wherein expression of the ubiquitin targeting protein is stimulated by the addition of a metal ion to the growth medium of the cells, wherein the stability of the target protein is controlled by the ubiquitin targeting protein; and

(iv) a fourth gene encoding a biomineralization protein, wherein the fourth gene is inactivated and wherein inactivation of the fourth gene enhances the transcriptional response of the metal-responsive element to added metal ions.

Thus, in a particularly preferred embodiment, the first gene is ROX1; the second gene, encoding for a target protein according to the invention, is controlled by an ANB1 promoter; the third gene is UBR1; and the fourth gene is SLF1.

Utilizing this preferred system, expression of the target protein gene is carried out in the absence of added metal ion. When it is desired to decrease or eliminate expression of the target protein gene, metal ions are added to the medium, which stimulate expression of the repressor and ubiquitin tarteting protein to a degree that is dependent upon the concentration of added metal ions and represses transcription of the target protein gene and reduces the stability of the protein. In the preferred system, expression of Rox1 and Ubr1 protein is induced by the addition of copper to the growth media, and thus, expression of the target protein is shut off. If the engineered S. cerevisiae strain containing the target protein gene under control of this repressible system stops growing and loses viability in the presence of copper, the target protein is shown to be essential and a cidal target.

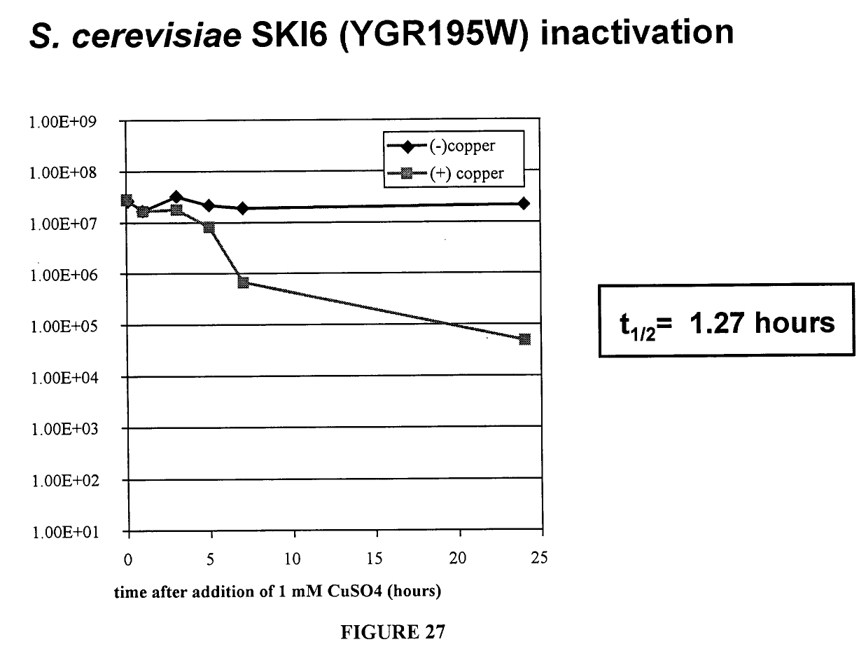

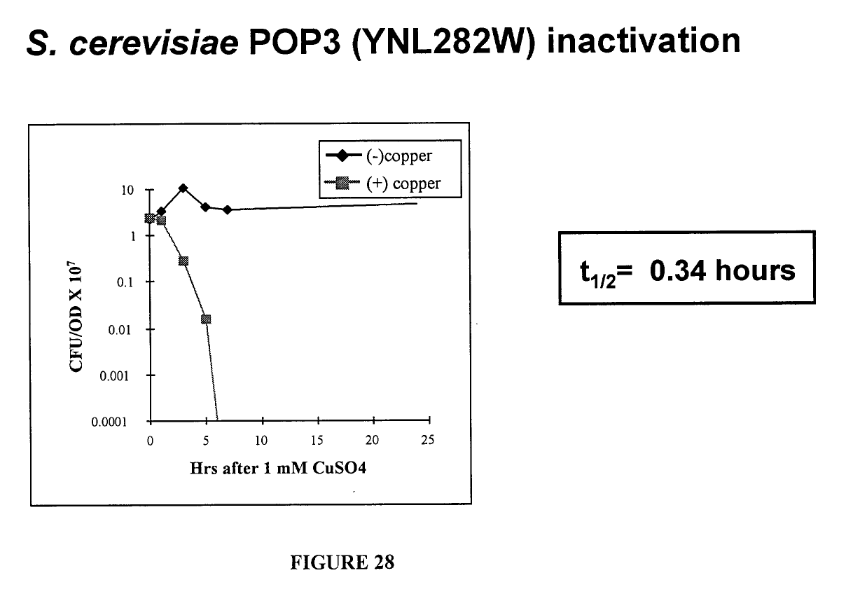

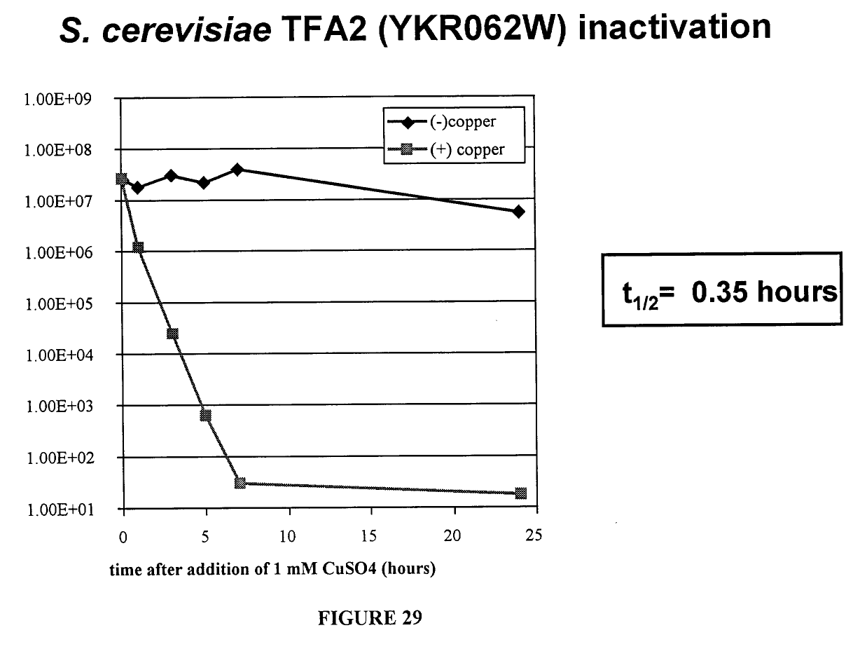

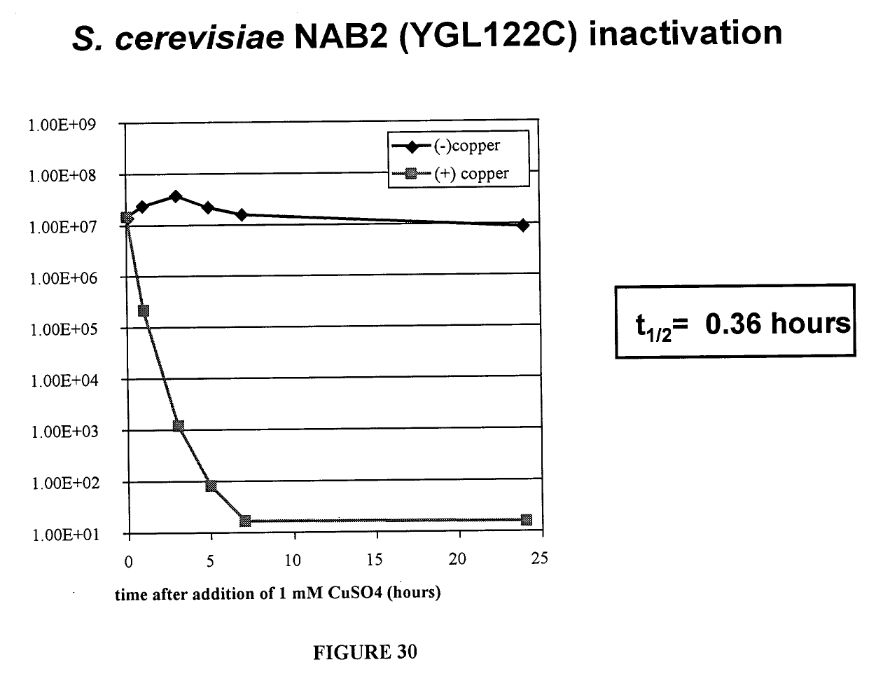

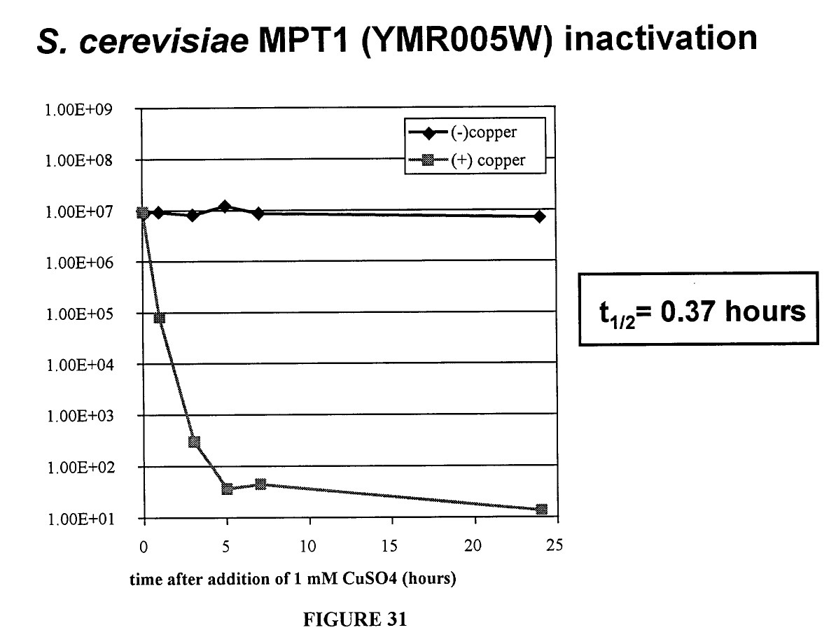

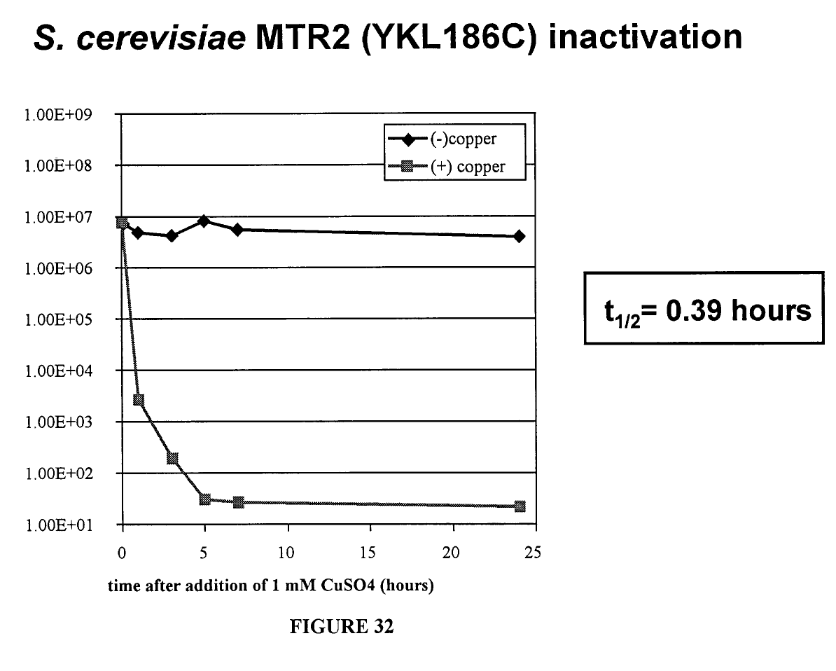

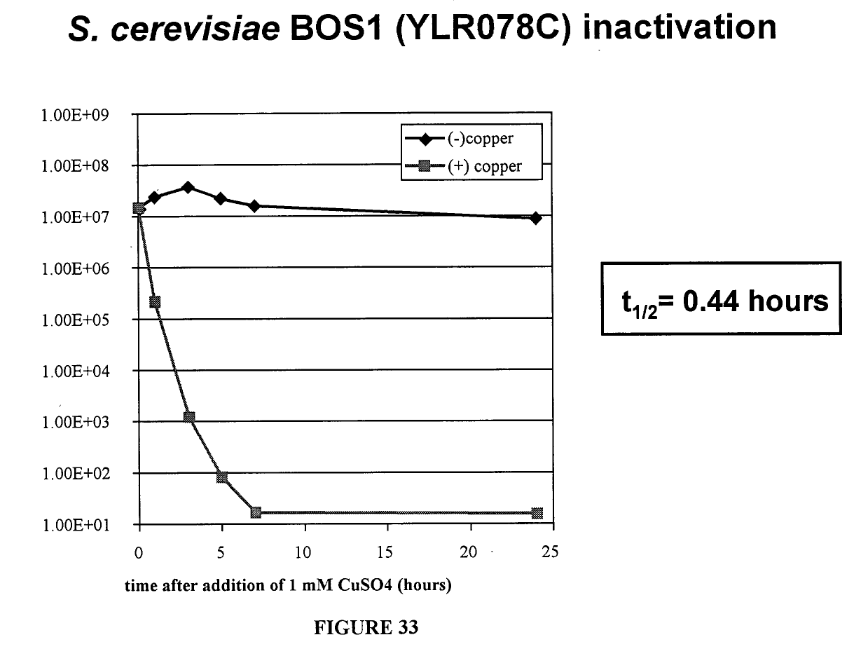

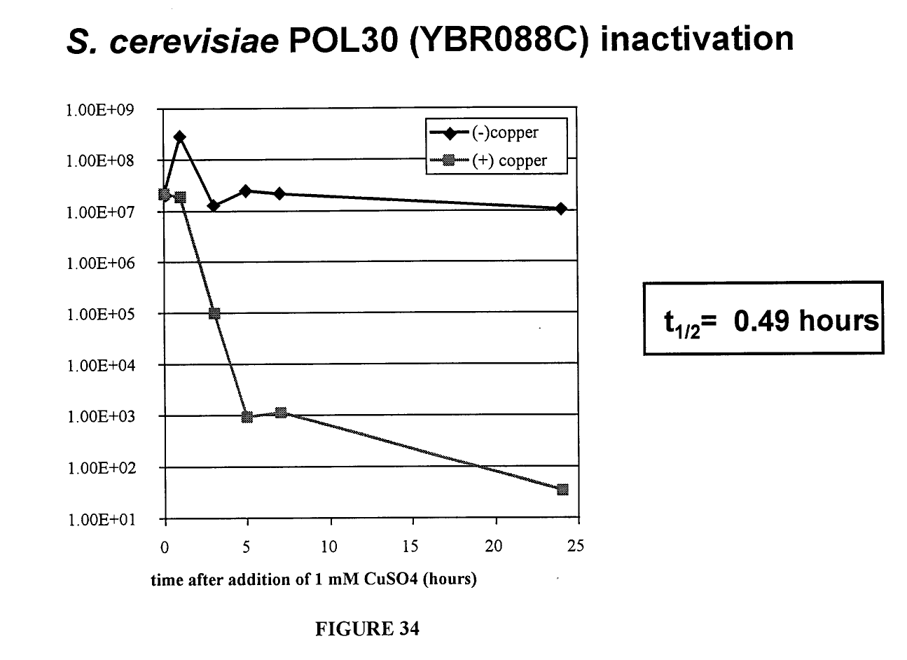

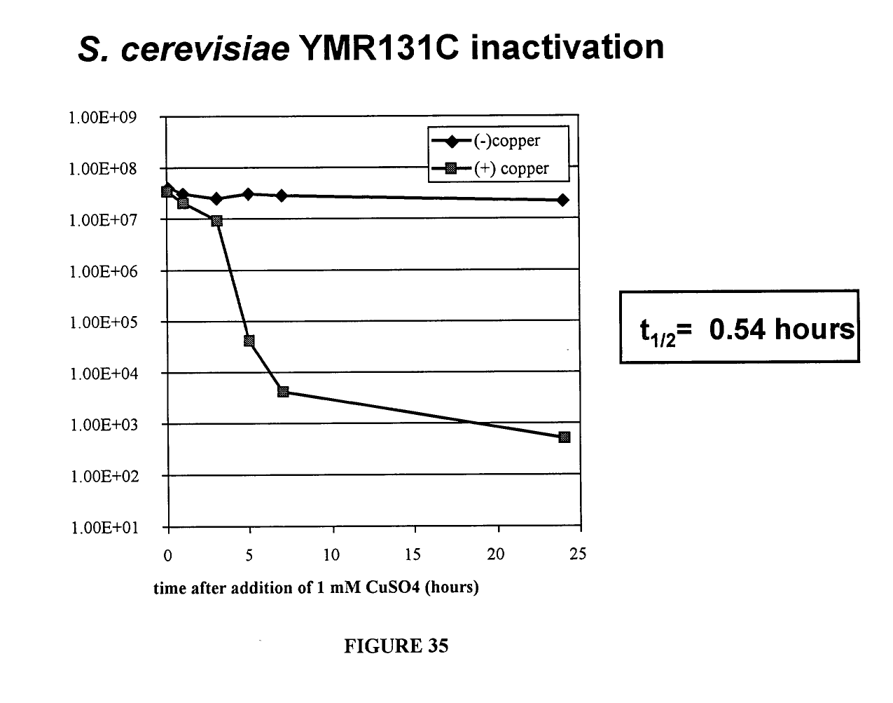

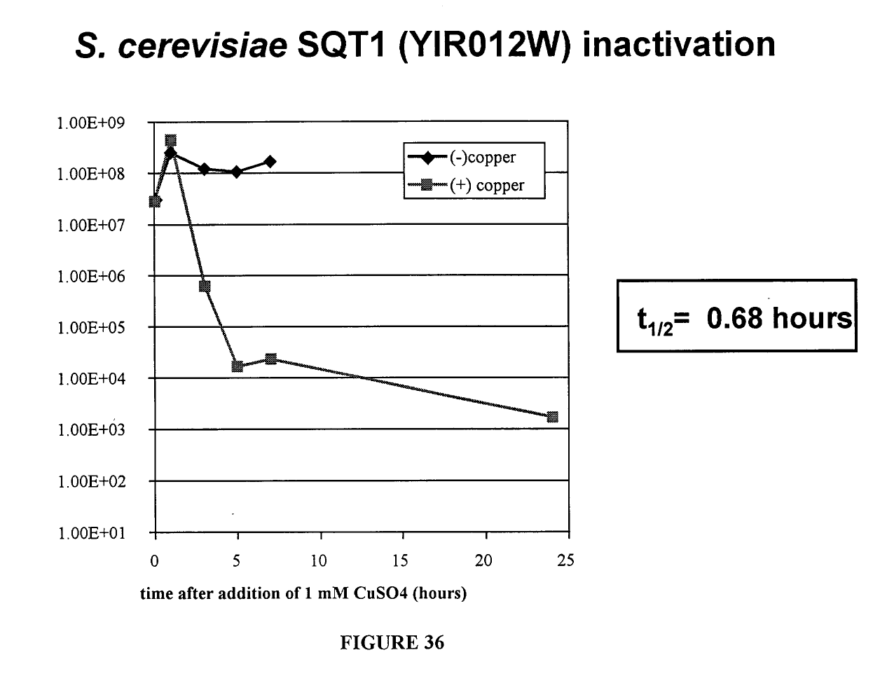

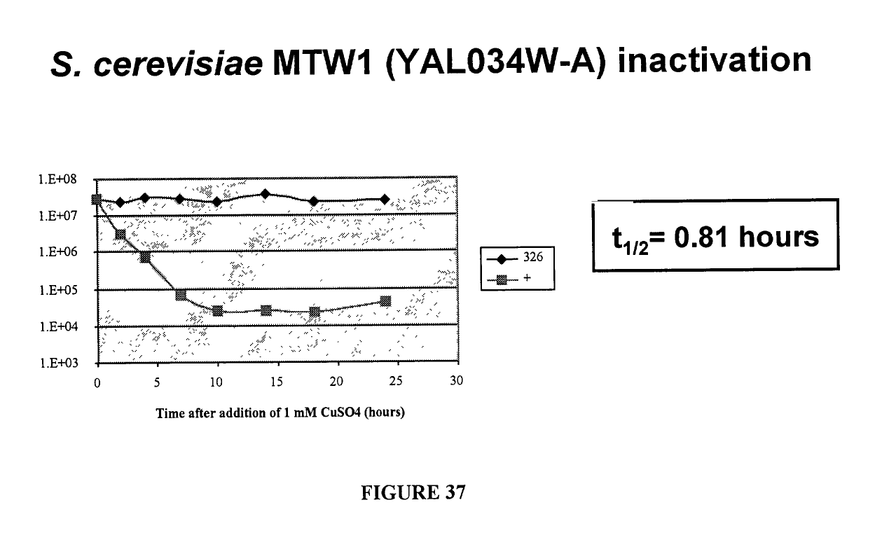

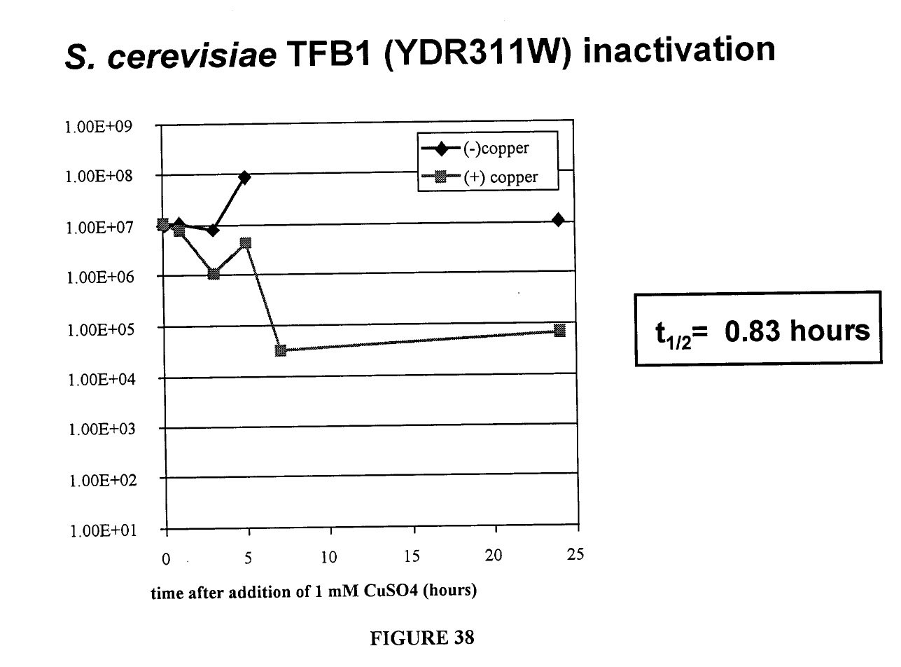

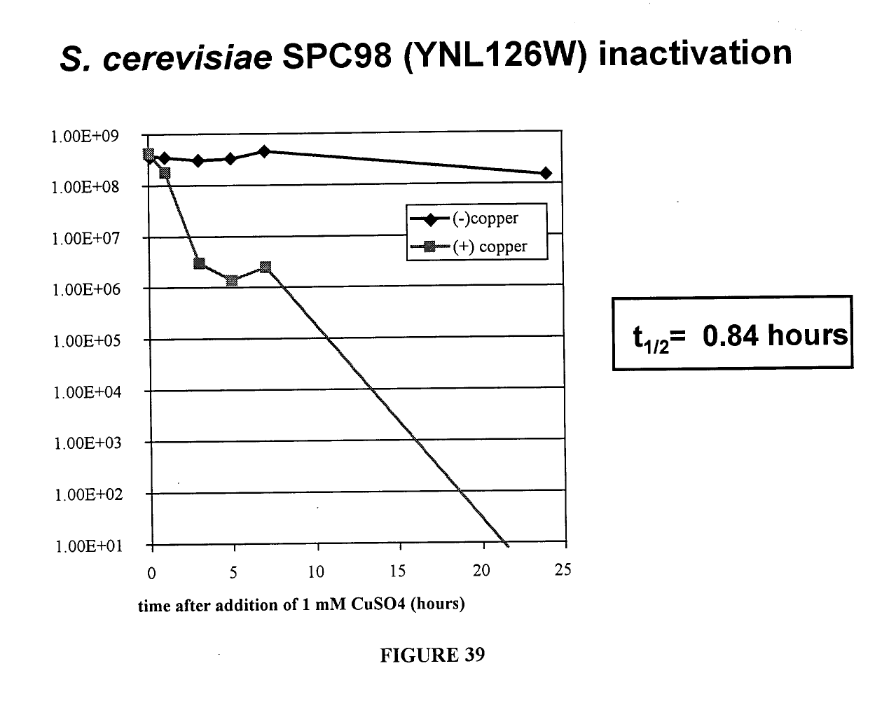

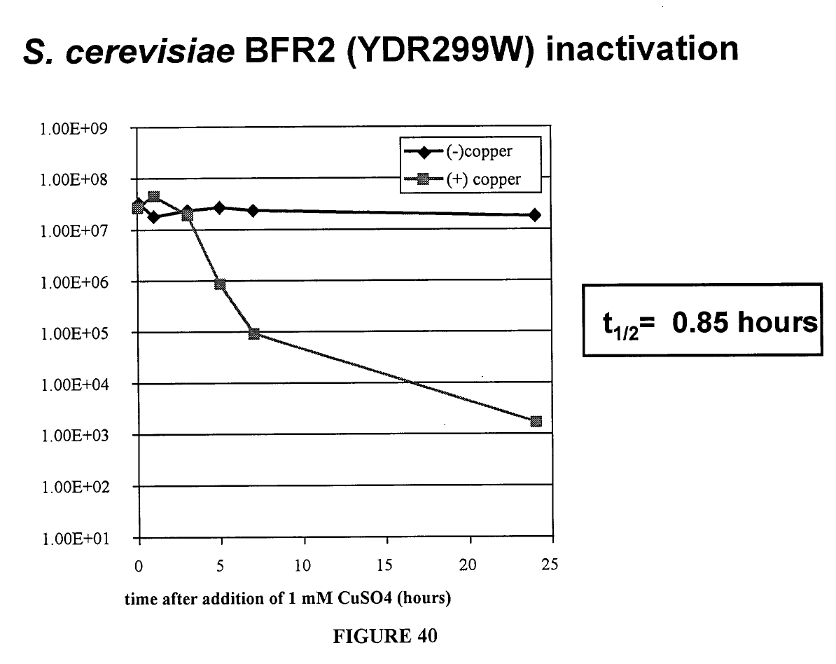

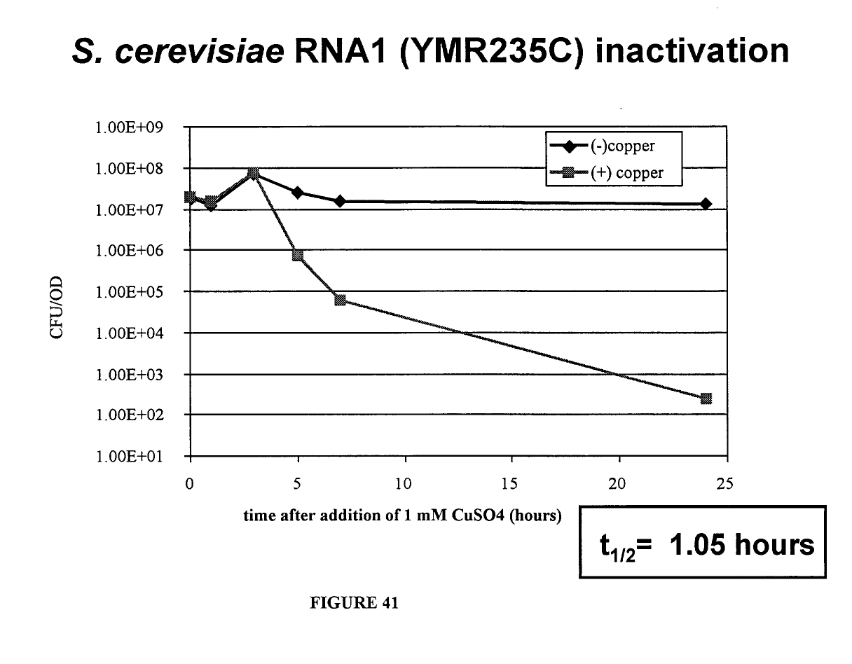

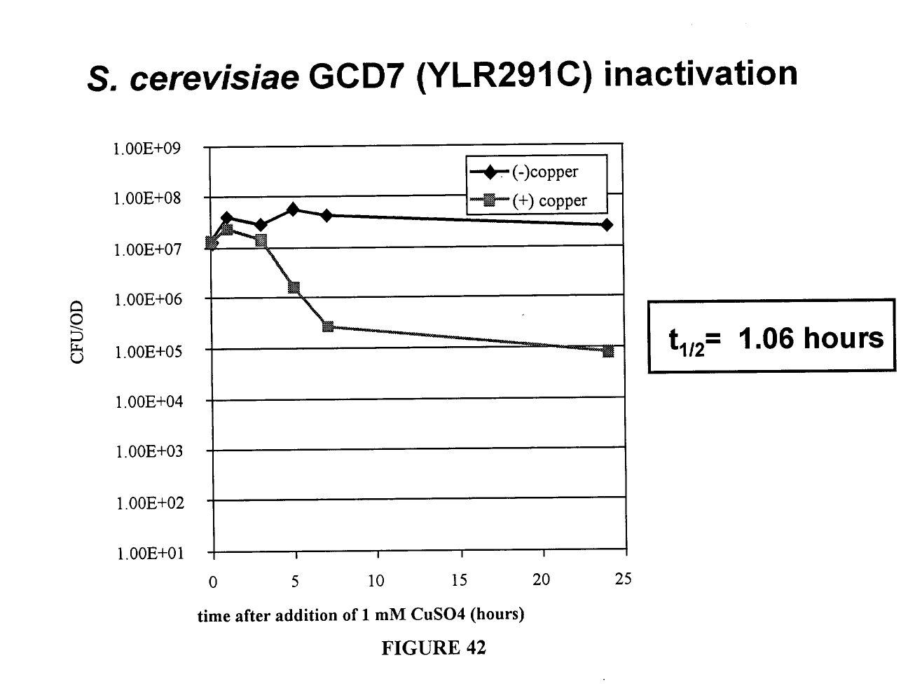

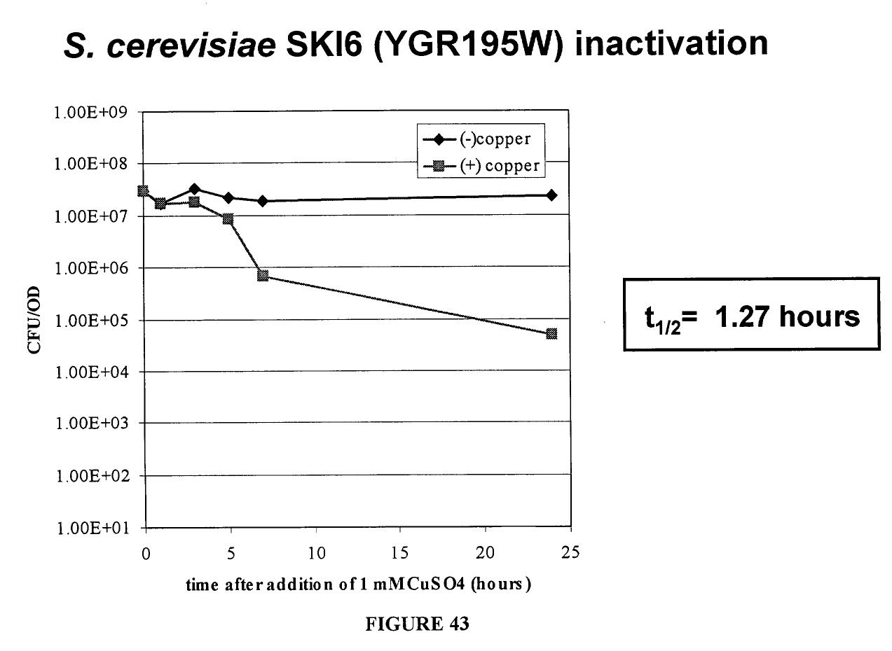

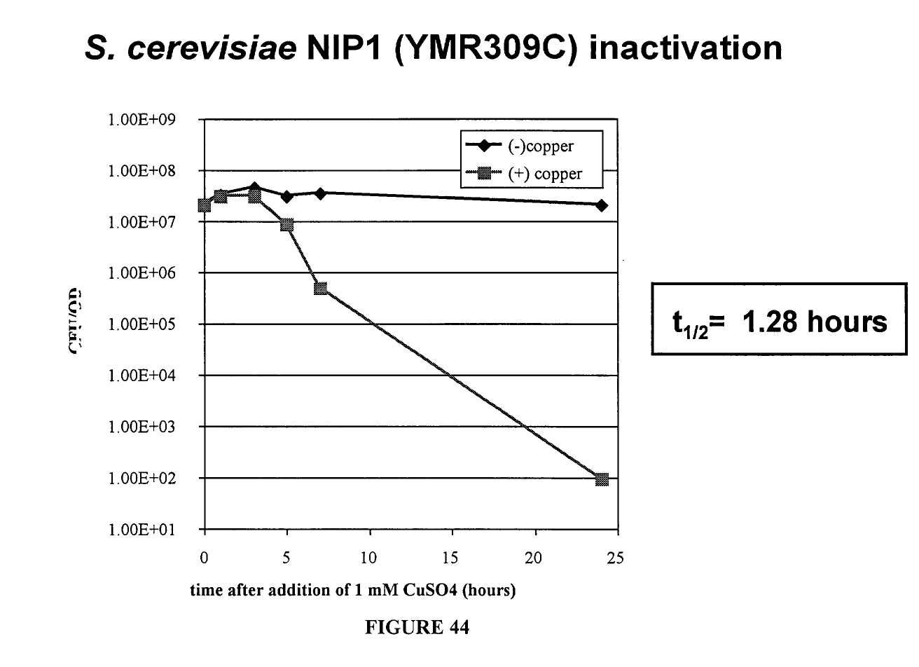

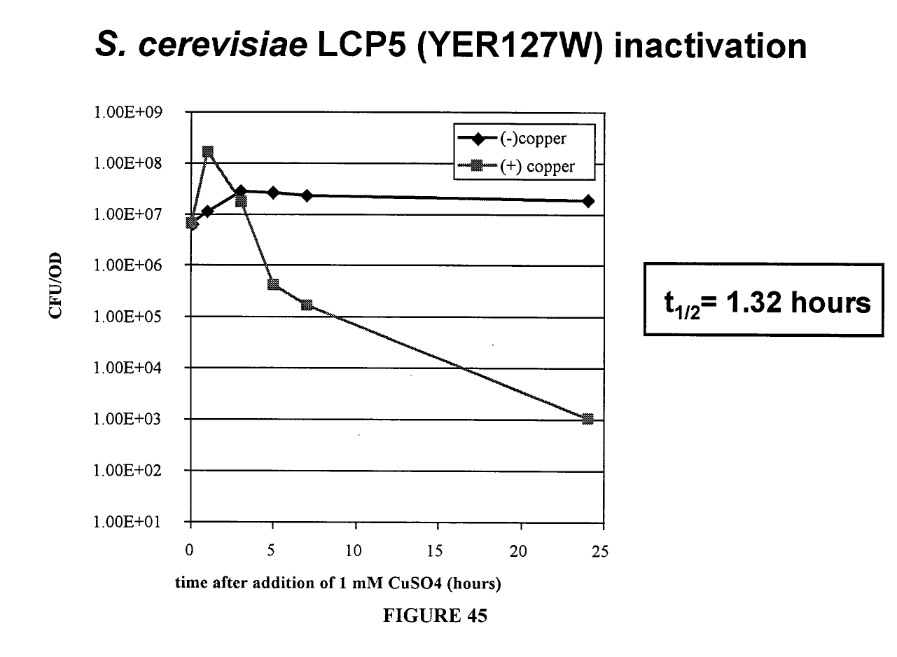

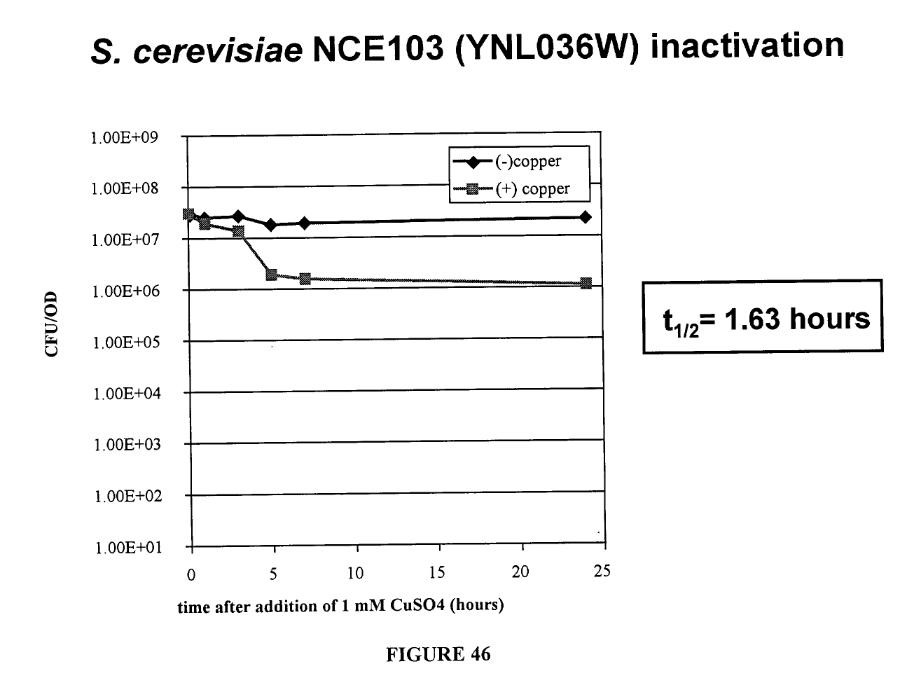

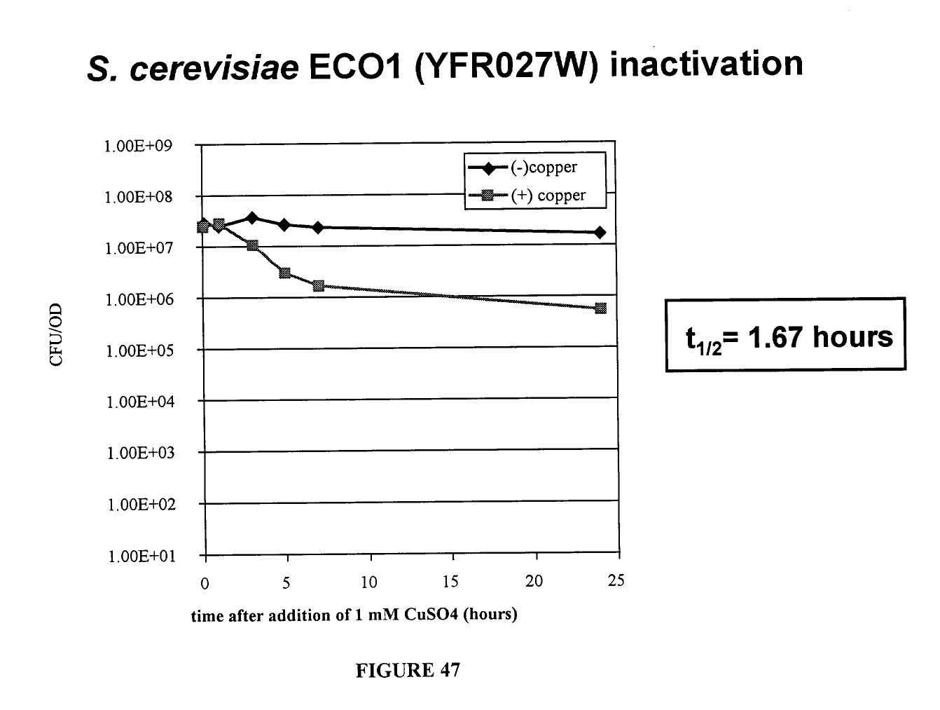

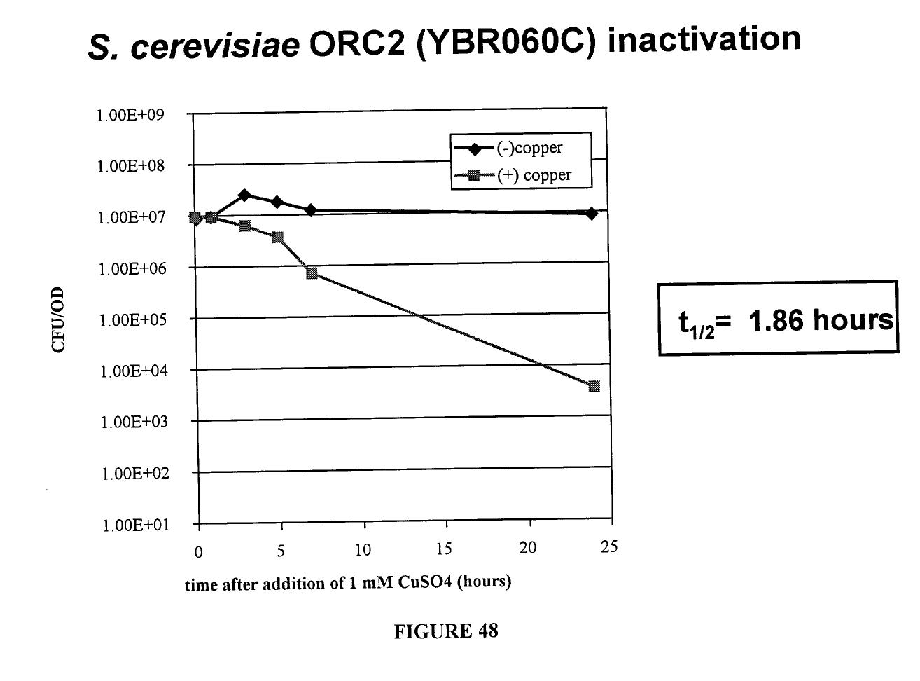

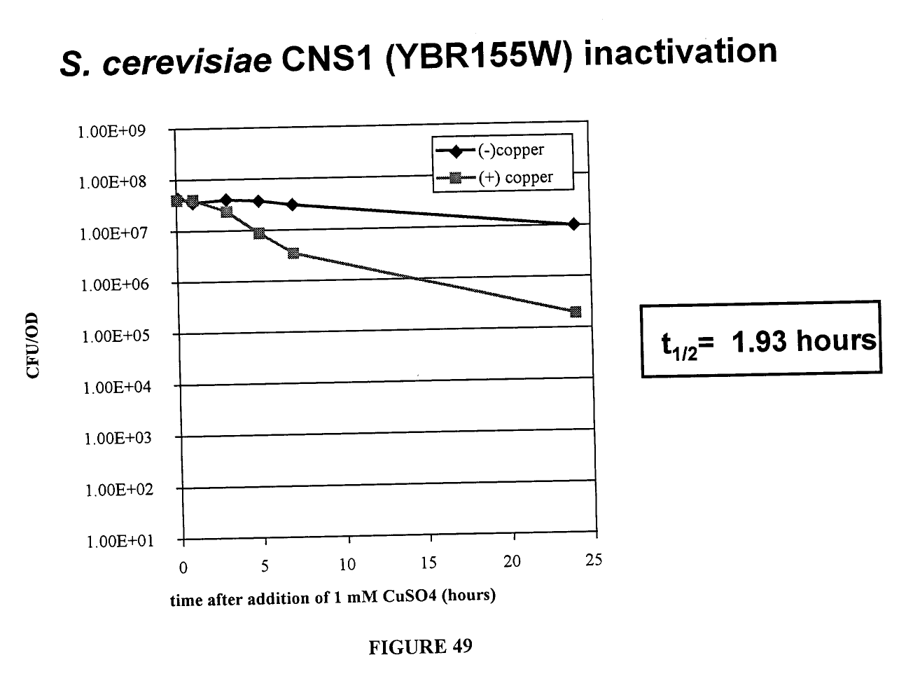

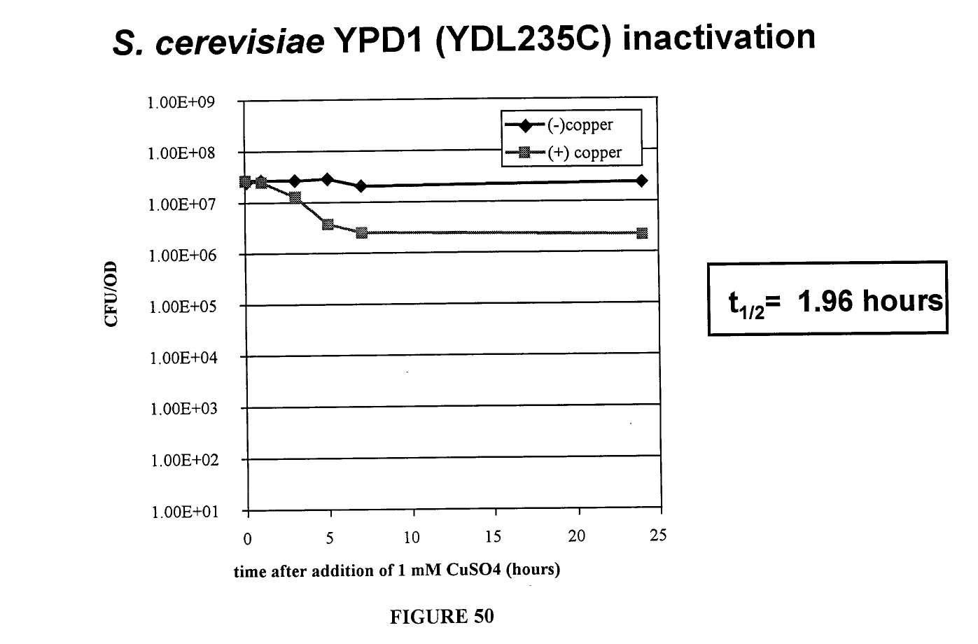

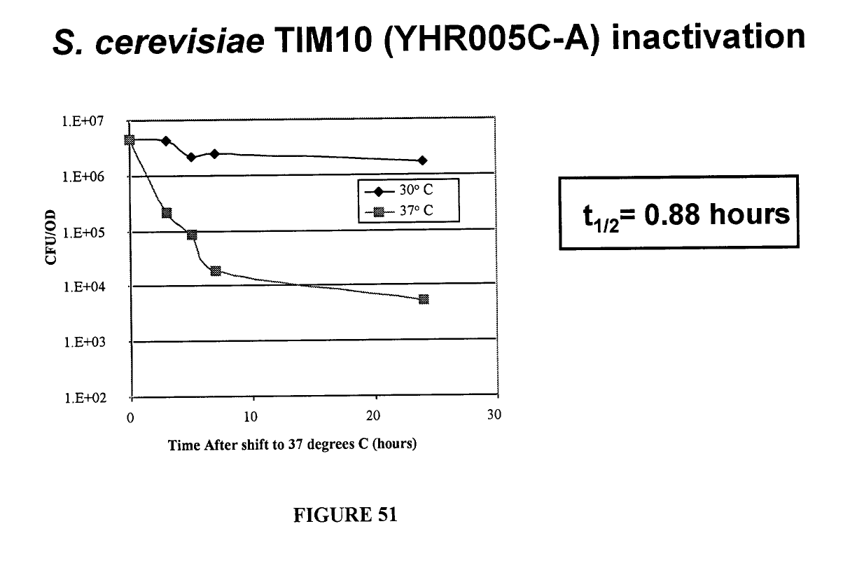

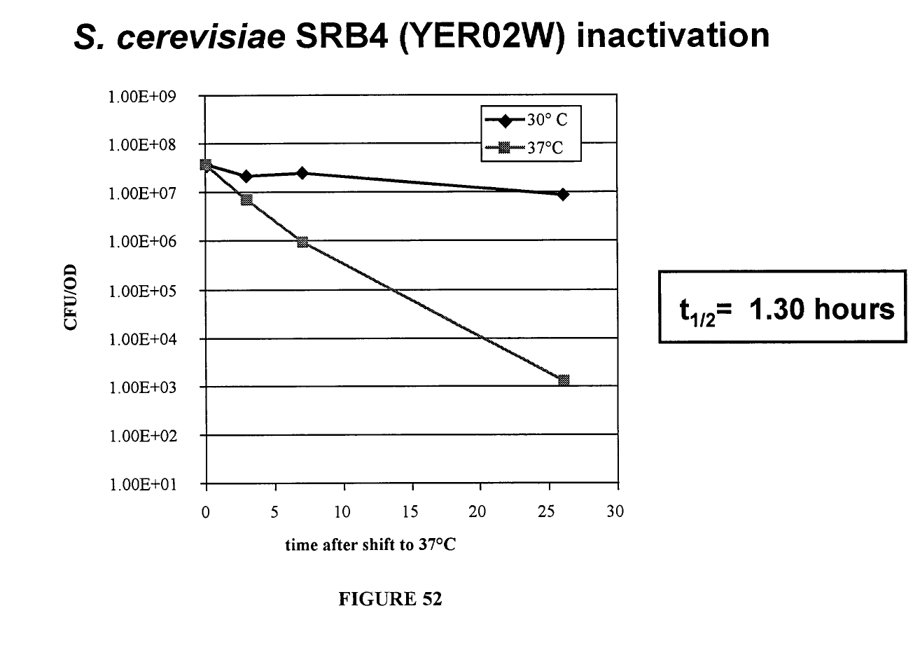

S. cerevisiae inactivation analyses of the target proteins described in Table 1 were conducted as described herein and in Example 1, and the results are presented in FIGS. 27-52.

Once the S. cerevisiae target protein has been shown to be both essential for growth and viability, and a cidal target in S. cerevisiae , the homologous C. albicans gene and or protein must then be analyzed to determine if either are essential for growth and can act as a potential cidal target in C. albicans. The C. albicans gene is identified by comparative sequence analysis. When a DNA fragment is required for some type of analysis (gene inactivation or protein expression) it is preferably obtained by PCR cloning using methods well known in the art (See for example, Eds. C. W. Dieffenbach and E. F. Dvekfler, PCR Primer:A Laboratory Manual Cold Spring Harbor Laboratory Press, Plainview, N.Y., 1995.)

### C. albicans Deletion Analysis

Determining if a particular gene or protein is essential for growth is carried out by determining if, when the gene or protein is inactivated in C. albicans, the cells will survive. Because C. albicans is a diploid fungus which, largely due to the absence of a sexual phase in its life cycle, is resistant to a considerable number of genetic techniques that are applicable to S. cerevisiae, DNA constructs are used to inactivate, or delete all, or a portion, of the gene of interest in C. albicans. Such constructs provide for the inactivation or deletion of the wild type allele by insertion of a genetic selection marker (for example a gene for auxotrophy or a resistance marker). This insertion is in general obtained by the method of gene conversion with the aid of linear deletion cassettes prepared by known methods of DNA manipulation as described above.

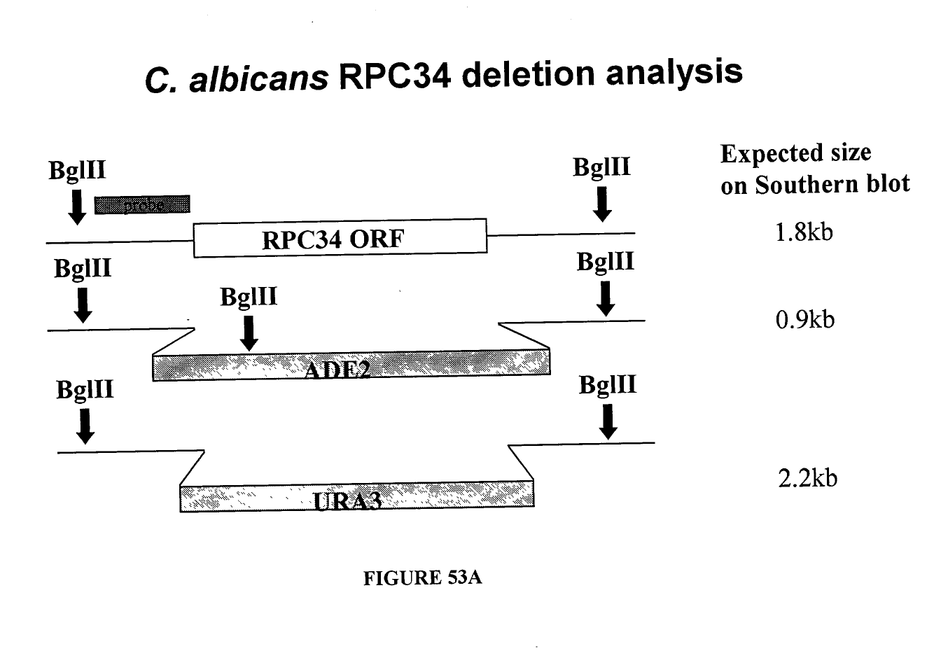

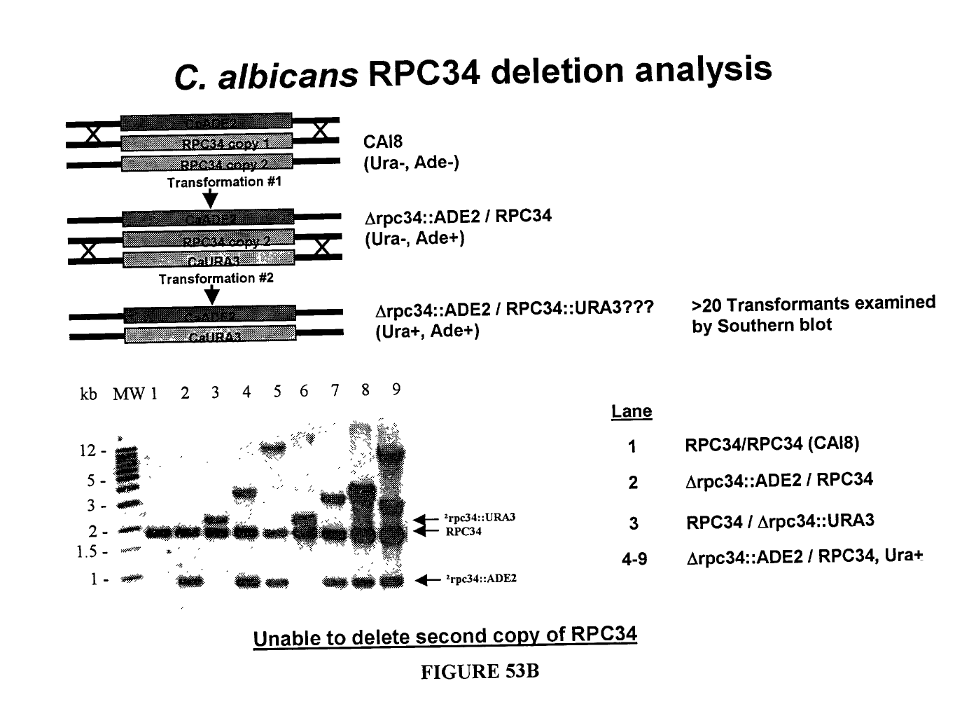

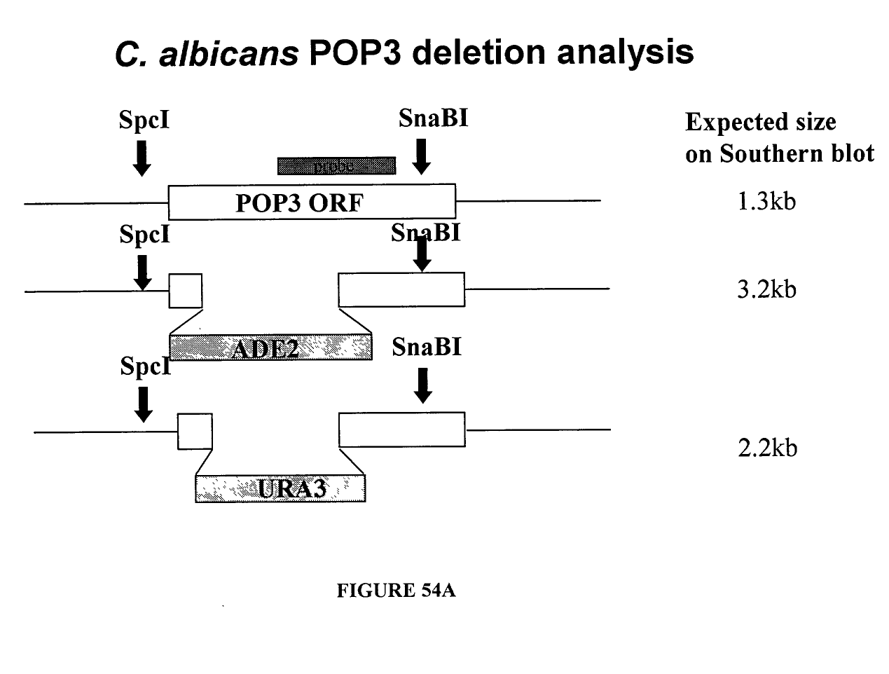

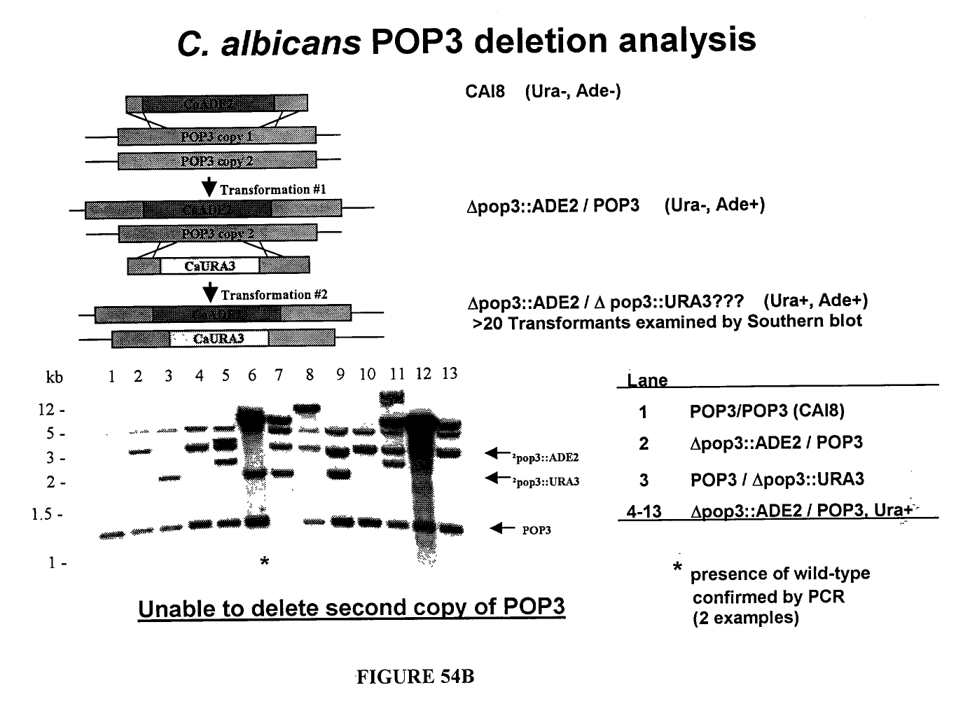

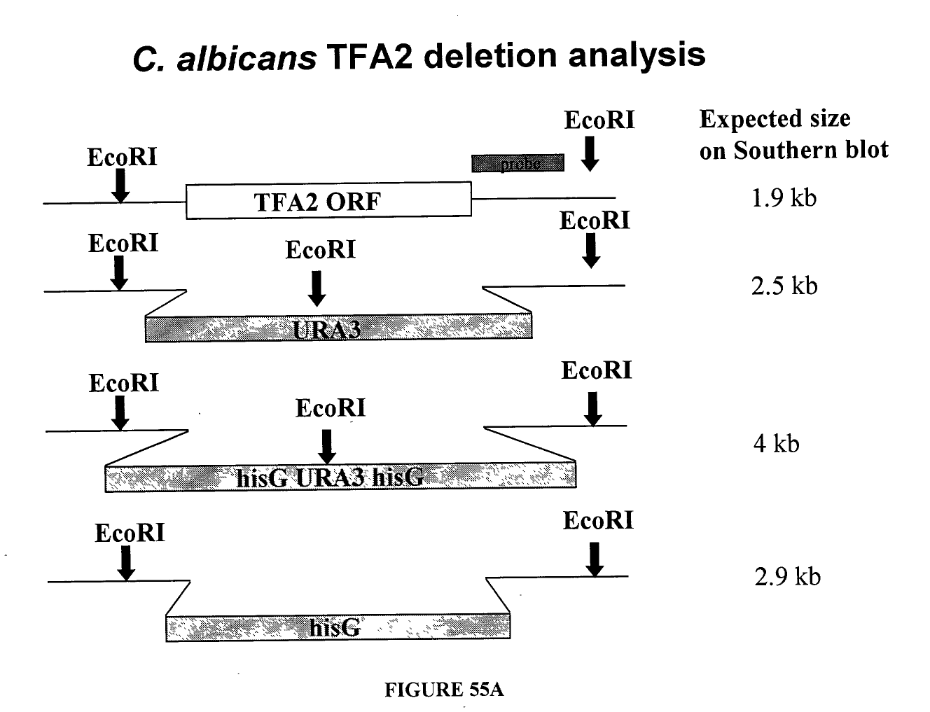

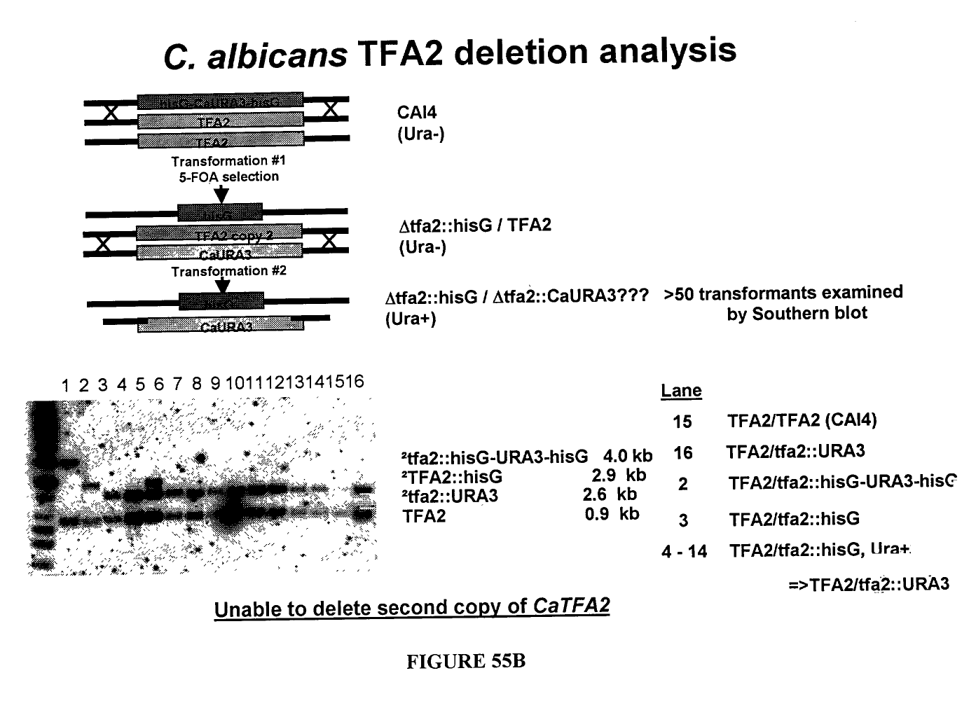

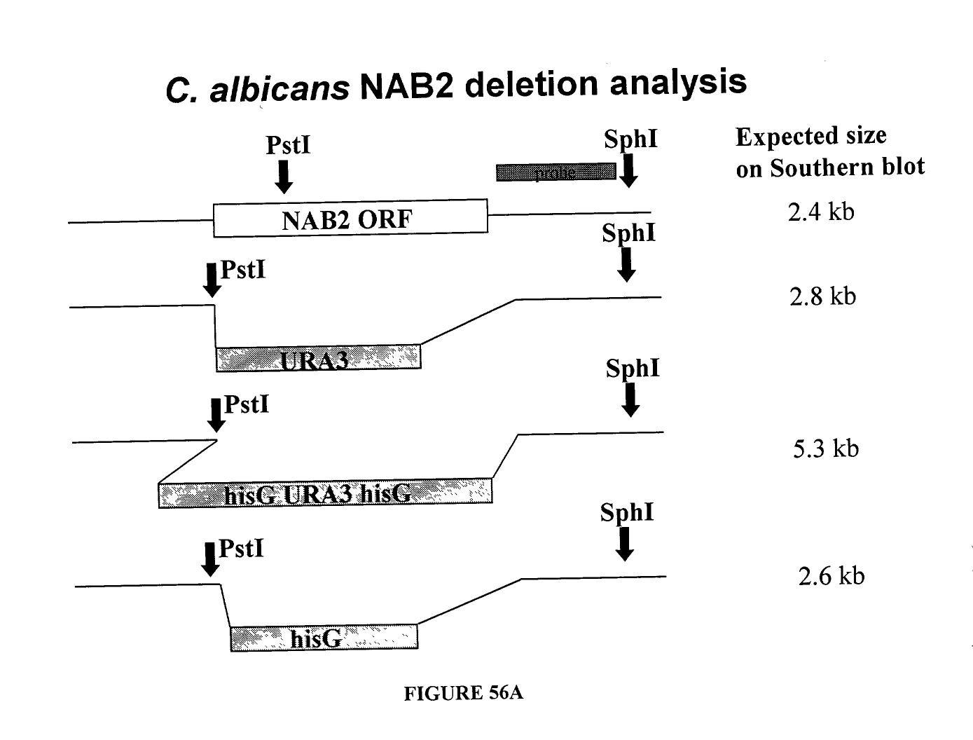

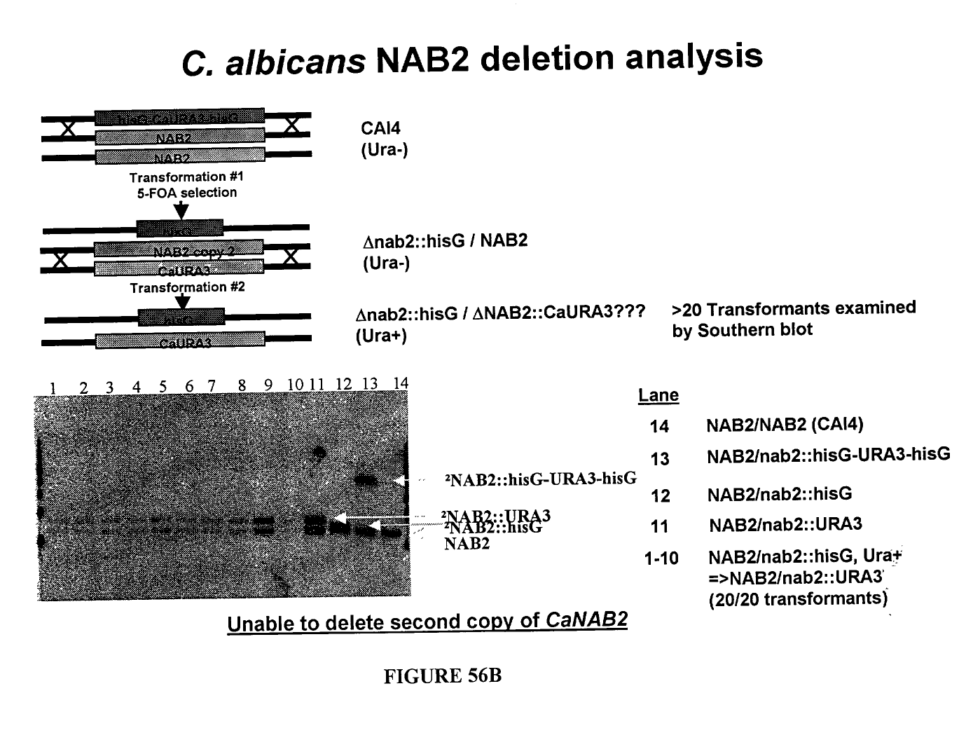

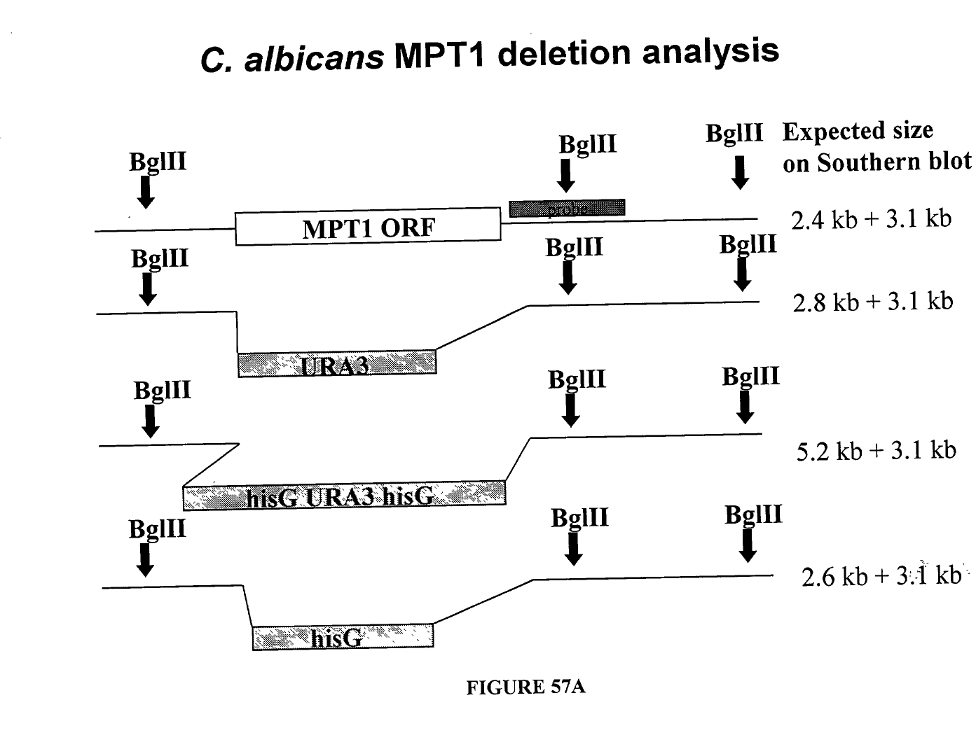

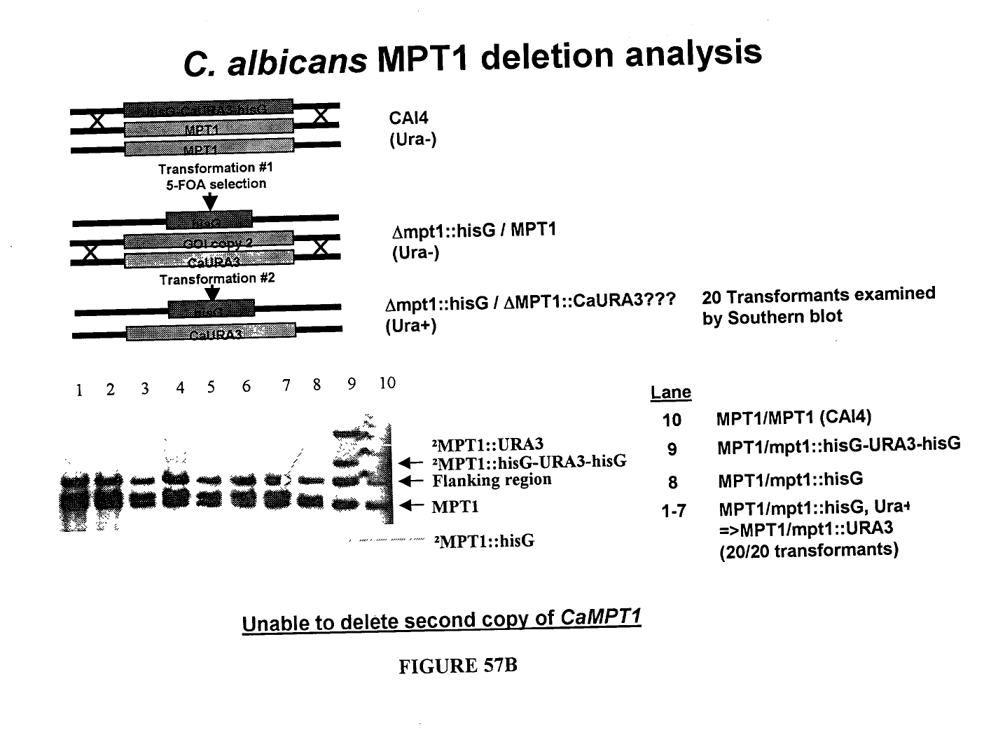

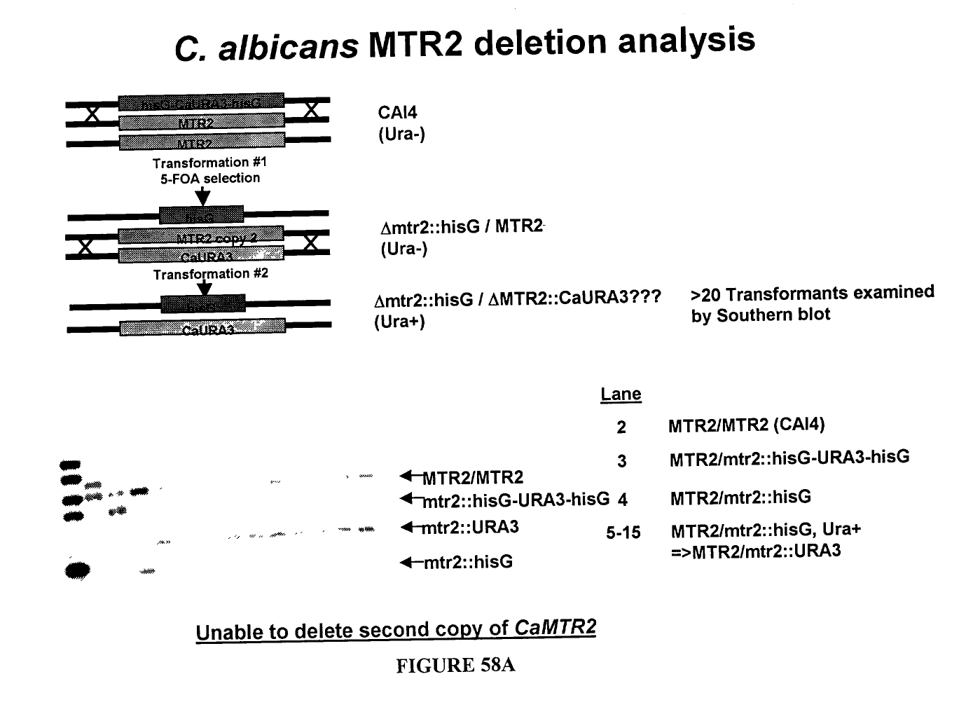

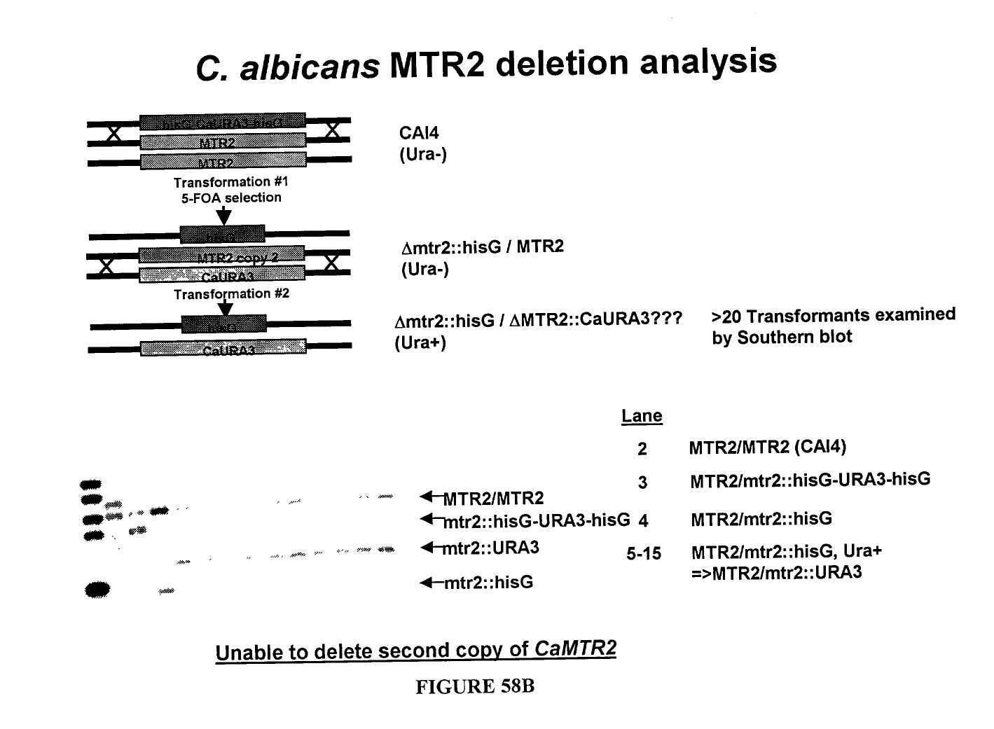

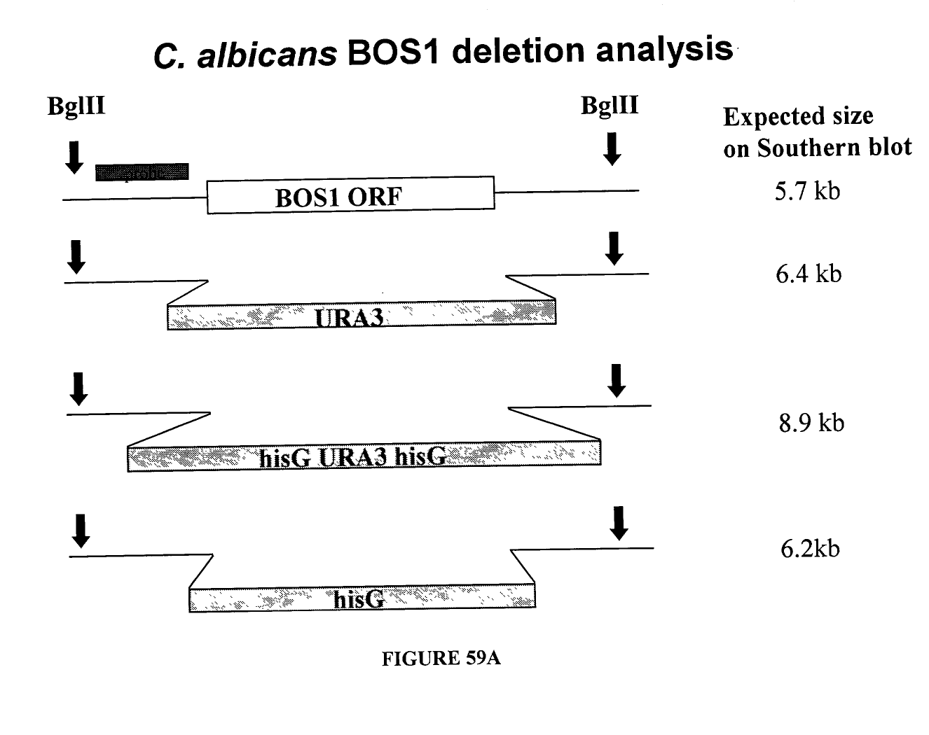

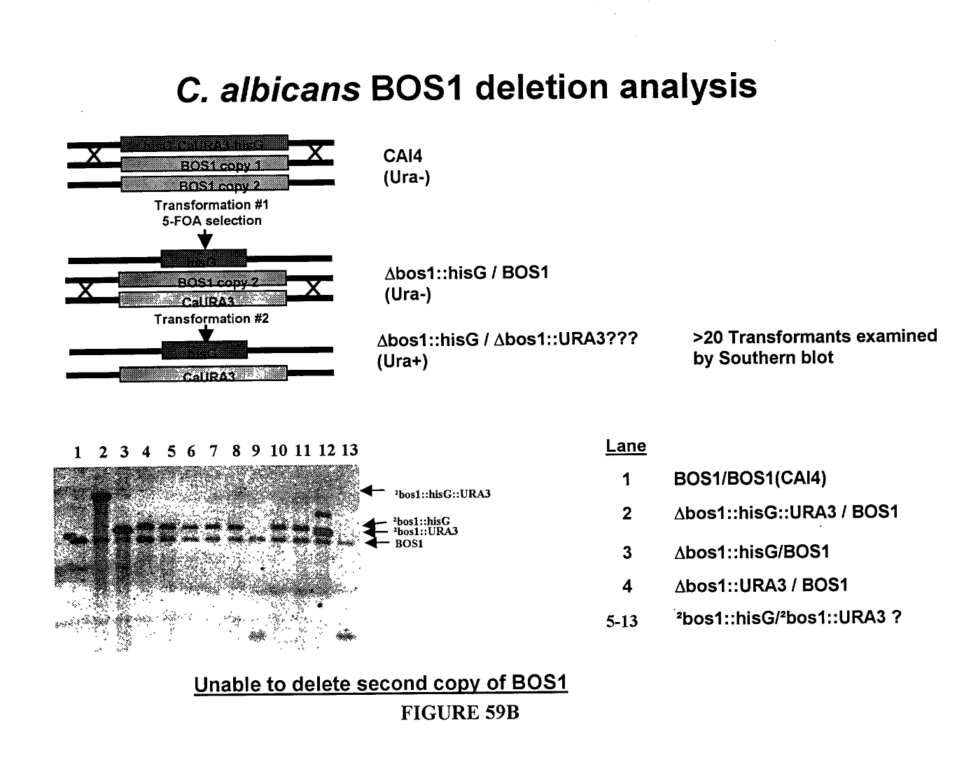

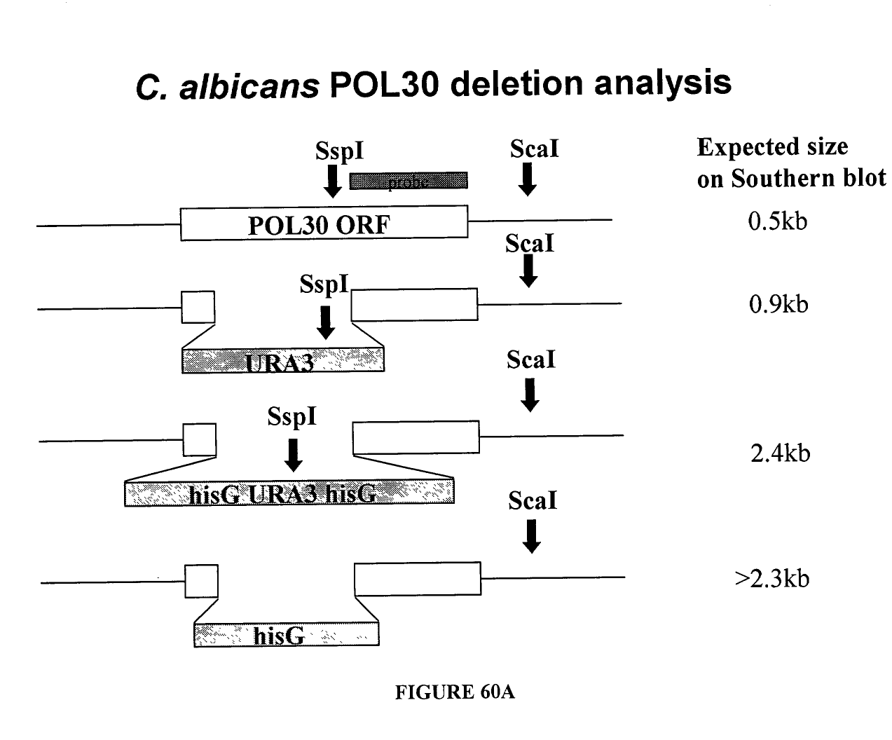

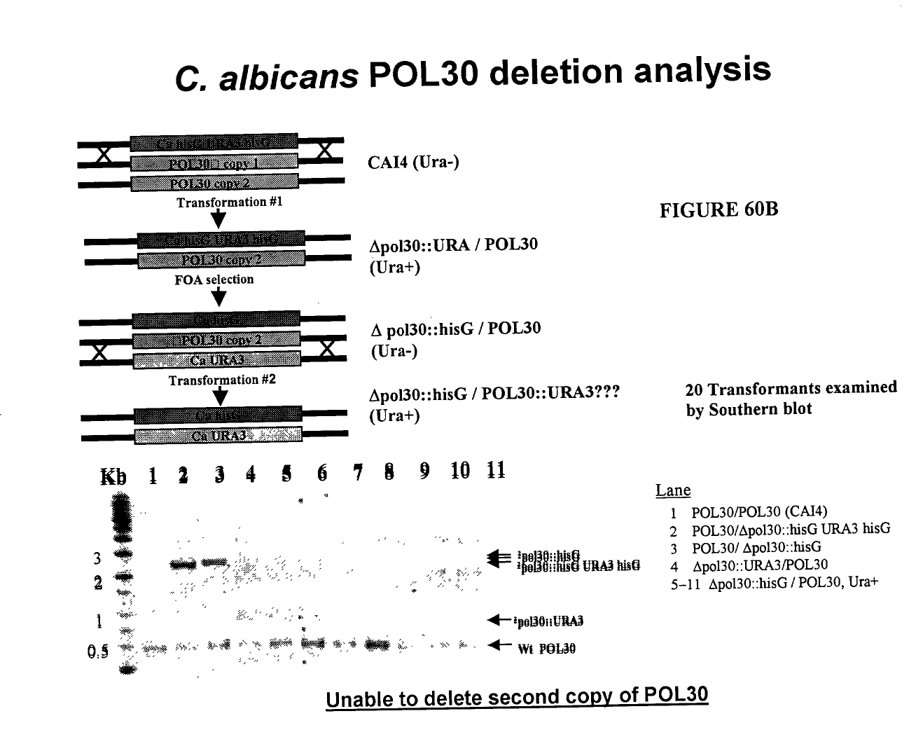

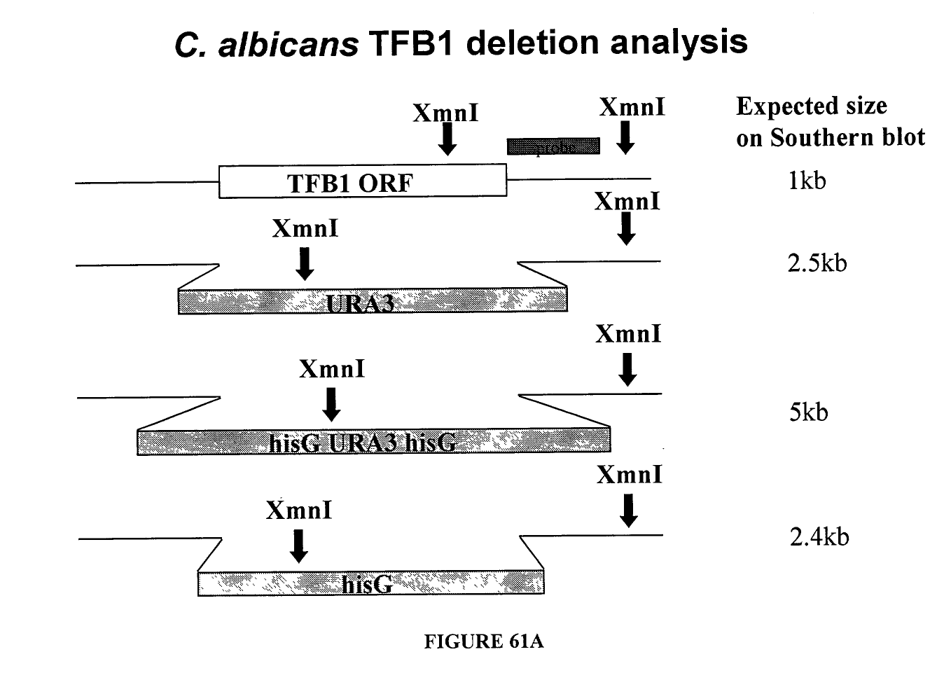

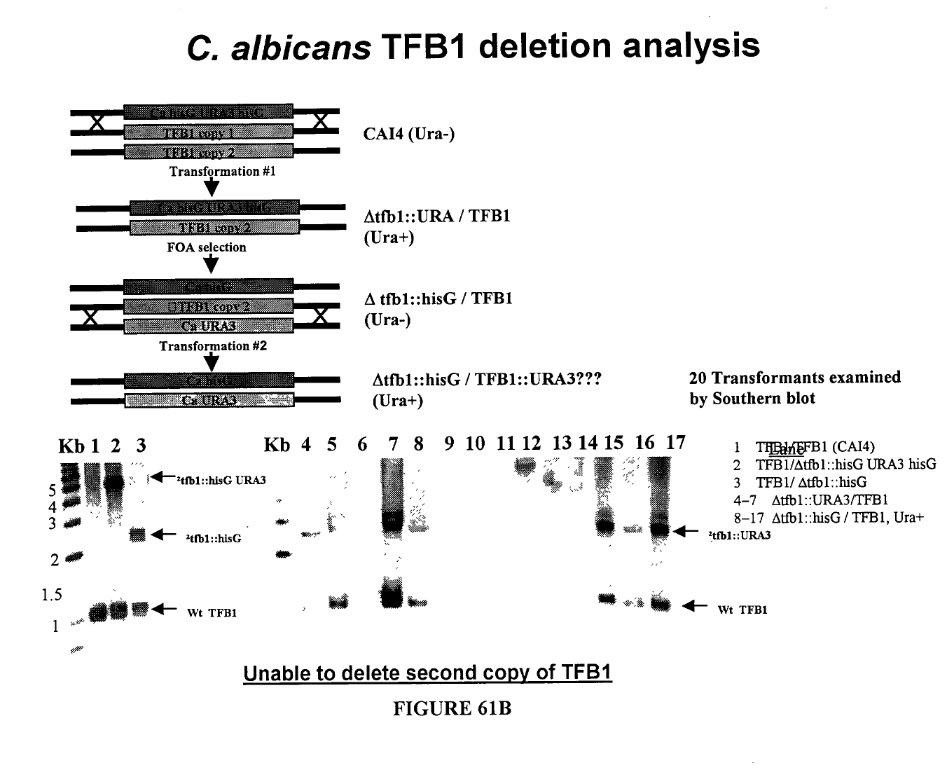

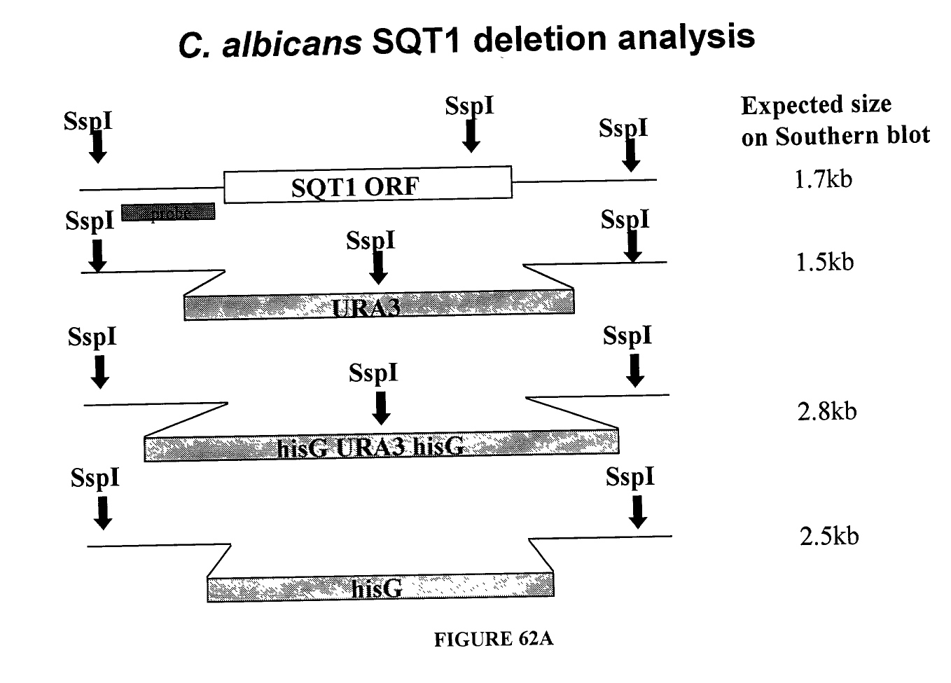

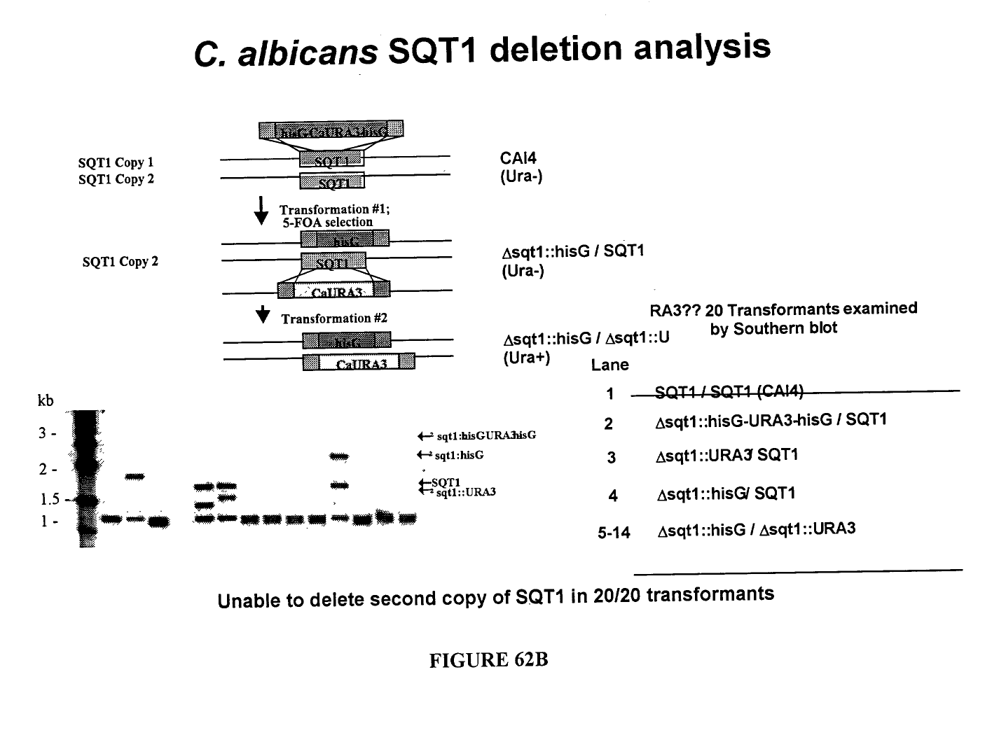

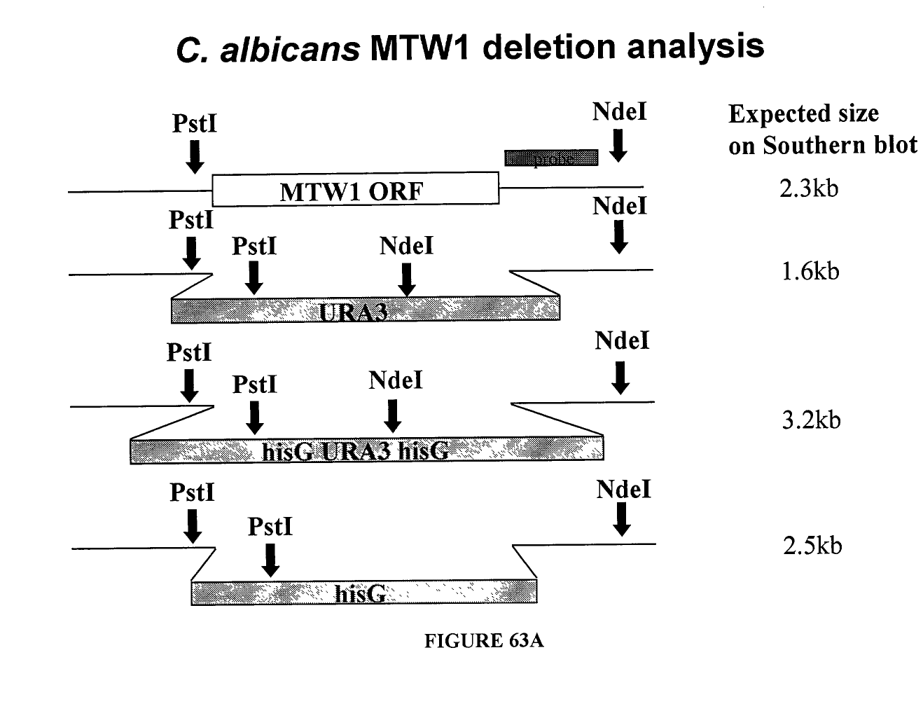

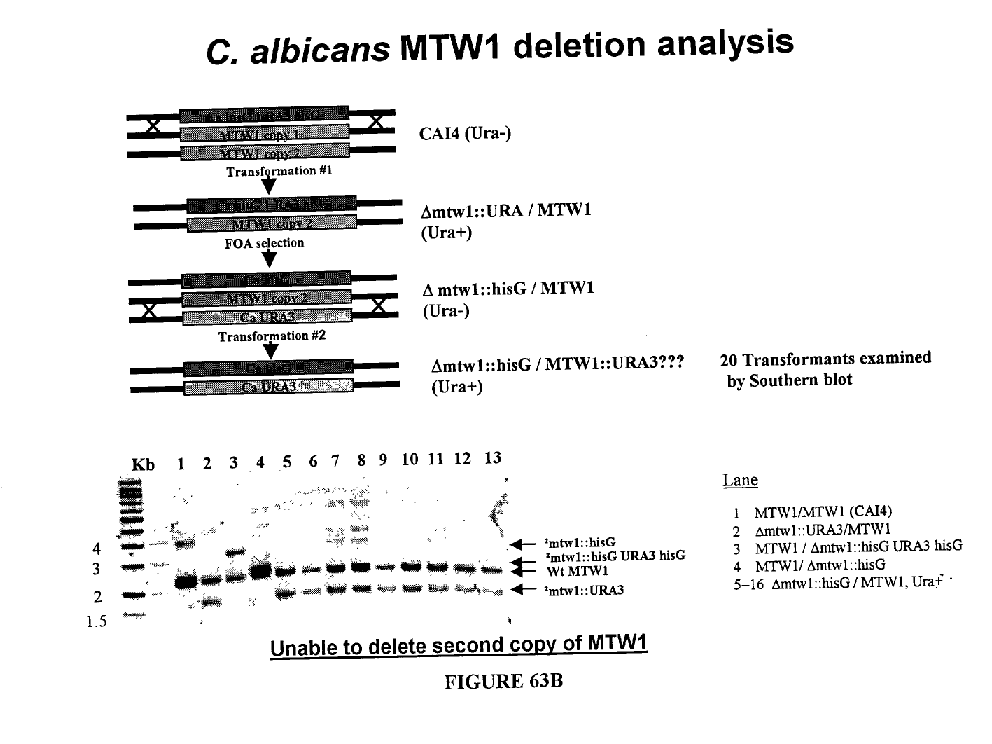

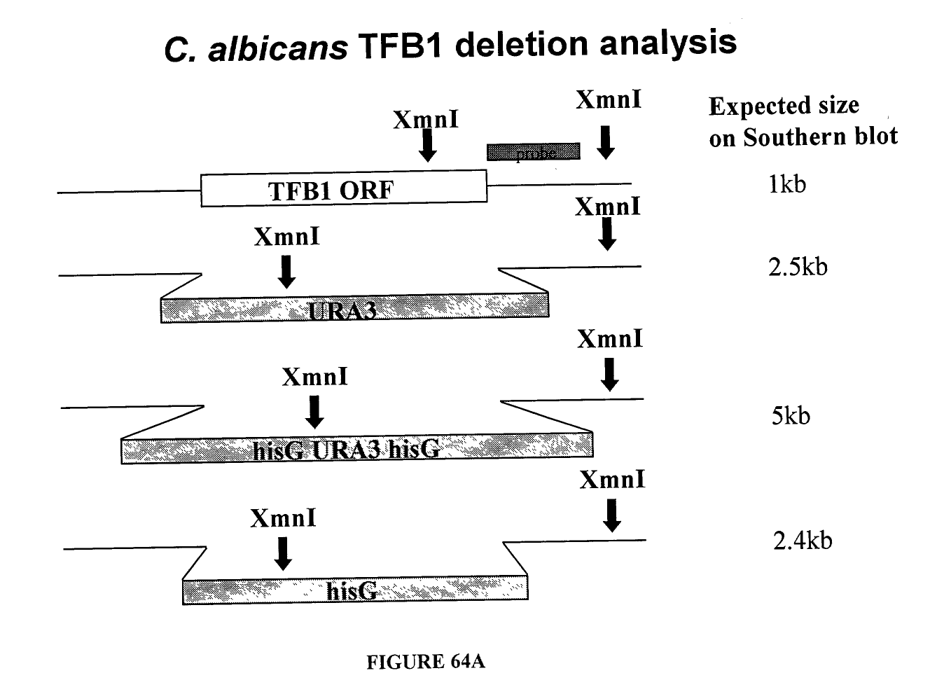

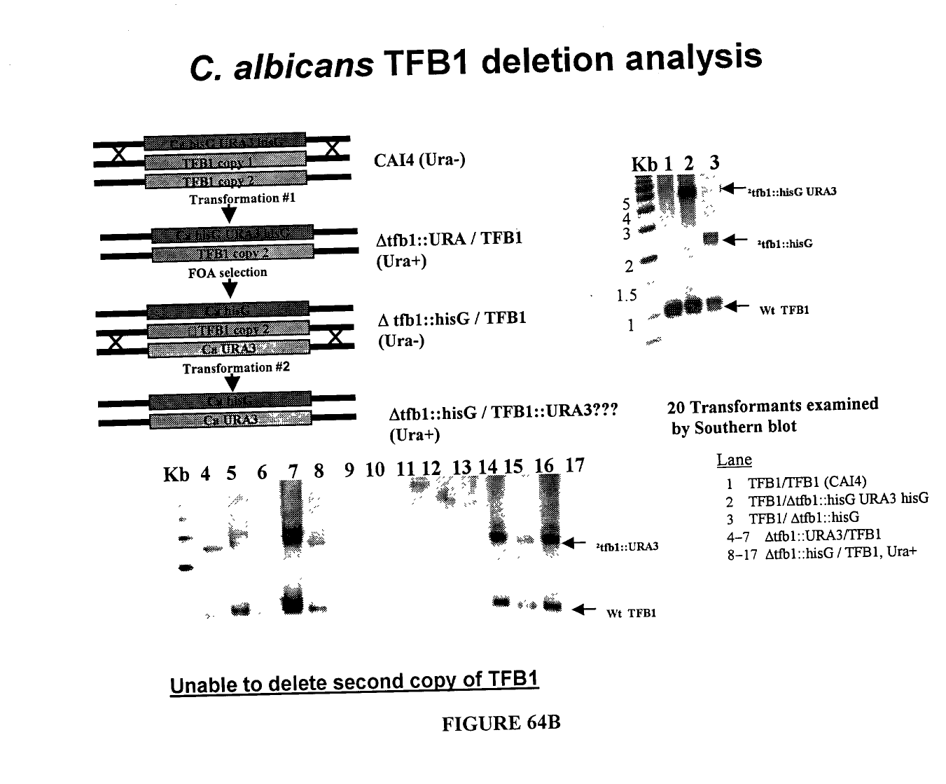

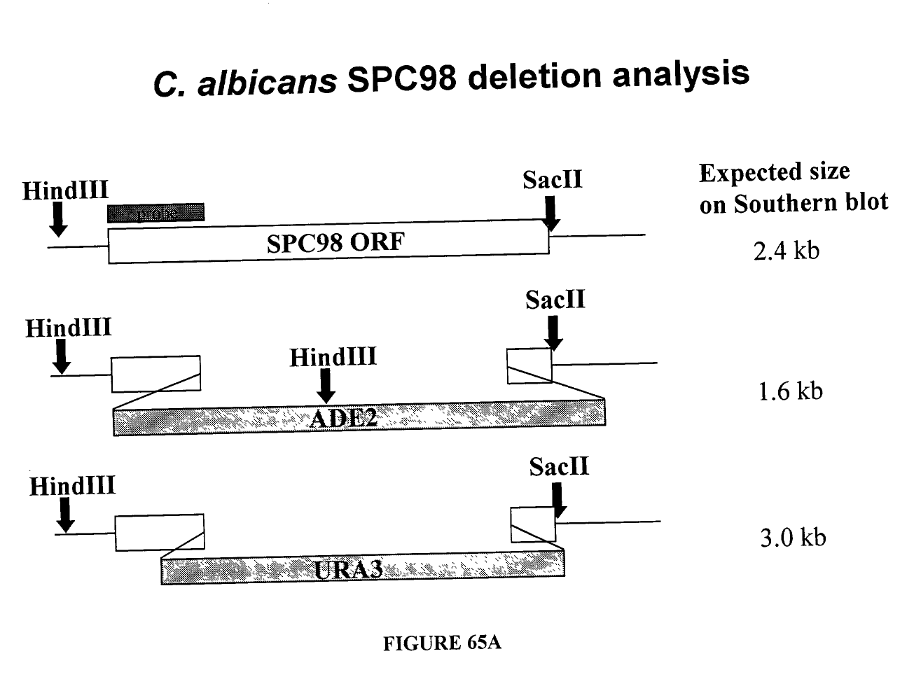

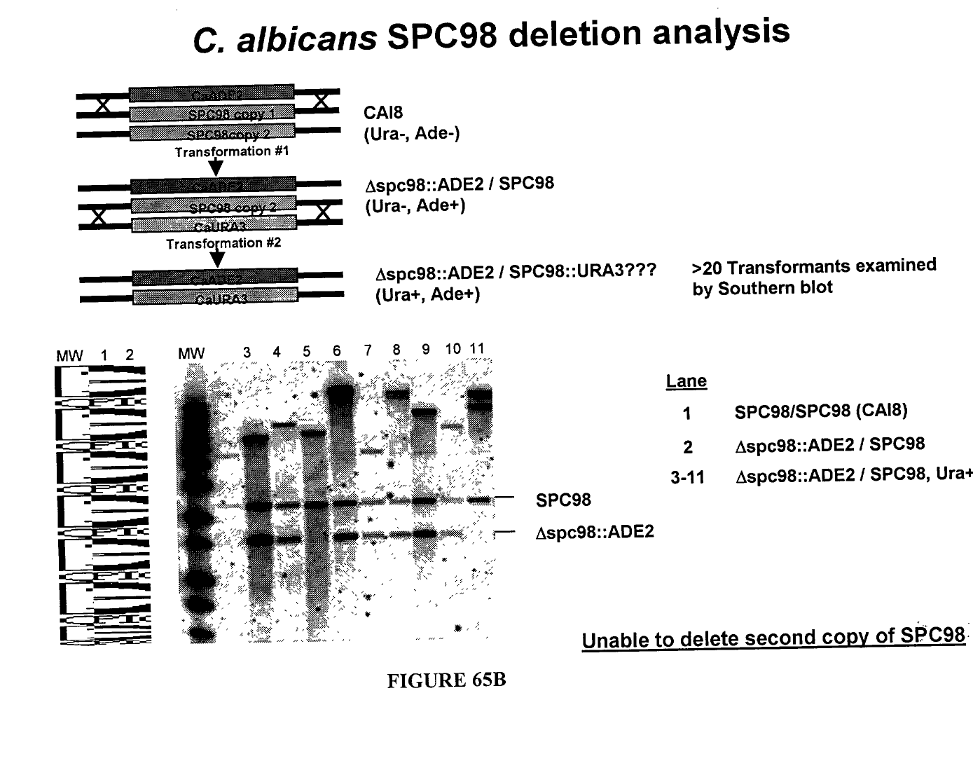

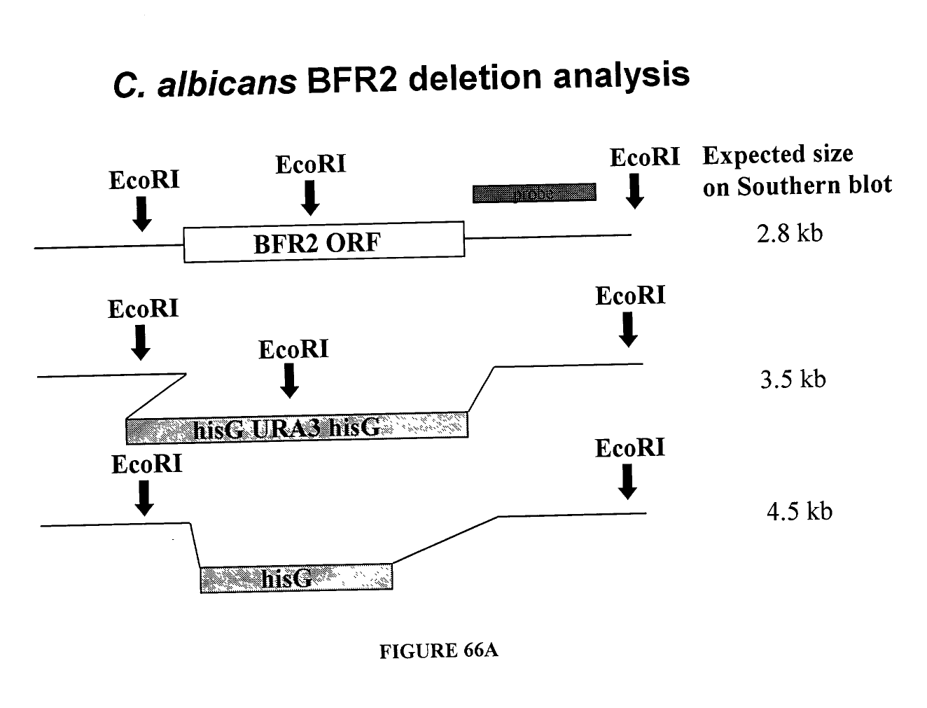

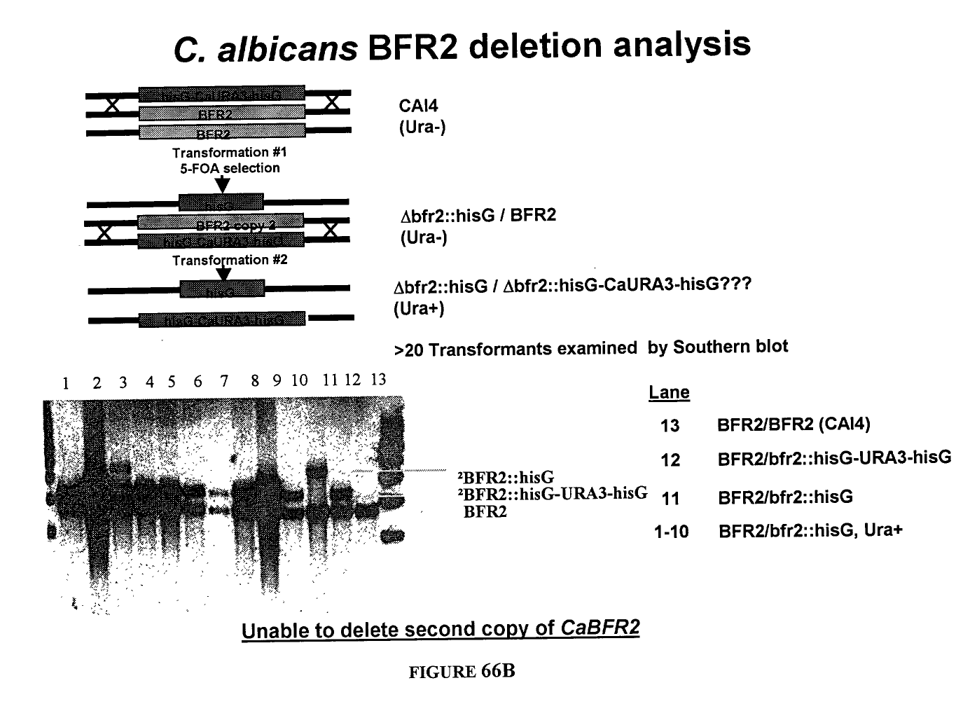

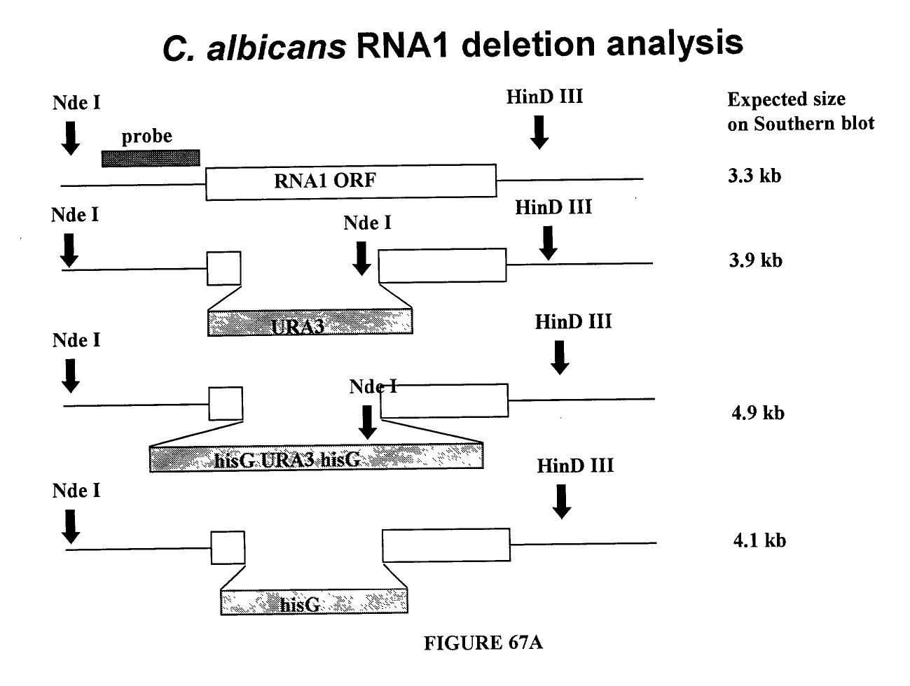

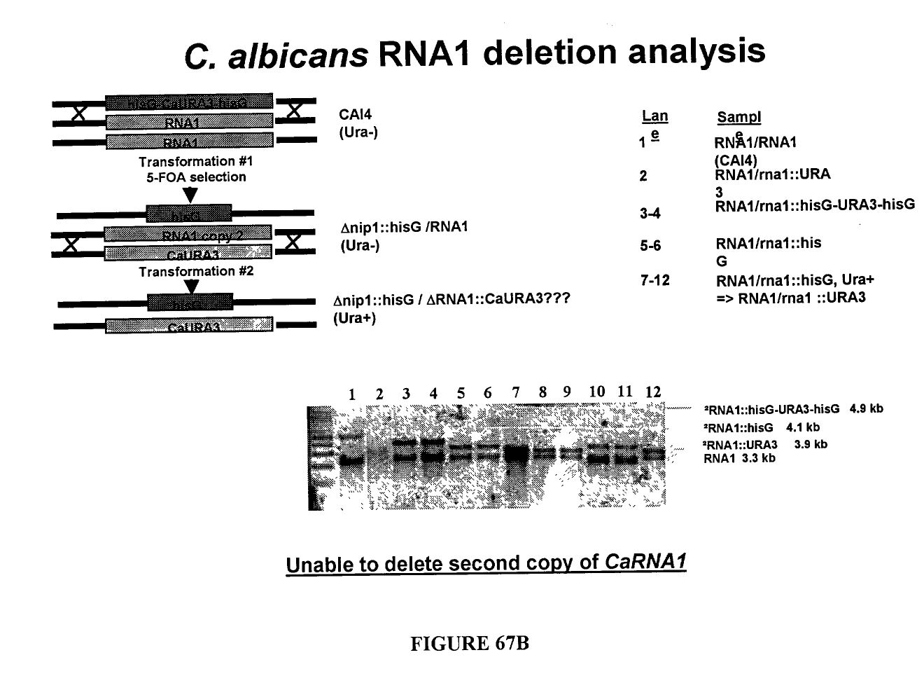

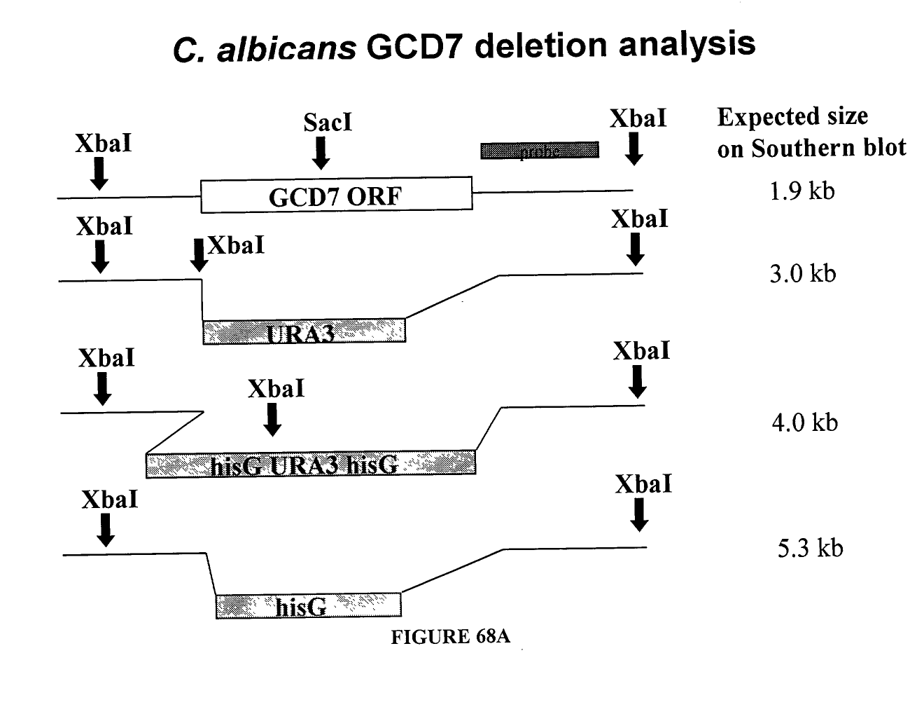

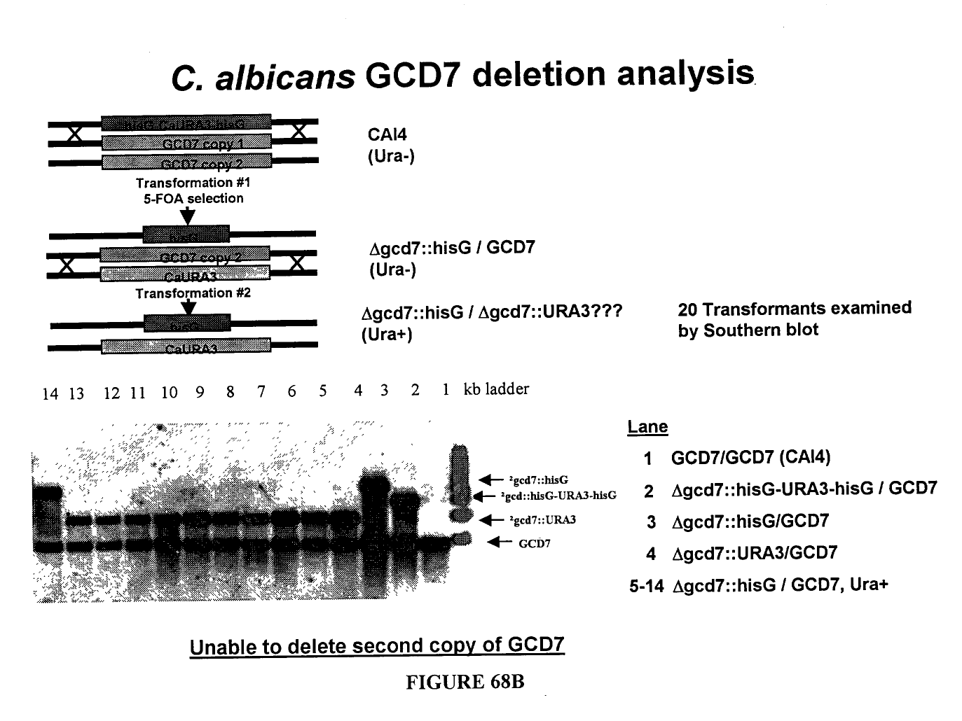

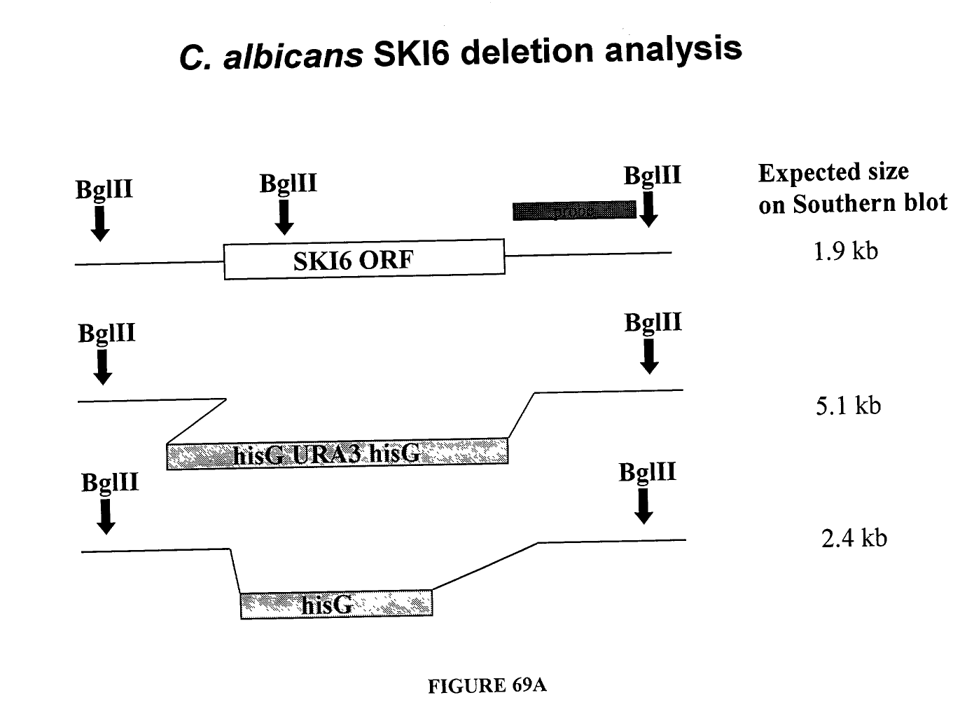

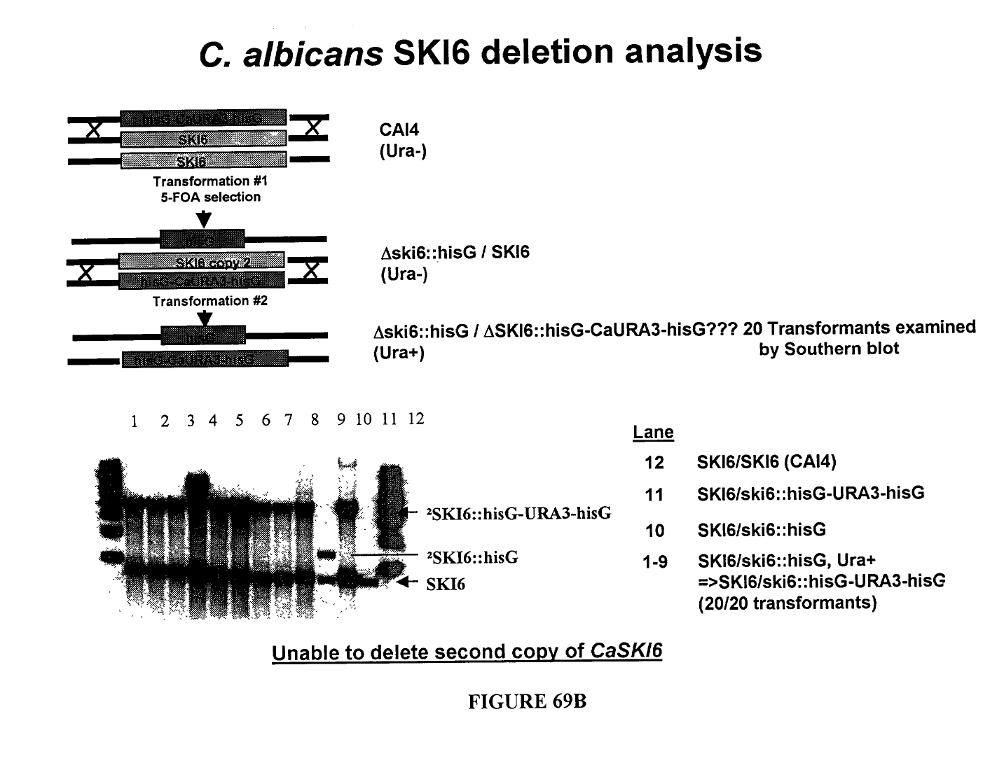

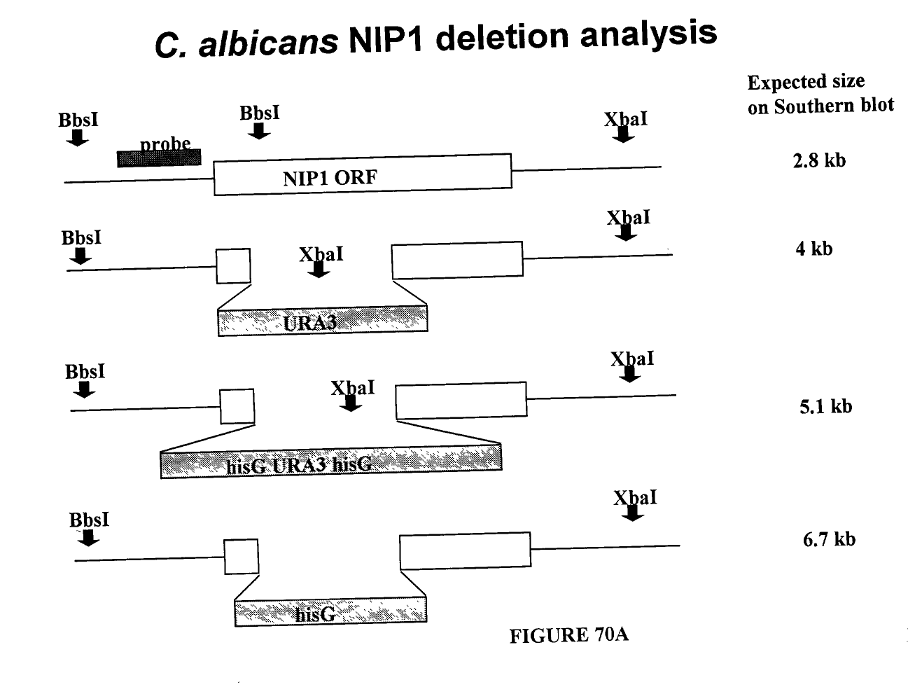

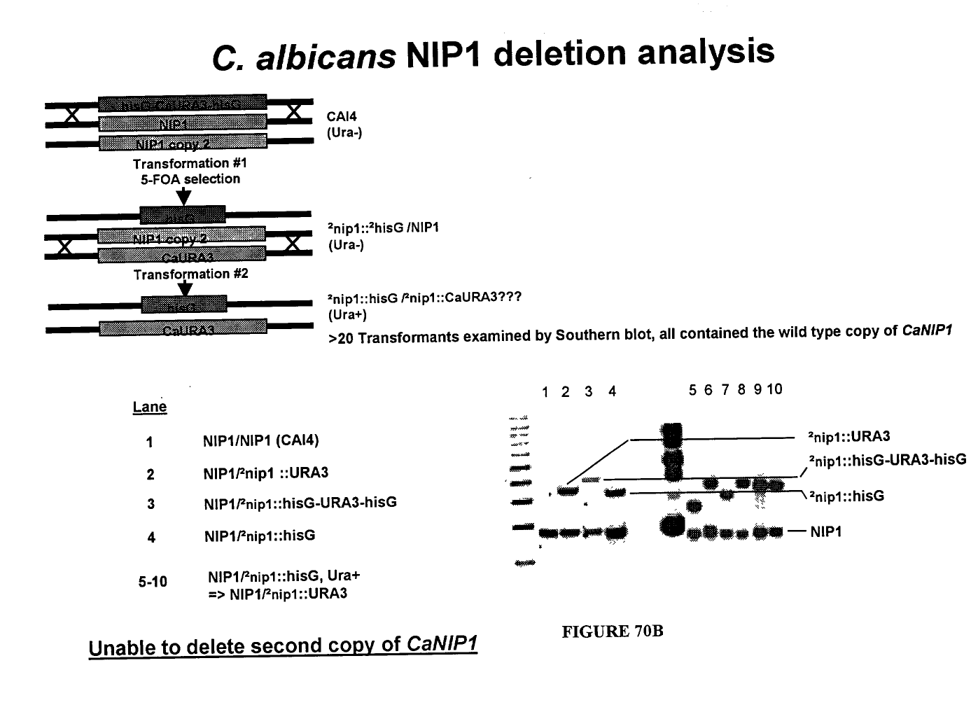

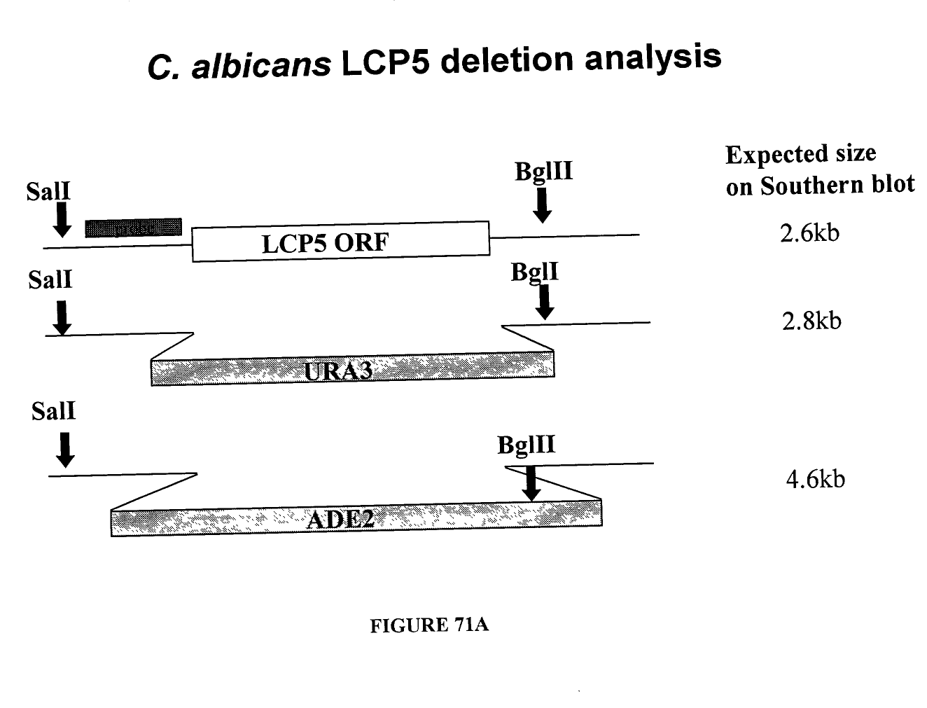

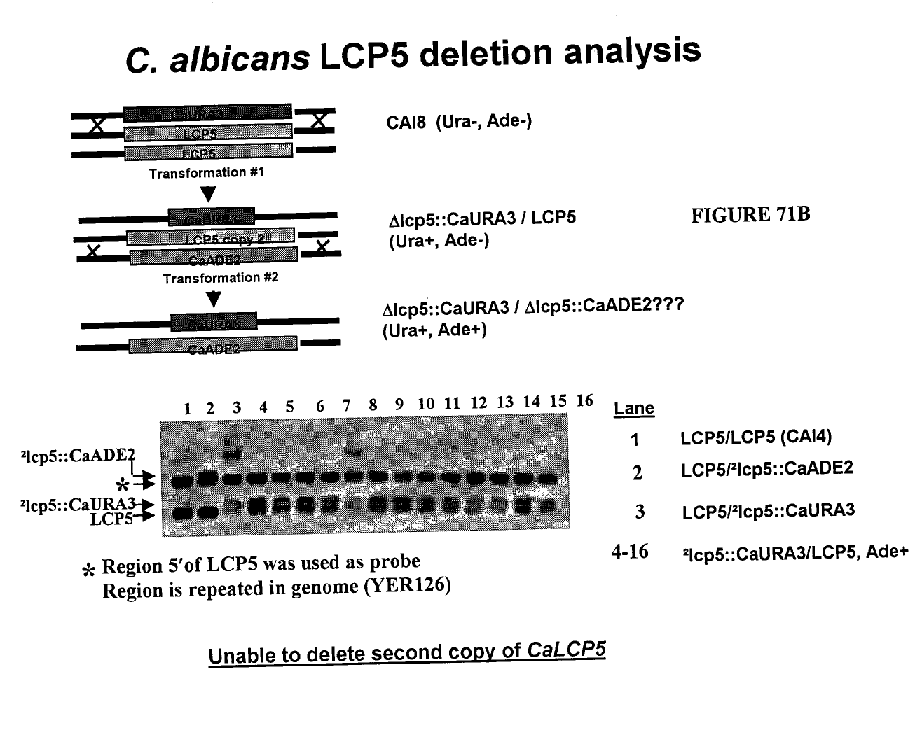

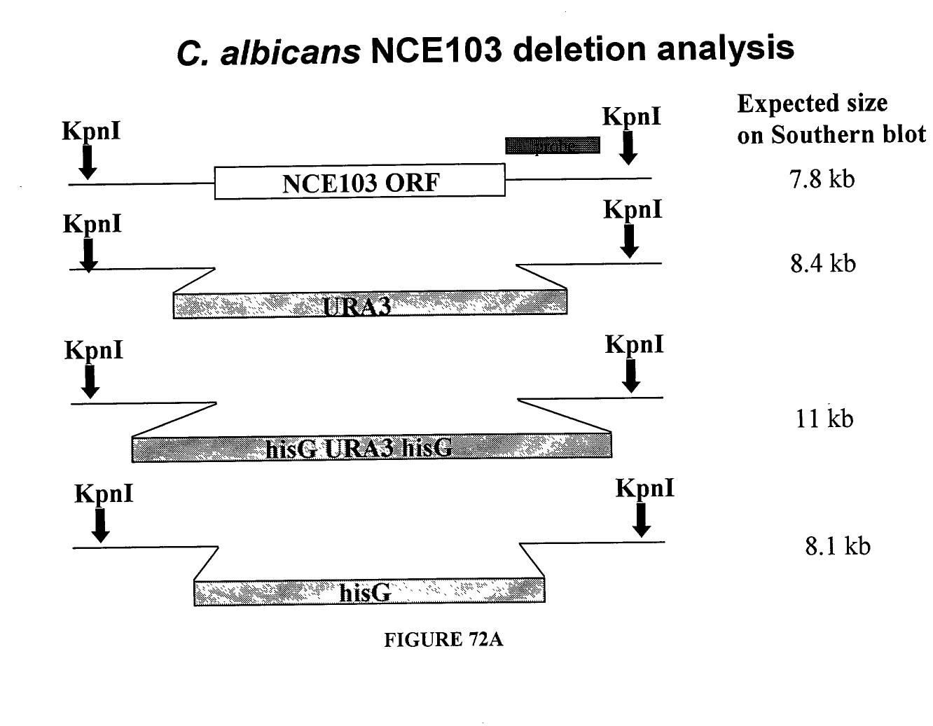

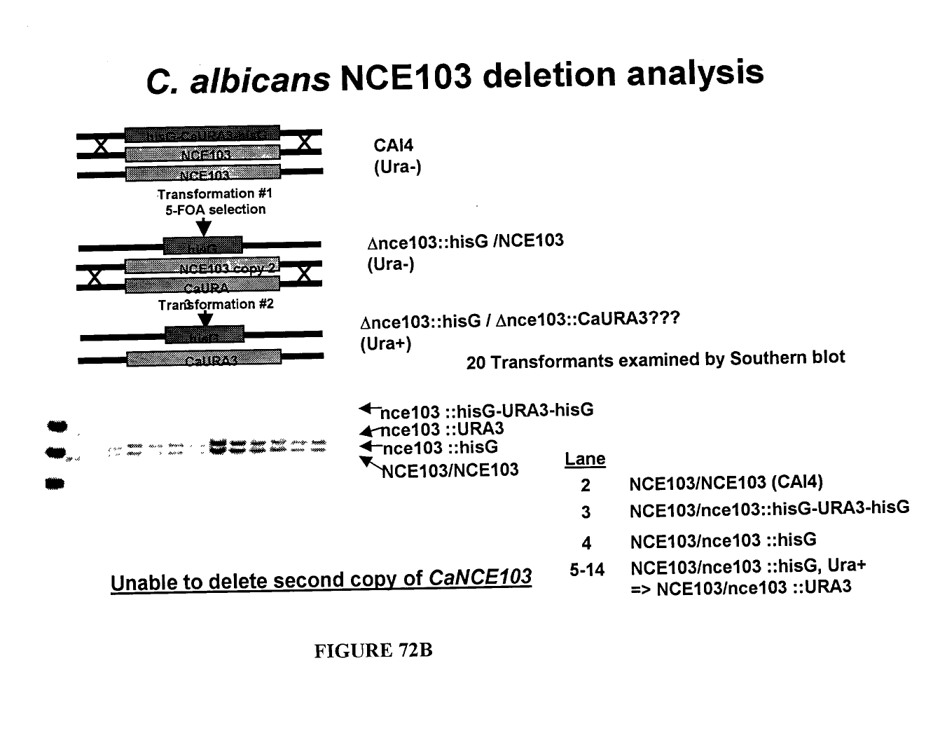

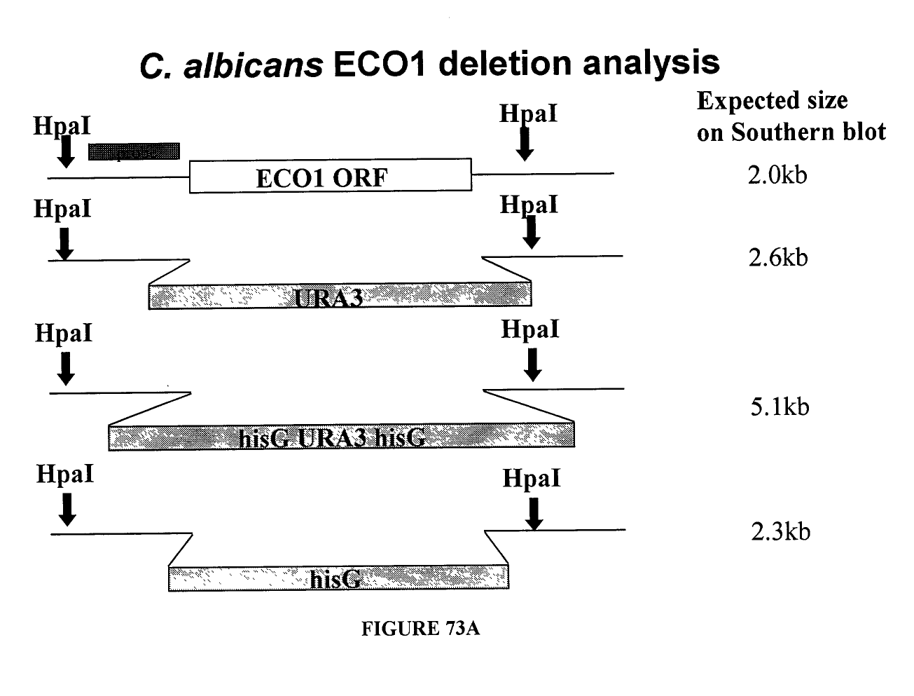

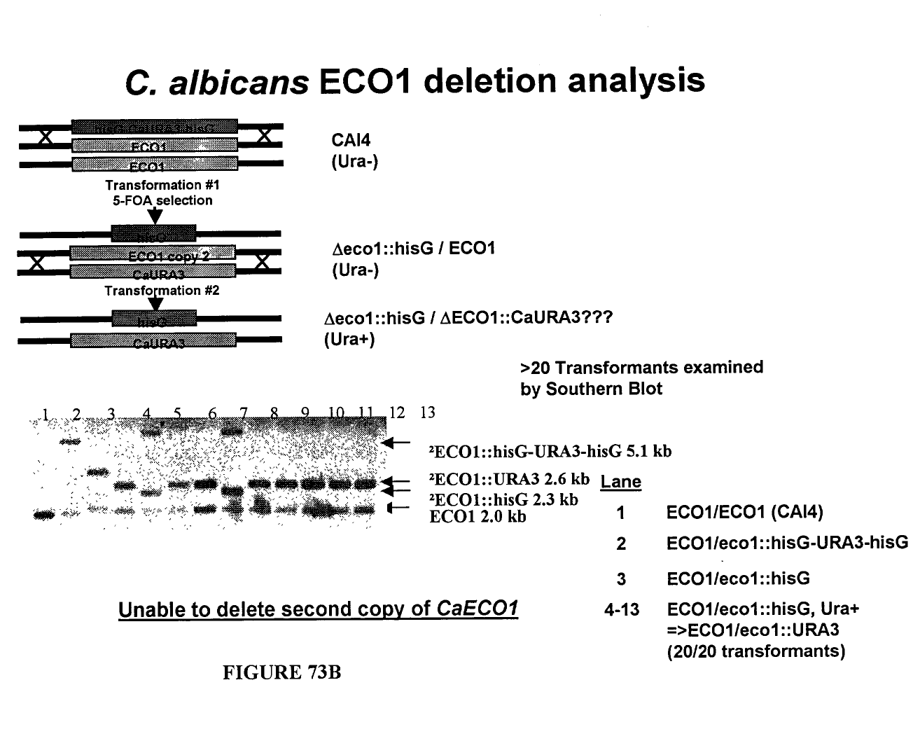

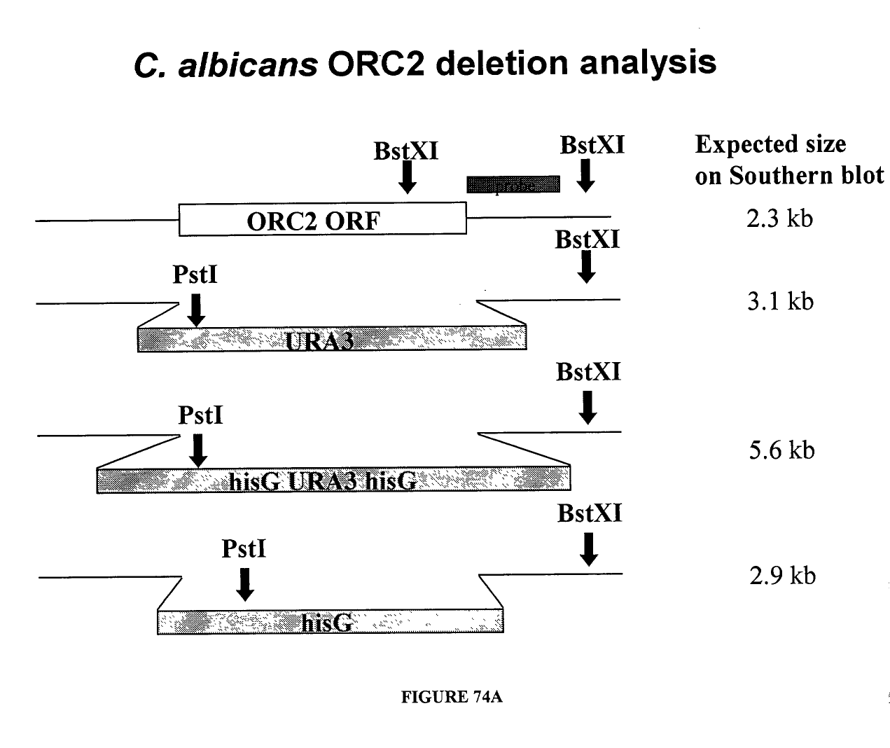

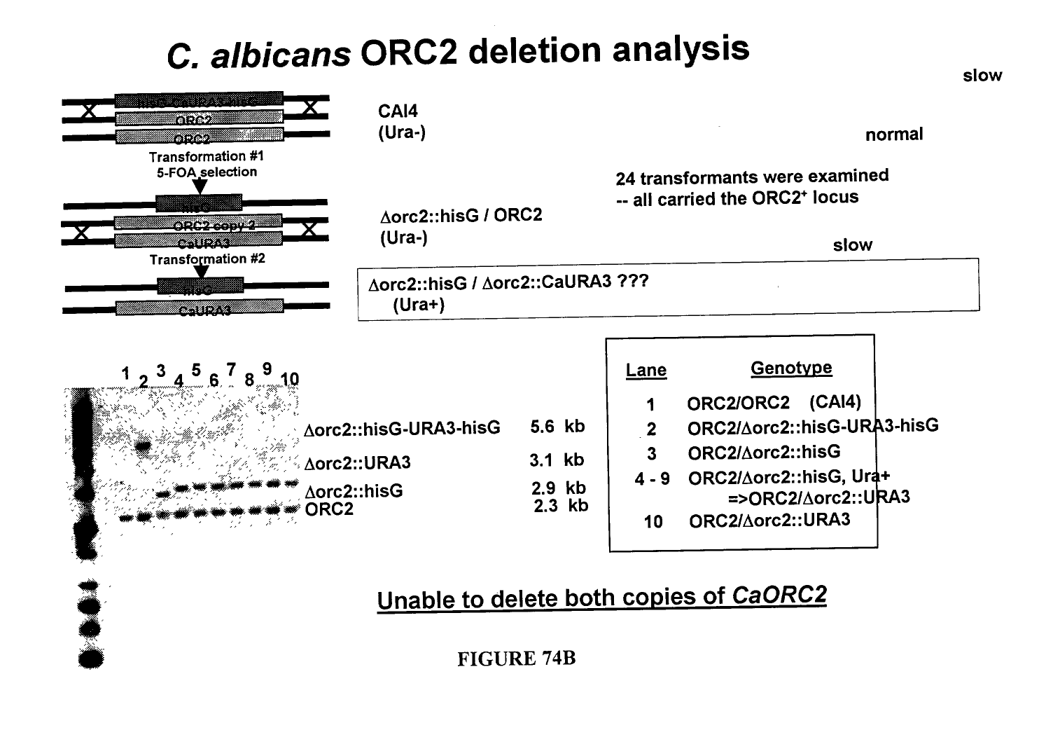

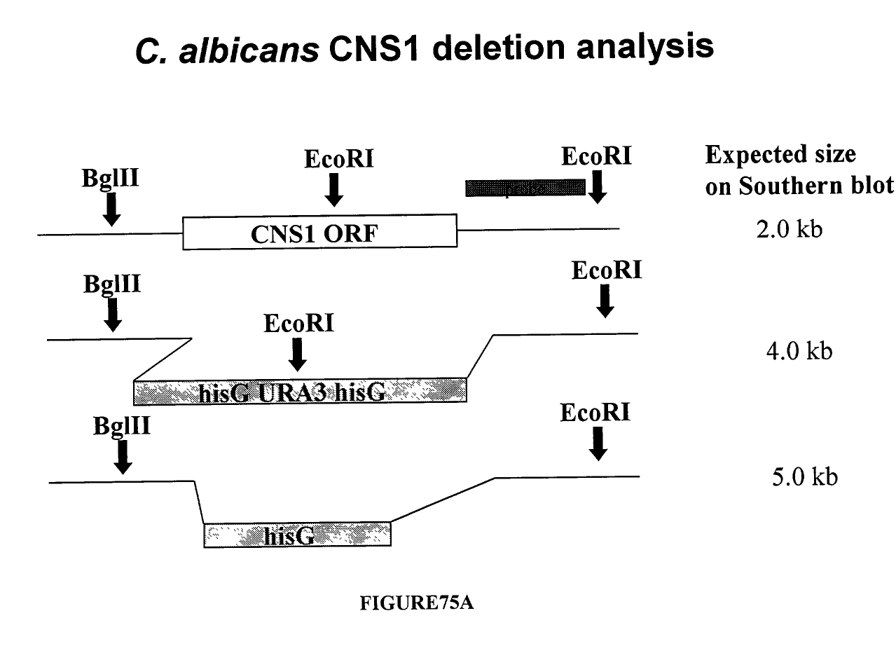

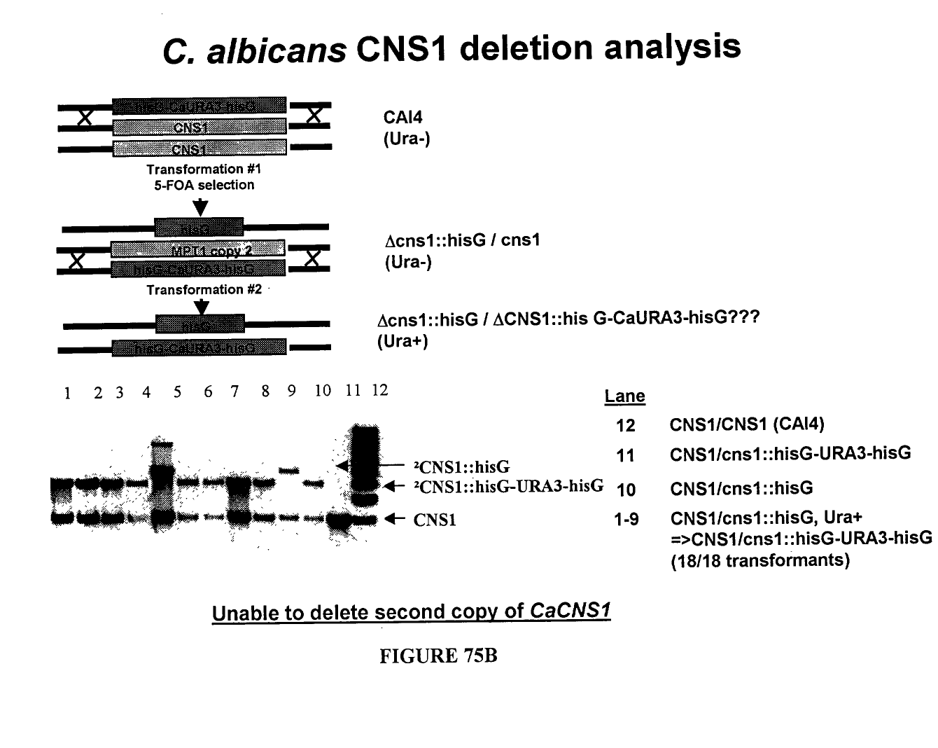

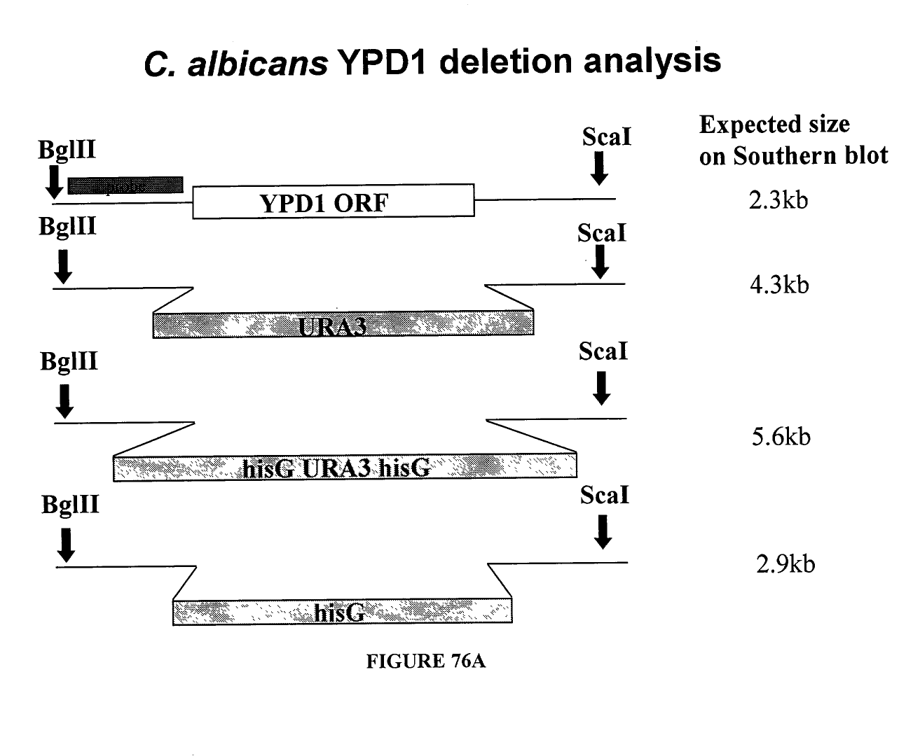

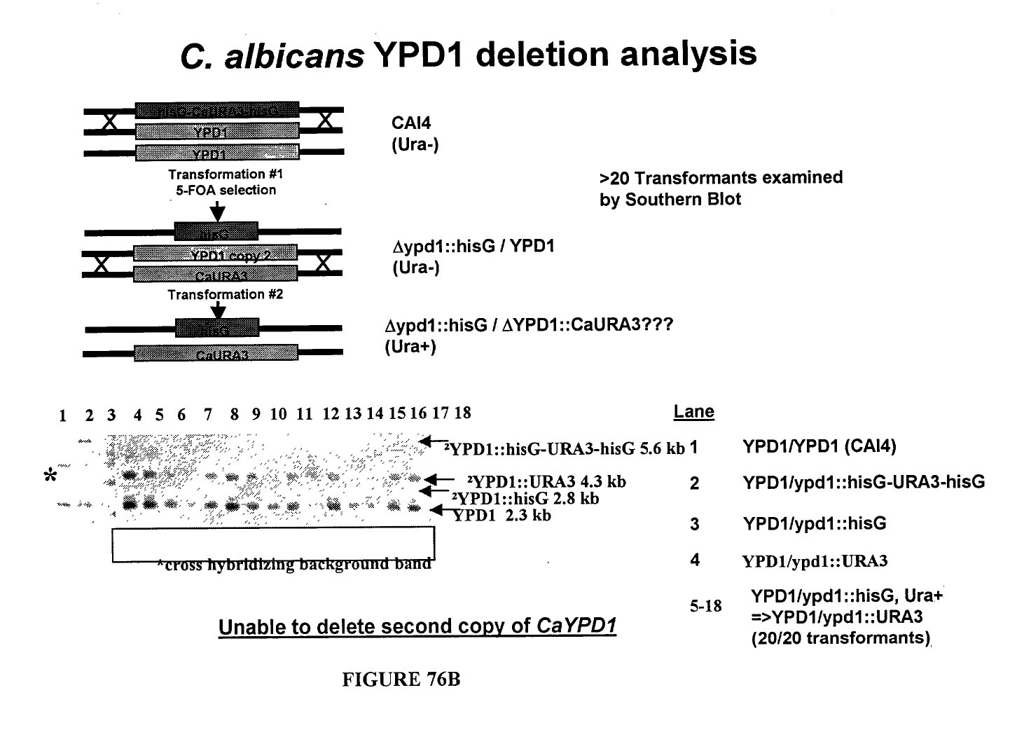

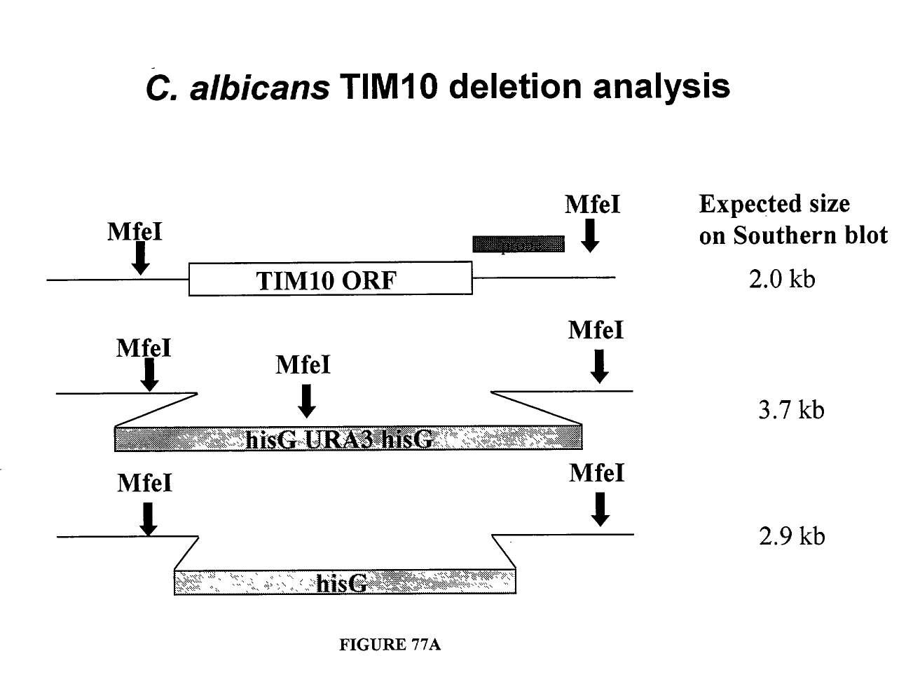

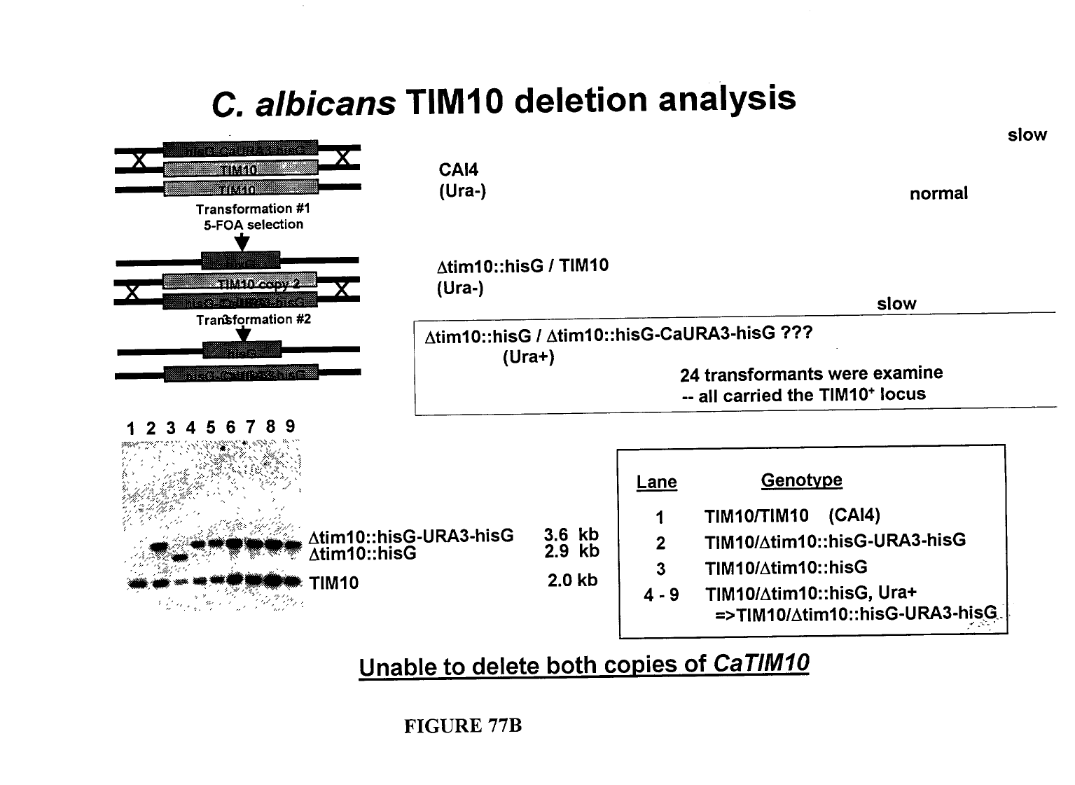

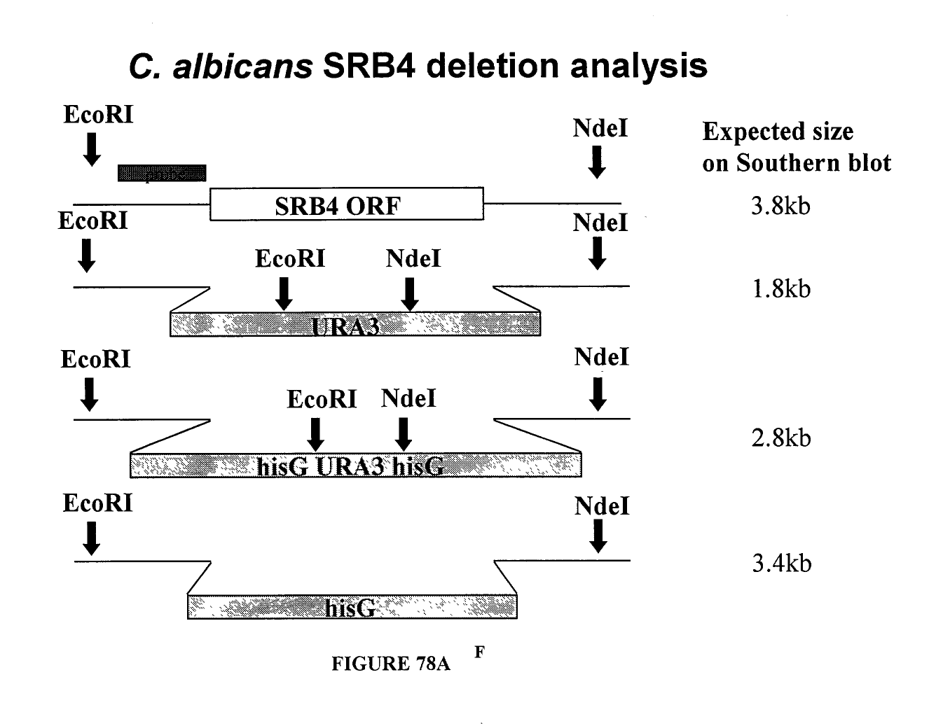

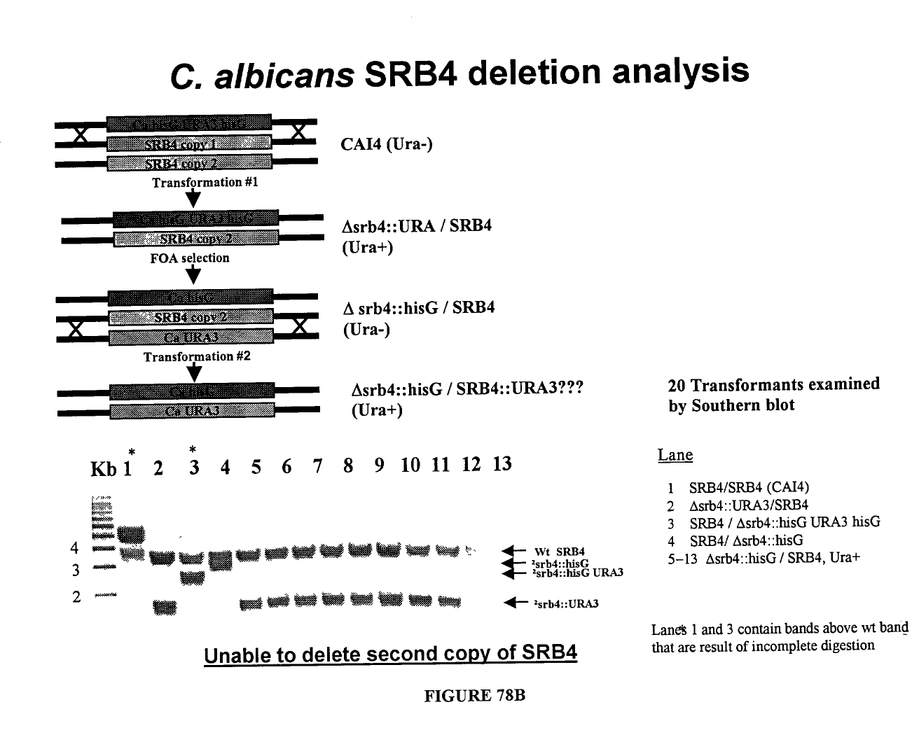

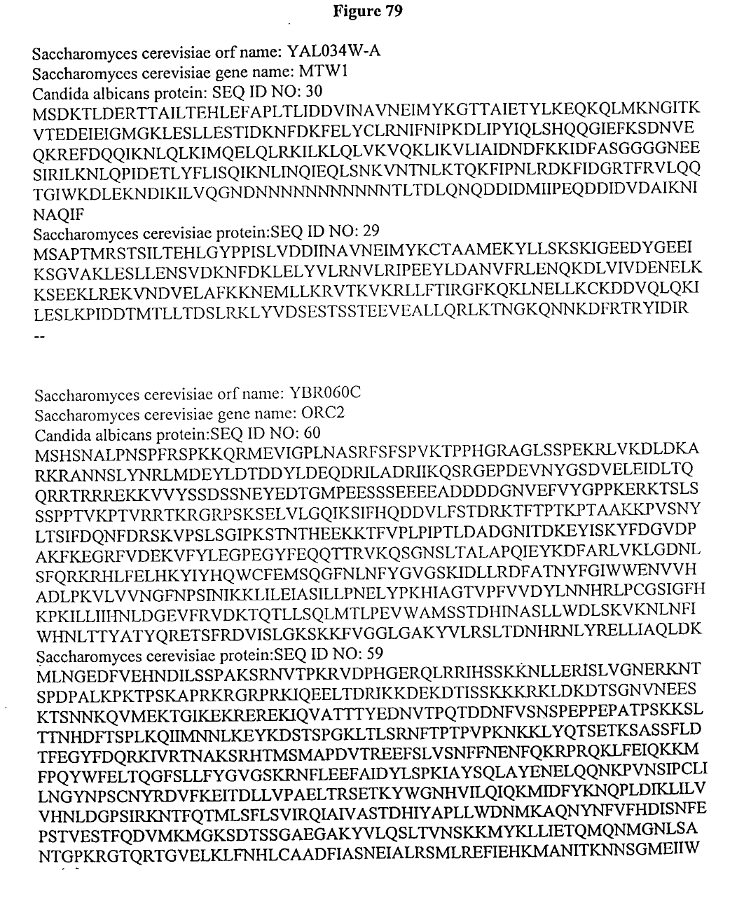

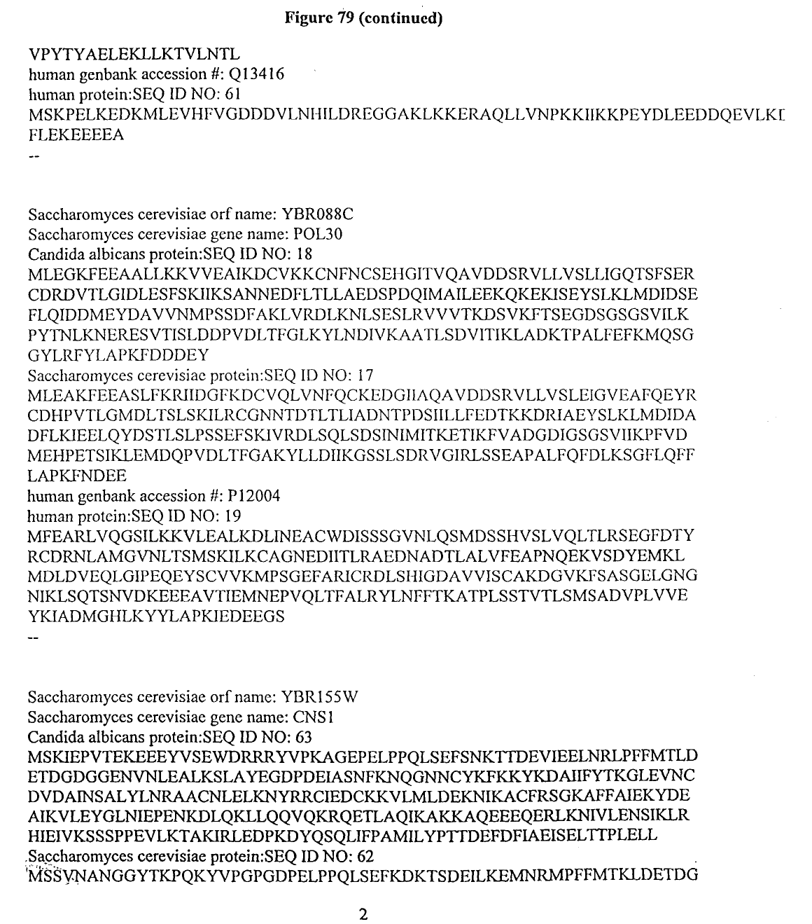

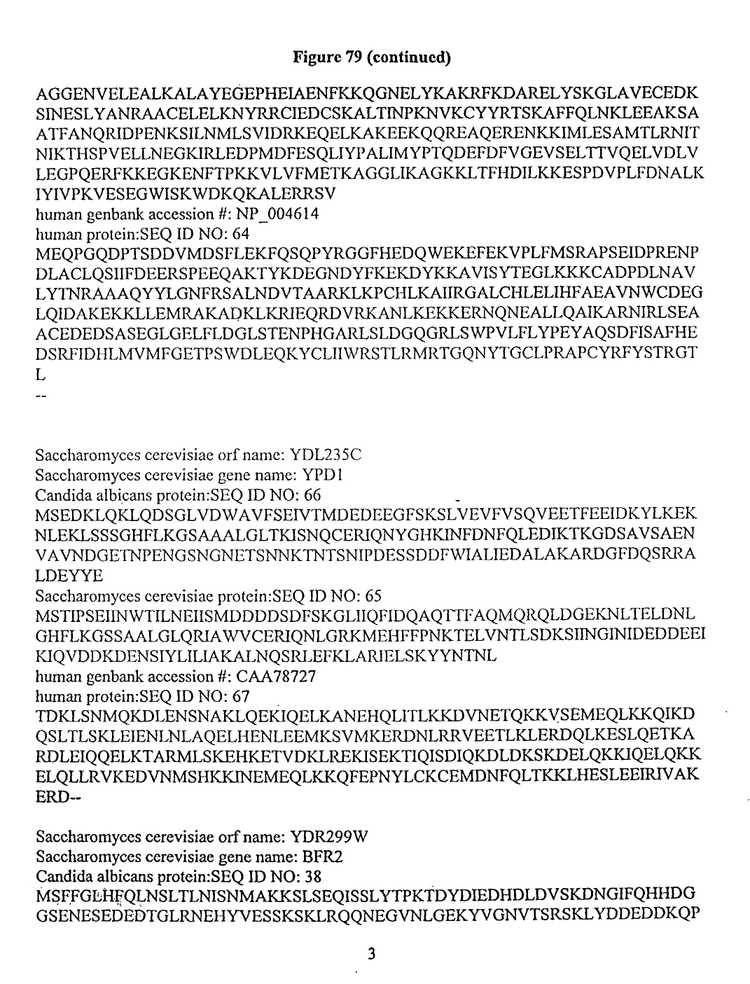

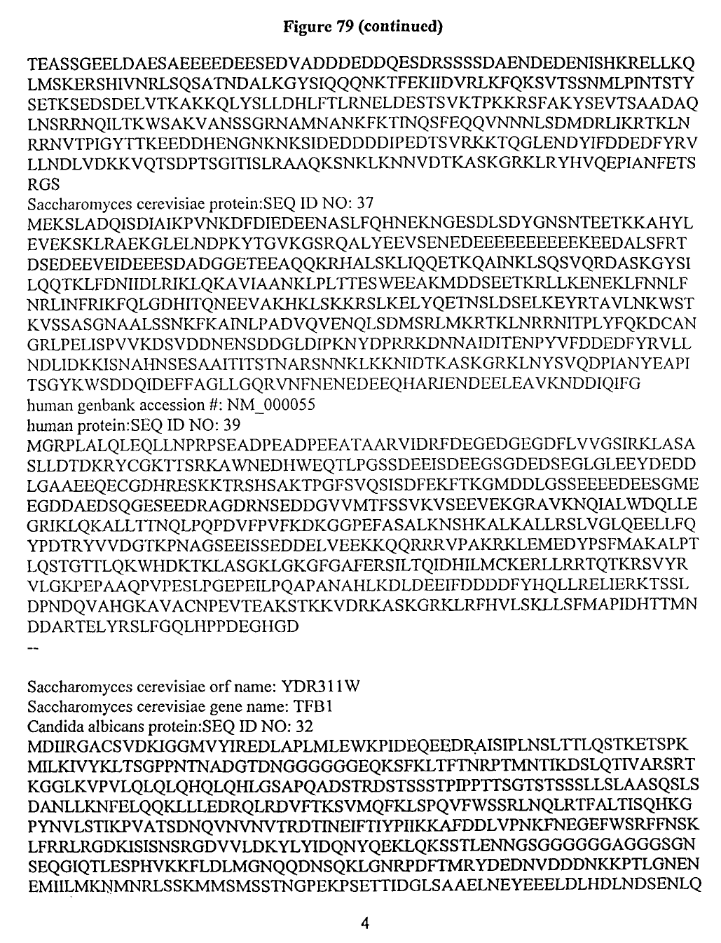

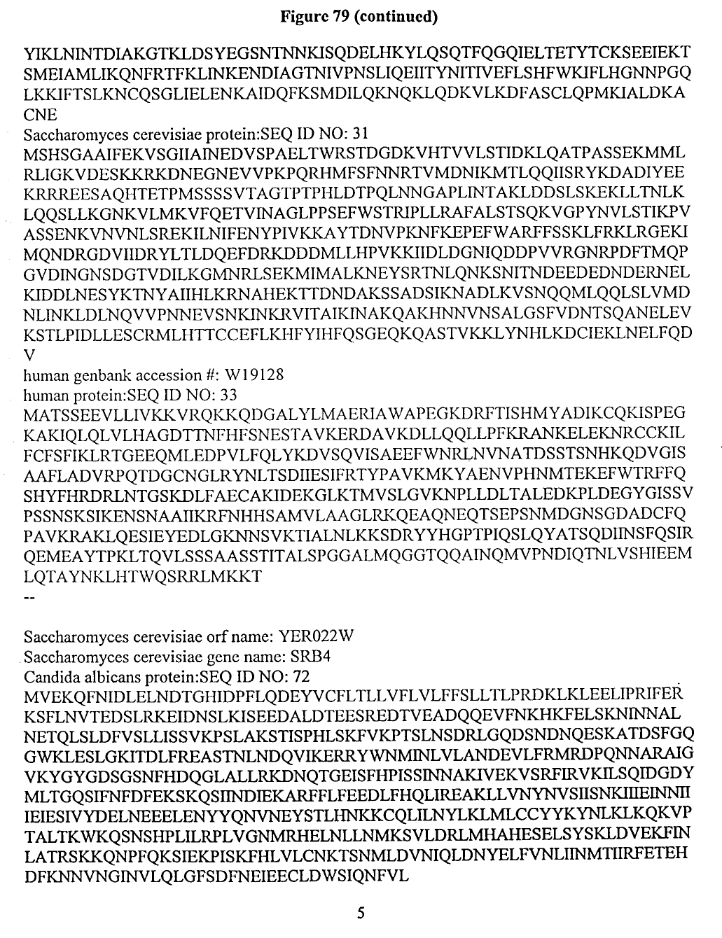

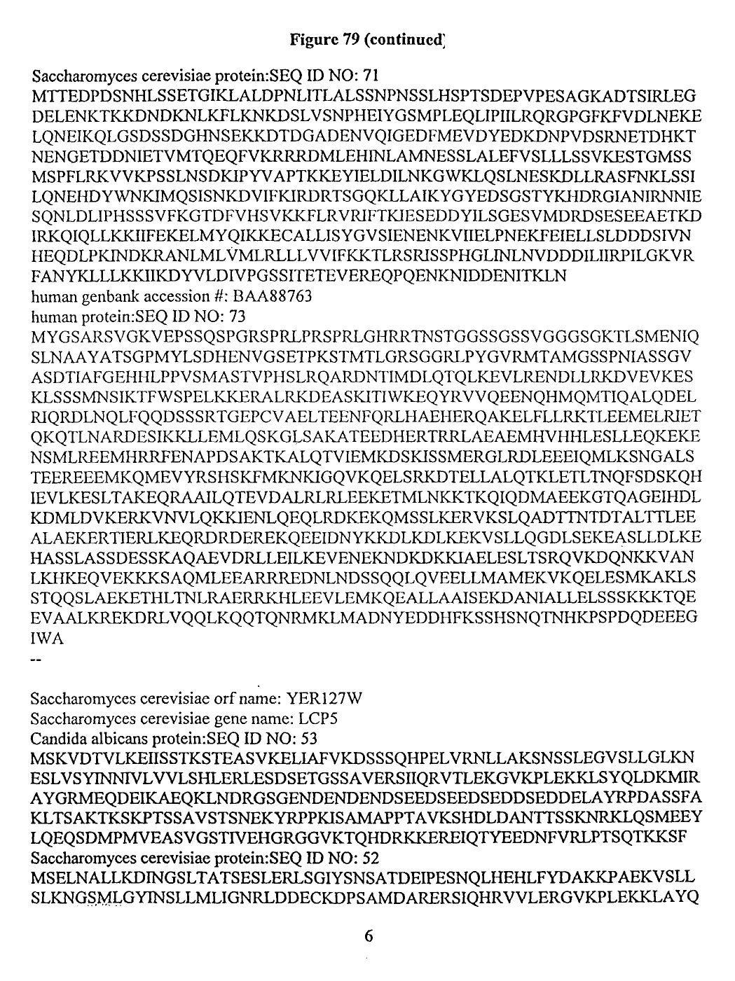

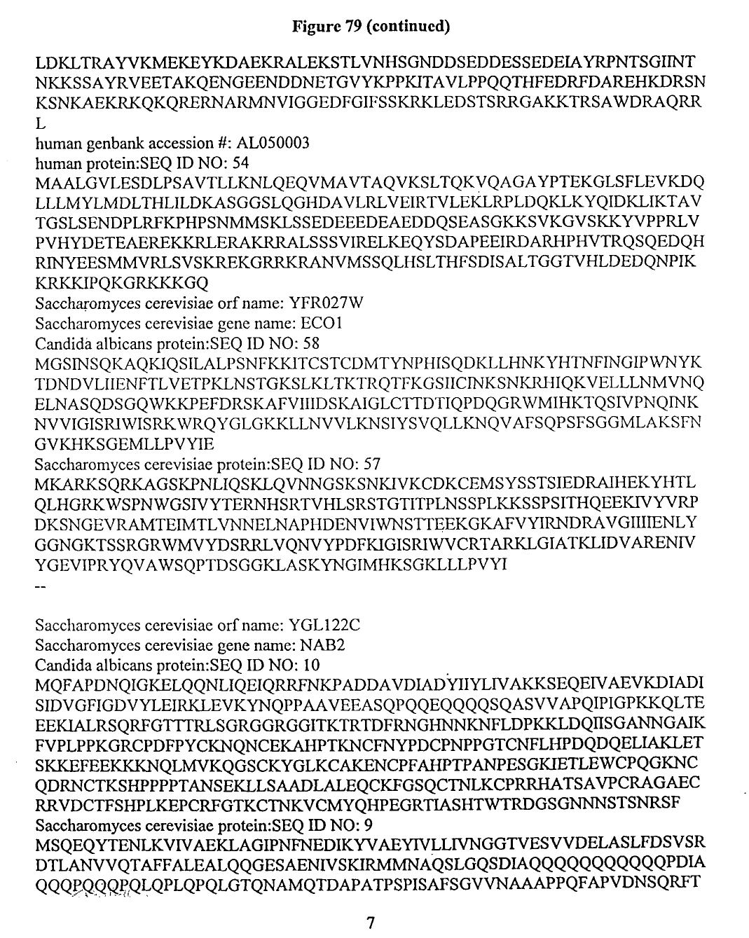

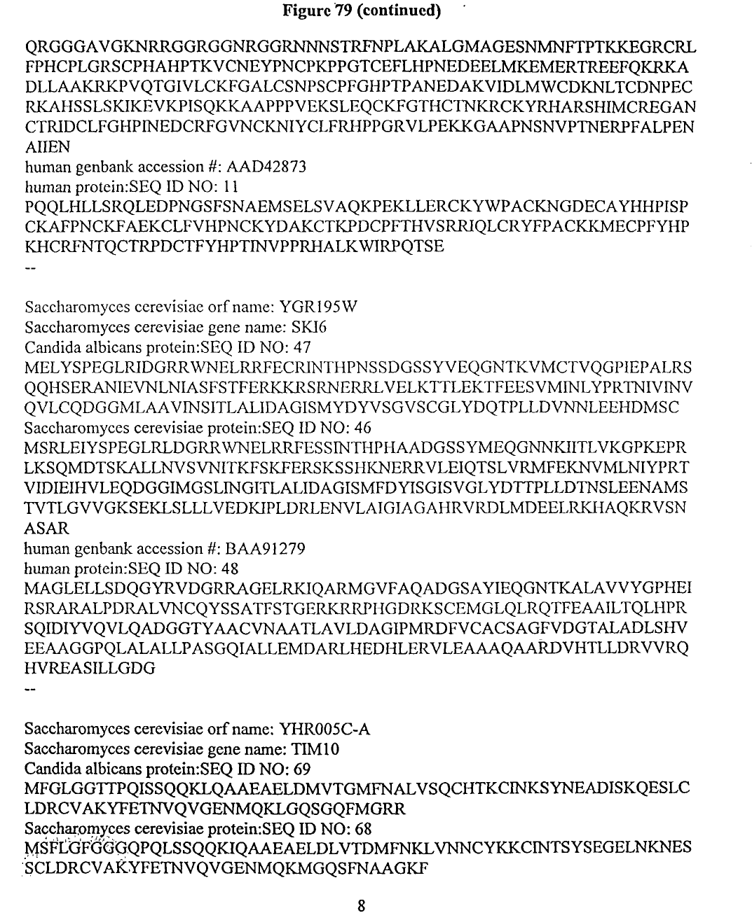

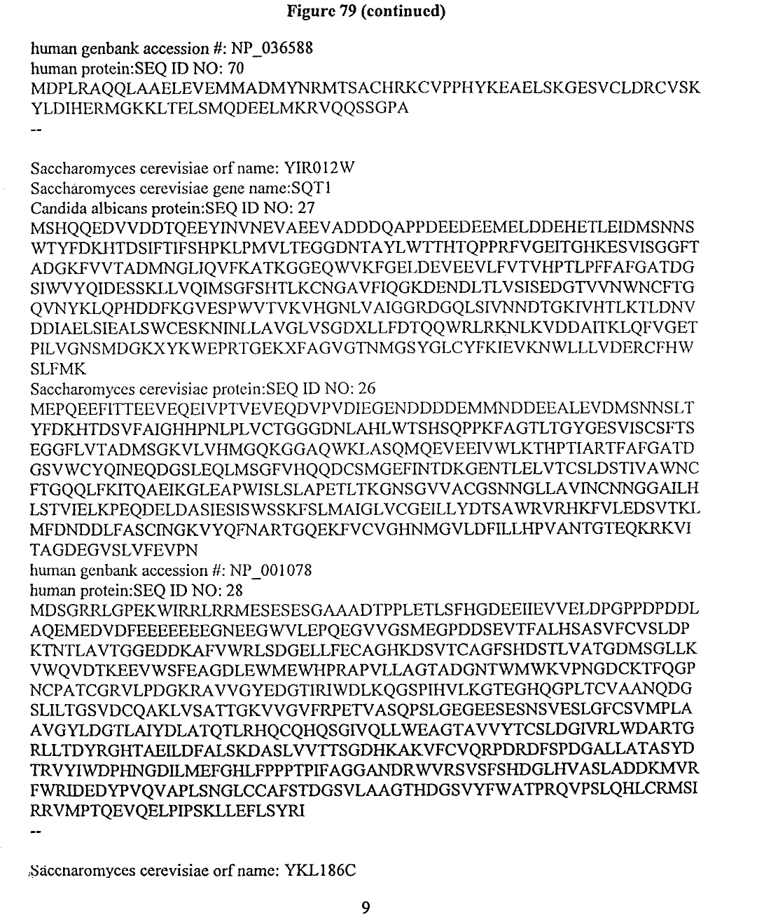

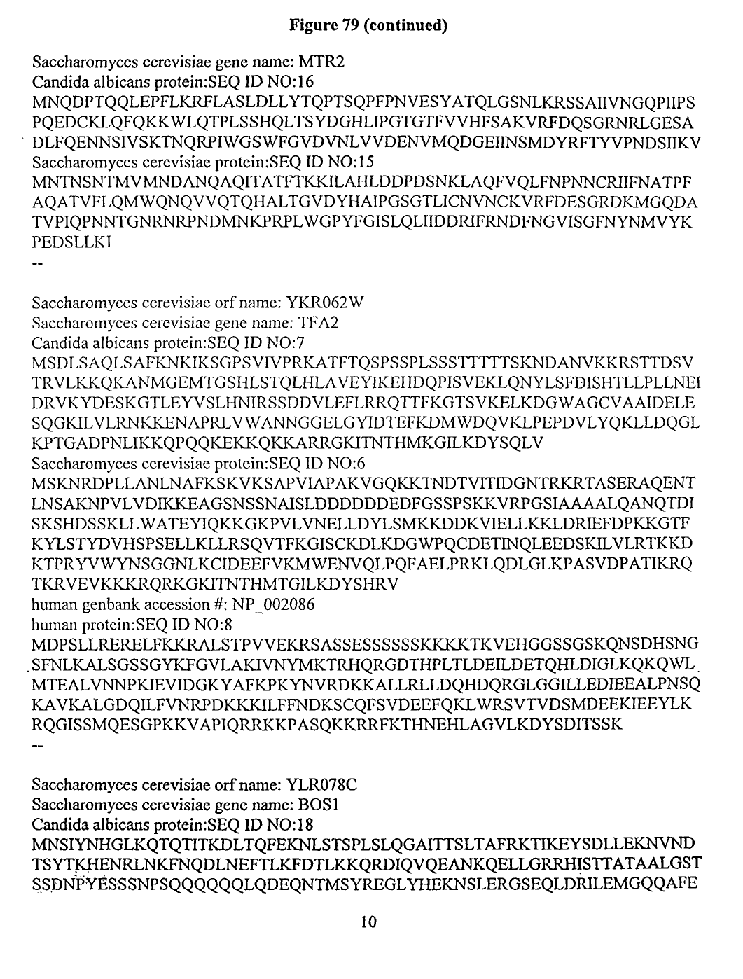

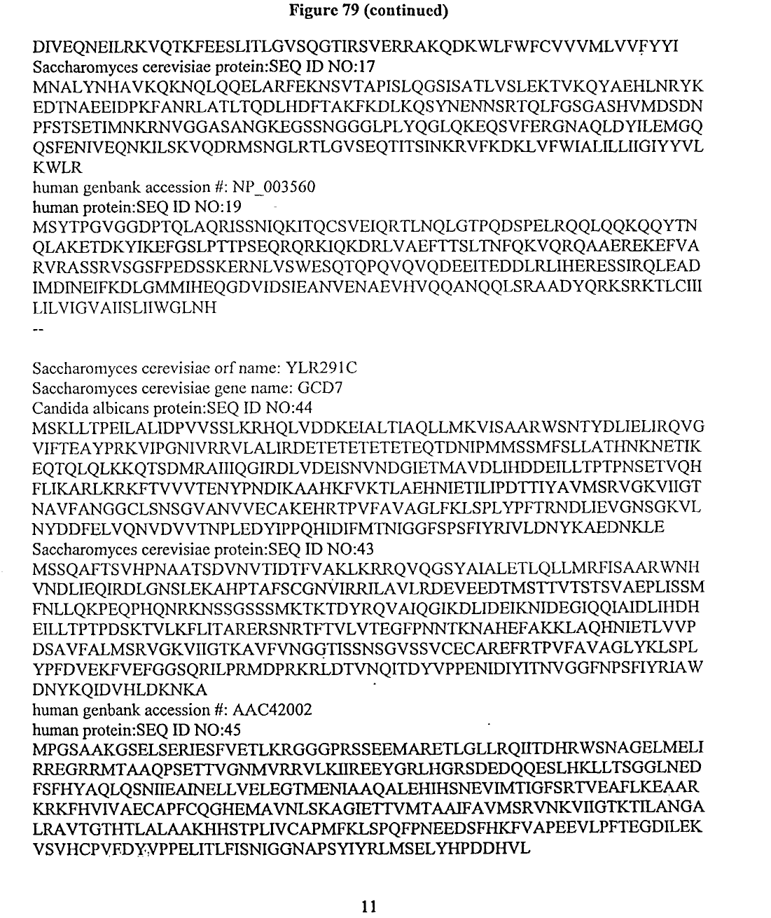

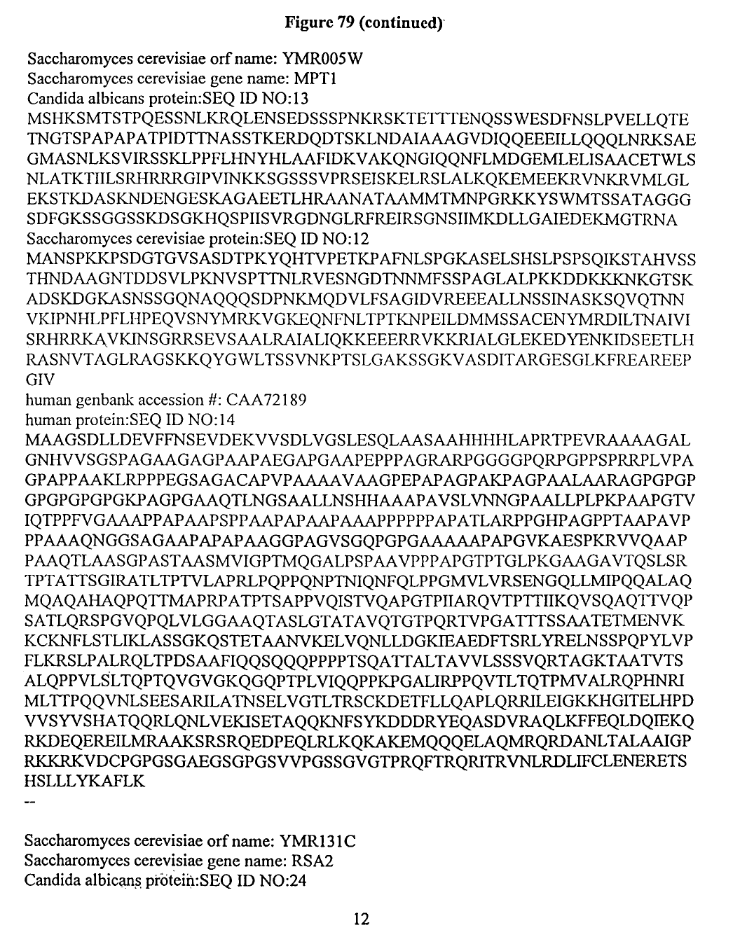

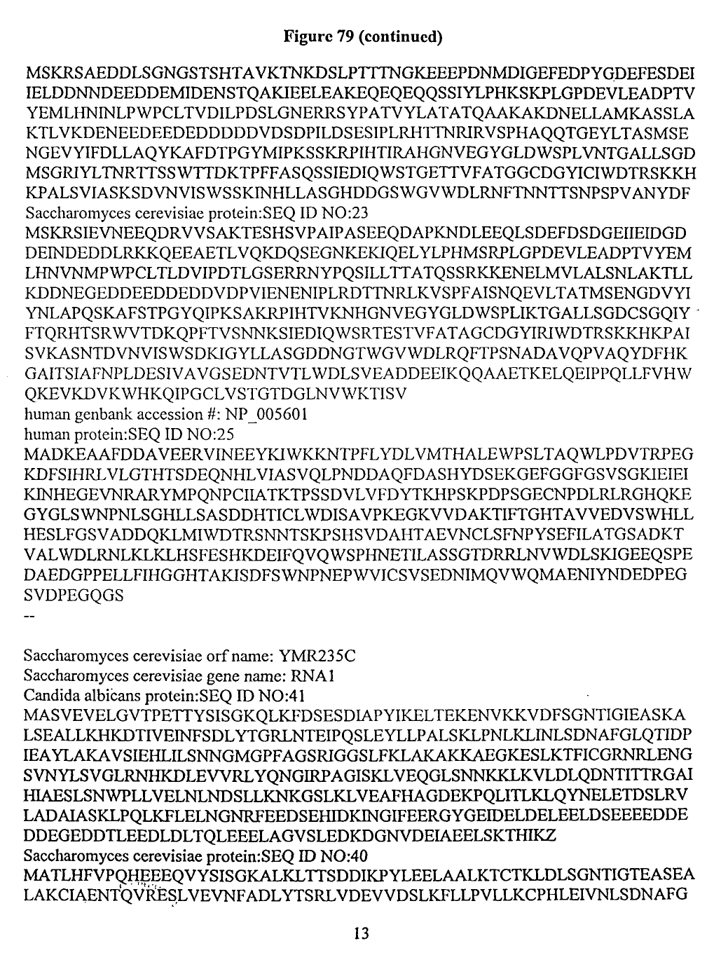

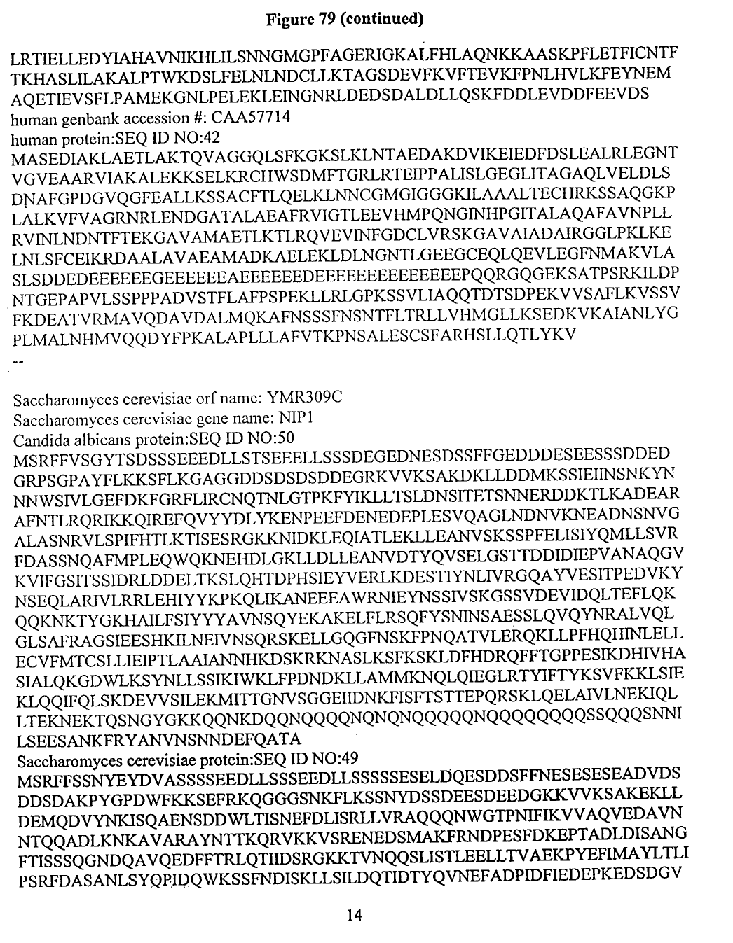

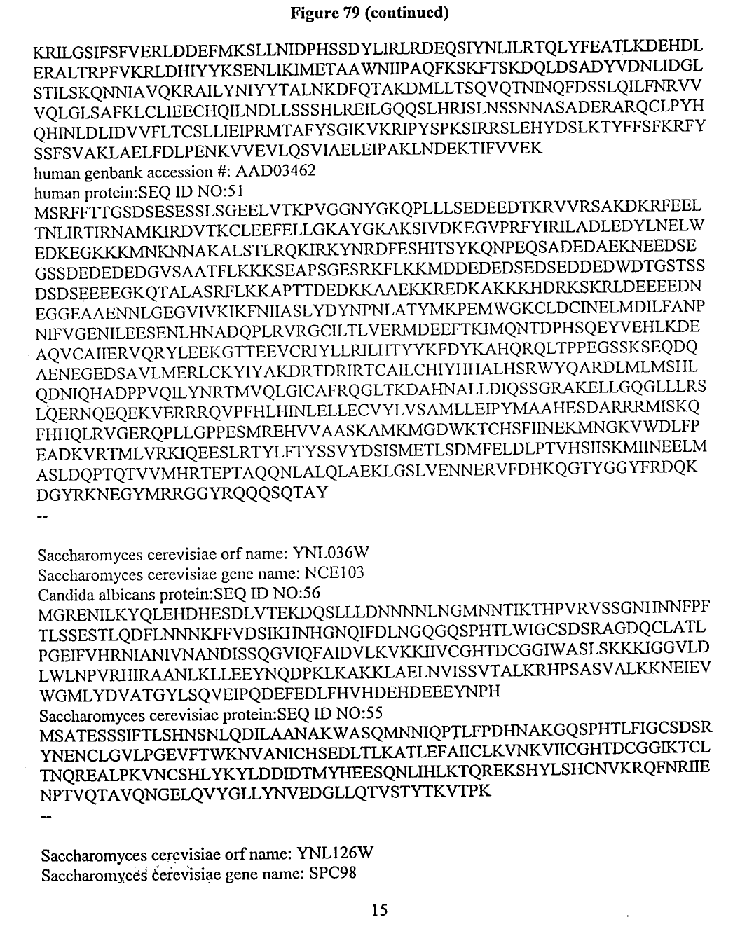

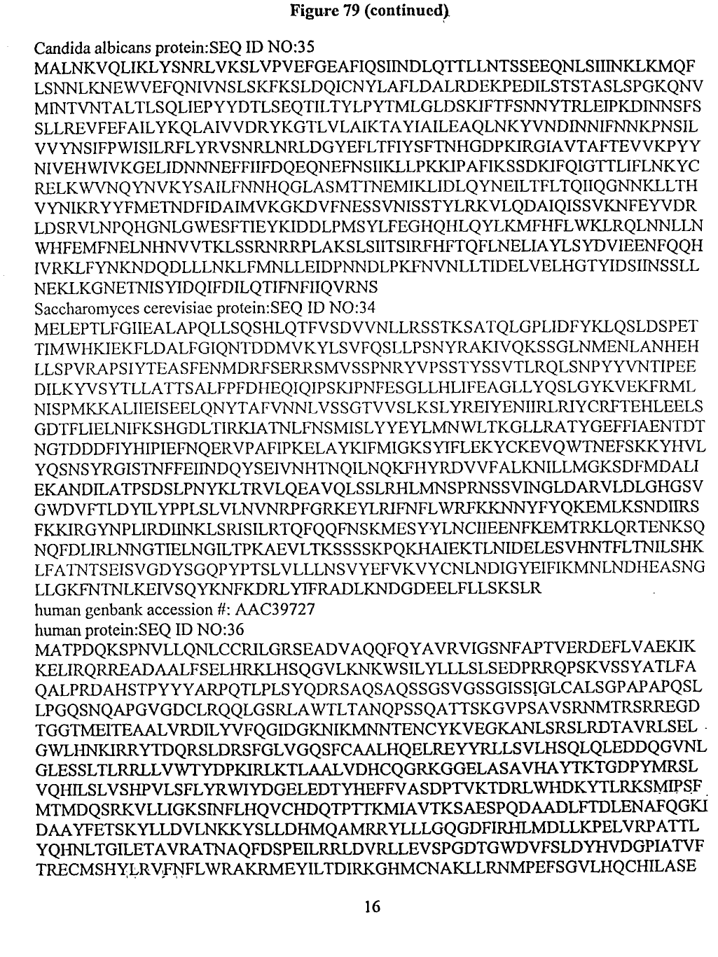

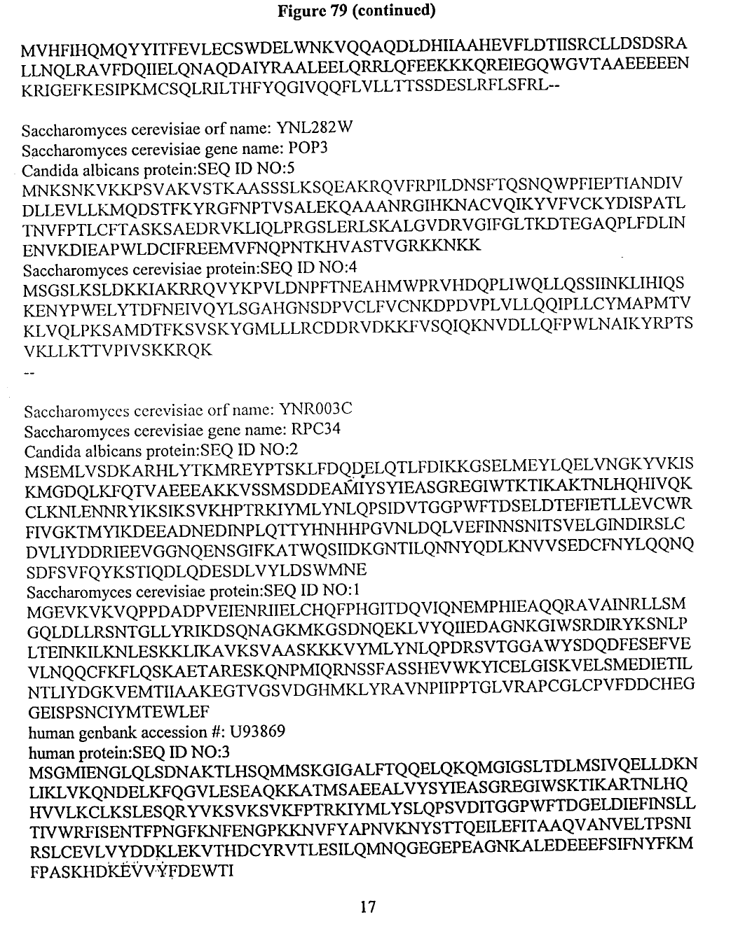

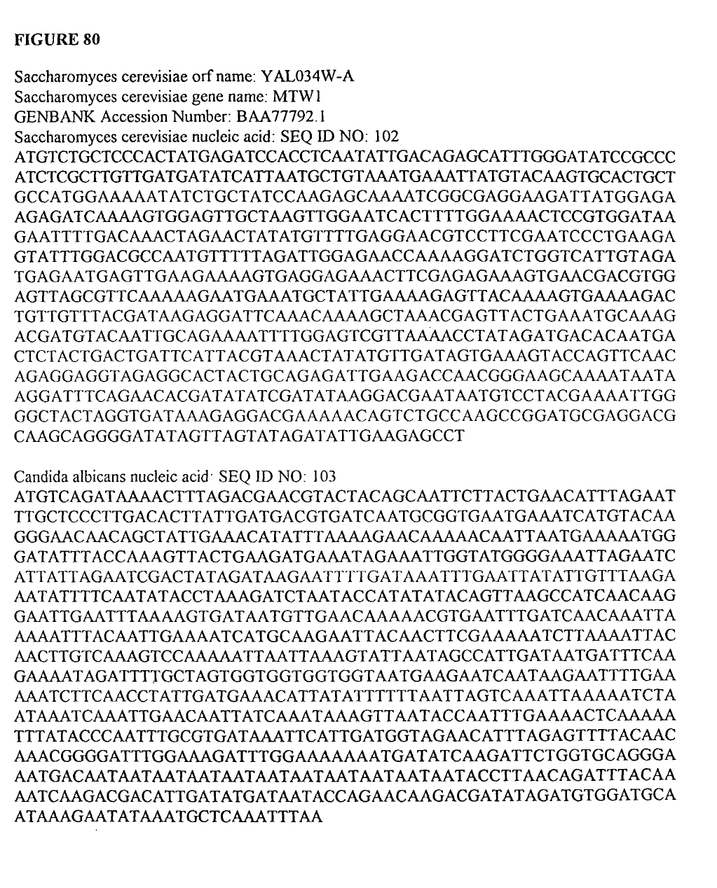

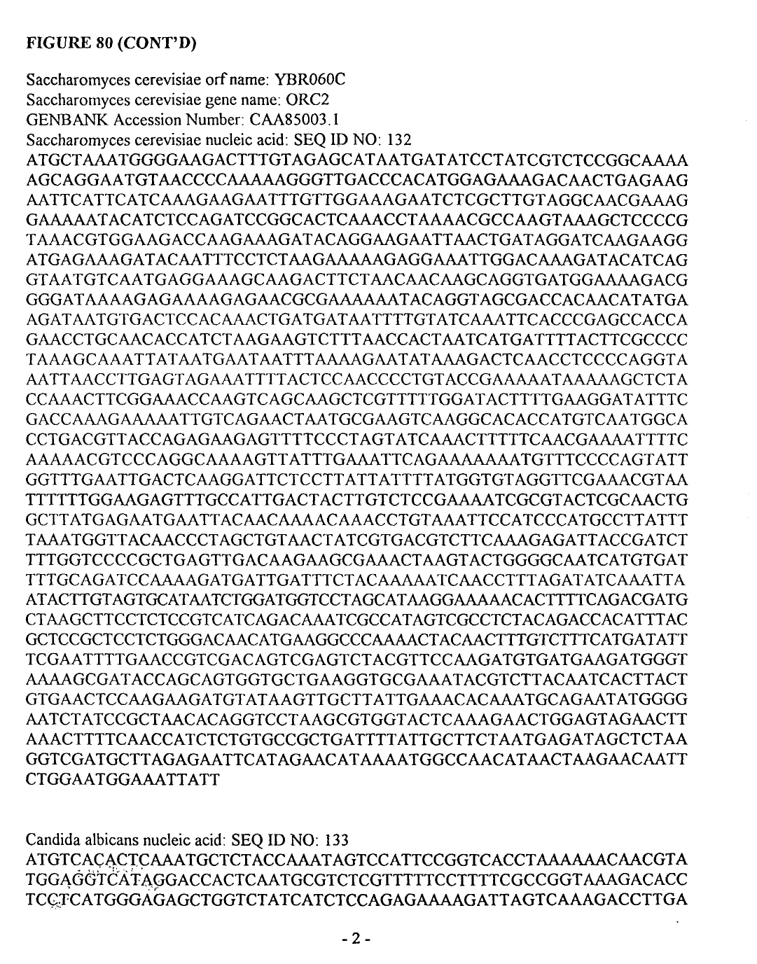

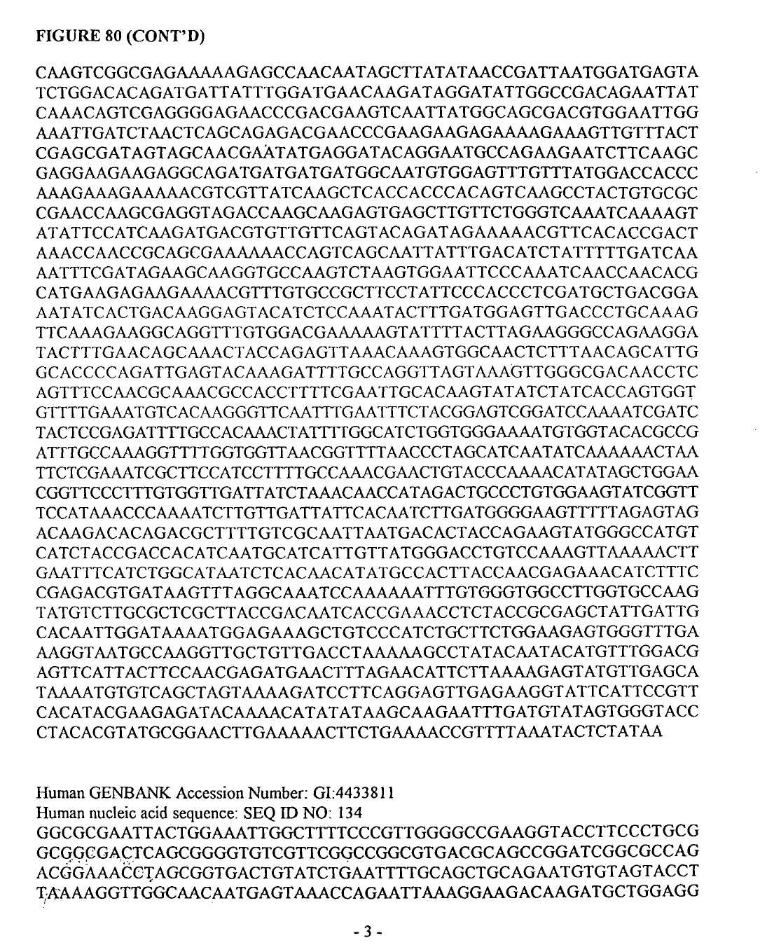

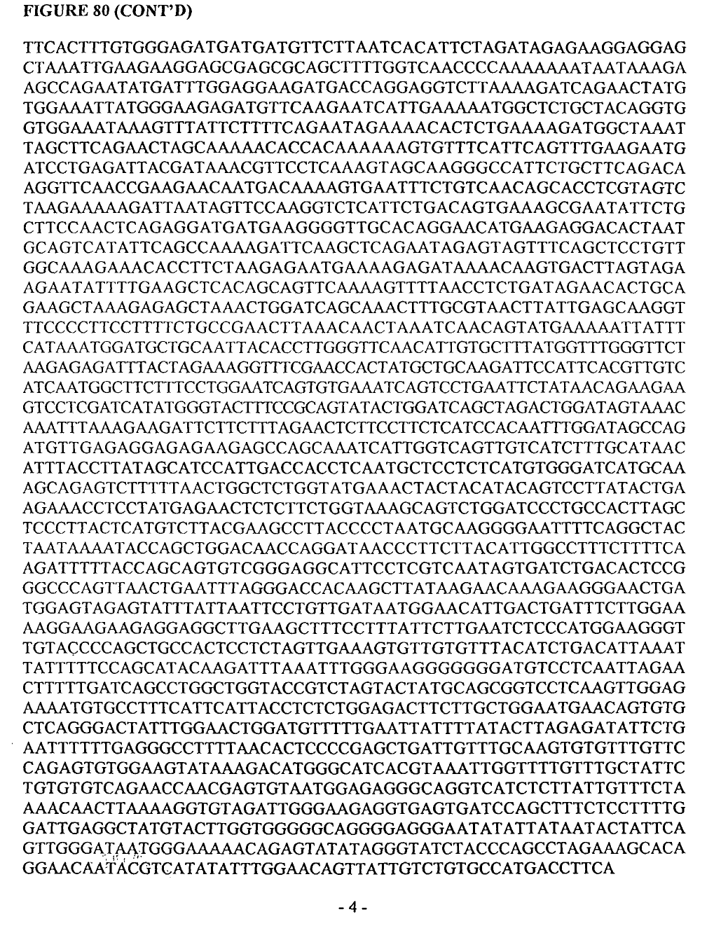

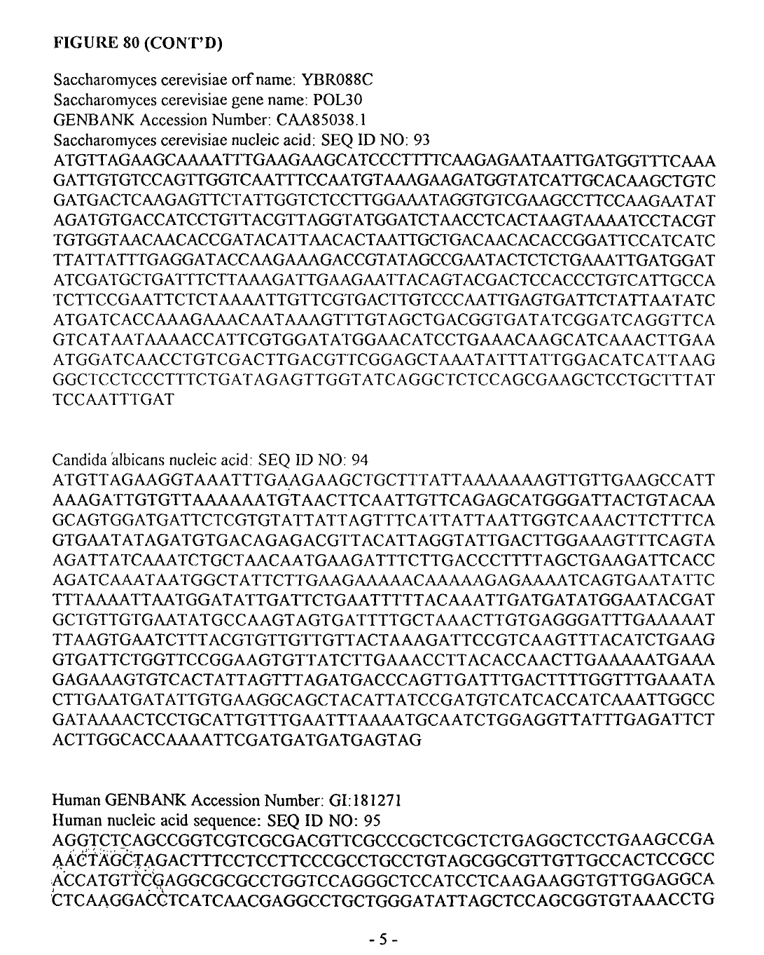

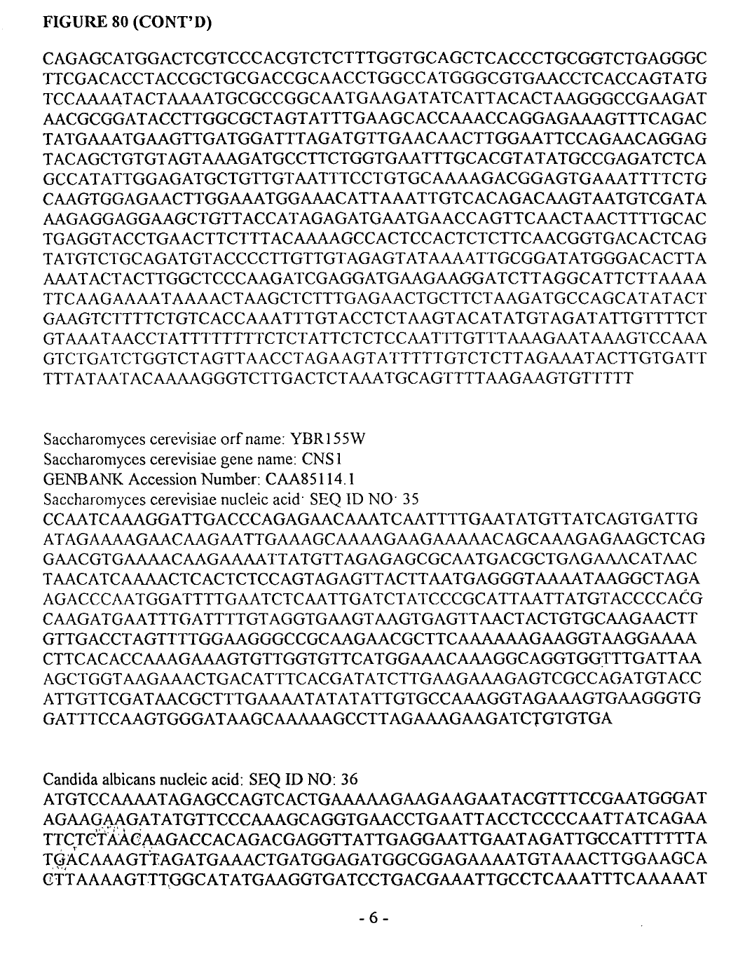

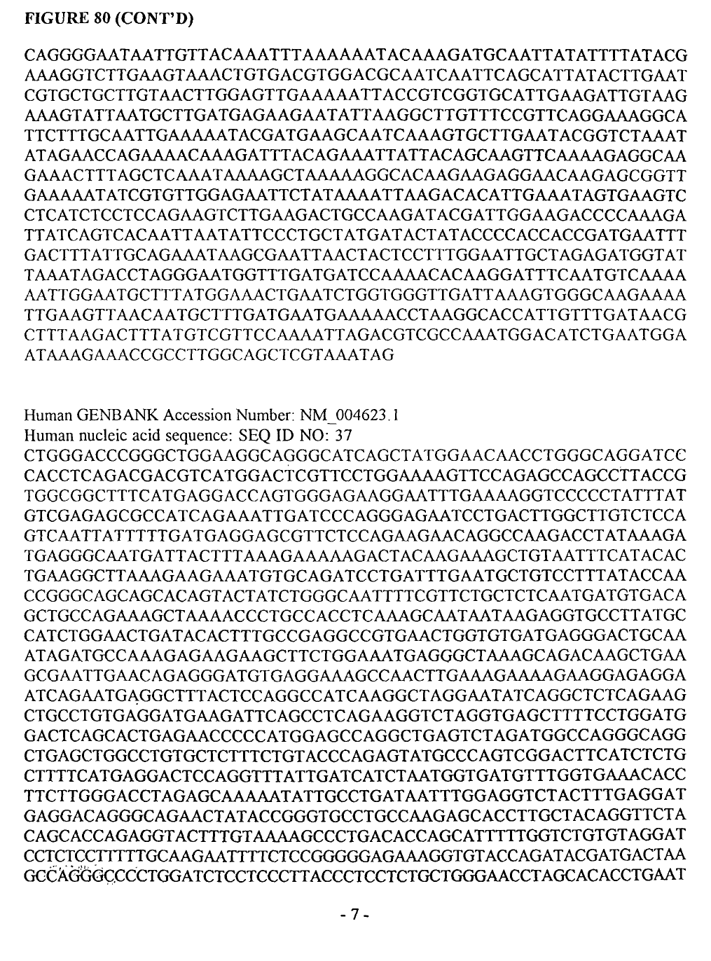

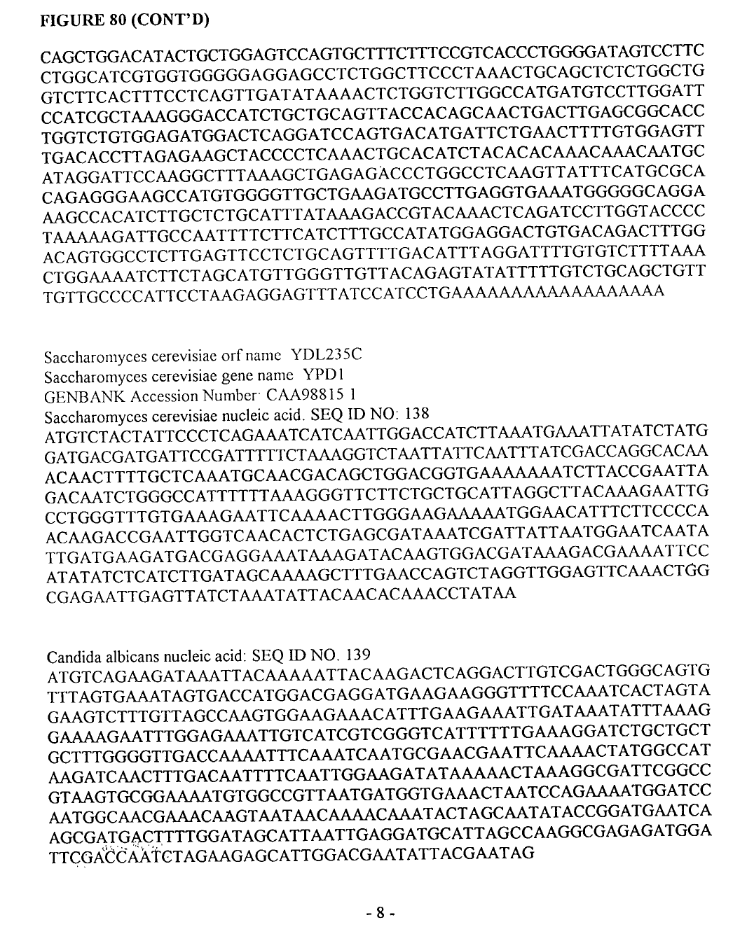

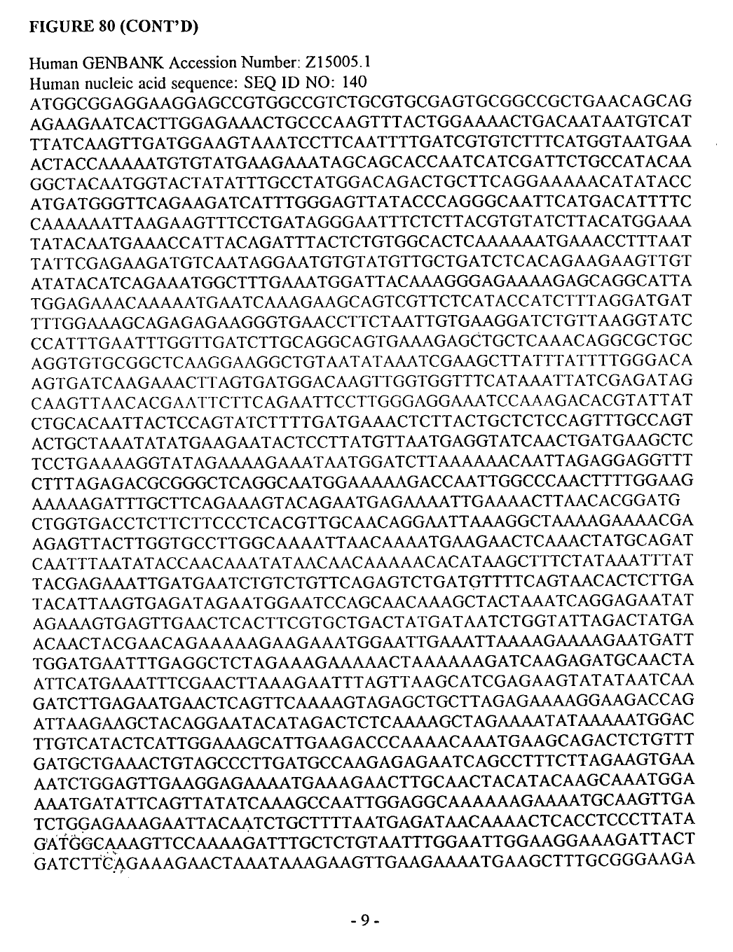

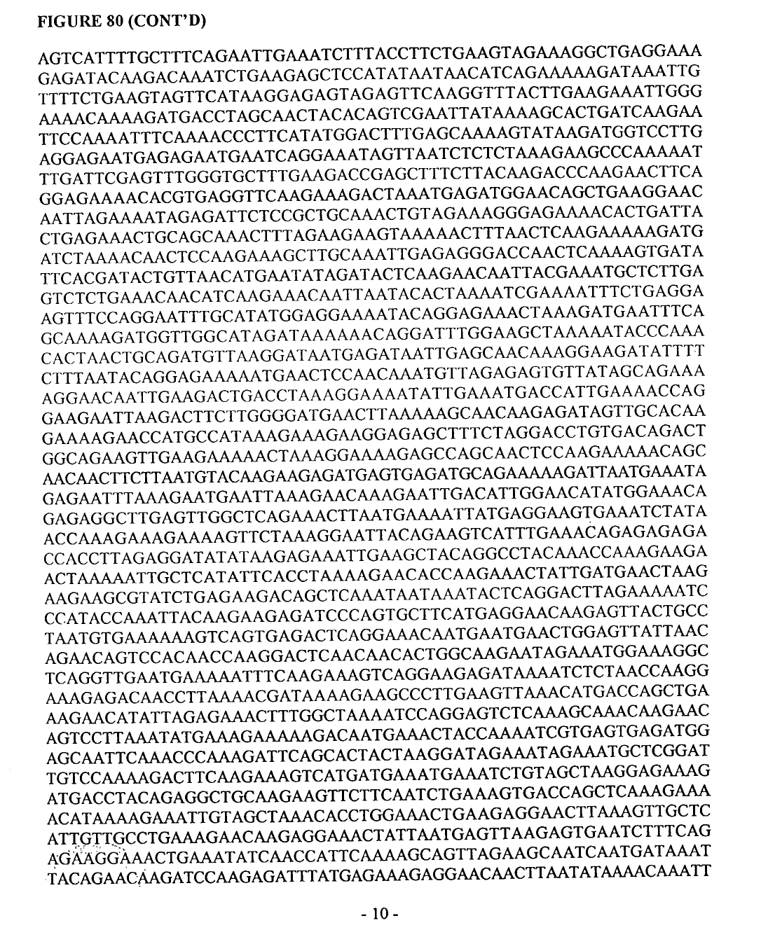

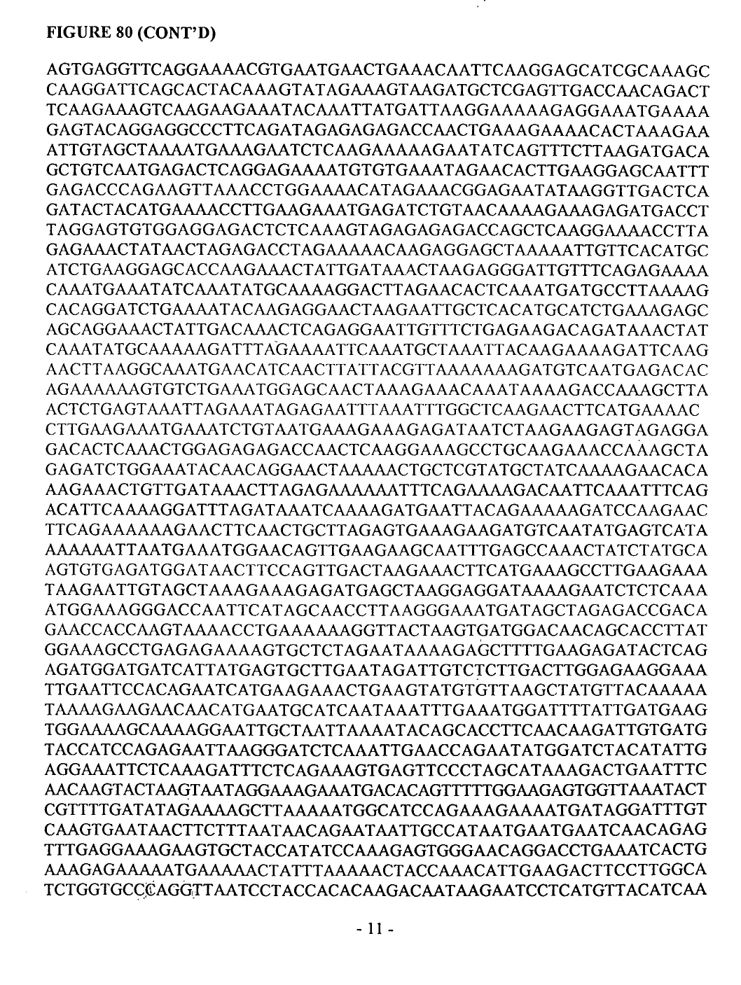

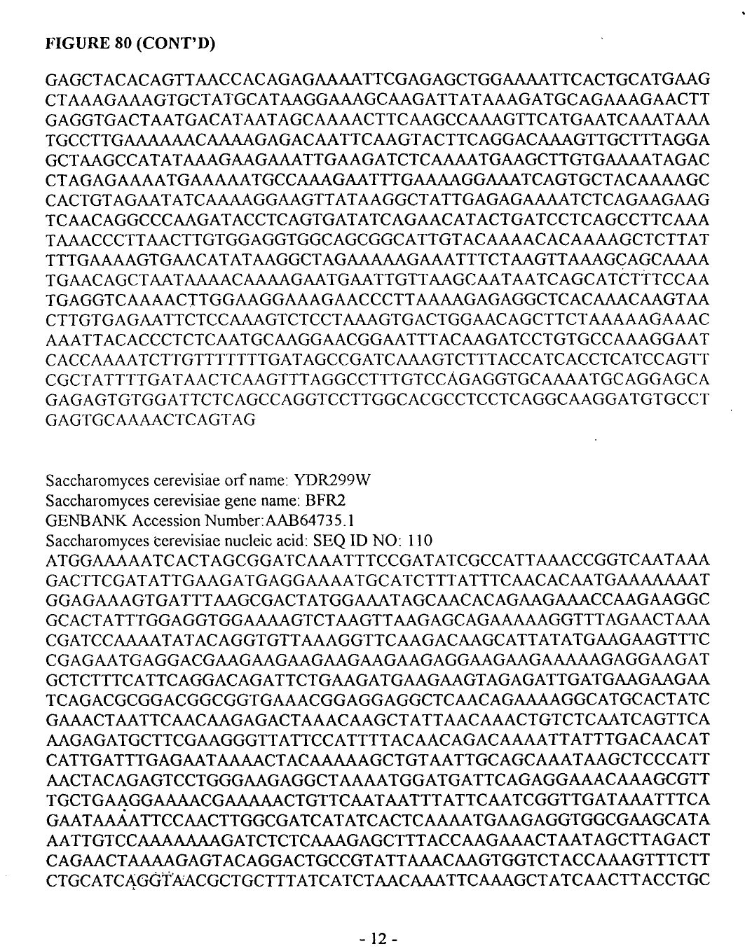

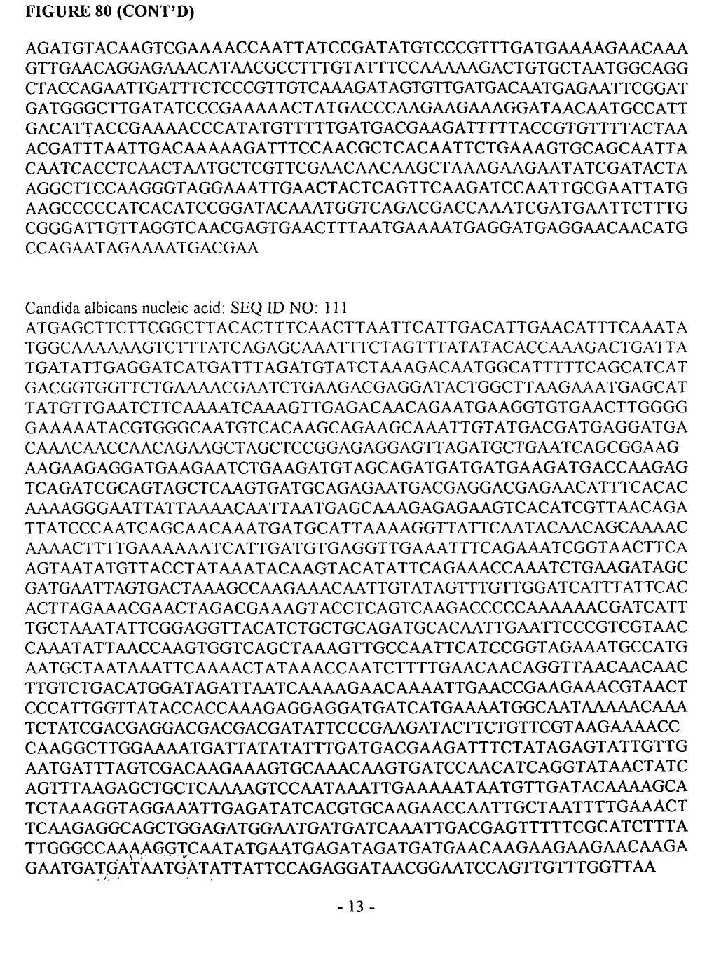

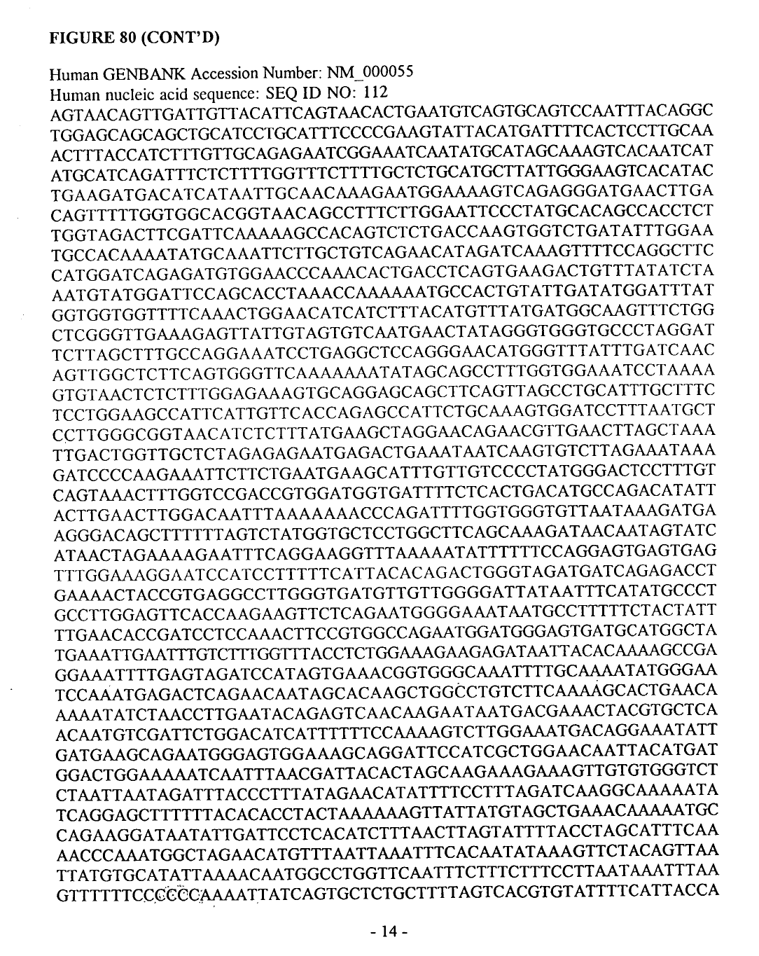

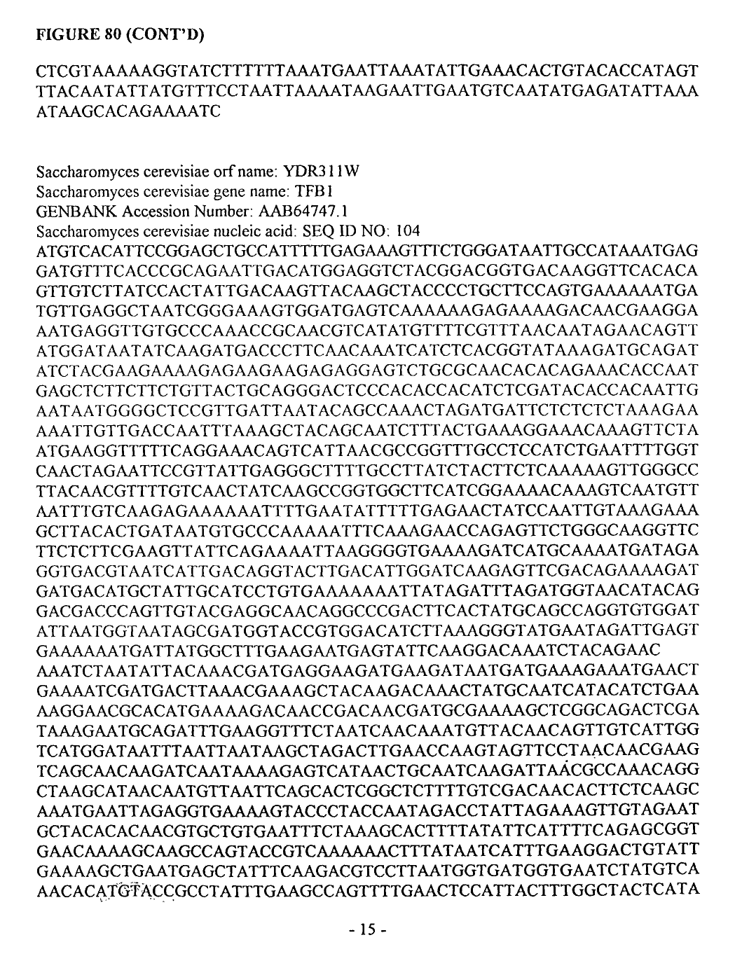

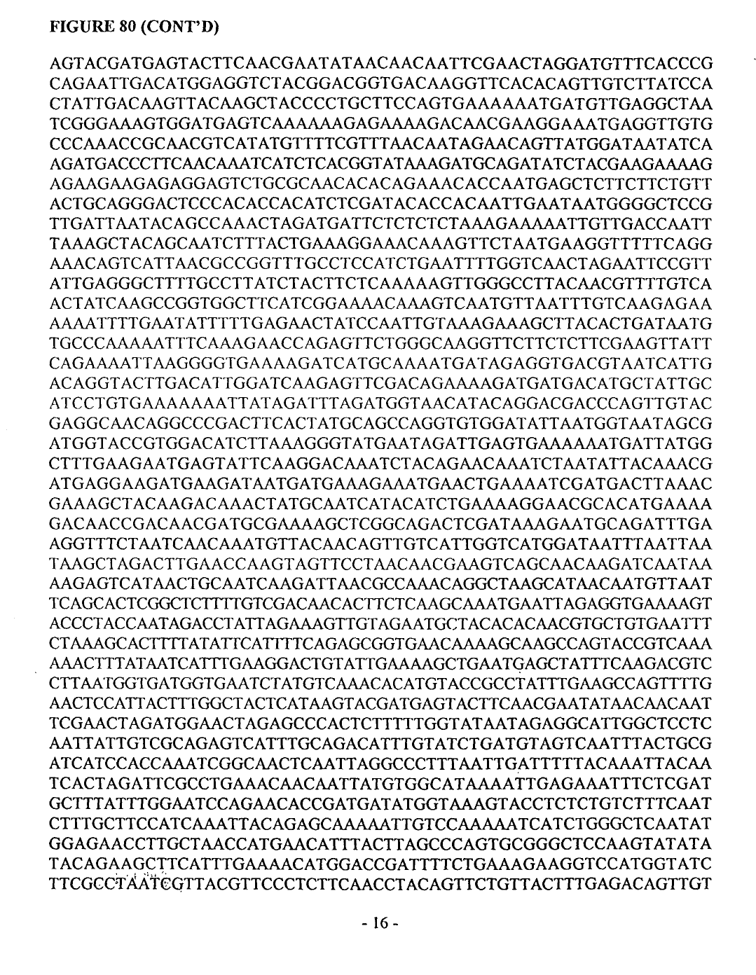

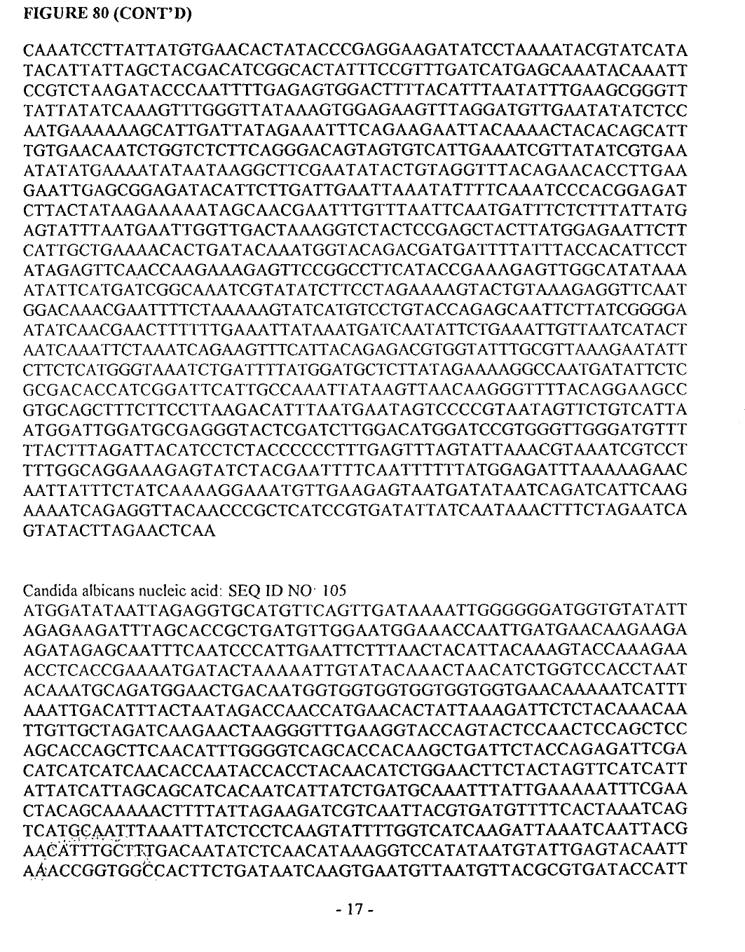

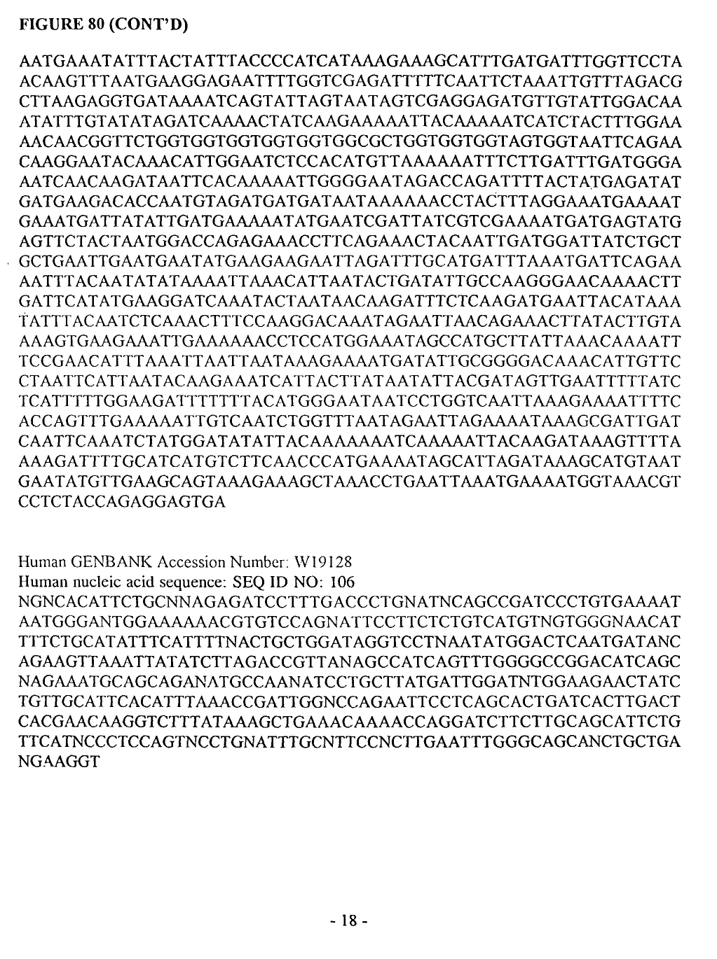

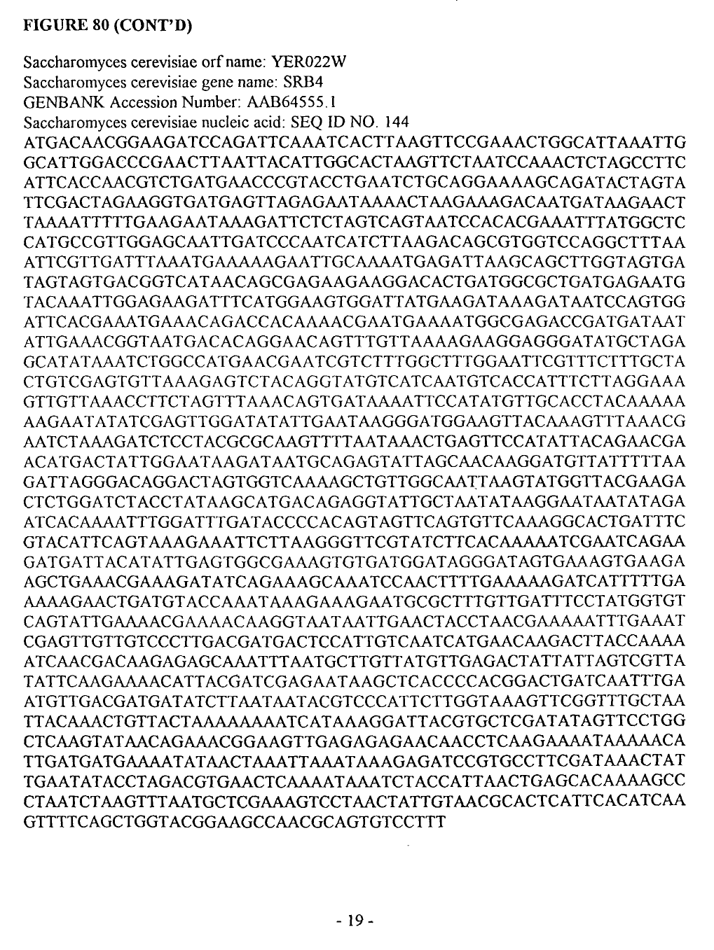

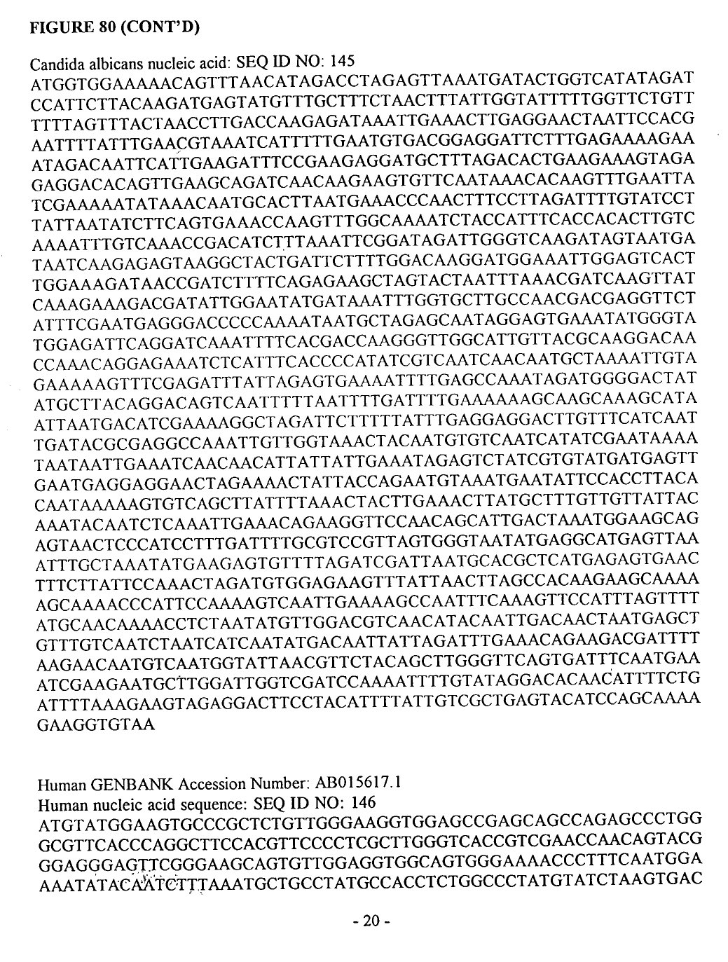

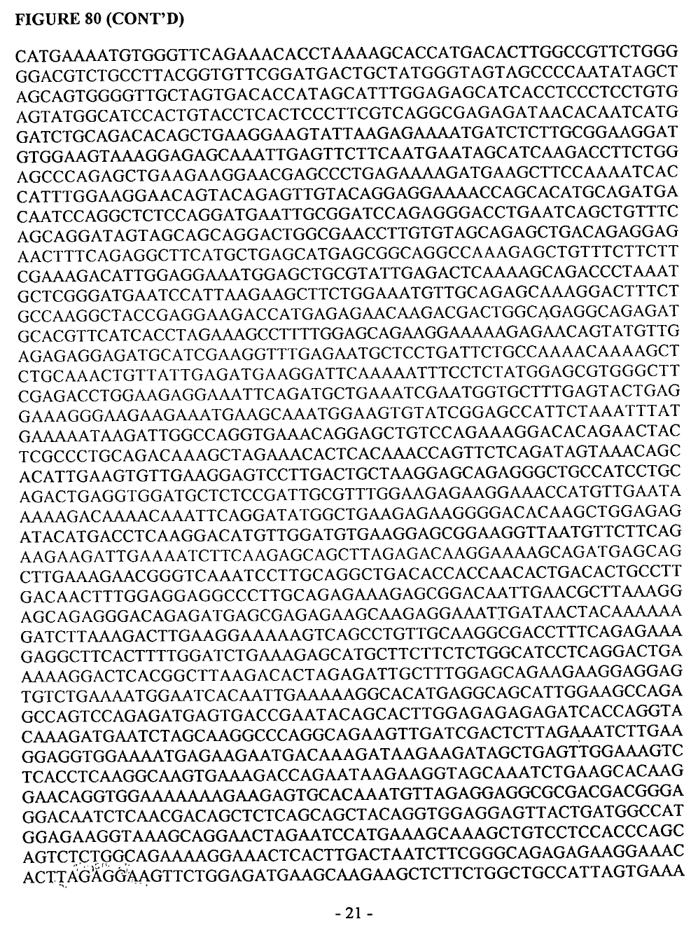

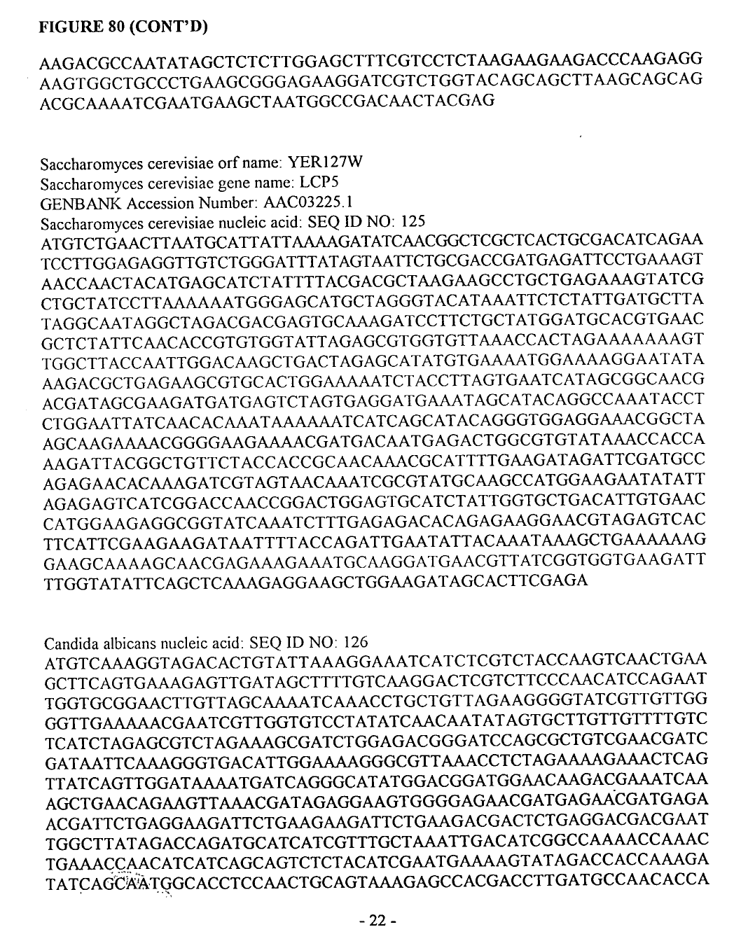

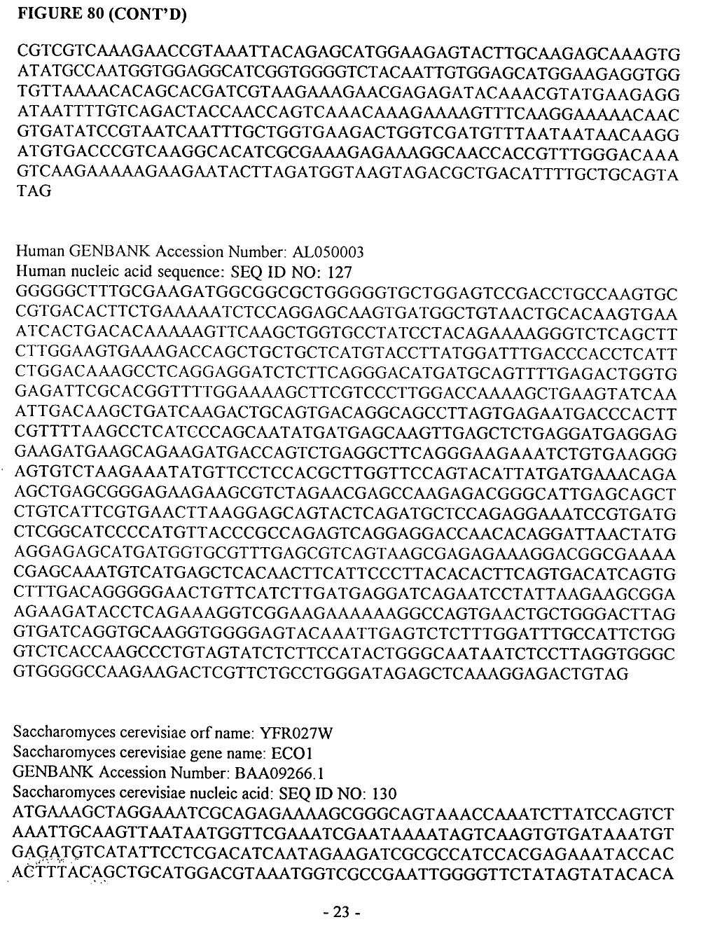

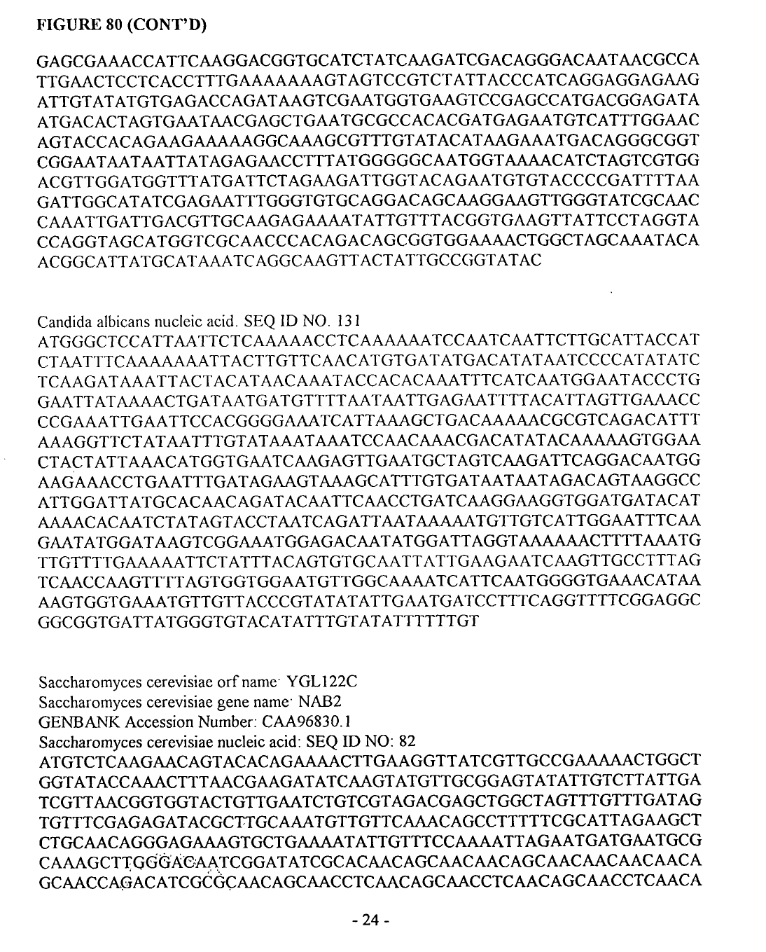

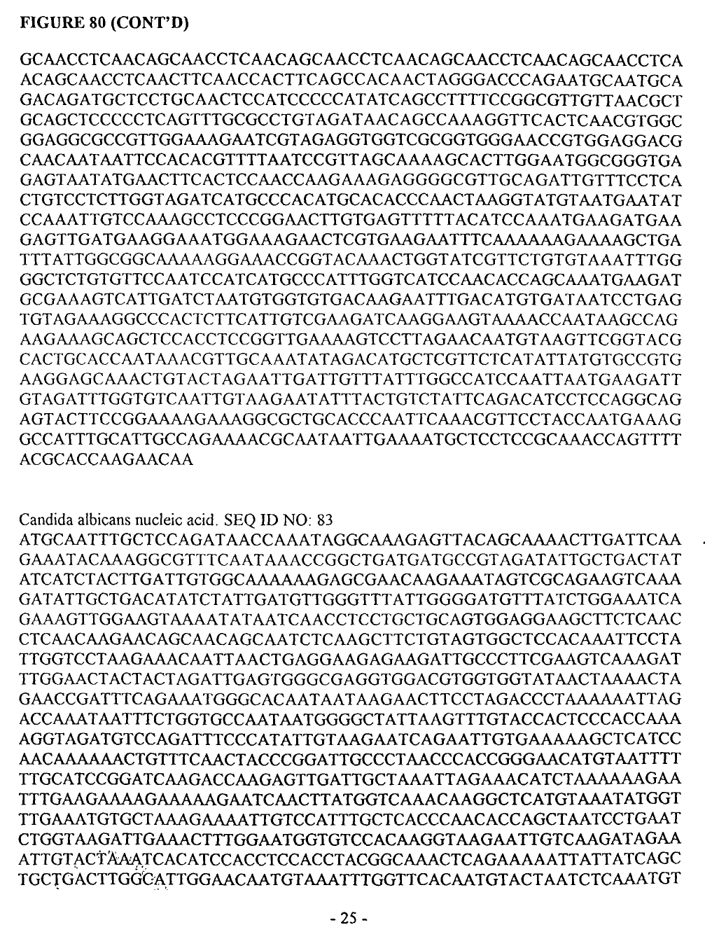

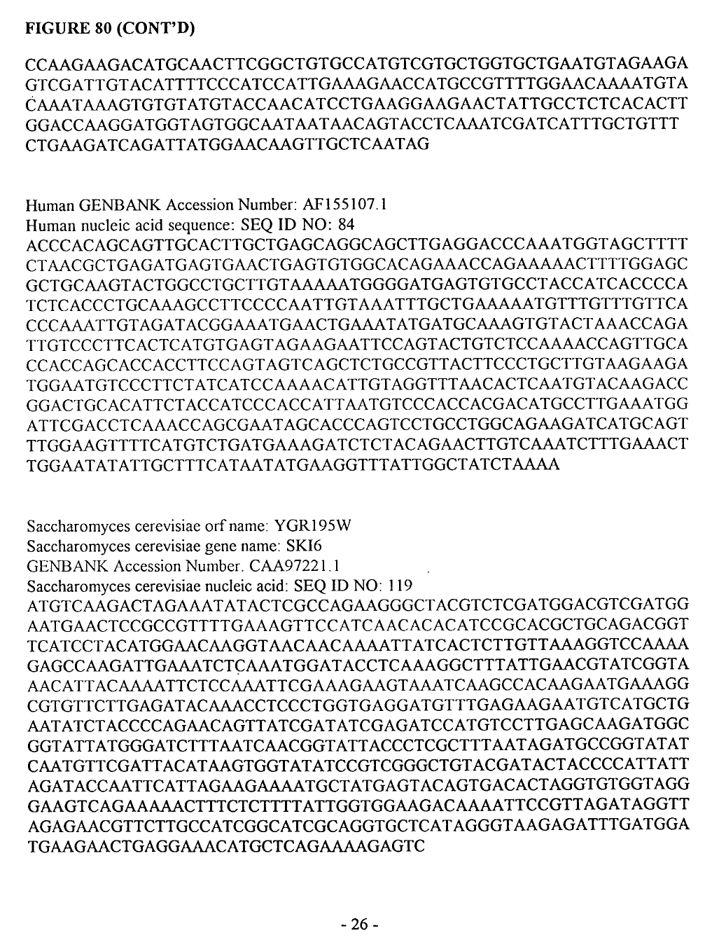

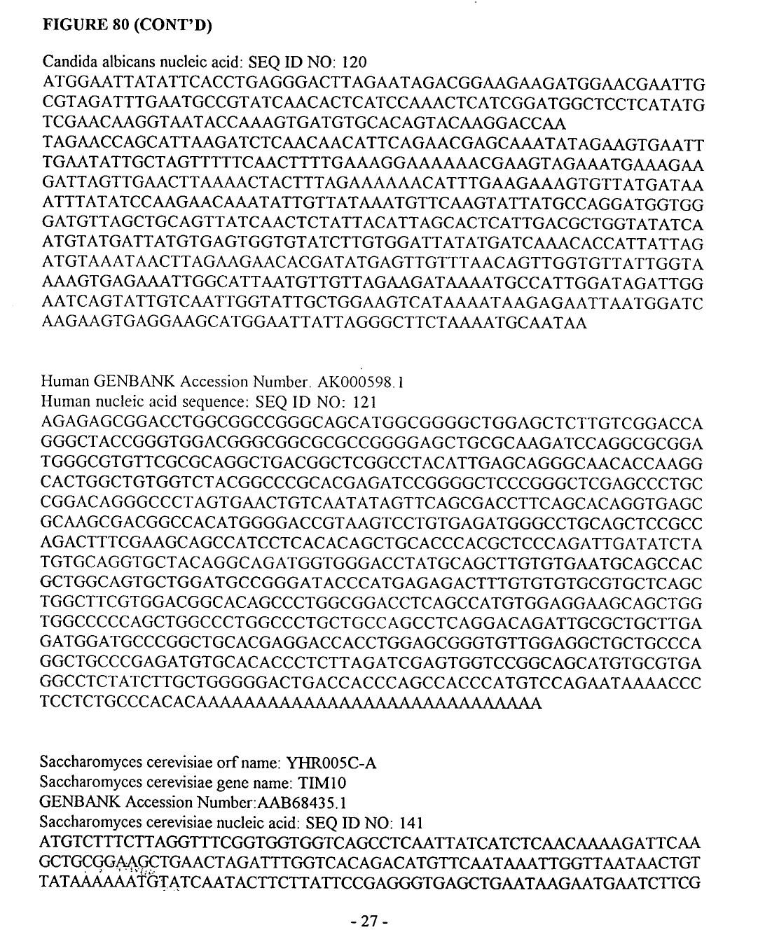

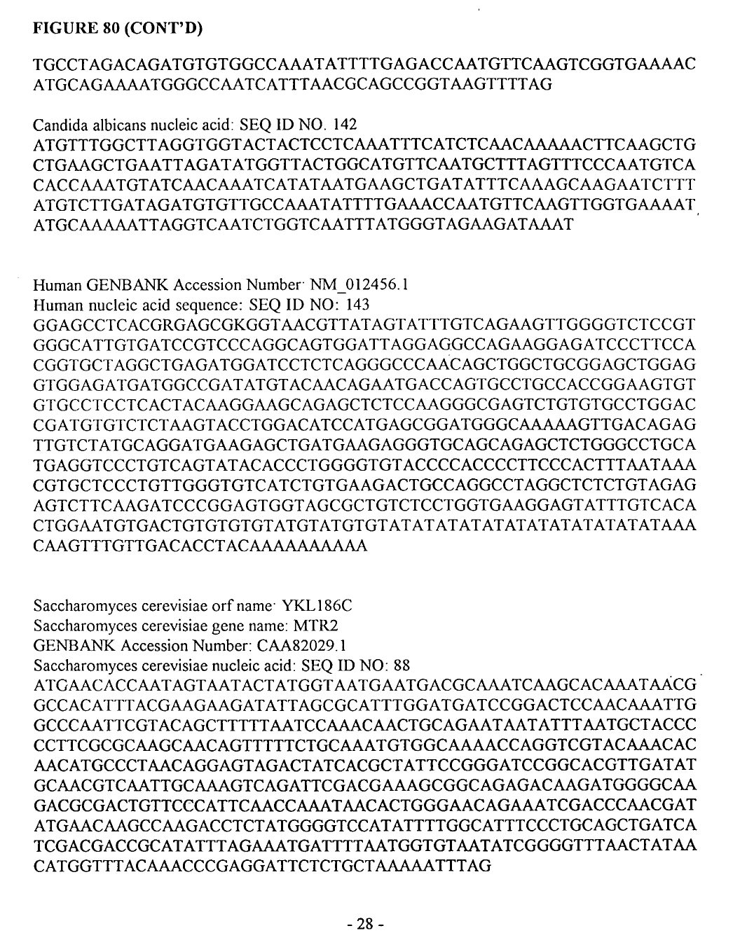

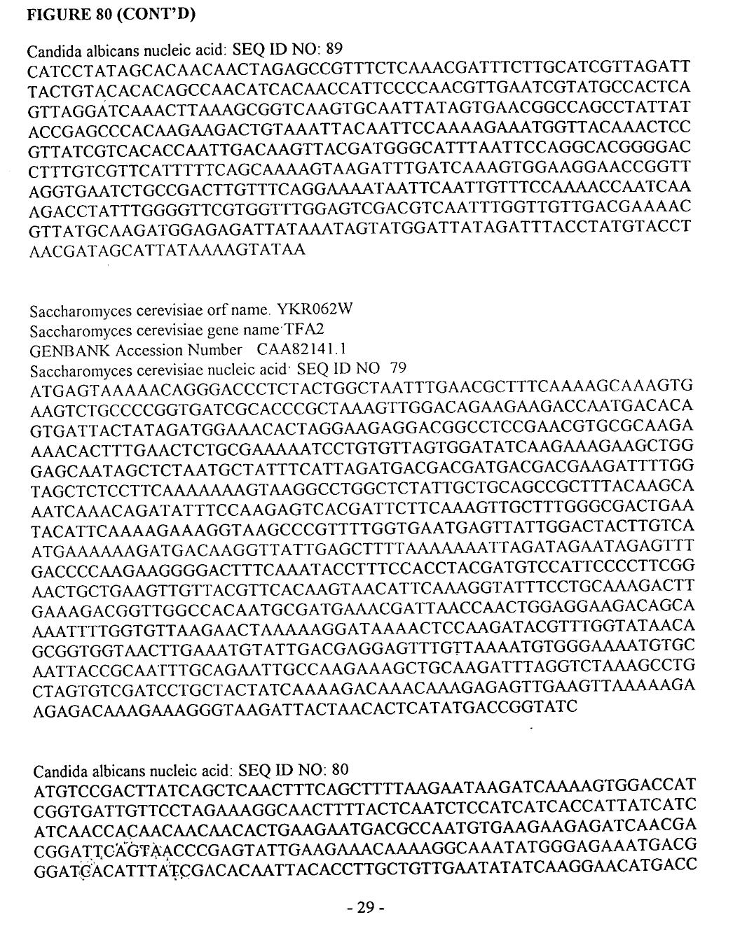

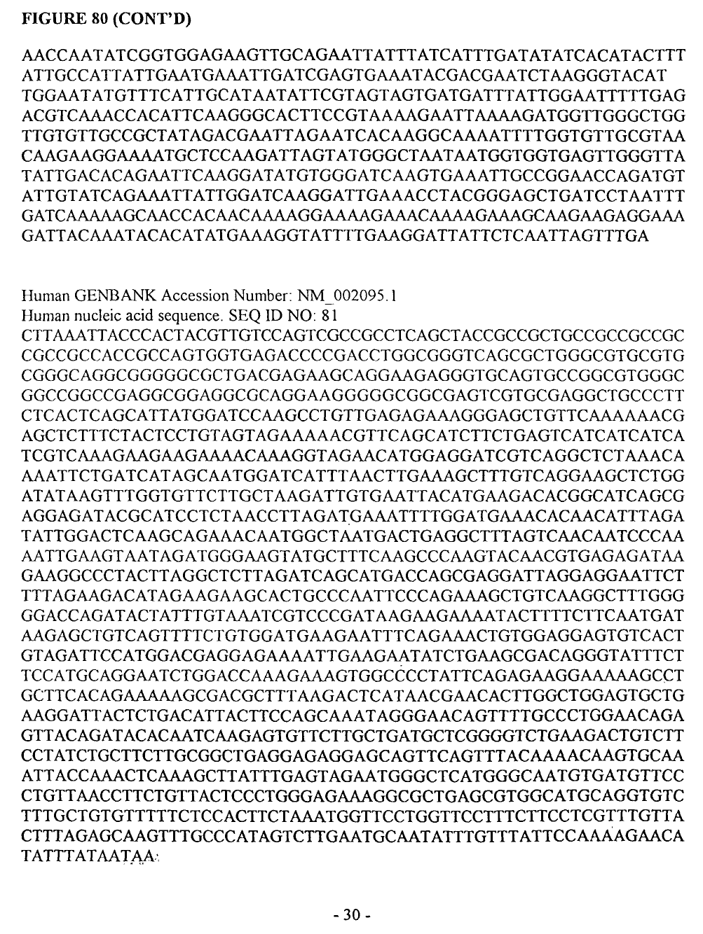

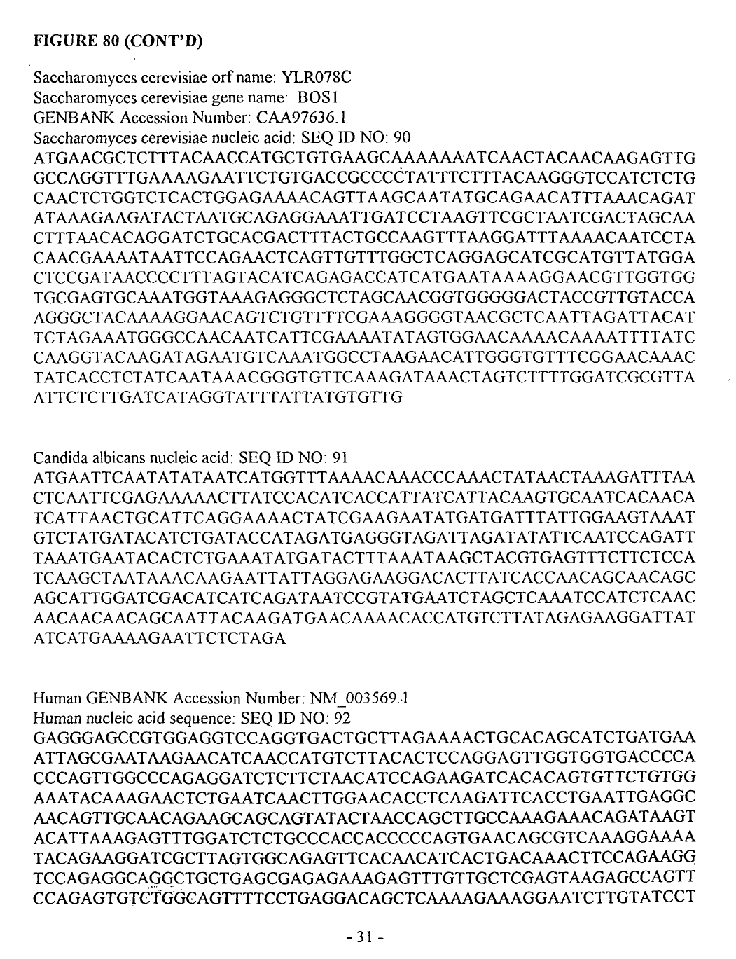

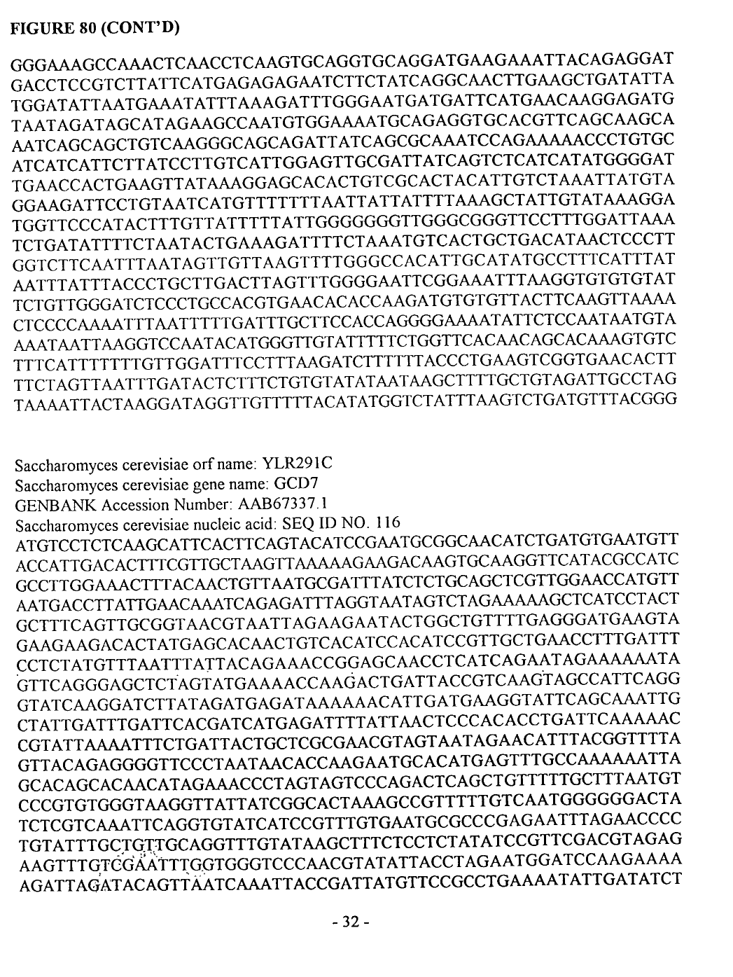

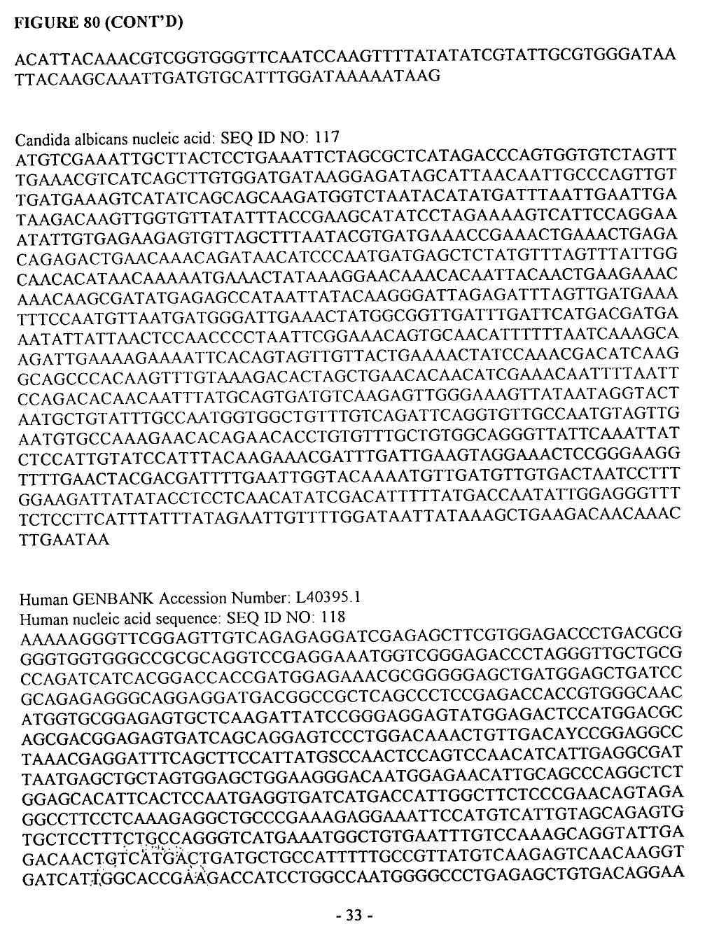

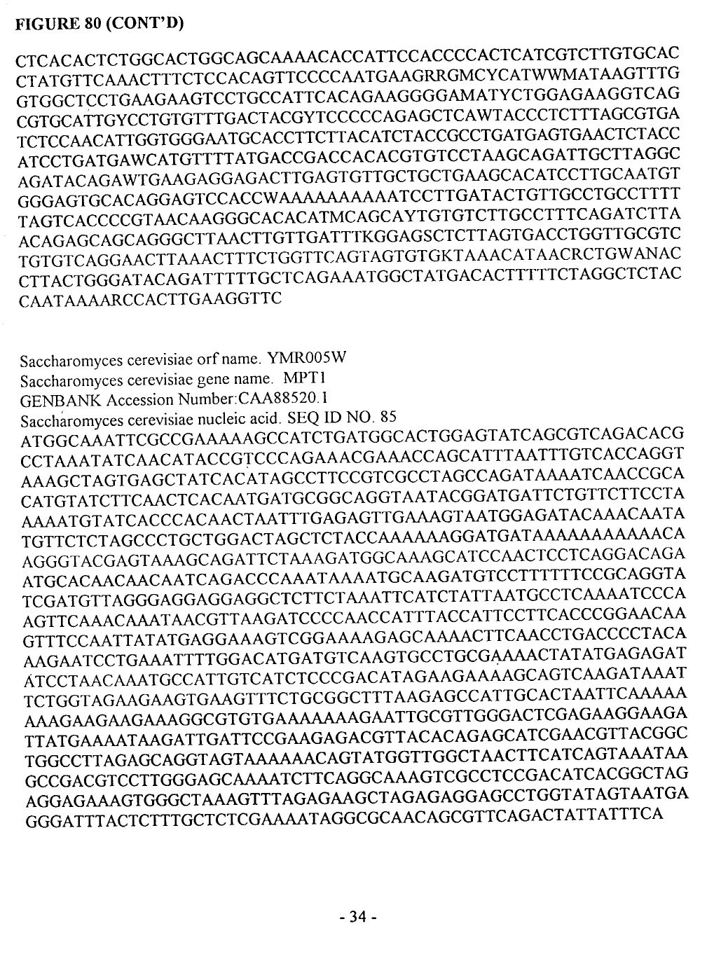

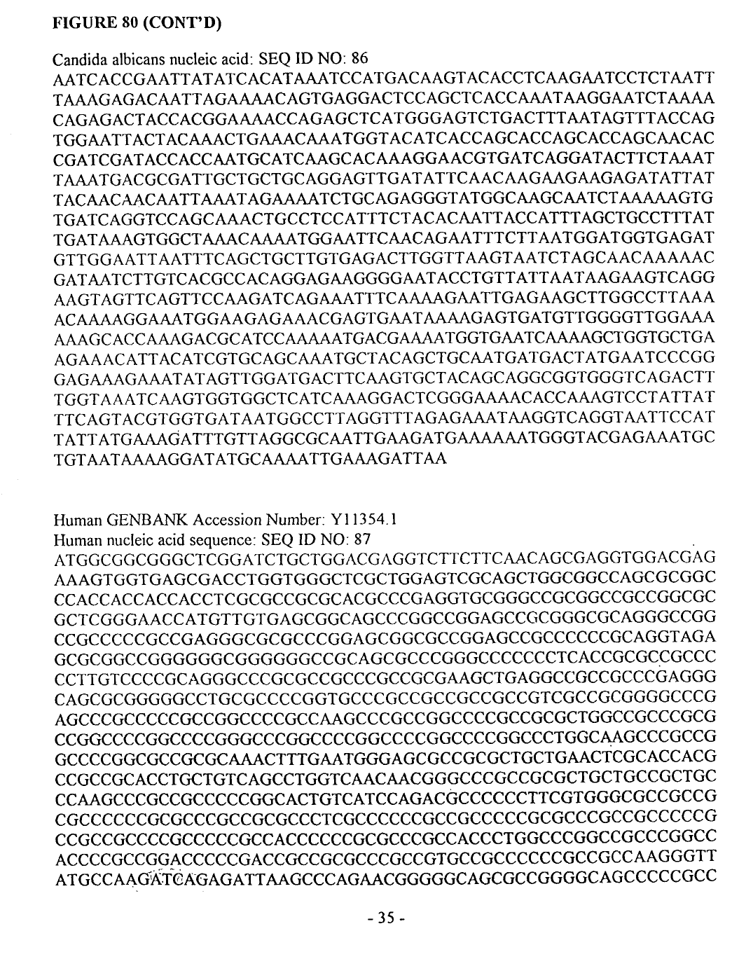

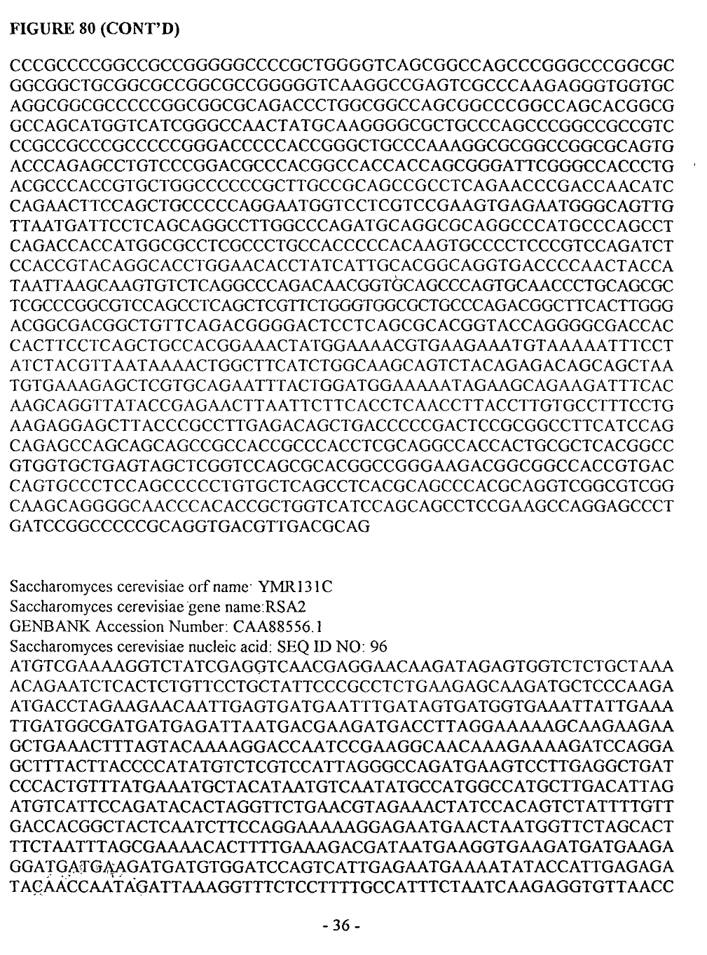

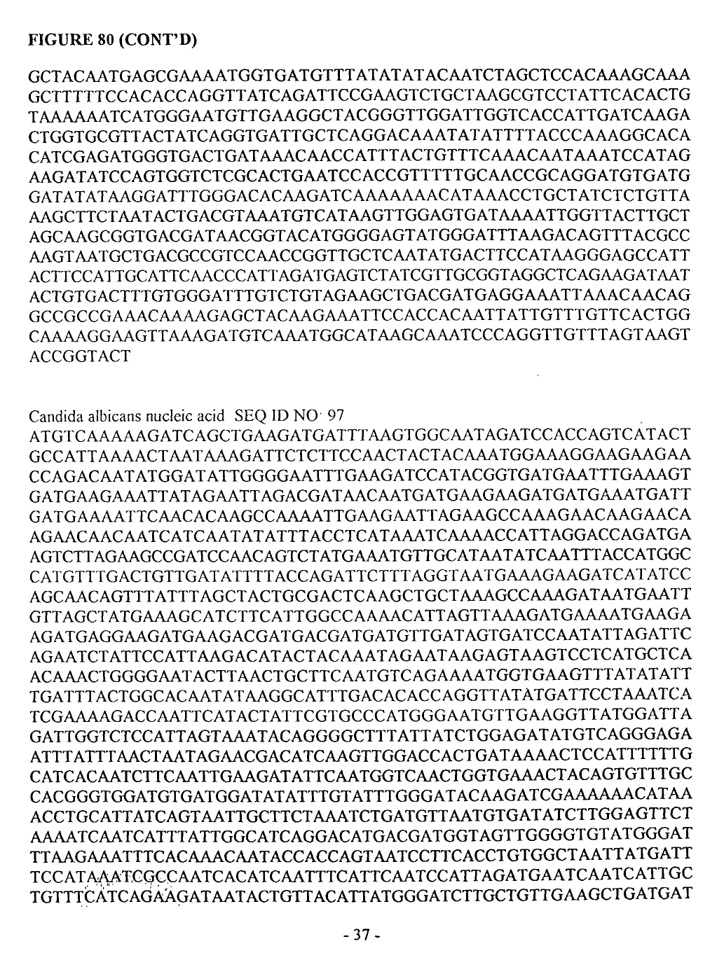

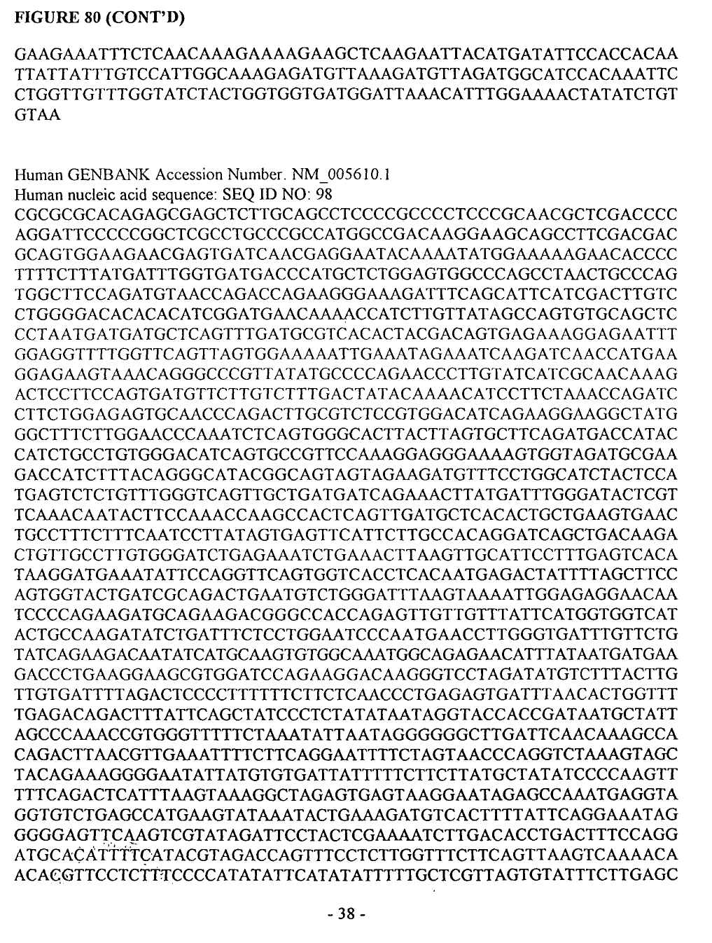

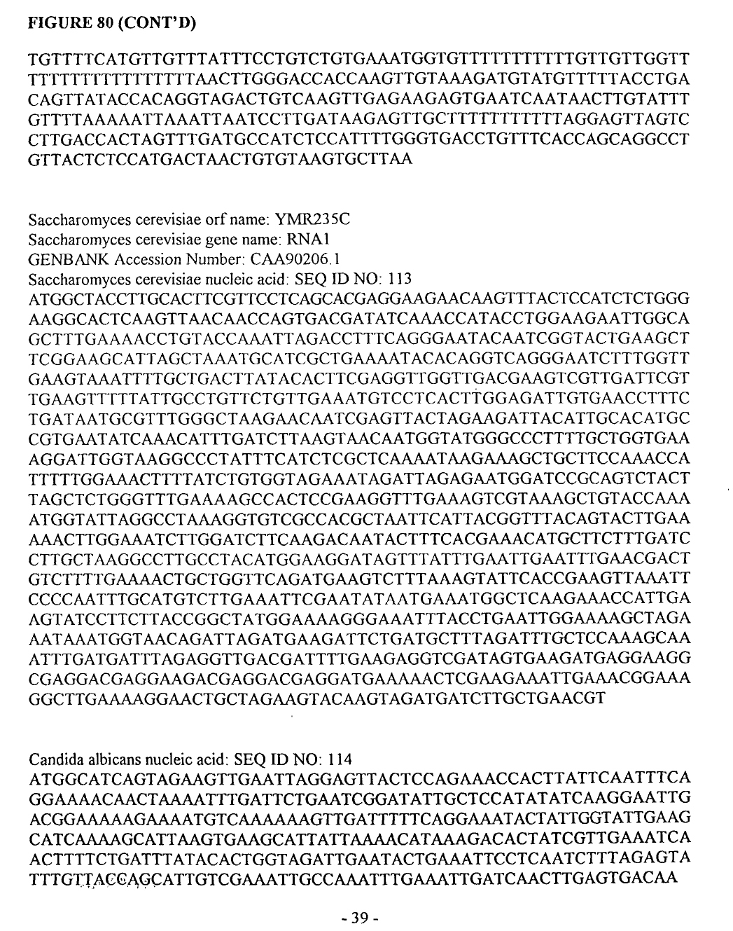

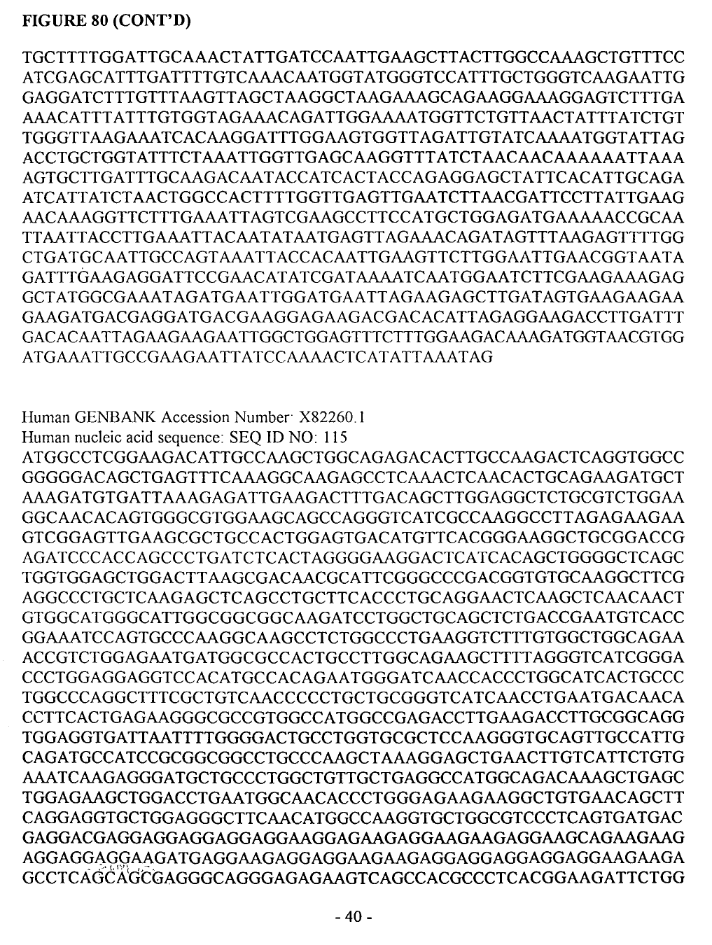

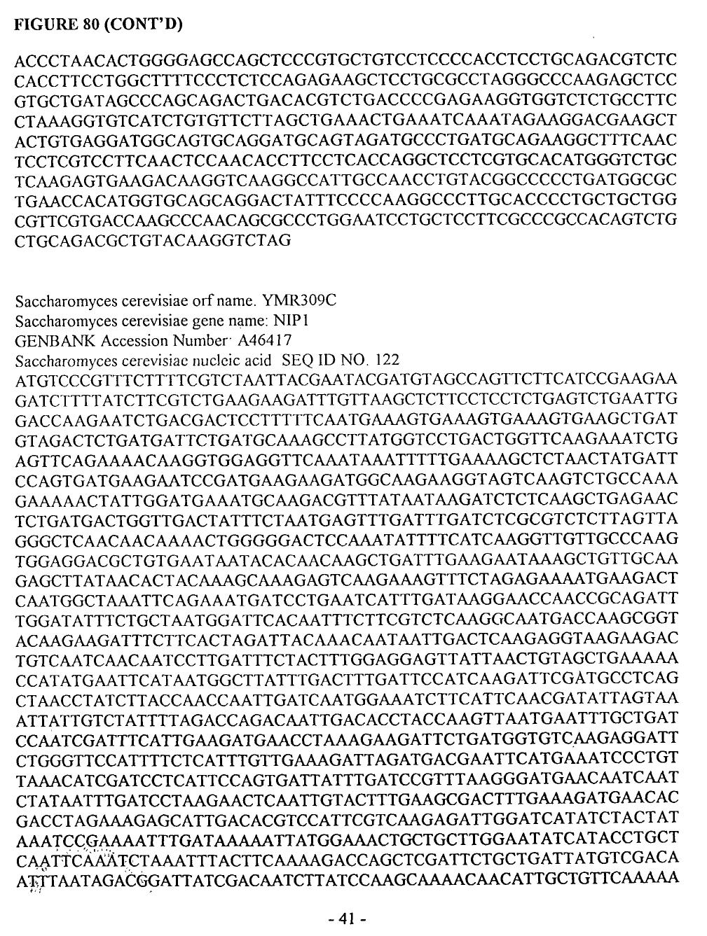

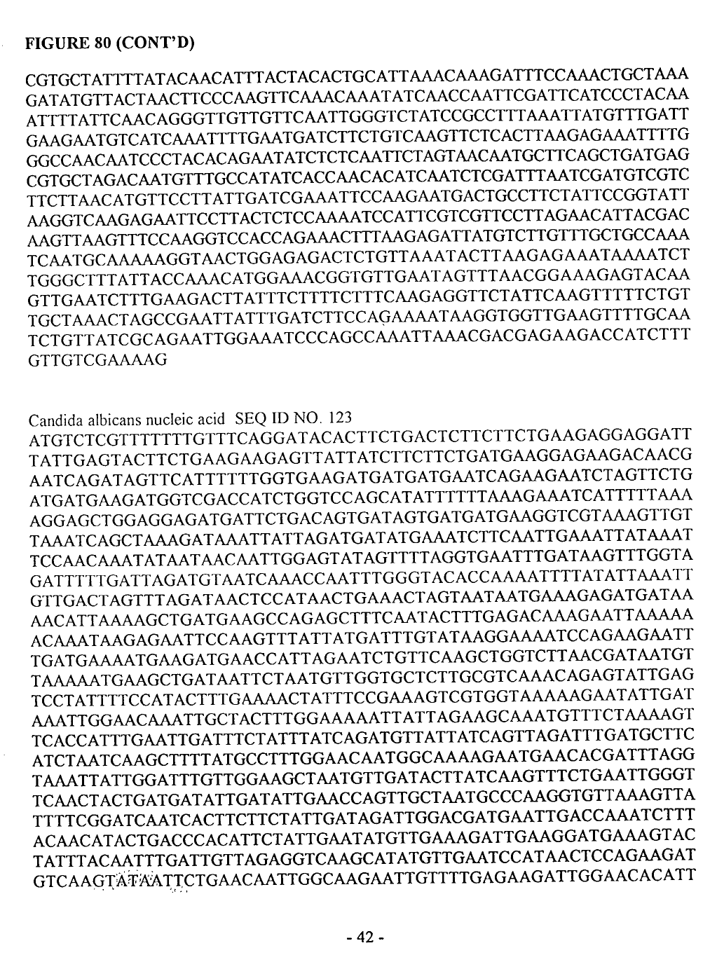

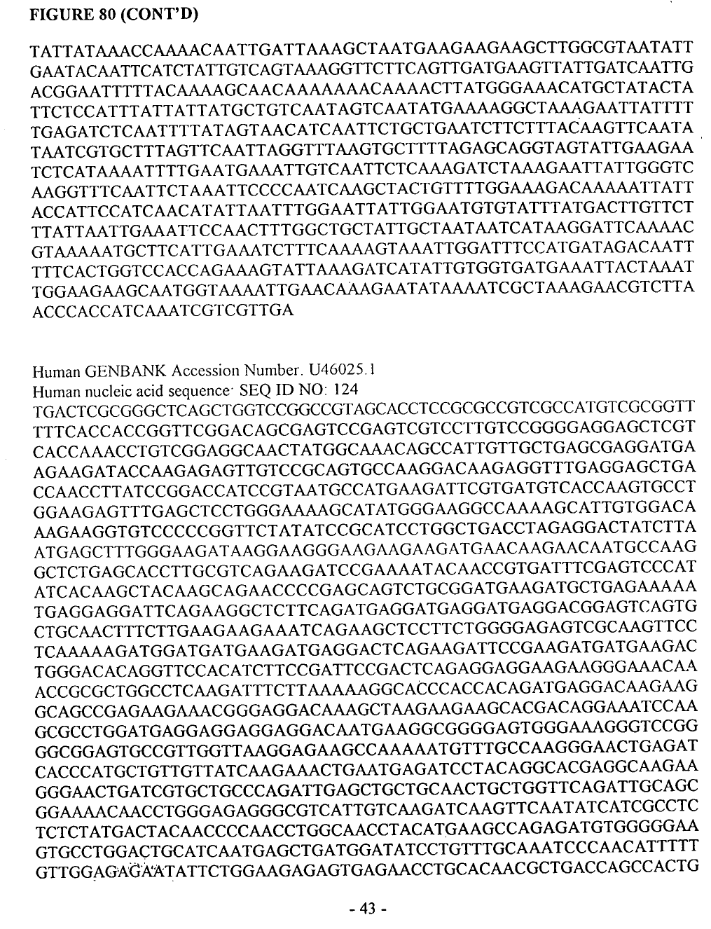

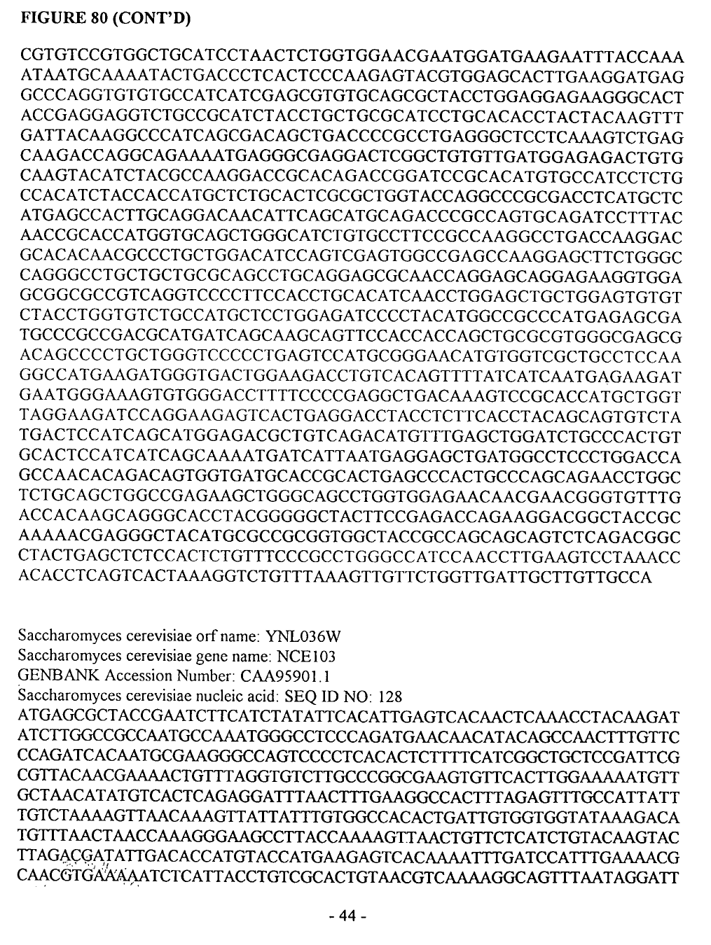

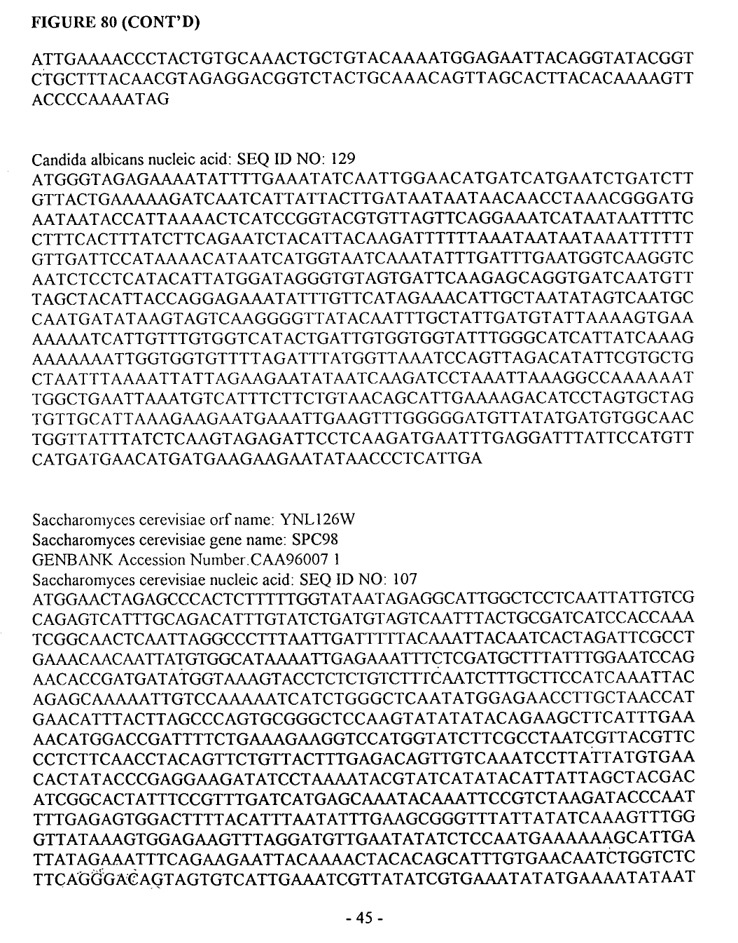

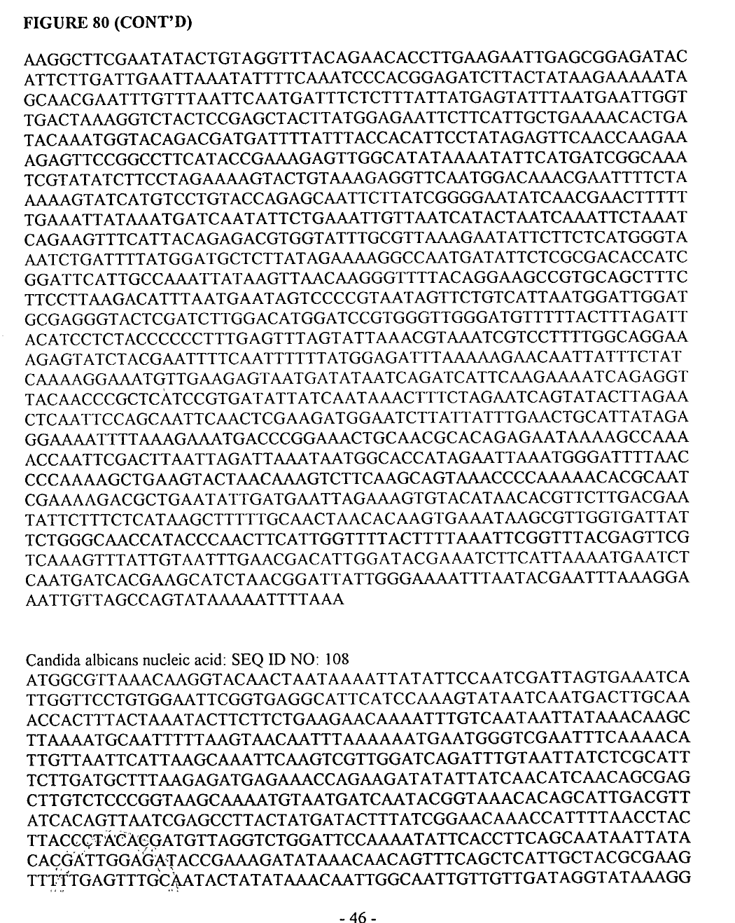

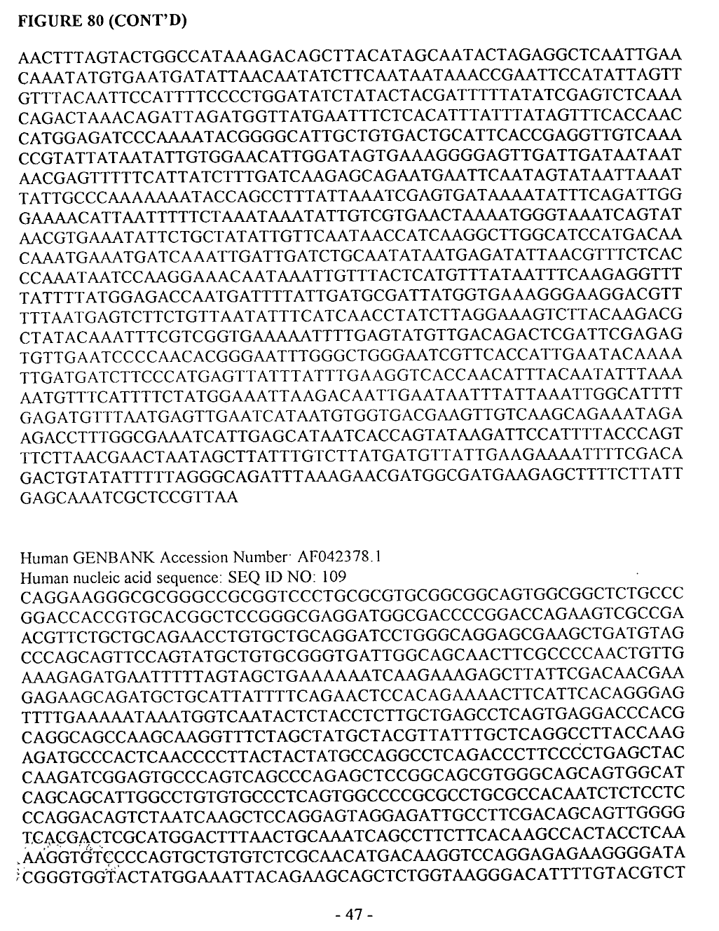

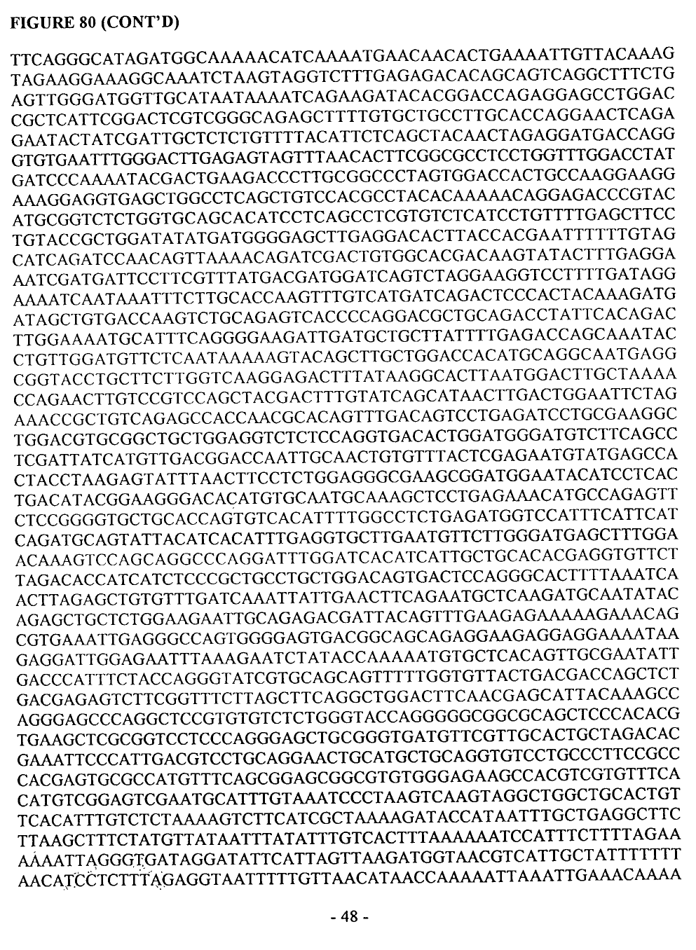

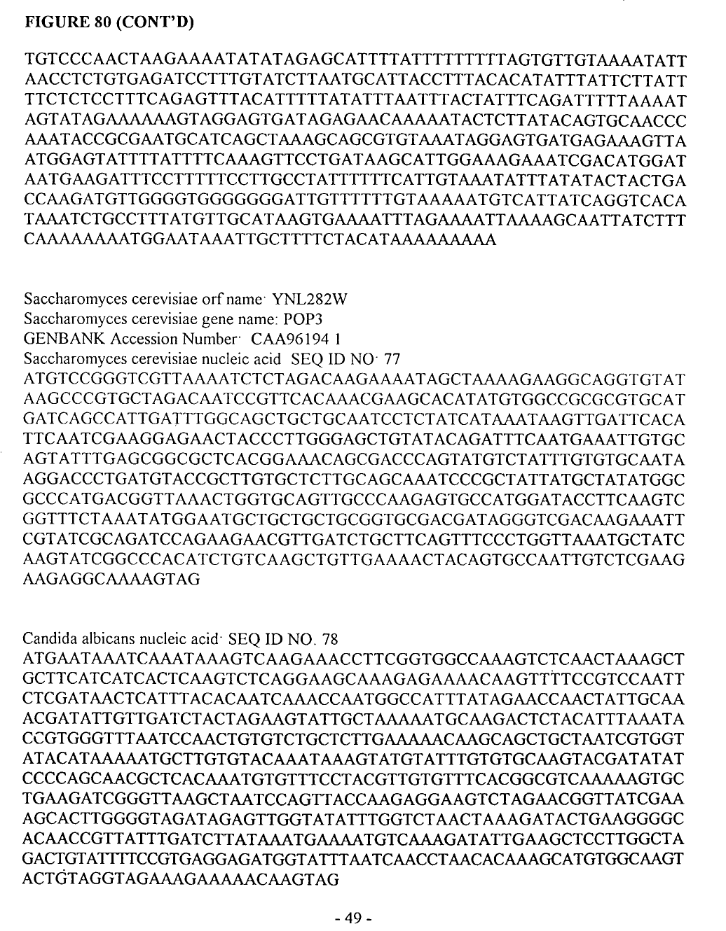

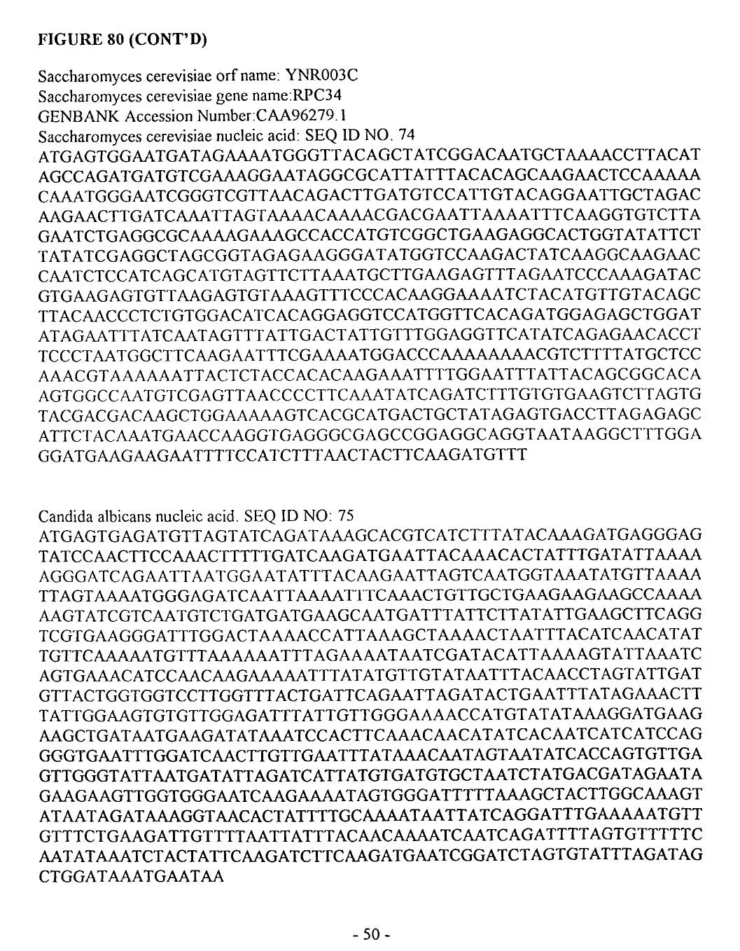

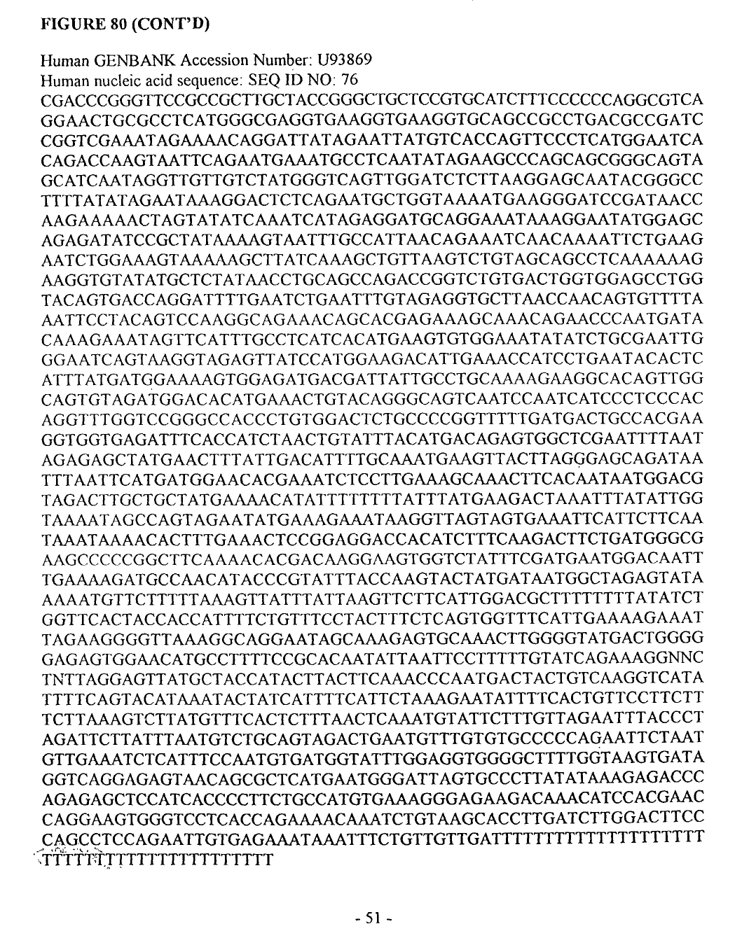

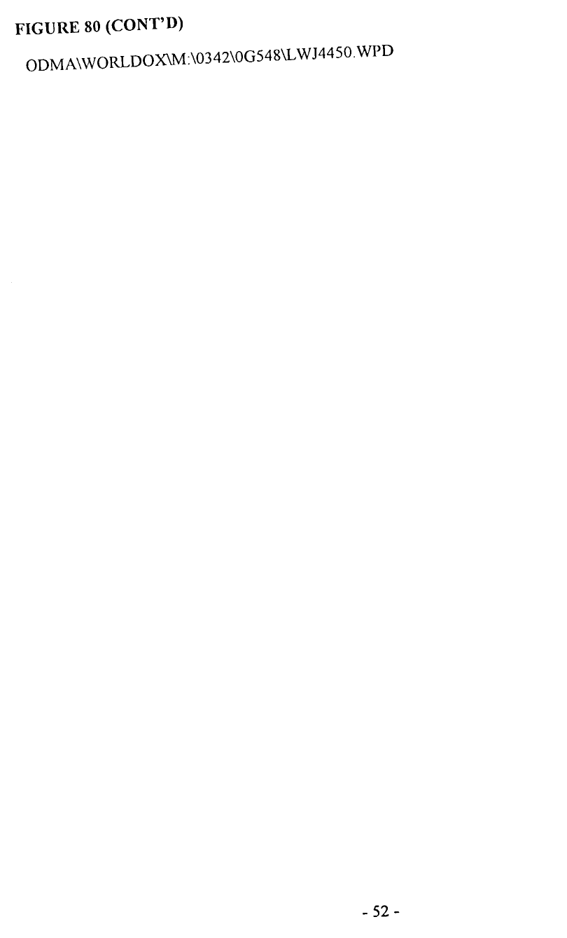

In one embodiment, in order to assess whether the target protein gene is essential for growth in C. albicans, plasmids can be used to construct a double disruptant strain according to the methods outlined in FIGS. 53-78. If a double disruptant strain can be produces, then the gene is determined to be non-essential. Methods used in these constructions employ common techniques employed in the genetic manipulation and screening of C. albicans.

One commonly used approach utilizes C. albicans strain CAI4 (Fonzi and Irwin, 1993) to generate a uridine auxotrophic strain of C. albicans transformed with linearized DNA fragments containing the Ca URA3 gene (able to confer uridine prototrophy upon transformants) flanked by identical HisG sequences. This HisG-CaURA3-HisG cassette is flanked by sequences upstream of the gene of interest on one side and downstream of it on the other side.

Prototrophic transformants have undergone replacement of one copy of the gene of interest with the HisG-CaURA3-HisG cassette. Auxotrophic, uridine requiring derivatives can be isolated by selecting for 5′ fluoro-orotic acid (FOA) resistance in the presence of uridine. The URA3 gene product converts FOA into fluorouracil which is toxic. FOA selection therefore allows one to select cells that have lost the URA3 gene upon cis-recombination of the two identical hisG flanking regions.

To determine if the gene of interest is essential for growth, a second disruption plasmid is used in order to attempt to inactivate the second copy of the gene. The CaURA3 gene, as described above, is able to confer uridine prototrophy upon transformants, and is flanked by identical HisG sequences. This HisG-CaURA3-HisG cassette is flanked by sequences upstream of the gene of interest on one side and downstream of the gene of interest on the other side. Generation of prototrophic transformants can occur by integration of the cassette into the non-disrupted allele of the gene of interest, by replacement of the hisG cassette with the CaURA3 cassette, or by non-homologous recombination events. Transformants that disrupt the second copy of the gene is proof that the gene of interest is not essential. In order to establish that a gene in C. albicans is essential for growth, at least 20 second round transformants should be analyzed. If analysis of 20 transformants demonstrates that the second copy of the gene is still present, this indicates that the gene is essential. All transformants are analyzed by Southern blotting. Candida albicans transformations are performed as described (Elble R., Biotechniques 1992; 13:18-20).

A second commonly used approach utilizes C. albicans strain CAI8 (Fonzi and Irwin, 1993). CAI8 is a uridine and adenine auxotrohic strain that can be converted to uridine and adenine prototophy by transformation with C. albicans URA3 (CaURA3) and C. albicans ADE2 (CaADE2), respectively.

Deletion of the first allele of the gene of interest is accomplished by transformation of CAI8 to adenine prototophy with a linearized DNA fragment containing the CaADE2 gene flanked by sequences upstream of the gene of interest on one site and downstream of it on the other site.

To determine if the gene of interest is essential for growth, a second disruption plasmid is used in order to attempt to inactivate the second copy of the gene. The CaURA3 is flanked by sequences upstream of the gene of interest on one site and downstream of it on the other site. Generation of adenine/uridine prototrophic transformants can occur by integration of the cassette into the non-disrupted allele of the gene of interest, or by non-homologous recombination events.. Transformants that disrupt the second copy of the gene is proof that the gene of interest is not essential. In order to establish that a gene in C. albicans is essential for growth at least 20 second round transformants should be analyzed. If analysis of 20 transformants shows that the second copy of the gene is still present and could not be deleted, which indicates that the gene is essential. All transformants are analyzed by Southern blotting. Candida albicans transformations are performed as previously described (Elble, 1992).

URA3 can be used for either of the selectable markers as described above with the CAI8 strain.

These types of analytical procedures can also be carried out by transitory inactivation of the gene of interest with adjustable promoters other than that described above with the Rox1 repressor protein. To achieve this, the native promoter of the gene is replaced by an adjustable promoter directly on the chromosome or on an extra chromosomal plasmid. One example of another adjustable promoter for use in this method is the CAL promoter or its derivatives, or the tetO promoter (Mumberg et al. 1994, Nucleic Acid Research, 22: 5767-5768; Belli et al. 1998, Yeast, 14: 1127-1138). The essential character of the gene studied can thus be observed, while the promoter used is repressed, either in the haploid strains in the yeast S. cerevisiae, or after inactivation of the second allele in the diploid microorganism (for example C. albicans ).

C. albicans deletion analyses were carried out for each of the target genes identified in Table 1, as described in this section and in Example 2. The results are presented in FIGS. 53-78, each figure representing a single target gene.

### Methods for Identifying Homologous Genes

From a known essential gene in a species, genes which are homologous or have the same function in another species of mycete can be identified. The methods known to those of ordinary skill in the art can be used to identify a homolog to a gene studied in another species of mycete (so-called “orthologous” genes) or genes having the same function as the gene studied. Examples of methods which can be used are given below. These methods are described in the following works which are hereby incorporated by reference herein: Sambrook et al. 1989, Molecular Cloning, Cold Spring Harbor Laboratory Press; Ausubel F. et al. eds. Short Protocols in Molecular Biology, 1995, Wiley; and Guthrie C. and Fink G. R. eds. Methods in Enzymology, Vol. 194, 1991, ‘Guide to Yeast Genetics and Molecular Biology’, Academic Press Inc.

Such methods include screening for homology or gene complementation to genomic or cDNA libraries of pathogenic mycetes, or PCR amplification of such library DNA using specific primers selected by virtue of their homology to the nucleotide sequence of interest.

The homologous DNA sequences of other mycetes as defined above can be isolated, in particular, by the PCR amplification methods known to those of ordinary skill in the art. A non-limiting of such PCR technique is carried out using degenerate nucleotide primers to amplify these homologous DNAs from genomic or cDNA libraries of the corresponding mycetes. The cDNAs can also be prepared from mRNAs isolated from mycetes of various species studied in the context of the present invention, directed to Saccharomyces cerevisiae and Candida albicans, namely Candida stellatoidea, Candida tropicalis, Candida parapsilosis, Candida krusei, Candida pseudotropicalis, Candida quillennondii, Candida glabrata, Candida lusianiae or Candida rugosa, or also mycetes of the type Aspergillus or Cryptococcus, and in particular, for example, Aspergillus fumigatus, Coccidioides immitis, Cryptococcus neoformans, Histoplasma capsulatum, Blastomyces dermatitidis, Paracoccidioides brasiliens and Sporothrix schenckil, or also mycetes of the classes of Phycomycetes or Eumycetes, in particular the sub-classes of Basidiomycetes, Ascomycetes, Mehiascomycetales (yeast) and Plectascales, Gymnascales (fungus of the skin and hair) or of the class of Hyphomycetes, in particular the sub-classes Conidiosporales and Thallosporales, and among these the following species: Mucor, Rhizopus, Coccidioides, Paracoccidioides (Blastomyces, brasiliensis), Endomyces (Blastomyces), Aspergillus, Menicilium. (Scopulariopsis), Trichophyton (Ctenomyces), Epidermophton, Microsporon, Piedraia, Hormodendron, Phialophora, Sporotrichon, Cryptococcus, Candida, Geotrichum, Trichosporon or also Toropsulosis.

Homologous polynucleotides can thus be obtained using the usual methods of cloning and screening, such as those of cloning and sequencing from fragments of chromosomal DNA extracted from cells. For example, to obtain such homologous polynucleotides, it is possible to start from a library of chromosomal DNA fragments. A probe corresponding to a radiolabeled oligonucleotide, preferably made up of 17 nucleotides or more and derived from a partial sequence, can be prepared. The clones containing a DNA identical to that of the probe can thus be identified under stringent conditions. By sequencing individual clones identified in this way using sequencing primers resulting from the original sequence, it is then possible to prolong the sequence in both directions to determine the sequence of the complete gene. Such sequencing can usually be carried out effectively using a double-stranded denatured DNA prepared from a plasmid. Such techniques are described by Maniatis, T., Frisch, E. F., and Sambrook as indicated above. (Laboratory Manual, Cold Spring Harbor, N.Y. (1989), in particular in 1.90 and 13.70 in the chapters on screening by hybridization and sequencing from double-stranded denatured DNA).

The genomic DNA or cDNA libraries can be prepared by known methods and the polynucleotide fragments obtained are integrated into an expression vector, for example a vector such as pRS423 or its derivatives, which can be used both in the bacterium E. coli and in S. cerevisiae. Screening of the library will be carried out by conventional methods of in situ hybridization on a replica of bacterial colonies. The hybridization conditions will be adapted to the stringency required for the reaction so that fragments more or less homologous with the gene studied are identified. The genes of other species of mycetes can also be identified by known so-called “gene complementation” methods. For example, a strain of S. cerevisiae in which an identified essential gene has been placed under the control of an adjustable promoter can be transformed by a representative sample of a DNA or cDNA library corresponding to the mycete studied. When yeasts are cultured under conditions such that the promoter is repressed, the only yeasts that can survive are the ones that carry a recombinant vector containing a sequence of the mycete studied which is functionally equivalent to the initial essential gene. The gene sequence in the mycete studied is then identified by isolating the recombinant vector and sequencing it by known methods. In the same way, the so called “plasmid shuffle” method allows selection of yeasts which have lost expression of the initial essential gene and contain a functionally equivalent sequence originating from another mycete.

This type of study can be performed on various species: the genes which are functionally equivalent or homologous in sequence to an essential gene can be isolated in other mycetes, and in particular in the various mycetes which are pathogenic to humans. For this, it is possible to use, in particular, mycetes belonging to the classes Zygomycetes, Basidiomycetes, Ascomycetes and Deuteromycetes. More particularly, the mycetes will belong to the sub-classes Candida spp., in particular Candida albicans, Candida glabrata, Candida tropicalis, Candida parapsilosis and Candida krusei. The mycetes will also belong to the sub-classes Aspergillus fumigatus, Coccidioides immitis, Cryptococcus neofonnans, Histoplasma capsulatum, Blastomyces dermatidis, Paracoccidioidesbrasiliensis and Sprorothrix schenckii.

### Inhibition of Fungal Growth

The present invention provides for a number of strategies to inhibit fungal growth by inhibiting the biological activity of the target proteins provided herein. As described above, these fungal target proteins are involved in a wide range of activities related to growth and viability, such as, but not limited to, DNA transcription, mRNA translation, mRNA and protein processing and trasport, cell division, growth regulation, cell cycle regulation, and other processes. Although the exact function of some target proteins is not yet known, the target proteins provided by the invention all have the common feature of being involved in fungal growth. In the section below, transcription is exemplified as one potential mechanism through which growth can be affected, but it is to be understood that other mechanisms not specifically described below can be used for studying and/or implementing growth inhibition using the methods described herein.

### Transcription

The present invention provides methods of modifying gene transcription by contacting a S. cerevisiae and/or C. albicans target protein with substances that bind to, or interact with, such a protein or the DNA/RNA encoding such a protein. These substances may modify the influence of the S. cerevisiae and/or C. albicans target protein on transcription, chromatin remodeling or other processes essential to gene transcription. Substances that bind to, or interact with, the S. cerevisiae and/or C. albicans target protein or the DNA/RNA encoding such a protein can prevent or enhance its biological activity, which may directly or indirectly inhibit fungal growth.

For example, anti-sense or non-sense nucleotide sequences that hybridize with the S. cerevisiae and/or C. albicans target protein DNA or RNA and either completely inhibit or decrease their translation or transcription can prevent and inhibit the transcription of other fungal genes. Alternatively, compounds that can bind to or interact with the S. cerevisiae and/or C. albicans target protein can prevent or enhance the function of the protein in the transcription process. These substances include antibodies that are reactive with and bind to either or both of the S. cerevisiae and/or C. albicans target proteins.

### Candidate Inhibitors

Once it has been determined that the target protein is a cidal target in Saccharomyces cerevisiae and essential for growth Candida albicans, the protein may be used as a cidal target in order to isolate candidate inhibitors of fungal growth and/or infection.

As noted above, a “candidate inhibitor,” as used herein, is any compound with a potential to inhibit, in Candida albicans or other fungal species, the biological activity of a target protein. Candidate inhibitor compounds are first identified in a primary screen against the C. albicans target protein. This primary screen may be affinity based, mechanistic (e.g., in vitro transcription assay), or cell-based (e.g., reporter assay). Such assays are described further below. A candidate inhibitor is tested in a concentration range that depends upon the molecular weight of the molecule and the type of assay. For example, for inhibition of protein/protein or protein/DNA complex formation or transcription elongation small molecules (as defined below) may be tested in a concentration range of 1 pg - 100 ug/mL, preferably at about 100 pg -20 ug/mL; large molecules, e.g., peptides, may be tested in the range of 10 ng -100 ug/mL, preferably 100 ng -10 ug/mL.

Inhibitors of Candida albicans growth or viability may target the C. albicans target proteins described herein, or it may target a protein or nucleic acid that interacts with the C. albicans target protein to prevent the natural biological interaction that occurs in vivo. An inhibitor identified as described herein must possess the property that at some concentration it will inhibit Candida albicans growth or viability, most preferably at the same concentration it will not significantly affect the growth of mammalian, particularly human, cells.

Candidate inhibitors include peptide and polypeptide inhibitors having an amino acid sequence based upon the C. albicans target protein sequences described herein. For example, a fragment of the C. albicans target protein may act to prevent the growth of wild type Candida albicans cells because it acts as a competitive inhibitor with respect to the C. albicans target protein binding to other proteins involved in Candida growth, e.g., chromatin binding, cell division, transcription, or another essential activity.

Inhibitory compounds to be tested are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means.

Compounds useful as inhibitors may be found within numerous chemical classes, though typically they are organic compounds, and preferably small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like. Other methods of stabilization may include encapsulation, for example, in liposomes, etc.

### Primary Inhibitor Screening High-Throughput Methods For Screening Inhibitors

In a preferred embodiment, a high-throughput screening protocol, also referred to as ATLAS, is used to survey a large number of test compounds for their ability to bind or otherwise interact with a C. albicans target protein. High-throughput screening methods are described in U.S. Pat. Nos. 5,585,277 and 5,679,582, in U.S. Ser. No. 08/547,889, and in the published PCT application PCT/US96/19698, and may be used for identifying a ligand that binds the target proteins described herein. According to these methods, a ligand, or a plurality of ligands for a C. albicans target protein is identified by its ability to influence the extent of folding or the rate of folding or unfolding of the target protein. Experimental conditions are chosen so that the target protein unfolds to a measurable extent, whether reversible or irreversible. If the test ligand binds to the target protein under these conditions, the relative amount of folded:unfolded target protein or the rate of folding or unfolding of the target protein in the presence of the test ligand will be different, i.e. higher or lower, than that observed in the absence of the test ligand. Thus, the method encompasses incubating the C. albicans target protein in the presence and absence of a plurality of test ligands under conditions in which (in the absence of ligand) the target protein would partially or totally unfold. This is followed by analysis of the absolute or relative amounts of folded vs. unfolded target protein or of the rate of folding or unfolding of the target protein.

An important feature of this method is that it will detect any compound that binds to any sequence or domain of the C. albicans target protein, and not only to sequences or domains that are intimately involved in a biological activity or function. The binding sequence, region, or domain may be present on the surface of the target protein when it is in its folded state, or may be buried in the interior of the protein. Some binding sites may only become accessible to ligand binding when the protein is partially or totally unfolded.

Briefly, to carry out this method, the test ligand or ligands are combined with the C. albicans target protein, and the mixture is maintained under appropriate conditions and for a sufficient time to allow binding of the test ligand. Experimental conditions are determined empirically. When testing test ligands, incubation conditions are chosen so that most ligand:target protein interactions would be expected to proceed to completion. The test ligand is present in molar excess relative to the target protein. The target protein can be in a soluble form, or, alternatively, can be bound to a solid phase matrix. The matrix may comprise without limitation beads, membrane filters, plastic surfaces, or other suitable solid supports.

In a preferred embodiment, binding of test ligand or ligands to the target protein is detected through the use of proteolysis. This assay is based on the increased susceptibility of unfolded, denatured polypeptides to protease digestion relative to that of folded proteins. In this case, the test ligand-target protein combination, and a control combination lacking the test ligand, are treated with one or more proteases that act preferentially upon unfolded target protein. After an appropriate period of incubation, the level of intact i.e. unproteolysed target protein is assessed using one of the methods described below e.g. gel electrophoresis and/or inmunoassay.

There are two possible outcomes that indicate that the test ligand has bound the target protein. Either 1) a significantly higher, or 2) a significantly lower absolute amount of intact or degraded protein may be observed in the presence of ligand than in its absence.

Proteases useful in practicing the present invention include without limitation trypsin, chymotrypsin, V8 protease, elastase, carboxypeptidase, proteinase K, thermolysin, papain and subtilisin (all of which can be obtained from Sigma Chemical Co., St. Louis, Mo.). The most important criterion in, selecting a protease or proteases for use in practicing the present invention is that the protease(s) must be capable of digesting the target protein under the chosen incubation conditions, and that this activity be preferentially directed towards the unfolded form of the protein. To avoid “false positive” results caused by test ligands that directly inhibit the protease, more than one protease, particularly proteases with different enzymatic mechanisms of action, can be used simultaneously or in parallel assays. In addition, co-factors that are required for the activity of the protease(s) are provided in excess, to avoid false positive results due to test ligands that may sequester these factors.

In a typical embodiment of this method, purified target protein is first taken up to a final concentration of about 1-100 g/mL in a buffer containing 50 mM Tris-HCl, pH 7.5, 10% DMSO, 50 mM NaCl, 10% glycerol, and 1.0 mM DTT. Proteases, such as, for example, proteinase K or thermolysin (proteases with distinct mechanisms of action), are then added individually to a final concentration of 0.2-10.0 g/mL. Parallel incubations are performed for different time periods ranging from 5 minutes to one hour, preferably 30 minutes, at 4° C., 15° C., 25° C., and 35° C. Reactions are terminated by addition of an appropriate protease inhibitor, such as, for example, phenylmethylsulfonyl chloride (PMSF) to a final concentration of 1 mM (for serine proteases), ethylenediaminotetraacetic acid (EDTA) to a final concentration of 20 mM (for metalloproteases), or iodoacetamide (for cysteine proteases). The amount of intact protein remaining in the reaction mixture at the end of the incubation period may then be assessed by any method, including without limitation polyacrylamide gel electrophoresis, ELISA, or binding to nitrocellulose filters. It will be understood that additional experiments employing a narrower range of temperatures can be performed to establish appropriate conditions. This protocol allows the selection of appropriate conditions (e.g., protease concentration and digestion temperature) that result in digestion of approximately 70% of the target protein within a 30 minute incubation period, indicating that a significant degree of unfolding has occurred.

In another embodiment, the relative amount of folded and unfolded target protein in the presence and absence of test ligand is assessed by measuring the relative amount of the protein that binds to an appropriate surface. This method takes advantage of the increased propensity of unfolded proteins to adhere to surfaces, which is due to the increased surface area, and decrease in masking of hydrophobic residues, that results from unfolding. If a test ligand binds the C. albicans target protein (i.e., is a ligand), it may stabilize the folded form of the target protein and decrease its binding to a solid surface. Alternatively, a ligand may stabilize the unfolded form of the protein and increase its binding to a solid surface.

Surfaces suitable for this purpose include without limitation microtiter plates constructed from a variety of treated or untreated plastics, plates treated for tissue culture or for high protein binding, nitrocellulose filters and PVDF filters.

In another embodiment, the extent to which folded and unfolded target protein are present in the test combination is assessed through the use of antibodies specific for either the unfolded state or the folded state of the protein i.e. denatured-specific (“DS”), or native-specific (“NS”) antibodies, respectively. (Breyer, J. Biol. Chem. 1989; 264(5):13348-13354). Polyclonal or monoclonal antibodies are prepared as described above. The resulting antibodies are screened for preferential binding to the C. albicans target protein in its denatured state. These antibodies are used to screen for inhibitors of these interactions.

In another embodiment, molecular chaperones are used to assess the relative levels of folded and unfolded protein in a test combination. Chaperones encompass known proteins that bind unfolded proteins as part of their normal physiological function. In this embodiment, a test combination containing the test ligand and the C. albicans target protein is exposed to a solid support e.g. microtiter plate or other suitable surface coated with a molecular chaperone, under conditions appropriate for binding the target protein with its ligand and binding of the molecular chaperone to unfolded target protein. The unfolded target protein in the solution will have a greater tendency to bind to the molecular chaperone-covered surface relative to the ligand-stabilized folded target protein. Thus, the ability of the test ligand to bind target protein can be determined by determining the amount of target protein remaining unbound, or the amount bound to the chaperone-coated surface. Alternatively, a competition assay for binding to molecular chaperones can be utilized.

Once conditions are established for high-throughput screening as described above, the protocol is repeated simultaneously with a large number of test ligands at concentrations ranging from, e.g., 20 to 200 M. Observation of at least a two-fold increase or decrease in the extent of digestion of the target protein signifies a “hit” compound, i.e., a ligand that binds the target protein. Preferred conditions are those in which between 0.1% and 1% of test ligands are identified as “hit” compounds using this procedure.

In yet another embodiment, the test and control combinations described above can be contacted with a conformation-sensitive probe containing a reporter molecule such as, e.g., a fluorescent molecule or radionucleotide, i.e., a probe that binds preferentially to the folded, unfolded, or molten globule state of the C. albicans target protein or whose reporter-mediated properties are in any way affected by the folding status of the C. albicans target protein.

### Phase Display Technology Screening

In addition to the high-throughput screening techniques described above, technologies for molecular identification can be employed in the identification of inhibitor molecules. One of these technologies is phage display technology (U.S. Pat. No. 5,403,484. Viruses Expressing Chimeric Binding Proteins). Phage display permits identification of a binding protein against a chosen target. Phage display is a protocol of molecular screening which utilizes recombinant bacteriophage. The technology involves transforming bacteriophage with a gene that encodes an appropriate ligand (in this case, a candidate inhibitor) capable of binding to the target molecule of interest. For the purposes of this disclosure, the target molecule may be a C. albicans target protein. The transformed bacteriophage (which preferably is tethered to a solid support) express the candidate inhibitor and display it on their phage coat. The cells or viruses bearing the candidate inhibitor which recognize the target molecule are isolated and amplified. The successful inhibitors are then characterized.

Phage display technology has advantages over standard affinity ligand screening technologies. The phage surface displays the microprotein ligand in a three dimensional conformation, more closely resembling its naturally occurring conformation. This allows for more specific and higher affinity binding for screening purposes.

### Biospecific Interaction Analysis Screening

Another relatively new screening technology which may be applied to the inhibitor screening assays of this invention is biospecific interaction analysis (BIAcore, Pharmacia Biosensor AB, Uppsala, Sweden). This technology is described in detail by Jonsson et al. (Biotechniques 11:5, 620-627 (1991)). Biospecific interaction analysis utilizes surface plasmon resonance (SPR) to monitor the adsorption of biomolecular complexes on a sensor chip. SPR measures the changes in refractive index of a polarized light directed at the surface of the sensor chip.

Specific ligands (i.e., candidate inhibitors) capable of binding to the target molecule of interest (i.e., a C. albicans target protein or a protein-protein or protein-DNA complex containing the C. albicans target protein) are immobilized to the sensor chip. In the presence of the target molecule, specific binding to the immobilized ligand occurs. The nascent immobilized ligand-target molecule complex causes a change in the refractive index of the polarized light and is detected on a diode array. Biospecific interaction analysis provides the advantages of; 1) allowing for label-free studies of molecular complex formation; 2) studying molecular interactions in real time as the assay is passed over the sensor chip; 3) detecting surface concentrations down to 10 pg/mm²; detecting interactions between two or more molecules; and 4) being fully automated (Biotechniques 11:5, 620-627 (1991)).

### Screening Through Use Of A Transcription Assay

In cases where the target protein has been identified as being required for transcription per se and/or elongation, the present invention encompasses the identification of agents useful in modulating fungal gene transcription, particularly the transcription of genes by RNA polymerase II in a target protein-dependent manner. Thus, if the target protein has been identified as being essential for transcription and/or elongation, inhibitors of Candida albicans growth and viability may also be screened either by measuring inhibition of any of the activities described above, or by assaying formation of a protein/DNA complex or inhibition of sporulation when cells are contacted with Candida albicans inhibitors.

### In Vitro Transcription Assay

If an essential target protein has been identified as being required for transcription, and it has been identified according to any of the screening methods described above, its activity and effect on transcription can be confirmed by adding it to an in vitro transcription reaction, and measuring its effect on the target protein-mediated activated transcription, using an in vitro transcription assay. For example, DNA of interest (i.e., DNA to be transcribed) can be admixed with (i) purified RNA polymerase II, (ii) the SRB proteins, (iii) transcription factors b, e, g or a, (iv) the C. albicans target protein and (v) the substance (ligand) to be tested. The mixture is maintained under conditions sufficient for transcription to occur. The resulting combination is referred to as a test mixture. DNA transcription can be assessed by determining the quantity of mRNA produced. Transcription is determined in the presence of the substance being tested and compared to DNA transcription in the absence of the test substance taking place under identical conditions (e.g., a control mixture). If transcription occurs to a lesser extent in the test mixture, (i.e., in the presence of the substance being evaluated) than in the control mixture, the substance may have interacted with one or more SRB proteins , or with the C. albicans target protein, preferably in such a manner as to inhibit transcription. If transcription occurs to a greater extent in the test mixture than in the control mixture, the substance has interacted in such a manner as to stimulate transcription.

Transcription of DNA sequences, or translation of mRNA sequences encoding the C. albicans target protein can also be inhibited or decreased by inhibitor compounds, resulting in decreased production of, or the complete absence of the C. albicans target protein. Gene transcription can be modified by introducing an effective amount of a substance into a cell that inhibits transcription of the gene encoding the C. albicans target protein, or that inhibits translation of mRNA encoding the C. albicans target protein. For example, antisense nucleotide sequences can be introduced into the cell that will hybridize with the gene encoding the target protein and inhibit transcription of the gene. (See, Current Protocols in Molecular Biology, Eds. Ausubel et al. Greene Publ. Assoc., Wiley-Interscience, NY, N.Y., 1997). Alternatively, an antisense sequence can be introduced into the cell that will interfere with translation of the mRNA encoding the C. albicans target protein.

### Secondary Screens—Measurement of Inhibition of Candida albicans Growth in Culture

Once a putative inhibitor has been identified in the primary screen or screens, it may be desirable to determine the effect of the inhibitor on the growth and/or viability of Candida albicans in culture. Methods for performing tests on fungal growth inhibition in culture are well-known in the art.

Non-limiting examples of such procedures test the candidate inhibitor compounds for antifungal activity against a panel of three strains: C. albicans, S. cerevisiae , and A. nidulans. One such procedure is based on the NCCLS M27A method (The National Committee for Clinical Laboratory Standards, Reference Method for Broth Microdilution Antifungal Susceptibility Testing of Yeasts; approved standard, 1997) to measure minimum inhibitory concentrations (MICs) and minimum fungicidal concentrations (MFCs). An overview of this of this protocol follows.

### Media

1 . Sabouraud dextrose agar (SDA): 10 g Bacto Neopeptone; 40 g Bacto Dextrose; 15 g Bacto Agar. Suspend contents in 1 liter of water and boil while stirring to dissolve completely. Autoclave for 15 minutes. SDA is conveniently sold as a powdered mix by DIFCO (Cat #0109-17-1).

2. Potato dextrose agar (PDA): 4 g Potato extract; 20 g Bacto Dextrose; 15 g Bacto Agar. Suspend contents in 1 liter of water and boil while stirring to dissolve completely. Autoclave for 15 minutes. PDA is conveniently sold as a powdered mix by DIFCO (Cat #0013-17-6).

3. RPMI-1640: 10.4 g powdered media (Sigma R-6504, w/ glutamine & w/o bicarbonate); 2.0 g NaHCO₃ (Sigma S-6297); 34.53 g MOPS buffer (Sigma M-6270). Dissolve powdered media and NaHCO₃ in 900 ml distilled water. Add MOPS and stir until dissolved. Adjust pH to 7.0 using 1N NaOH. Bring final volume to 1 liter, filter sterilize, and store at 4° C.

4. RPMI-1640 with 12.5% mouse serum: 10.4 g powdered media (Sigma R-6504, w/ glutamine & w/o bicarbonate); 2.0 g NaHCO₃ (Sigma S-6297); 34.53 g MOPS buffer (Sigma M-6270); 50 ml mouse serum (Sigma S-7273). Dissolve powdered media and NaHCO₃ in 750 ml distilled water. Add MOPS and stir until dissolved. Adjust pH to 7.0 using 1N NaOH and bring volume to 875 ml. Remove 350 ml and add to it 50 ml of mouse serum. Bring remaining volume of media (525 ml) to 600 ml with the addition of 75 ml of distilled water. Filter sterilize each solution and store at 4° C.

### Inoculum Preparation

1. Yeasts: Yeasts (Saccharonyces cerevisiae and Candida albicans) are cultured on Sabouraud dextrose agar (SDA) plates in a 35° C. incubator. Strains on SDA plates are stored at 4° C. and used as working stock cultures. Working stock plates are prepared once a month from frozen stocks of cells. Inoculum for susceptibility testing is prepared from fresh 24 hour cultures. 5-10 colonies are scraped from the plate and suspended in three milliliters of sterile 0.85% saline (8.5 g/liter NaCl). The cell density of the solution is determined by measuring the absorbance in a spectrophotometer (Shimadzu UV-1201S UV-VIS Spectrophotometer) set at 600 nm. An absorbance value between 0.1 and 0.4 is required for an accurate reading.

For C. albicans, e.g., strain ATCC 10231, 1.0 OD₆₀₀ unit is approximately 10⁷ cells per ml while for Saccharomyces cerevisiae strain CTY552 1.0 OD₆₀₀ unit is slightly less than 10⁷ cells per ml. Dilute the cell suspension with the appropriate medium (typically RPMI-1640) to OD₆₀₀=0.0003 for Candida and OD600=0.0004 for Saccharomyces. The diluted suspension should contain approximately 3×10³ cells per ml (this is a 2× concentration inoculum). Two 100 ul aliquots of this dilution should be spread on SDA plates and incubated at 35° C. for 1-2 days to determine the precise number of colony forming units. An acceptable range for the inoculum (2×) is 1-5×10³ cfu/ml (100-500 for 100 ul). Following two-fold dilution of the inoculum with compound, the final concentration of cells will be 0.5-2.5×10³ per ml. The inoculum should be kept at 4° C. and used within a few hours.

2. Filamentous fungi: Filamentous fungi (Aspergillus spp.) should be cultured on Potato dextrose agar (PDA) plates in a 35° C. incubator. A fresh plate should be started from frozen cell stocks once a month. Inoculum of Aspergillus for susceptibility testing is prepared from plates incubated at 35° C. for 5 days. Colonies are covered with five ml of sterile 0.85% saline (8.5 g/liter NaCl) and gently rocked for 10-15 minutes. To dislodge the conidia, use an automatic pipettor to gently wash over the colonies. The saline solution is removed from the plate and the heavy particles allowed to settle for 3-5 minutes. The upper suspension is removed and vortexed for 15 sec. The turbidity of the solution is determined by measuring the absorbance in a spectrophotometer (Shimadzu UV-1201S UV-VIS Spectrophotometer) set at 600 nm. An absorbance value between 0.1 and 0.4 is required for an accurate reading.

Dilute the cell suspension with the appropriate medium (typically RPMI-1640) to OD₆₀₀=0.0004. The final suspension should contain approximately 3×103 cfu per ml (this is a 2× concentration inoculum). Two 100 ul aliquots of this dilution should be spread on SDA plates and incubated at 35° C. for 1-2 days to determine the precise number of colony forming units. An acceptable range for the inoculum (2×) is 1-5×10³ cfu/ml (100-500 for 100 ul). Following two-fold dilution of the inoculum with compound, the final concentration of cells will be 0.5-2.5×10³ per ml. The inoculum should be kept at 4° C. and used within a few hours.

### Compound Preparation

Stock solutions and concentrations tested will vary from compound to compound. In general, though, stock solutions of 12.8 mg/ml in DMSO (Sigma D-8779) should be prepared. This will allow for a 128 ug/ml starting test concentration containing 1% DMSO. Stock solutions should be stored at −20° C. and dilutions for antifungal testing should be freshly prepared before each assay.

For compounds of unknown activity or ones with MIC values of >4 ug/ml, a range of concentrations from 128 ug/ml to 0.125 ug/ml should be used. More active compounds, such as Amphotericin B (Sigma A2411) and Itraconazole (Research Diagnostics Inc. cat#30.211.44), require a lower range of concentrations (16 ug/ml to 0.016 ug/ml). Stock solutions of Amphotericin B and Itraconazole should be prepared at 1.6 mg/ml in DMSO. Amphotericin B is sold as a powder that is approximately 80% Amphotericin B. Stock solutions should be made accordingly (2.0 mg of powder for a 1 ml solution of 1.6 mg/ml Amphotericin B).

Stock solutions of control compounds (1.6 mg/ml, Amphotericin B or Itraconazole) are initially diluted in medium to a concentration of 32 ug/ml while stock solutions of test compounds (typically 12.8 mg/ml) are diluted to 256 ug/ml. Both of these (control and test compounds) represent 1:50 dilutions. For an assay with three fungal strains, 40 microliters of a stock solution should be diluted to 2.0 ml with room temperature medium. If a stock solution of a test compound is not at 12.8 mg/ml, the appropriate dilution must be calculated. Serial dilutions will be produced (see below) using these initial dilutions. Addition of cells to compound will produce an additional two-fold dilution.

Natural product extracts are tested at concentrations ranging from 200 to 204,800 fold dilution of the extract based upon the initial culture volume. The extract should first be diluted 100 fold then serial dilutions produced as directed below.

### Assay Setup

Antifungal susceptibility tests should be setup in polystyrene, 96-well, flat bottom plates (Costar 9017). To every well in columns 2-12 is added 100 microliters of media. An electronic multichannel (12) pipettor with no tip on channel one makes this job simple. To every well in column one is added 200 microliters of diluted compound (32 ug/ml for Amphotericin B and Itraconazole controls, 256 ug/ml for test compounds, 100-fold dilution for natural product extracts). A manual multichannel (8) pipettor is then used to set up a series of 2-fold dilutions. 100 microliters is removed from each well of column one and mixed with 100 microliters in column 2. This is done successively (column two to column three etc.) to produce a set of 11 serial dilutions (column 12 is a drug free control).

To every well in two rows, 100 ul of inoculum (2×) of a single strain is added. To the final two rows on the plate (G & H), only media is added. Addition of inoculum is best accomplished using an electronic multichannel (12) pipettor. This setup (see below) creates a starting cell density of 500-2500 per ml (100-500 per well) and drug concentration ranging from 16 ug/ml to 0.016 ug/ml for controls (Amphotericin B and Itraconazole), 128 ug/ml to 0.125 ug/ml for pure test compounds, and 200 to 204,800-fold dilutions for natural product extracts.

It is important to determine the number of colony forming units (CFUs) present in each strain inoculum (2×). Two 100 ul aliquots of each inoculum (2×) should be spread on SDA plates and incubated at 35° C. for 1-2 days to determine the precise number of colony forming units. An acceptable range for the inoculum (2×) is 1-5×10³ cfu/ml (100-500 for 100 ul). Following two-fold dilution of the inoculum with compound, the final concentration of cells will be 0.5-2.5×10³ per ml. The plates should then be placed in a dark, 35° C. incubator for 48 hours.

### Modified Assay Setup for Low Solubility Compounds

Some compounds are not very soluble in aqueous media even at low concentrations and dilution artifacts can result from precipitation of the compounds. To avoid such problems a series of two fold dilutions at 100 times the final concentration is prepared from the stock solution in the same solvent (typically DMSO). Each intermediate solution is then diluted to final strength with 1× inoculum.

This type of assay setup involves making a series of 11, 2-fold dilutions in DMSO ranging from 12,800 ug/ml to 12.5 ug/ml for test compounds and 1600 ug/ml to 1.6 ug/ml for control compounds (Amphotericin B and itraconazole). Two microliters of diluted compound are placed into each well of the appropriate column (12,800 ug/ml in column 1, down to 12.5 ug/ml in column 11, and DMSO to column 12). To every well in two rows, 200 ul of inoculum (1×) of a single strain is added. To the final two rows on the plate (G & H), only media (200 ul) is added. Addition of inoculum is best accomplished using an electronic multichannel (12) pipettor. Final concentrations of cells and compounds are the same as described above for the standard assay setup. Please note that the inoculum in this assay is at 1× concentration, while the inoculum for the assay described above is a 2× concentrate. The 1× inoculum is made by adding an equal volume of media to the 2× inoculum.

NCCLS recommends using this type of assay setup for insoluble compounds, including Amphotericin B and Itraconazole. While we are able to obtain reasonably consistent results for Amphotericin B and Itraconazole using the standard assay setup, some test compounds may benefit from doing the serial dilutions in DMSO. Compounds that form heavy precipitates upon dilution to media should be considered for this assay, particularly if the compound seems to be a promising candidate or inconsistent results are obtained in the standard assay.

### Reading the Results

Minimum Inhibitory Concentration (MIC): The MIC is the lowest concentration of an antifungal agent that inhibits growth of the organism. For Amphotericin B, the lowest drug concentration which gives no visible growth is the MIC. For Itraconazole (and other azoles), the lowest drug concentration which reduces growth to ≦20% of the growth control (column 12) is the MIC.

For test compounds that give a sharp endpoint (like Amphotericin B), the lowest drug concentration which gives no visible growth is the MIC. For test compounds that give a trailing effect on inhibition of cell growth (like the azoles), the lowest drug concentration which reduces growth to ≦20% of the growth control (as determined by measurement of turbidity) is the MIC.

The turbidity of each well is determined by measuring the absorbance at 415 nm on a plate reader (BIO-RAD Model 3550-UV). The rows containing no cells (G & H) serve as a control for absorbance. Column 12, containing no compound, serves as the growth control.

Minimum Fungicidal Concentration (MFC): The MFC is the lowest concentration of an antifungal agent that results in an inviable culture. Two slightly different standards and assays are applied, depending on the circumstances. For each of the two methods, though, culture viability should be determined beginning with the drug dilution immediately below the MIC and continuing through to the highest drug concentration.

The first and more rigorous standard considers a culture to be inviable if it contains ≦1% of the colony forming units of the starting culture. This is determined by completely removing the cells from a well of the microtiter plate and placing them in a microfuge tube containing 1.3 ml of RPMI media. The cells are spun for 2 minutes, supernatant poured off, cells resuspended in the remaining media, and spread on an SDA plate. The plate is incubated at 35° C. for 1-2 days, and the colonies counted. These numbers are compared to the original cfu count from day 1 of the assay.

A second, simpler method is more practical for processing a large number of samples and is the method that we routinely use. Following resuspension of the cells by pipetting, 15 microliters is spotted directly to an SDA plate and incubated for 2 days at 35° C. A culture is considered inviable if no colonies form on the plate. While this method is much simpler than the one above, it is less quantitative and no efforts are made to wash the compound away from the cells before plating. One may observe inhibition of growth on the agar plate if a compound is still present at high enough concentrations

The control compound Amphotericin B is a cidal drug and the MIC is typically equal to the MFC. Itraconazole, in contrast, is a static drug and viable cells should be recovered from wells containing compound at concentrations well above the MIC.

### Quality Control

Cell density of the inoculum (2×) must be between 1 and 5×10³ cfu/ml (100-500 cfu per 100 microliters). Starting cell concentration in the assay will be 0.5 to 2.5×10³ cfu/ml.

Acceptable MIC range values (ug/ml):

| | Am B | Itraconazole |
|---|---|---|
| Candida albicans, e.g., ATCC 10231 | 0.25-1.0 | 0.25-1.0 |
| Saccharomyces cerevisiae, e.g., CTY552 | 0.25-1.0 | 0.25-1.0 |
| Aspergillus nidulans, e.g., NRRL 194 (ATCC 38163) | 0.5-2.0 | 0.25-1.0 |

If the starting cell density or MIC values do not fall within the acceptable range, all results in the assay for the particular strain in question are considered invalid and the assay should be repeated.

### Secondary Screens—Mechanistic Assays

The preferred inhibitor compounds of the invention are those which possess antifungal activity, although compounds with significant activity in an in vitro mechanism-based assay may be considered for further development. Such secondary assays are performed to determine the mechanism of action of these compounds. Such secondary mechanistic assays include in vitro experiments, as well as and in vivo experiments in fungi, to determine the mechanistic inhibitory activity of these compounds. The precise nature of these assays will depend on the target.

Compounds that prevent cell growth through inhibition of the target protein are considered for further development.

### Counterscreening in Other Species

In parallel to secondary screen assays, counterscreens are performed to determine if the compounds inhibit the activity of any human homolog. The precise nature of the counterscreen(s) will depend on the nature of the target protein. These counterscreens may include an affinity assay to determine if the compound binds the human homolog or an in vitro or cell-based mechanistic assay to determine if the compound inhibits the activity of the human protein.

Cytotoxicity studies on mammalian cells are also performed to determine if the compound is toxic to mammalian cells in culture. Compounds that do not bind to and/or inhibit the activity of the human homolog will be considered for further development.

### Transcription Inhibition Counterscreen Using Human Homolog

When the essential target protein has been identified as being required for growth and as an inhibitor of Candida albicans according to one or more of the assays described herein, it may be tested further in order to determine its effect on the host organism. In the development of useful antifungal compounds for human therapeutics, it is desirable that such compounds act as effective agents in inhibiting the viability of the fungal pathogen while not significantly inhibiting human cell systems. Specifically, inhibitors of Candida albicans identified in any one of the above described assays may be counterscreened for inhibition of a human homolog of the target protein.

If available, the human gene encoding for the target protein can be expressed and purified utilizing published methods and its homology to the yeast target protein homolog(s). The human homolog can be contacted with candidate inhibitor in assays such as those described above using a human cell culture system. The effectiveness of a C. albicans inhibitor as a human therapeutic is determined as one which exhibits a low level of inhibition against its human homolog relative to the level of inhibition with respect to the C. albicans target protein. For example, it is preferred that the amount of inhibition by a given inhibitor of the human homolog in a human system be no more than 20% with respect to the amount of inhibition of the C. albicans target protein.

Such inhibitors are “selective inhibitors” of the C. albicans target protein which “selectively inhibit” C. albicans biological activity. The lack of effect of a test compound on mammalian transcription or other growth-related mechanisms is tested by replacing yeast components with an analogous human in vitro transcription system as in e.g. Manley et al. Proc.Natl.Acad.Sci. USA 77:3855, 1980.

An example of one such mammalian cytotoxicity screening method is described in Example 3.

### Chemical Analoging

It is important to note that some compounds may prove to be cytotoxic, but not inhibitory of the activity of the human homolog. Compounds that exhibit such non-target based cytotoxicity are still considered for further development. Chemical analoging efforts may be used to separate the target-based antifungal activity from the non-target-based cytotoxicity activity.

Chemical analoging is also used to identify compounds with improved antifungal activity and reduced cytotoxicity. The secondary assays and counterscreens described above are used in parallel with antifungal assays to ensure that compounds remain active against the appropriate target, i.e., remain inhibitory with the same mechanism of action.

Antifungal testing against a broad spectrum of fungal species and a large number of isolates is also performed at this point. The broad spectrum of fungal species will include those resistant to existing therapeutics, e.g., Amphotericin B and various azoles such as, for example, intraconzole and fluconazole. Compounds which inhibit growth of fungi, particularly Candida and Aspergillus species, at a concentration of 4 ug/ml or less, exhibit minimal cytotoxicity, and have a confirmed mechanism of action are considered for further development..

### Preclinical Development of Candidate Drugs

Subsequent preclinical development of compounds includes, but is not limited to: formulation, toxicology, pharmacokinetics, animal efficacy studies, and medicinal chemistry. Compounds with the desired characteristics are selected for clinical trials in human subjects.

### Dosage and Pharmaceutical Formulations

For therapeutic uses, inhibitors identified as described herein may be administered in a pharmaceutically acceptable/biologically compatible formulation. The compositions of the present invention can be administered in dosages and by techniques well known to those skilled in the medical, veterinary, and agricultural arts taking into consideration such factors as the age, sex, weight, species and condition of the particular patient, and the route of administration. The compositions of the present invention can be administered alone or in combination, or can be co-administered or sequentially administered with additional antifungal agents, such as, e.g., nystatin, amphotericin B, flucytosine and the various antifungal azoles.

Such pharmaceutical compositions can be used in particular for treatment of topical and systemic fungal infections and can be administered bucally, rectally, parenterally or locally by topical application to the skin and the mucous membranes, or by intravenous or intramuscular injection. These compositions can be solid or liquid and can be in any of the pharmaceutical forms generally used in human medicine, such as, for example, simple or coated tablets, capsules, granules, suppositories, injectable preparations, ointments, creams, gels and aerosol preparations. The pharmaceutical compositions of the invention are prepared by the usual methods known to those of ordinary skill in the art. The active principle can be incorporated in them with excipients usually employed in pharmaceutical compositions, such as talc, gum arabic, lactose, starch, magnesium stearate, cacao butter, aqueous or non-aqueous vehicles, fatty substances of animal or plant origin, paraffin derivatives, glycols, various wetting, dispersing or emulsifying agents and preservatives.

Liquid preparations are useful for 1) mucosal administration, e.g., oral, nasal, anal, vaginal, peroral, intragastric administration and the like, in the form of solutions, suspensions, syrups, elixirs; and 2) topical administration e.g., in the form of a cream, ointment, lotion or spray. Further, liquid pharmaceutical formulations comprising the inhibitors to be used for parenteral, subcutaneous, intradermal, intramuscular, intravenous administrations, and the like, such as sterile solutions, suspensions or emulsions, e.g, for administration by injection, can be formulated without undue experimentation.

In order for a composition to be administered to an animal or human, and for any particular method of administration, it is preferred to determine the toxicity, such as by determining the lethal dose (LD) and LD₅₀ in a suitable animal model, e.g., mouse; the dosage of the composition(s), and the concentration of components in the composition; and the timing of administration in order to maximize the antiviral and/or antimicrobial response. Such factors can be determined without undue experimentation by such methods as titrations and analysis of sera for antibodies or antigens, e.g., by ELISA and/or EFFIT analysis. Such determinations do not require undue experimentation from the knowledge of the skilled artisan, the present disclosure and the documents cited herein.

The formulations can be administered in a pharmaceutically effective amount and/or an antifungal effective amount, taking into account such factors as the relative activity and toxicity for the target indication, e.g., antifungal activity, as well as the route of administration, and the age, sex, weight, species and condition of the particular patient.

As discussed above, the pharmaceutical compositions of the present invention can be solutions, suspensions, emulsions, syrups, elixirs, capsules, tablets, creams, lotions and the like. The compositions may contain a suitable carrier, diluent, or excipient, such as sterile water, physiological saline, glucose, or the like. Moreover, the compositions can also be lyophilized, and/or may contain auxiliary substances, such as wetting or emulsifying agents, pH buffering agents, adjuvants, gelling or viscosity enhancing additives, preservatives, flavoring agents, colors, and the like, depending upon the route of administration and the preparation desired. Standard texts, such as “Remington's Pharmaceutical Science”, 17th Ed., 1985, incorporated herein by reference, may be consulted to prepare suitable preparations, without undue experimentation.

The amount of inhibitor administered will be determined according to the degree of pathogenic infection and whether the infection is systemic or localized, and will typically be in the range of about 1 ug-100 mg/kg body weight. Where the inhibitor is a peptide or polypeptide, it will be administered in the range of about 100-500 ug/mL per dose. A single dose of inhibitor or multiple doses, daily, weekly, or intermittently, is contemplated according to the invention.

The route of administration will be chosen by the physician, and may be topical, oral, transdermal, nasal, rectal, intravenous, intramuscular, or subcutaneous.

The following examples are intended as non-limiting illustration of the present invention.

### EXAMPLE 1

### S. cerevisiae Inactivation Analysis

### Yeast Genomic DNA Preparation

This protocol can be used to prepare genomic DNA from Candida albicans cultures as well as Saccharomyces cerevisiae. Streak a yeast stock culture from a glycerol stock to a YPD (Bio101 Cat# 4001-242) plate and incubated for 48 hours at 30° C. Pick a single, distinct colony into 5 ml of YPD media (Bio100 Cat# 4001-042), and incubate overnight at 30° C. in a roller drum. Cells from 1 ml of this culture are pelleted with a 5 second spin in a microcentrifuge. The cells are washed one time with 1 ml TE (10 mM Tris-Cl, pH 8.0, 1 mM EDTA) and respun. Resuspend the pellet in 0.2 ml Extraction Buffer (2% TritonX100, 1% SDS, 100 mM NaCl, 10 mM Tris pH 7.5 and 1 mM EDTA) and add 0.2 ml phenol/chloroform/isoamyl (25:24:1, v:v:v). Add 0.3 g acid washed 400 micron glass beads. Vortex for 5 minutes. Add 0.2 ml TE; spin in a microcentrifuge for 10 minutes at 10-13 krpm. Remove the aqueous phase to a fresh tube. Precipitate with 2.5 volumes absolute ethanol. Spin and resuspend the pellet in 400 ul TE plus 3 ul of a 10 mg/ml RNase A stock. Incubate at 37° C. for 5 minutes. Add 10 ul 4 M ammonium acetate and 1 ml absolute ethanol. Mix by inversion and centrifuge for 8 minutes in a microcentrifuge. Air dry the pellet and resuspend in 50 ul TE. Store at 40° C. The solution may appear somewhat cloudy. Before diluting this stock for use in PCR reactions or Southern blotting, vortex the stock sample briefly.

Alternately, the YeaStar Genomic DNA Kit is available from Zymo Research (Cat. # D2002). It has the advantage of avoiding the use of glass beads and phenol:choloroform mixtures, and produces very clean genomic DNA, although in some cases it has proven to be a somewhat less reproducible method than that detailed above.

### Transformation of S. cerevisiae

Streak strain to a rich media plate (such as YPD) and incubate at 30° C. for 48 hours. Pick a single distinct colony to 2-5 ml YPD media and incubate overnight on a roller drum. Dilute to A600=0.2 in 200 ml YPD and incubate at 30° C. until A₆₀₀=0.8 (about 4 hours growth under normal circumstances). Divide the culture into 4 sterile 50 ml tubes. Centrifuge at medium low speed, for instance in a Beckman JT-6 at 3000 rpm for 5 minutes. Resuspend and combine the pellets in 20 ml H₂O. Re-centrifuge. Resuspend the pellet in 10 ml TEL (10 mM Tris pH 7.5, 1 mM EDTA, 0.1 M lithium acetate). Recentrifuge again and resuspend in 2 ml TEL. Competent cells are stable at room temperature for up to four hours. If you wish to make frozen stocks, you may add sterile glycerol (from a 50% stock) to a final concentration of 15%, then freeze by placing in a −80° C. freezer (do not quick freeze in liquid nitrogen or dry ice/ethanol bath). The frozen competent cells can be expected to be 3-5 fold less competent than freshly made competent cells.

Add 100 μg well sheared single stranded carrier DNA and the 30 μl digested plasmid DNA to a clean eppendorf tube. Add 100 ml competent cells and mix. Add 0.8 ml PLATE (40% PEG-3350 10 mMTris pH7.5, 1 mM EDTA, 0.1 M lithium acetate) and mix well. Incubate 30 minutes at 30° C. Heat shock 20 minutes at 42° C. Centrifuge for 5 seconds in a microcentrifuge and remove the supernatant. Wash the pellet with 1 ml TE, spin again and plate the pellet in a minimal volume (<50 μl) onto selective media such as (−)HIS plates.

TEL and PLATE solutions are available commercially (SIGMA Cat. T-0809 and P-8966), and seem to be stable at room temperature. We have found that for TEL and PLATE made in the laboratory, the solutions work best if made fresh the day of the transformation from stock solutions of Tris-Cl, EDTA, PEG-3350 and lithium acetate.

After 48 to 72 hours incubation at 30° C., depending on the growth rate of the specific strain, individual colonies are coordinately struck with a sterile toothpick to two identically arrayed plates, one of which is (−)HIS and one of which is (−)HIS (+)Cu. Pick at least 12 colonies in this manner and incubate the resultant plates for 48-72 hours (again, depending on the strain growth rate) at 30° C. Be sure to pick a colony or two of CUY106 as a positive control for growth on the (−) HIS (+) Cu plate. After incubation, the plates are scored for growth. In the case of true copper sensitive strains, there will be a clear lack of growth on the (−) HIS (+) Cu plates, and clear growth on the (−)HIS plates.

### Copper Titration

Single colonies from a selective plate (see above) are picked to CSM media (Bio101 Cat. # 4500-022) and grown overnight at 30° C. in a roller drum. The use of Bio101 CSM appears to be critical to the reproducibility of the titrations. Cultures are diluted to A600 =0.2 and are 2 ml portions are aliquoted to sterile capped culture tubes. From a 500 mM stock, copper sulfate is added to each tube to final concentrations of 0 uM ((−) copper control), 10 uM, 20 uM, 50 uM, 100 uM, 200 uM, 500 uM 1.0 mM, 1.5 mM and 2.0 mM. The ten tubes are incubated at 30° C. on a roller drum for 16-20 hours. The A600 of each aliquot is measured, and the results are graphed on a semi-log plot: Y axis=A600 of sample normalized to the A600 of the (−)copper control (linear scale). X axis=concentration of CuSO₄ (log scale).

### Copper Time Course

Single colonies from a selective plate (see above) are picked to CSM media (Bio101 Cat. # 4500-022) and grown overnight at 30° C. in a roller drum. As is the case for the copper titrations, the use of Bio101 CSM appears to be critical to the reproducibility of the copper time courses. Cultures are diluted in 25 ml of CSM to A600=0.02-0.1. the cultures are split evenly between two sterile 50 ml tubes and allowed to grow in a shaker/incubator for 1 hour at 30° C. Addition of 1 mM copper sulfate (from a 500 mM sterile stock) to one of the cultures defines the 0 time point. At each time point, a 1.2 ml aliquot is taken from each culture for analysis, and the cultures are quickly returned to incubation at 30° C. with shaking. The exception to this is the 0 time point, at which time only the culture which does not receive added copper is assayed as the data point for both the cultures. Part of each aliquot is used to measure the A600, while the rest is used to perform a serial 10-fold dilution series: 100 ul of the aliquot is diluted serially with 900 ul aliquots of sterile water. Fresh pipette tips are used for each step of the dilution series, representing 101, 102, 103, 104 and 105 fold dilutions of the original culture. Appropriate dilutions are plated to YPD plates, and the plates are marked with a strain identifier, time point, whether or not they contain copper, and which dilution has been plated. Plates are placed at 30° C. and checked both 48 and 72 hours after they have been plated; visible colonies are counted and normalized to colonies per ml of original culture, based on the dilution factor, and the plating factor (since only 100 ul and not 1 ml was plated).

Appropriate dilutions to plate to YPD:

For CUY106, and other copper insensitive strains:

At 0 time point: 10³, 10⁴, 10⁵

At time points less than 10 hours: 10³, 10⁴, 10⁵

At time points greater than 10 hours: 10⁴, 10⁵, 10⁶

For genes of unknown cidality:

At 0 time point: 10³, 10⁴, 10⁵

At time points less than 10 hours: 10¹, 10³, 10⁵

At time points greater than 10 hours: 10⁰, 10², 10⁴, 10⁶

The 100 dilution o refers to a plating of 100 μl of undiluted culture. It is recommended for cultures containing copper sulfate that the undiluted samples be spun in a microcentrifuge for 5 seconds and the pellets resuspended in sterile water before plating to YPD in order to avoid contamination by copper sulfate. For all other samples in the dilution series, this extra step has proven unnecessary. In cases of extreme cell non-survival, a second time course is recommended, to confirm the results of the first. In this case, the appropriate dilutions to plate will depend on the results of the original experiment: static effects may be more carefully assayed by biasing towards greater dilutions, while large fungicidal effects can be captured with lesser dilutions at the later time points. In some cases, we have found that concentration of the culture is necessary (for instance, concentration of 1 ml to a volume of 100 μl or even 10 ml to 100 μl to obtain a measurable number of live cells following exposure to copper (the latter case requires adjustments to the volumes used in the experiment to accommodate the large volumes needed).

Results from S. cerevisiae inactivation analyses for the target genes described in Table 1 are shown in FIGS. 27-53.

### EXAMPLE 2

### C. albicans Transformation

From a single colony on a plate, grow up a 1 ml overnight culture of Candida albicans in YPD supplemented with 20 μg/ml uridine. at 30° C. with agitation. Dilute the culture into 50 ml uridine-supplemented YPD and grow at 30° C. with agitation. When the A₅₄₀ of the culture reaches 2, cool the cells on ice for 10 minutes, then Centrifuge at 5000 rpm for 10 minutes at 4° C. Wash the pellet two times with 10 ml TE and recentrifuge each time. Resuspend the pellet in 1 ml TELD (10 mM Tris-Cl, 1 mM EDTA, pH 7.5, 0.01 M lithium acetate, 0.01 M DTT). It is important to make TELD fresh from 10× stocks of each of the components (10× DTT should be stored frozen). Spin briefly in a microcentrifuge. Resuspend the pellet in 200 ul TELD. This is sufficient competent yeast for 4 transformations. To a fresh tube add: 50 μl competent yeast preparation, 5 μl 10 mg/ml carrier DNA (Clontech) , 1-2 μl of digested and gel purified plasmid fragment (at 1-2 μg/ml), 300 μl of PEG Solution TELD (10 mM Tris-Cl, 1 mM EDTA, pH 7.5, 0.01 M lithium acetate, 0.01 M DTT, 40% PEG4000 (VWR Cat. # 9727-2)). Mix by inversion. Incubate 30 min at 30° C., then heat shock 20 minutes at 42° C. Spin 15 seconds in a microcentrifuge. Resuspend the pellet in 200 μl TE and spread on (−)URA plates.

### EXAMPLE 3

### Mammalian Cell Cytotoxicity Screen

### Reagents

From ATCC: CV-1 fibroblast cell line originated from a male African monkey kidney. Cat. No.: CCL-70

From Gibco BRL:

Dulbecco's modifed Eagle's Medium (“DMEM”) 1× liquid. Cat. No.: 11965-065

Dulbecco's modifed Eagle's Medium without Phenol red. Cat. No.:11054-020

Fetal bovine serum Cat. No.: 26140-079

Gentamicin reagent solution Cat. No.:15710-015

Trypsin-EDTA Cat. No.: 25300-54

From Sigma:

In vitro toxicology assay kit, XTT based. Cat. No.:TOX-21.

(XTT is 2,3-bis(2-Methoxy-4-nitro-5-sulfophenoyl)-2H-tetrazolium-5-carboxyarilideinn salt)

### Procedure

1. Split CV-1 cell at 1:20 using DMEM medium supplemented with 10% FBS and 10 g/ml gentamycin.

2. Three days after the splitting, CV-1 cell should reach about 80-90% confluency.

3. Aspirate the medium out and add 5 ml of PBS.

4. Add 3 ml of trypsin and let stand for 3 minutes. Add 2 ml of DMEM to inactivate the trypsin.

5. Take 0.5 ml of cell and diluted with 10 ml of DMEM. This should make the cell concentration in the range 0.5-1.5×10⁵ cells/ml.

6. Add 100 μl cell suspension to row 2-8 of 96 well plates. Add medium only to row 1.

7. Incubate cells for 24 hours.

8. Make1:50 dilution of the compound to be tested with concentration of 12.8 mg/ml.

9. Add 300 ul to column 1 from row 4 to row 8.

10. Row 1 and row 2 of column 1 should be filled with 300 μl medium only. Row 3 of column 1 should be filled with 300 μl medium with 2% DMSO so that final concentration of DMSO will start with 1%.

11. Fill columns 2 ,3, 4, 5 and 6 with 200 μl DMEM medium.

12. Make a 1 to 3 serial dilution from column 1 to column 6.

13. Take out 100 μl of each different conc of compound into the cell plate from column 1 to 6 and duplicate with 7-12.

14. Incubate the cells for another 24 hours.

15. Dissolve 5 mg of XTT into 25 ml of DMEM medium without phenol red.

16. Take out the compound solution by aspiration.

17. Wash the 96 well plate with 300 μl PBS and sit for 3 minutes.

18. Add 100 μl XTT solution to column 1-6 and add DMEM medium (without phenol red) to column 7-12.

19. Measure O.D.⁴⁵⁰ and subtract O.D.⁶⁵⁰ at the plate reader. Also, take time points at 1 hr intervals for 4 hours.

b 20. Split the CV-1 cells 1:20 using DMEM medium supplemented with 10% FBS and 10% genamycin.

XTT is a measure of mitochondrial activity and, therefore, is considered a reasonable measure of cell growth and viability. After subtracting the OD690 from OD 450, each compound-treated datapoint shall be compared with that of no-compound treatment and this determines the percentage of growth. The percentage of inhibition is defined as one minus the percentage of growth. Percentage of inhibition is plotted vs compound concentration. TC₅₀ is defined as the compound concentration that inhibits cell growth by 50%. The data from the cytotoxicity assay together with the results of the antifungal assays can be used to calculate a therapeutic ratio (TC₅₀/MIC). The higher this ratio, the more attractive the compound. Analoging and medicinal chemistry can be used to improve this ratio.

All of the references identified hereinabove, are hereby expressly incorporated herein by reference to the extent that they describe, set forth, provide a basis for or enable compositions and/or methods which may be important to the practice of one or more embodiments of the present inventions.

We claim:

1. A method of screening or testing a candidate anti-fungal compound for interaction with an essential protein, comprising;
a) providing an essential protein selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said essential protein with said one or more test compounds; and
d) determining the interaction of the test compound with said essential protein.

2. The method of claim 1, wherein said essential protein comprises a fragment, a function-conservative variant, a fragment or an active fragment of the essential protein.

3. A method of screening or testing a candidate anti-fungal compound for modulation of activity of an essential protein, comprising;
a) providing an essential protein selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said essential protein with said one or more test compounds;and
d) determining the modulation of activity of said essential protein in the presence of said test compound.

4. The method of claim 3, wherein said essential protein comprises a fragment, a function-conservative variant, a fragment or an active fragment of the essential protein.

5. A method of screening or testing a candidate anti-fungal compound for interaction with an essential protein in a culture of cells, comprising;
a) providing an essential protein within a culture of cells that express said essential protein is selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said culture of cells with said one or more test compounds;and
d) determining the interaction said test compound with said essential protein.

6. The method of claim 5, wherein said culture of cells comprises bacterial cells, fungal cells, yeast cells or mammalian cells.

7. The method of claim 5, wherein said culture of cells comprises recombinant cells.

8. The method of claim 5, wherein when expression or function of said essential protein is reduced or blocked, growth rate of a fungus expressing said essential protein is inhibited.

9. The method of claim 5, wherein when expression or function of said essential protein is reduced or blocked, viability of a fungus expressing said essential protein becomes reduced.

10. The method of claim 5, wherein said essential protein comprises a fragment, a function-conservative variant, a fragment or an active fragment of the essential protein.

11. A method of screening or testing a candidate anti-fungal compound for effects on growth or viability of a culture of cells, comprising;
a) providing an essential protein within a culture of cells that express an essential protein selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said culture of cells with said one or more test compounds;and
d) determining the effects on the growth or viability of said culture of cells.

12. The method of claim 11, wherein said culture of cells comprises fungal cells or yeast cells.

13. The method of claim 11, wherein said culture of cells comprises recombinant cells.

14. The method of claim 11, wherein when expression or function of said essential protein is reduced or blocked,the growth rate of a fungus expressing said essential protein is inhibited.

15. The method of claim 11, wherein when expression or function of said essential protein is reduced or blocked.viability of a fungus expressing said essential protein is reduced.

16. The method of claim 11, wherein said essential protein comprises a fragment, a function-conservative variant, a fragment or an active fragment of said essential protein.

17. A method of screening or testing a candidate anti-fungal compound for interaction with an essential protein in a non-human animal, comprising;
a) providing a non-human animal with a cell or group of cells expressing an essential protein selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said non-human animal with said one or more test compounds;and
d) determining the interaction of said test compound with said essential protein.

18. The method of claim 17, wherein when the interaction of said test compound with said essential protein reduces or blocks expression or function of said essential growth rate of a fungus expressing said essential protein is inhibited.

19. The method of claim 17, wherein when the interaction of said test compound with said essential protein reduces or blocks expression or function of said essential, viability of a fungus expressing said essential protein is reduced.

20. The method of claim 17, wherein said essential protein comprises a fragment, a function-conservative variant, a fragment or an active fragment of the essential protein.

21. A method of screening or testing the effects of a candidate anti-fungal compound on growth or viability of a cell or group of cells expressing an essential protein in a non-human animal, comprising;
a) providing the non-human animal with the cell or group of cells expressing an essential protein selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said test animal with said one or more test compounds;and
d) determining the effects on the growth or viability of said cell or group of cells.

22. The method of claim 21, wherein when expression or function of said essential protein is reduced or blocked growth rate of a fungus expressing said essential protein is inhibited.

23. The method of claim 21, wherein when expression or function of said essential protein is reduced or blocked, viability of a fungus expressing said essential protein becomes reduced.

24. The method of claim 21, wherein said essential protein comprises a fragment, a function-conservative variant, a fragment or an active fragment of said essential protein.

25. The method of claim 3, wherein the modulation of activity comprises modulation of fungal gene transcription.

26. The method of claim 5, wherein the interaction is assessed by binding of said test compound with said essential protein or activity of said essential protein in the presence of said test compound.

27. The method of claim 17, wherein the interaction is assessed by binding of said test compound with said essential protein or activity of said essential protein in the presence of said test compound.

28. A method of screening or testing a candidate anti-fungal compound for binding with an essential protein, comprising;
a) providing an essential protein selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said essential protein with said one or more test compounds;and
d) determining the binding of the test compound with said essential protein.

29. A method of screening or testing a candidate anti-fungal compound for modulation transcription of a gene encoding an essential protein, comprising;
a) providing a gene encoding an essential protein selected from the group consisting of RPC34, POP3, TFA2, NAB2, MPT1, MTR2, BOS1, POL30, YMR131C, SQT1, MTW1, TFB1, SPC98, BFR2, RNA1, GCD7, SKI6, NIP1, LCP5, NCE103, ECO1, ORC2, CNS1, YPD1, TIM10 and SRB4;
b) providing one or more test compounds;
c) contacting said gene with said one or more test compounds; and
d) determining the modulation of transcription of said gene of said essential protein in the presence of said test compound
